(19) United States
(12) Patent Application Publication
(10) Pub. No.: US 2024/0377394 A1
(43) Pub. Date: Nov. 14, 2024

(54) Cell Selection Methods and Related Compositions

(71) Applicant: THE BOARD OF TRUSTEES OF THE LELAND STANFORD JUNIOR UNIVERSITY, Stanford, CA (US)

(72) Inventors: Louai Labanieh, Palo Alto, CA (US); Crystal Mackall, Stanford, CA (US); Robbie Majzner, Palo Alto, CA (US)

(21) Appl. No.: 18/284,827

(22) PCT Filed: Apr. 6, 2022
(86) PCT No.: PCT/US2022/023725

§ 371 Date: Sep. 28, 2023

Related U.S. Application Data
(60) Provisional application No. 63/171,841, filed on Apr. 7, 2021.

(51) Int. Cl. G01N 33/569; C07K 14/705

(52) U.S. Cl. CPC G01N 33/56966; C07K 14/70517; C07K 2319/02; C07K 2319/03; C07K 2319/04; G01N 2333/91102; G01N 2333/96411

(57) ABSTRACT

Provided are methods of selecting for cells that comprise two or more separate expression constructs. In certain embodiments, the methods comprise contacting a population of cells with two or more separate expression constructs under conditions in which the two or more expression constructs are delivered to cells of the population of cells. The two or more separate expression constructs comprise a first expression construct that encodes a fusion protein comprising a selection marker, a protein localization tag, and a protease cleavage site disposed between the selection marker and the protein localization tag. The second expression construct encodes a protein required for cell surface expression of the selection marker. Such methods further comprise selecting for cells exhibiting cell surface expression of the selection marker. Related cells, compositions, kits and therapeutic methods are also provided.

### CROSS-REFERENCE TO RELATED APPLICATIONS

This application claims the benefit of U.S. Provisional Patent Application No. 63/171,841, filed Apr. 7, 2021, which application is incorporated herein by reference in its entirety.

### INCORPORATION BY REFERENCE OF SEQUENCE LISTING PROVIDED AS A TEXT FILE

A Sequence Listing is provided herewith in a text file, (STAN-1819WO_SEQ_LIST_ST25), created on Apr. 5, 2022 and having a size of 420,000 bytes of file. The contents of the text file are incorporated herein by reference in its entirety.

### INTRODUCTION

There is a current lack of technologies that can be used to purify cells engineered with multiple genetic modifications. Current limitations in payload capacity require the use of multiple expression constructs for delivering transgenes. Serial sorting on multiple surface markers is expensive, time consuming, and results in massive cell losses. These purification problems impose limitations on the ability to engineer cells, e.g., for cell-based therapies. Improved approaches for purifying cells engineered with multiple genetic modifications are therefore needed.

### SUMMARY

Provided are methods of selecting for cells that comprise two or more separate expression constructs. In certain embodiments, the methods comprise contacting a population of cells with two or more separate expression constructs under conditions in which the two or more expression constructs are delivered to cells of the population of cells. The two or more separate expression constructs comprise a first expression construct that encodes a fusion protein comprising a selection marker, a protein localization tag, and a protease cleavage site disposed between the selection marker and the protein localization tag. The second expression construct encodes a protein required for cell surface expression of the selection marker. Such methods further comprise selecting for cells exhibiting cell surface expression of the selection marker. Related cells, compositions, kits, and therapeutic methods are also provided.

### BRIEF DESCRIPTION OF THE FIGURES

FIG. 1A-1B: 1A: Schematic illustration of a two-way cell selection system according to some embodiments of the present disclosure. The selection systems of the present disclosure are sometimes referred to herein as “STASH select” systems by virtue of the selection marker being “stashed” intracellularly in the absence of the desired combination of expression constructs being present in the cell. 1B: Schematic illustration of two separate expression constructs (or “expression constructs” as used interchangeably herein) encoding the components of the selection system shown in 1A, which two separate expression constructs are required for cell surface expression of the selection marker.

FIG. 2A-2B: 2A: Schematic illustration of AND gate logic that can be performed using the selection systems of the present disclosure. Cells which satisfy the two input requirements (expression of construct A and expression of construct B) result in the output surface expression of the selection marker. 2B: Schematic illustration of the four possible outcomes of cells which have been exposed to construct A and construct B. Cells which express only construct A have a selection marker which is retained intracellularly. Cells which express only construct B have a protease which is retained intracellularly. Cells which express both construct A and construct B have a selection marker which is expressed on the surface of the cell which can be used for enrichment and detection.

FIG. 3A-3B: 3A: Schematic illustration of separate expression constructs of a two-way cell selection system according to some embodiments of the present disclosure. 3B: Flow cytometry data demonstrating high cell surface expression of the selection marker only in the presence of both expression constructs.

FIG. 4A-4B: 4A: Schematic illustration of separate expression constructs of a cell selection system according to some embodiments of the present disclosure. 4B: Flow cytometry data demonstrating high cell surface expression of the selection marker only in the presence of both expression constructs.

FIG. 5A-5B: 5A: Schematic illustration of a three-way cell selection system according to some embodiments of the present disclosure. 5B: Schematic illustration of three separate expression constructs encoding the components of the selection system shown in 5A, which three separate expression constructs are required for cell surface expression of the selection marker.

FIG. 6A-6B: 6A: Schematic illustration of three separate expression constructs of a three-way cell selection system according to some embodiments of the present disclosure. 6B: Flow cytometry data demonstrating high cell surface expression of the selection marker only in the presence of all three expression constructs.

FIG. 7A-7B: 7A: Schematic illustration of a five-way cell selection system according to some embodiments of the present disclosure. 7B: Schematic illustration of five separate expression constructs encoding the components of the selection system shown in 7A, which five separate expression constructs are required for cell surface expression of the selection marker.

FIG. 8A-8B: 8A: Schematic illustration of five separate expression constructs of a five-way cell selection system according to some embodiments of the present disclosure. 8B: Flow cytometry data demonstrating high cell surface expression of the selection marker in cells positive for all five expression constructs.

FIG. 9A-9B: 9A: Schematic illustration of a two-way cell selection system according to some embodiments of the present disclosure. In this example, a truncated receptor (here, truncated EGFR, or “EGFRt”) serves as the selection marker. 9B (adapted from Labanieh et al. (2018) Nature Biomedical Engineering 2:377-391): Schematic illustration of the truncated receptor serving as suicide switch. In this example, cells comprising both expression constructs express the truncated receptor on their surface. Subsequent to administration of the cells to an individual for therapeutic purposes, the cells may be ablated if desired by administration of an antibody specific for the truncated receptor. In the particular example shown in FIG. 9, the suicide switch is truncated EGFR (EGFRt), and the cells may be ablated by administration of an anti-EGFR antibody such as Cetuximab.

FIG. 10A-10C: 10A: Schematic illustration of two separate expression constructs of a two-way cell selection system according to some embodiments of the present disclosure. 10B-10C: Flow cytometry data assessing surface expression of the selection marker (here, EGFRt) when employing a particular ER localization tag.

FIG. 11A-11B: 11A: Schematic illustration of a two-way cell selection system according to some embodiments of the present disclosure. In this example, a truncated receptor (here, truncated EGFR, or “EGFRt”) serves as the selection marker. 11B: Sequences of fusion proteins comprising a hinge domain, a transmembrane domain, various ER localization tags, and a protease cleavage site disposed between the transmembrane domain and the particular ER localization tag.

FIG. 12A-12B: 12A: Schematic illustration of a two-way cell selection system according to some embodiments of the present disclosure. In this example, a truncated receptor (here, truncated EGFR, or “EGFRt”) serves as the selection marker. 12B: Sequences of fusion proteins comprising a transmembrane domain, an intracellular domain (ICD) of various ER-resident membrane proteins, and a protease cleavage site disposed between the transmembrane domain and the particular intracellular domain. In FIG. 12B, each ER-resident protein is a human ER-resident protein except for those of constructs 506 and 507.

FIG. 13A-13E: 13A: Schematic illustration of two separate expression constructs of a two-way cell selection system according to some embodiments of the present disclosure. 13B-13E: Flow cytometry data showing the identification of high-performing constructs among the various ER localization tags employed.

FIG. 14A-14B: 14A: An example workflow for selecting (or “purifying”) cells exhibiting cell surface expression of the selection marker according to some embodiments of the present disclosure. Magnetic activated cell sorting (MACS)-based selection is employed in this example. In this particular example, the selection marker is EGFRt. 14B: A plot of the percentage of double positive (BFP+ GFP+) cells from the purified cell fraction after MACS-based selection. The data demonstrate that a number of the EGFRt-STASH ER localization tag variants can be used to isolate highly pure double positive populations using a single selection marker.

FIG. 15A-15E: Flow cytometry data demonstrating a high degree of purity of double positive cell populations for a number of ER localization tag variants after EGFR MACS-based selection.

FIG. 16A-16B: 16A: Schematic illustration of two separate expression constructs of a two-way cell selection system according to some embodiments of the present disclosure. 16B: Flow cytometry data showing a high degree of purity of double positive cell populations for a particular ER localization tag variant after EGFR MACS-based selection.

FIG. 17A-17B: 17A: Schematic illustration of two separate expression constructs of a two-way cell selection system according to some embodiments of the present disclosure. 17B: Flow cytometry data showing a high degree of purity of double positive cell populations for a particular ER localization tag variant after EGFR MACS-based selection.

FIG. 18A-18B: 18A: Schematic illustration of two separate expression constructs of a two-way cell selection system according to some embodiments of the present disclosure. 18B: Flow cytometry data showing a high degree of purity of double positive cell populations for a particular ER localization tag variant after EGFR MACS-based selection.

FIG. 19A-19B: 19A: Schematic illustration of two separate expression constructs of a two-way cell selection system according to some embodiments of the present disclosure. 19B: Flow cytometry data showing a high degree of purity of double positive cell populations for a particular ER localization tag variant after EGFR MACS-based selection.

FIG. 20A-20D: 20A: Schematic illustration of a three-way cell selection system according to some embodiments of the present disclosure. 20B: Schematic illustration of three separate expression constructs encoding the components of the selection system shown in 20A, which three separate expression constructs are required for cell surface expression of the selection marker. 20C-20D: Flow cytometry data demonstrating a highly pure population of tri-specific cells (here, tri-specific CAR-T cells) transduced with the three separate expression constructs.

FIG. 21A-21B: 21A: Schematic illustration of a two-way cell selection system according to some embodiments of the present disclosure. 21B: Schematic illustration of a two-way cell selection system according to some embodiments of the present disclosure.

FIG. 22A-22C: 22A: Schematic illustration of two separate expression constructs of a two-way cell selection system according to some embodiments of the present disclosure. 22B-22C: Flow cytometry data demonstrating the selection of double positive cells using the selection marker.

FIGS. 23-30: Schematic illustrations of three-, four-, five-, six-, seven-, eight-, nine- and ten-way cell selection systems, respectively, according to some embodiments of the present disclosure.

FIGS. 31-33: Schematic illustrations of five-, nine- and thirteen-way cell selection systems, respectively, according to some embodiments of the present disclosure.

FIG. 34: Flow cytometry histograms of surface CD34 staining using the QBEnd/10 antibody on primary human T cells. As can be seen in the data, only double positive cells display high surface expression of the C34 epitope.

FIG. 35: Flow cytometry histograms showing surface EGFR expression on primary human T cells transduced with a EGFRt-STASH variant and a TEV protease bearing a CISD2 ER retention tag.

FIG. 36: Flow cytometry histograms showing surface EGFRt expression on primary human T cells transduced with the three-way STASH Select system using a EGFRt-STASH variant bearing a CD8α or CD28 Tm domain and a CISD2 ER retention signal.

FIG. 37: Flow cytometry histograms showing surface EGFR expression on primary human T cells transduced with the three-way STASH Select system using a EGFRt-STASH variant bearing a CD8α or CD28 Tm domain and an IBV S protein retention signal.

FIG. 38: Flow cytometry histograms showing surface EGFR expression on primary human T cells transduced with the three-way STASH Select system using a EGFRt-STASH variant bearing a CD8α or CD28 Tm domain and a degron fused to the adenovirus E3-19K retention signal.

FIG. 39A-39B: 39A: Schematic illustration of three separate expression constructs of a three-way cell selection system according to some embodiments of the present disclosure. 39B: Flow plot histograms showing surface expression of EGFR, cJun, CD19.BBz, and HER2.BBz CAR.

FIG. 40A-40B: 40A: a series of flow plots of primary human T cells demonstrating BFP and GFP expression after staining with anti-EGFR-biotin at the dilution indicated above the flow plot and MACS selection. Employed in this example was the variant 497 ER retention tag. 40B: a bar plot showing the yield of double positive cells after MACS selection for the samples shown in 40A.

FIG. 41A-41B: 41A: a series of flow plots of primary human T cells demonstrating BFP and GFP expression after staining with anti-EGFR-biotin at the dilution indicated above the flow plot and MACS selection. Employed in this example was the variant 501 ER retention tag. 41B: a bar plot showing the yield of double positive cells after MACS selection for the samples shown in 41A.

FIG. 42A-42D: A series of flow plots demonstrating BFP and GFP expression in primary human T cells.

FIG. 43A-43E: A series of flow plots demonstrating surface EGFR expression in primary human T cells.

FIG. 44: A series of flow plots demonstrating surface EGFR expression in primary human T cells transduced with the EGFR STASH variant indicated above each flow plot and a minimized TEV protease construct.

FIG. 45A-45D: Data demonstrating the identification of human proteases that find use in the STASH Select system. FIG. 45D indicates the amino acid sequence (#834-#837 SEQ ID NOs: 132-135, respectively; #839-#840 SEQ ID NOs: 136-137, respectively) of the protease cleavage sites used.

FIG. 46A-46C: Schematic illustration and data demonstrating a two-way STASH Select using a combination of CRISPR knock-in and retroviral gene delivery methods.

FIG. 47A-47C: A series of flow plots demonstrating surface EGFR expression in primary human T cells in a two-way STASH Select system with various EGFR truncations.

### DETAILED DESCRIPTION

Before the methods and compositions of the present disclosure are described in greater detail, it is to be understood that the methods and compositions are not limited to particular embodiments described, as such may, of course, vary. It is also to be understood that the terminology used herein is for the purpose of describing particular embodiments only, and is not intended to be limiting, since the scope of the methods and compositions will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions. The upper and lower limits of these smaller ranges may independently be included in the smaller ranges and are also encompassed within the methods and compositions, subject to any specifically excluded limit in the stated range.

Where the stated range includes one or both of the limits, ranges excluding either or both of those included limits are also included in the methods and compositions.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods and compositions belong. Although any methods and compositions similar or equivalent to those described herein can also be used in the practice or testing of the methods and compositions, representative illustrative methods and compositions are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods and compositions are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods and compositions, which are, for clarity, described in the context of separate embodiments, may also be provided in combination in a single embodiment. Conversely, various features of the methods and compositions, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods and compositions and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Cell Selection Methods

Aspects of the present disclosure include methods of selecting for cells that comprise two or more separate expression constructs. The methods find use in a variety of applications including both research and clinical applications. By way of example, the methods find use in any application in which it is desirable to efficiently engineer and select for cells comprising multiple genetic modifications (e.g., transgenic modifications, gene knockouts, and/or the like), where such genetic modifications are difficult or not feasible using a single expression construct, e.g., due to the limitations in expression construct payload capacity. The methods of the present disclosure enable the selection of cells comprising the multiple desired genetic modification using a single selection marker. That is, cell surface expression of a single selection marker is the readout for cells comprising each of the desired multiple genetic modifications, obviating the need for serial sorting on multiple surface markers to obtain cells comprising the multiple modifications. Cells comprising the multiple desired genetic modifications can be readily selected (sometimes referred to herein as “purified” or “enriched”) based on the single selection marker using existing reagents and systems for magnetic-activated cell sorting (MACS), fluorescence-activated cell sorting (FACS), and the like.

According to some embodiments, the multiple genetic modifications find use in the context of cell-based therapies, such that the methods of the present disclosure find use in producing and selecting cells for such therapies. Non-limiting examples of genetic modifications that find use in cell-based therapies include transgenic modification to express a recombinant receptor (e.g., a chimeric antigen receptor (CAR), a T cell receptor (TCR), etc.) that targets undesirable cells (e.g., cancer cells) when administered to an individual, transgenic and/or knockout modifications that reduce immunogenicity of the engineered cells upon administration to an individual, transgenic and/or knockout modifications that confer upon the cells resistance to cell exhaustion upon administration to an individual, transgenic and/or knockout modifications that enhance the effectiveness of the cells in the tumor microenvironment (TME) for treatment of solid tumors, and/or the like. Any desired combination of such modifications may be made and selected for according to the methods of the present disclosure.

The selection approaches of the present disclosure are sometimes referred to herein as the “STASH selection system”, “STASH select”, etc. by virtue of the selection marker being “stashed” intracellularly in the absence of the desired combination of expression constructs being present in the cell. According to the selection system, one of the expression constructs encodes a fusion protein comprising the selection marker, a protein localization tag, and a protease cleavage site disposed between the selection marker and the protein localization tag. In the absence of one or more additional expression constructs which provide a protease capable of cleaving the protease cleavage site, the selection marker remains localized to (i.e., retained or “stashed” at) the intracellular location (e.g., organelle) determined by the particular protein localization tag employed. When the one or more additional expression constructs are present in the cell, thereby providing a protease capable of cleaving the protease cleavage site, the selection marker is cleaved from the protein localization tag and traffics to the surface of the cell, such that the cell comprising the desired multiple genetic modifications exhibits cell surface expression of the selection marker. The selection systems of the present disclosure are modular and include configurations such that the delivery to the cell of 2 or more, 3 or more, 4 or more, 5 or more, 6 or more, 7 or more, 8 or more, 9 or more, or 10 or more separate expression constructs (each of which may provide a desired genetic modification, e.g., transgene, targeted gene knockout, and/or the like) is required to provide the protease activity necessary for cell surface expression of the selection marker.

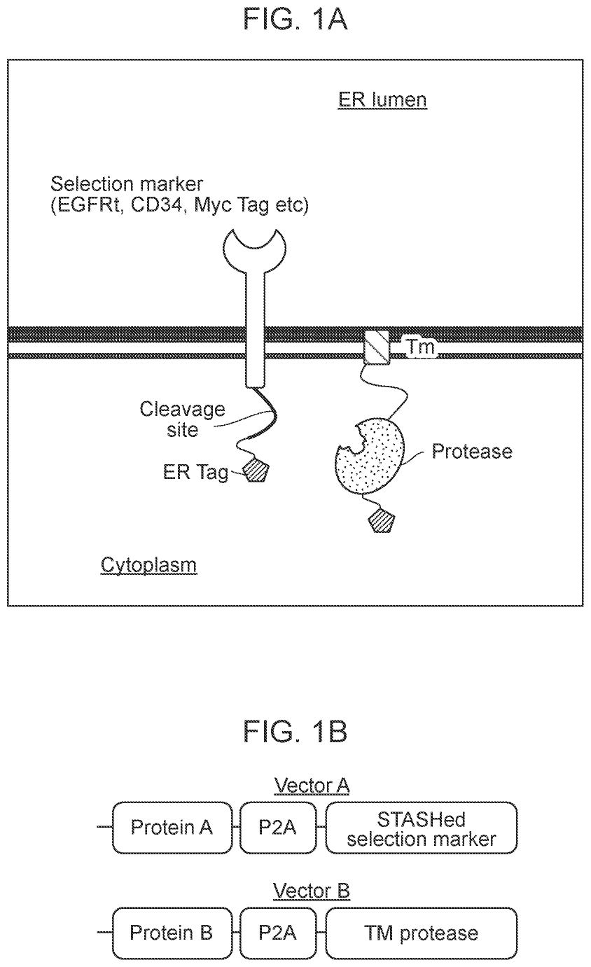

Shown in FIG. 1A is an example cell selection system (a “two-way” system) in which the delivery of two expression constructs to the cell is required for cell surface expression of the selection marker. In this particular example, an epitope-based selection marker (EGFRt, CD34, Myc tag, etc.) is fused to a protease cleavage site and an intracellular localization (or “retention”) tag, e.g., an endoplasmic reticulum (ER) localization tag. A co-expressed protease that localizes intracellularly recognizes the protease cleavage site, and cleaves the fusion protein at the protease cleavage site. This cleavage event liberates the selection marker from the retention tag and allows the selection marker to translocate to the surface of the cell. The surface-expressed selection tag can then be used as a selection handle to isolate cells expressing both the selection marker and the protease. Schematically illustrated in FIG. 1B are the two expression constructs that encode the components of the two-way selection system illustrated in FIG. 1A. In this example, the two expression constructs each encode a protein of interest (protein A and protein B from constructs A and B, respectively), such that cell surface expression of the selection marker is a marker for cells that express proteins of interest A and B, and such cells may be selected for (enriched, purified) using the single selection marker. As shown in FIG. 1B, a ribosome skipping site (in this example, P2A from porcine teschovirus) may be provided to allows for bicistronic expression of the protein of interest and the selection system component. That is, a ribosome skipping site enables the expression of a protein of interest and a component of the selection system as separate proteins.

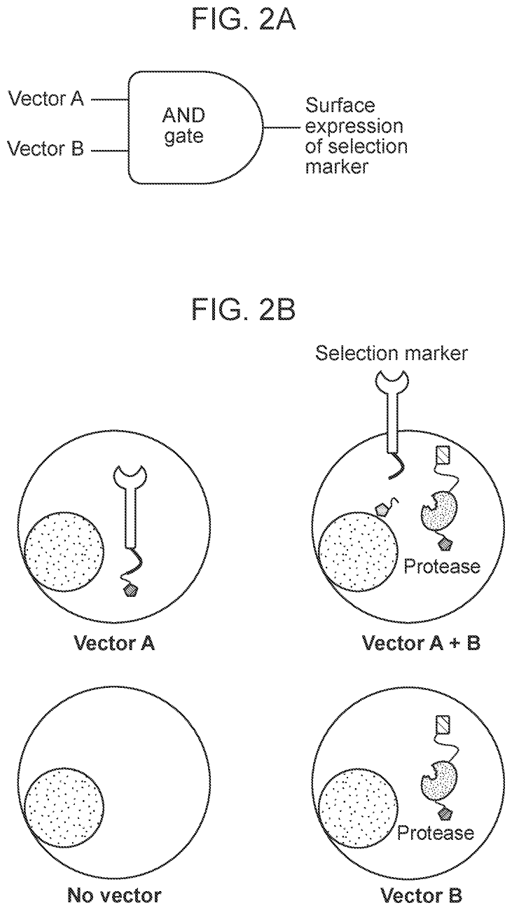

FIG. 2A schematically illustrates AND gate logic that can be performed using the STASH

Select system. Cells which satisfy the two input requirements (expression of construct A and expression of construct B) result in the output surface expression of the selection marker. FIG. 2B schematically illustrates the four possible outcomes of cells which have been exposed to construct A and construct B. Cells which express only construct A have a selection marker which is retained intracellularly. Cells which express only construct B have a protease which is retained intracellularly. Cells which express both construct A and construct B have a selection marker which is expressed on the surface of the cell which can be used for detection and enrichment.

In certain embodiments, the methods of the present disclosure comprise contacting a population of cells with two or more separate expression constructs under conditions in which the two or more expression constructs are delivered to cells of the population of cells. The two or more separate expression constructs comprise a first expression construct that encodes a fusion protein comprising a selection marker, a protein localization tag, and a protease cleavage site disposed between the selection marker and the protein localization tag. The two or more separate expression constructs further comprise a second expression construct that encodes a protein required for cell surface expression of the selection marker. The methods further comprise selecting for cells exhibiting cell surface expression of the selection marker.

The contacting step may comprise contacting the population of cells with the two or more separate expression constructs simultaneously, e.g., by combining the cells and each of the two or more separate expression constructs in a single mixture under conditions suitable for delivery (e.g., transfection, transduction, etc.) of each of the two or more separate expression constructs into cells of the population of cells. The contacting step may comprise contacting the population of cells with the two or more separate expression constructs sequentially, e.g., where the population of cells is first combined with less than each of the two or more separate expression constructs under expression construct delivery conditions, followed by combining the population of cells with the remaining two or more separate expression constructs in one or more further combining steps under suitable conditions.

A variety of suitable approaches and conditions for the delivery of expression constructs to cells are known. According to some embodiments, the two or more separate expression constructs are delivered to cells of the population of cells by microinjection, transfection, lipofection, heat-shock, electroporation, transduction, gene gun, DEAE-dextran-mediated transfer, and/or the like. In certain embodiments, the two or more separate expression constructs are introduced into cells of the population of cells by AAV transduction. The AAV vector may comprise ITRs from AAV2, and a serotype from any one of AAV1, AAV2, AAV3, AAV4, AAV5, AAV6, AAV7, AAV8, AAV9, or AAV 10. According to some embodiments, the AAV vector comprises ITRs from AAV2 and a serotype from AAV6. In certain embodiments, the nucleic acid (e.g., expression vector) encoding the CAR is introduced into the cell (e.g., a T cell) by lentiviral or retroviral transduction. The lentiviral vector backbone may be derived from HIV-1, HIV-2, visna-maedi virus (VMV) virus, caprine arthritis-encephalitis virus (CAEV), equine infectious anemia virus (EIAV), feline immunodeficiency virus (FIV), bovine immune deficiency virus (BIV), or simian immunodeficiency virus (SIV). The lentiviral vector may be integration competent or an integrase deficient lentiviral vector (TDLV). In one embodiment, IDLV vectors including an HIV-based vector backbone (i.e., HIV cis-acting sequence elements) are employed. Non-limiting example approaches for the preparation of retroviral expression constructs and the transduction of cells with such constructs is provided in the Experimental section hereinbelow.

As used herein, an “expression construct” is a circular or linear polynucleotide (a polymer composed of naturally-occurring and/or non-naturally-occurring nucleotides) comprising a region that encodes a component of the cell selection system (e.g., a fusion protein comprising a selection marker, a protein localization tag, and a protease cleavage site; and/or a protein required for cell surface expression of the selection marker) operably linked to a suitable promoter, e.g., a constitutive or inducible promoter. In some embodiments, expression of the cell selection system component is under the control of one or more exogenous (including heterologous) regulatory elements, e.g., promoter, enhancer, etc., present in the expression construct, and operably linked to the region encoding the cell selection system component, prior to contacting with the population of cells. In some embodiments, expression of the cell selection system component may be controlled by one or more endogenous regulatory elements, e.g., promoter, enhancer, etc., at or near a genomic locus into which the expression construct is inserted.

One or more of the two or more separate expression constructs may further comprise one or more regions encoding one or more proteins of interest (e.g., any of the proteins of interest described elsewhere herein), each operably linked to a suitable promoter, where the promoter may be a single shared promoter among each of the protein-encoding regions of the expression construct (including the cell selection system component), or at least one of the protein-encoding regions may be operably linked to a promoter which is not shared with any other protein-encoding region of the expression construct. In certain embodiments, when an expression construct comprises one or more protein-encoding regions in addition to the region encoding the component of the cell selection system, the expression construct may be configured to allow for polycistronic expression of two or more (e.g., each) of the protein-encoding regions. That is, two or more (e.g., each) of the proteins encoded by the expression construct may be expressed as separate proteins from the same promoter. In certain embodiments, the expression construct includes a ribosome skipping site to allow for polycistronic expression of two or more (e.g., each) of the protein-encoding regions. A non-limiting example of a suitable ribosome skipping site which may be incorporated into expression constructs is the P2A ribosome skipping site from porcine teschovirus.

The expression constructs (e.g., vectors) can be suitable for replication and integration in prokaryotes, eukaryotes, or both. The expression constructs may contain functionally appropriately oriented transcription and translation terminators, initiation sequences, and promoters useful for regulation of the expression of the nucleic acid encoding the component of the cell selection system. The expression constructs optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

To obtain high levels of expression of a cloned nucleic acid it is common to construct expression constructs which typically contain a strong promoter to direct transcription, a ribosome binding site for translational initiation, and a transcription/translation terminator, each in functional orientation to each other and to the protein-encoding sequence. Examples of regulatory regions suitable for this purpose in E. coli are the promoter and operator region of the E. coli tryptophan biosynthetic pathway, the leftward promoter of phage lambda (P_{L}), and the L-arabinose (araBAD) operon. The inclusion of selection markers in DNA vectors transformed in E. coli is also useful. Examples of such markers include genes specifying resistance to ampicillin, tetracycline, or chloramphenicol. Expression systems for expressing the selection system components are available using, for example, E. coli, Bacillus sp. and Salmonella. E. coli systems may also be used. Nucleic acids encoding the selection system components. Transducing cells with nucleic acids can involve, for example, incubating lipidic microparticles containing nucleic acids with cells or incubating viral vectors containing nucleic acids with cells within the host range of the vector.

The two or more expression constructs are “separate”, meaning that none of the two or more expression constructs are part of the same polynucleotide molecule.

In certain embodiments, upon delivery of the two or more separate expression constructs to cells of the population of cells, one or more of the expression constructs are episomal (e.g., extra-chromosomal), where by “episome” or “episomal” is meant a polynucleotide that replicates independently of the cell's chromosomal DNA. A non-limiting example of an episome that may be employed in the present methods is a plasmid.

According to some embodiments, upon delivery of the two or more separate expression constructs to cells of the population of cells, one or more of the expression constructs integrates into the genome of the cell. In certain embodiments, one or more of the expression constructs are adapted for site-specific integration into the genome. For example, an expression construct may be adapted for site-specific integration into the genome, where the site-specific integration inactivates a target gene within the genome of the cell. By way of example, the site-specific integration may knock-out the target gene by knock-in of the expression construct. Any suitable approach for site-specific gene editing and functional integration may be employed. Functional integration of an expression construct may be achieved through various means, including through the use of integrating vectors, including viral and non-viral vectors. In some instances, a retroviral vector, e.g., a lentiviral vector, may be employed. In some instances, a non-retroviral integrating vector may be employed. An integrating vector may be contacted with the cells in a suitable transduction medium, at a suitable concentration (or multiplicity of infection), and for a suitable time for the vector to infect the target cells, facilitating functional integration of the expression construct. Non-limiting examples of useful viral vectors include retroviral vectors, lentiviral vectors, adenoviral (Ad) vectors, adeno-associated virus (AAV) vectors, hybrid Ad-AAV vector systems, and the like.

Strategies for site-specific integration that find use in the methods of the present disclosure include those that employ homologous recombination, nonhomologous end-joining (NHEJ), and/or the like. Such strategies may employ a non-naturally occurring or engineered nuclease, including, but not limited to, zinc-ringer nucleases (ZNFs), meganucleases, transcription activator-like effector nucleases (TALENs)), or a CRISPR-Cas system. Eukaryotic cells utilize two distinct DNA repair mechanisms in response to DNA double strand breaks (DSBs): Homologous recombination (HR) and nonhomologous end-joining (NHEJ). Mechanistically, HR is an error-free DNA repair mechanism because it requires a homologous template to repair the damaged DNA strand. Because of its homology-based mechanism, HR has been used as a tool to site-specifically engineer the genome. Gene targeting by HR requires the use of two homology arms that flank the transgene/target site of interest. HR efficiency can be increased by the introduction of DSBs at the target site using specific rare-cutting endonucleases. The discovery of this phenomenon prompted the development of methods to create site-specific DSBs in the genome of different species. Various chimeric enzymes have been designed for this purpose over the last decade, namely ZFNs, meganucleases, and TALENs. ZFNs are modular chimeric proteins that contain a ZF-based DNA binding domain (DBD) and a FokI nuclease domain. DBD is usually composed of three ZF domains, each with 3-base pair specificity; the FokI nuclease domain provides a DNA nicking activity, which is targeted by two flanking ZFNs. Owing to the modular nature of the DBD, any site in a genome could be targeted. TALENs are similar to ZFNs except that the DBD is derived from transcription activator-like effectors (TALEs). The TALE DBD is modular, and it is composed of 34-residue repeats, and its DNA specificity is determined by the number and order of repeats. Each repeat binds a single nucleotide in the target sequence through only two residues.

The methods of the present disclosure may be performed on any cell populations of interest. In certain embodiments, the population of cells is a population of prokaryotic cells (e.g., bacteria), a population of yeast cells, a population of insect (e.g., drosophila) cells, a population of amphibian (e.g., frog, e.g., Xenopus) cells, a population of plant cells, etc. According to some embodiments, the population of cells is a population of mammalian cells. Mammalian cells of interest include human cells, rodent cells, and the like. According to some embodiments, the population of cells is a population of peripheral blood mononuclear cells (PBMCs). In certain embodiments, the population of cells is a population of immune cells. The population of immune cells may comprise one or any combination of T cells, B cells, natural killer (NK) cells, macrophages, monocytes, neutrophils, dendritic cells, mast cells, basophils, eosinophils. When the immune cells comprise T cells, the T cells may comprise one or any combination of naive T cells (T_{N}), cytotoxic T cells (T_{CTL}), memory T cells (T_{MEM}), T memory stem cells (T_{SCM}), central memory T cells (T_{CM}), effector memory T cells (T_{EM}), tissue resident memory T cells (T_{RM}), effector T cells (T_{EFF}), regulatory T cells (T_{REGs}), helper T cells, CD4+ T cells, CD8+ T cells, virus-specific T cells, alpha beta T cells (T_{αβ}), gamma delta T cells (T_{γδ}). According to some embodiments, the population of cells is a population of cells comprises stem cells, e.g., mammalian (e.g., human) stem cells. For example, the population of cells may comprise embryonic stem (ES) cells, adult stem cells, hematopoietic stem cells (HSCs), induced pluripotent stem cells (iPSCs), mesenchymal stem cells (MSCs), neural stem cells (NSCs), or any combination thereof.

### Protein Localization Tags

As used herein, the term “protein localization tag” refers to an amino acid sequence that directs the cellular localization of the fusion protein comprising the selection marker (and optionally, any other cell selection system components expressed by the two or more separate expression constructs) to a particular cellular compartment. In certain embodiments, the protein localization tag is selected from an endoplasmic reticulum (ER) localization tag, a Golgi apparatus (Golgi) localization tag, a lysosome localization tag, a plasma membrane localization tag, a mitochondria localization tag, a peroxisome localization tag, a cytosolic localization tag, and a nuclear localization tag.

The fusion protein comprising the selection marker (and optionally, any other cell selection system component(s) expressed by the two or more separate expression constructs) may include any suitable protein localization tag for directing localization to the desired cellular compartment. In some embodiments, when two or more cell selection system components comprise a protein localization tag, the protein localization tag of each component may direct each component to the same cellular compartment (e.g., organelle). For example, in certain embodiments, when two or more cell selection system components comprise a protein localization tag, the protein localization tags are identical or substantially identical to each other.

Suitable protein localization tags are known. In certain embodiments, a cell selection system component includes a protein localization tag in LocSigDB (a database of protein localization signals/tags available at genome.unmc.edu/LocSigDB/and described in Negi et al. (2015) Database, Volume 2015:1-7); DBSubLoc (a database of protein subcellular localization—available at bioinfo.tsinghua.edu.cn/dbsubloc.html); LOCATE (a mammalian protein subcellular localization database available at locate.imb.uq.edu.au); LocDB (a protein localization database available at rostlab.org/services/locDB); eSLDB (a eukaryotic subcellular localization database available at gpcr.biocomp.unibo.it/esldb); and/or any other convenient database of protein localization tags. According to some embodiments, the protein localization tag is located at the N-terminus of the cell selection system component. For example, there are naturally-occurring N-terminal protein localization tags for type II membrane proteins (see, e.g., Schutz et al. (1994) EMBO J. 13 (7): 1696-1705) and other proteins.

According to some embodiments, the protein localization tag is an ER localization tag. In certain embodiments, the ER localization tag comprises the amino acid sequence KKMP. A non-limiting example of an ER localization tag that may be included in a cell selection system component of the present disclosure is an ER localization tag comprising 85% or greater, 90% or greater, or 100% amino acid sequence identity to an ER localization tag comprising, consisting of, or present within, an amino acid sequence selected from LYKYKSRRSFIDEKKMP (SEQ ID NO: 1); AEKDEL (SEQ ID NO:2); EQKLISEEDLKDEL (SEQ ID NO:3); GGGGSGGGGSKDEL (SEQ ID NO:4); GGGGSGGGGSGGGGSGGGGSKDEL ID (SEQ NO: 5); GGGGSGGGGSGGGGSGGGGSAEKDEL (SEQ ID NO:6); KYKSRRSFIEEKKMP (SEQ ID NO: 7); LKYKSRRSFIEEKKMP (SEQ ID NO:8); LYKYKSRRSFIEEKKMP (SEQ ID NO:9); LYCKYKSRRSFIEEKKMP (SEQ ID NO:10); LYCNKYKSRRSFIEEKKMP (SEQ ID NO:11); LYCNKYKSRRSFIDEKKMP (SEQ ID NO:12); LYEQKLISEEDLKYKSRRSFIEEKKMP (SEQ ID NO: 13); LYCYPYDVPDYAKYKSRRSFIEEKKMP (SEQ ID NO:14); LYKKLETFKKTN (SEQ ID NO: 15); LYEQKLISEEDLKKLETFKKTN (SEQ ID NO:16); LYYQRL (SEQ ID NO:17); LYEQKLISEEDLYQRL (SEQ ID NO:18); LYKRKIIAFALEGKRSKVTRRPKASDYQRL (SEQ ID NO: 19); LYRNIKCD (SEQ ID NO:20); and LYEQKLISEEDLRNIKCD (SEQ ID NO:21). Another example of an ER localization tag that may be included in a cell selection system component of the present disclosure is an ER localization tag comprising 85% or greater, 90% or greater, 95% or greater, or 100% amino acid sequence identity to an ER localization tag comprising, consisting of, or present within, an amino acid sequence selected from:

(SEQ ID NO: 22)
PKKKQQKDSLINLKIQKENPKVVNEINIEDLCLTKAAYCRCWRSKTFPA
CDGSHNKHNELTGDNVGPLILKKKEV;
(SEQ ID NO: 23)
QMRHLKSFFEAKKLV;
(SEQ ID NO: 24)
AYRQRQHQDMPAPRPPGPRPAPPQQEGPPEQQPPQ;
(SEQ ID NO: 25)
HMKEKEKSD;
(SEQ ID NO: 26)
CFRKLAKTGKKKKRD;
(SEQ ID NO: 27)
KCCAYGYRKCLGKKGRVKKAHKSKTH;
(SEQ ID NO: 28)
YLSTCKDSKKKAE;
(SEQ ID NO: 29)
RLTTDVDPDLDQDED;
(SEQ ID NO: 30)
KYKSRRSFIDEKKMP;
(SEQ ID NO: 31)
MTGCCGCCCGCFGIIPLMSKCGKKSSYYTTFDNDVVIEQYRPKKSV;
(SEQ ID NO: 32)
NRSPRNRKPRRE;
(SEQ ID NO: 9)
LYKYKSRRSFIEEKKMP;
(SEQ ID NO: 33)
TKVLKGKKLSLPA;
(SEQ ID NO: 34)
KSNRHKDGFHRLRGHHDEYEDEIRMMSTGSKKSLLSHEFQDETDTEETL
YSSKH;
AND
(SEQ ID NO: 35)
KCGKKSSYYTTFDNDVVIEQYRPKKSV.

In certain embodiments, the protein localization tag is a Golgi localization tag. A non-limiting example of a Golgi localization tag that may be included in a cell selection system component of the present disclosure is a Golgi localization tag comprising the amino acid sequence YQRL (SEQ ID NO:36).

According to some embodiments, the protein localization tag is a lysosome localization tag. A non-limiting example of a lysosome localization tag that may be included in a cell selection system component of the present disclosure is a lysosome localization tag comprising the amino acid sequence KFERQ (SEQ ID NO:37).

### Protease Cleavage Sites and Proteases

As described above, the first expression construct encodes a fusion protein comprising a protease cleavage site disposed between the selection marker and the protein localization tag. The term “cleavage site” refers to the bond (e.g., a scissile bond) cleaved by an agent, e.g., a protease. A cleavage site for a protease includes the specific amino acid sequence recognized by the protease during proteolytic cleavage and may include surrounding amino acids (e.g., from one to six amino acids) on either side of the scissile bond, which bind to the active site of the protease and are needed for recognition as a substrate. In some embodiments, the cleavage site is provided as a cleavable linker, where “cleavable linker” refers to a linker including the protease cleavage site. A cleavable linker is typically cleavable under physiological conditions.

According to some embodiments, the protease cleavage site is a viral protease cleavage site. Non-limiting examples of viral protease cleavage sites include cleavage sites for potyviral family proteases. Potyviral family proteases of interest include Tobacco Etch Virus (TEV) protease, plum pox virus protease (PPVp), soybean mosaic virus protease (SbMVp), sunflower mild mosaic virus protease (SuMMVp), tobacco vein mottling virus protease (TVMVp), and West Nile virus protease (WNVp). In certain embodiments, the viral protease cleavage site is a TEV protease cleavage site. The amino acid sequence of an example TEV protease cleavage site is ENLYFQS. The amino acid sequence of an example TEV protease is the following:

(SEQ ID NO: 38)
GESLFKGPRDYNPISSTICHLTNESDGHTTSLYGIGFGPFIITNKHLFR
RNNGTLLVQSLHGVFKVKNTTTLQQHLIDGRDMIIIRMPKDFPPFPQKL
KFREPQREERICLVTTNFQTKSMSSMVSDTSCTFPSSDGIFWKHWIQTK
DGQCGSPLVSTRDGFIVGIHSASNFTNTNNYFTSVPKNFMELLTNQEAQ
QWVSGWRLNADSVLWGGHKVFMVKPEEPFQPVKEATQLMN

In some embodiments, the protease is a TEV protease comprising the amino acid sequence set forth above, or is a functional (proteolytic) variant thereof having 70% or greater, 75% or greater, 80% or greater, 85% or greater, 90% or greater, 95% or greater, or 99% or greater amino acid sequence identity to such a sequence, and/or a functional (proteolytic) fragment thereof such as a fragment having a length of from 100 to 125, 125 to 150, 150 to 175, 175 to 200, 200 to 225, or from 225 to 235 amino acids. Such a protease may be provided by two or more (e.g., two) complementary fragments of the protease, wherein the two or more (e.g., two) complementary fragments form an active protease complex.

According to some embodiments, the viral protease cleavage site is for an HCV protease. In certain embodiments, the viral protease cleavage site is for a viral protease derived from HCV nonstructural protein 3 (NS3). NS3 consists of an N-terminal serine protease domain and a C-terminal helicase domain. By “derived from HCV NS3” is meant the protease is the serine protease domain of HCV NS3 or a proteolytically active variant thereof capable of cleaving a cleavage site for the serine protease domain of HCV NS3. The protease domain of NS3 forms a heterodimer with the HCV nonstructural protein 4A (NS4A), which activates proteolytic activity. A protease derived from HCV NS3 may include the entire NS3 protein or a proteolytically active fragment thereof, and may further include a cofactor polypeptide, such as a cofactor polypeptide derived from HCV nonstructural protein 4A (NS4A), e.g., an activating NS4A region. NS3 protease is highly selective and can be inhibited by a number of non-toxic, cell-permeable drugs, which are currently available for use in humans. NS3 protease inhibitors that may be employed include, but are not limited to, simeprevir, danoprevir, asunaprevir, ciluprevir, boceprevir, sovaprevir, paritaprevir, telaprevir, grazoprevir, and any combination thereof. Non-limiting examples of proteases derived from HCV NS3 are provided below.

Example Proteases Derived from HCV NS3

(SEQ ID NO: 39)
APITAYAQQTRGLLGCIITSLTGRDKNQVEGEVQIVSTATQTFLATCIN
GVCWAVYHGAGTRTIASPKGPVIQMYTNVDQDLVGWPAPQGSRSLTPCT
CGSSDLYLVTRHADVIPVRRRGDSRGSLLSPRPISYLKGSSGGPLLCPA
GHAVGLFRAAVCTRGVAKAVDFIPVENLETTMRSPVFTD
(SEQ ID NO: 40)
APITAYAQQTRGLLGCIITSLTGRDKNQVEGEVQIMSTATQTFLATCIN
GVCWTVYHGAGTRTIASPKGPVIQMYTNVDQDLVGWPAPQGSRSLTPCT
CGSSDLYLVTRHADVIPVRRRGDGRGSLLSPRPISYLKGSSGGPLLCPA
GHAVGLFRAAVCTRGVAKAVDFIPVENLETTMRSPVFTD
(SEQ ID NO: 41)
APITAYAQQTRGLLGCIITSLTGRDKNQVEGEVQIVSTATQTFLATCIN
GVCWTVYHGAGTRTIASPKGPVIQMYTNVDQDLVGWPAPQGSRSLTPCT
CGSSDLYLVTRHADVIPVRRRGDSRGSLLSPRPISYLKGSSGGPLLCPA
GHAVGLFRAAVCTRGVAKAVDFIPVENLETTMRSPVFTD

In some embodiments, the protease comprises one of the sequences set forth above, or is a functional (proteolytic) variant thereof having 70% or greater, 75% or greater, 80% or greater, 85% or greater, 90% or greater, 95% or greater, or 99% or greater amino acid sequence identity to one of such sequences, and/or a functional (proteolytic) fragment thereof such as a fragment having a length of from 100 to 185, 120 to 185, 140 to 185, 160 to 185, 170 to 185, from 180 to 185, from 182 to 185, or from 184 to 185 amino acids.

In some embodiments, the protease cleavage site is a viral protease cleavage site. For example, when a protease derived from HCV NS3 is employed, the cleavage site should comprise an NS3 protease cleavage site. An NS3 protease cleavage site may include the four junctions between nonstructural (NS) proteins of the HCV polyprotein normally cleaved by the NS3 protease during HCV infection, including the NS3/NS4A, NS4A/NS4B, NS4B/NS5A, and NS5A/NS5B junction cleavage sites. For a description of NS3 protease and representative sequences of its cleavage sites for various strains of HCV, see, e.g., Hepatitis C Viruses: Genomes and Molecular Biology (S. L. Tan ed., Taylor & Francis, 2006), Chapter 6, pp. 163-206; the disclosure of which is incorporated herein by reference in its entirety.

In some embodiments, the protease is derived from HCV NS3 and engineered to include one or more amino acid substitutions relative to an HCV NS3 protease amino acid sequence set forth above. For example, the protease may include a substitution at the position corresponding to position 54 of the amino acid sequence APITAYAQQTRGLLGCIITSLTGRDKNQVEGEVQIVSTATQTFLATCINGVCWAVYHGAGTRTIA SPKGPVIQMYTNVDQDLVGWPAPQGSRSLTPCTCGSSDLYLVTRHADVIPVRRRGDSRGSLL SPRPISYLKGSSGGPLLCPAGHAVGLFRAAVCTRGVAKAVDFIPVENLETTMRSPVFTD (SEQ ID NO: 39). In some embodiments, such a substitution is a threonine to alanine substitution.

NS3 nucleic acid and protein sequences may be derived from HCV, including any isolate of HCV having any genotype (e.g., genotypes 1-7) or subtype. A number of NS3 nucleic acid and protein sequences are known and described, e.g., in U.S. Ser. No. 15/737,712, the disclosure of which is incorporated herein by reference in their entirety for all purposes. Additional representative NS3 sequences are listed in the National Center for Biotechnology Information (NCBI) database. See, for example, NCBI entries: Accession Nos. YP_001491553, YP_001469631, YP_001469632, NP_803144, NP_671491, YP_001469634, YP_001469630, YP_001469633, ADA68311, ADA68307, AFP99000, AFP98987, ADA68322, AFP99033, ADA68330, AFP99056, AFP99041, CBF60982, CBF60817, AHH29575, AIZ00747, AIZ00744, ABI36969, ABN05226, KF516075, KF516074, KF516056, AB826684, AB826683, JX171009, JX171008, JX171000, EU847455, EF154714, GU085487, JX171065, and JX171063; all of which sequences are herein incorporated by reference. Any of these sequences or functional variants thereof having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, or 99% or greater amino acid sequence identity to any one of these sequences, or proteolytic fragments thereof, may be employed.

NS4A nucleic acid and protein sequences may be derived from HCV, including any isolate of HCV having any genotype (e.g., seven genotypes 1-7) or subtype. A number of NS4A nucleic acid and protein sequences are known. Representative NS4A sequences are listed in the National Center for Biotechnology Information (NCBI) database. See, for example, NCBI entries: Accession Nos. NP_751925, YP_001491554, GU945462, HQ822054, FJ932208, FJ932207, FJ932205, and FJ932199; all of which sequences (as entered by the date of filing of this application) are herein incorporated by reference. Any of these sequences or functional variants thereof having 70% or greater, 75% or greater, 80% or greater, 85% or greater, 90% or greater, 95% or greater, or 99% or greater amino acid sequence identity to any one of these sequences, or proteolytic fragments thereof, may be employed.

HCV polyprotein nucleic acid and protein sequences may be derived from HCV, including any isolate of HCV having any genotype (e.g., genotypes 1-7) or subtype. A number of HCV polyprotein nucleic acid and protein sequences are known. Representative HCV polyprotein sequences are listed in the National Center for Biotechnology Information (NCBI) database. See, for example, NCBI entries: Accession Nos. YP_001469631, NP_671491, YP_001469633, YP_001469630, YP_001469634, YP_001469632, NC_009824, NC_004102, NC_009825, NC_009827, NC_009823, NC_009826, and EF108306; all of which sequences (as entered by the date of filing of this application) are herein incorporated by reference. Any of these sequences or functional variants thereof having 70% or greater, 75% or greater, 80% or greater, 85% or greater, 90% or greater, 95% or greater, or 99% or greater amino acid sequence identity to any one of these sequences, or proteolytic fragments thereof, may be employed.

In some embodiments, the protease is derived from HCV NS3 and the cleavage site includes an NS3 protease cleavage site. An NS3 protease cleavage site may include the HCV polyprotein NS3/NS4A, NS4A/NS4B, NS4B/NS5A, and NS5A/NS5B junction cleavage sites. Representative HCV NS4A/4B protease cleavage sites include DEMEECSQH and DEMEECSQH. Representative HCV NS5A/5B protease cleavage sites include EDVVPCSMG and EDVVPCSMGS. A representative NS4B/5A protease cleavage site is ECTTPCSGSWL. Additional NS3 protease cleavage sites that may be included in a recombinant polypeptide of the present disclosure include those described in Shiryaev et al. (2012) PLOS One 7 (4): e35759.

In certain embodiments, the protease cleavage site is a human protease cleavage site. Non-limiting examples of human protease cleavage sites include cleavages sites for a human kallikrein (KLK) protease, human enterokinase protease, human thrombin, a human matrix metalloprotease (MMP), human urokinase-type plasminogen activator receptor (uPAR), human plasmin, or human cathepsin. According to some embodiments, the protease cleavage site is a cleavage site for a human kallikrein (KLK) protease, non-limiting examples of which include human KLK3 (UniProtKB-Q546G3), human KLK4 (UniProtKB-Q9Y5K2), human KLK6 (UniProtKB-Q92876), human KLK8 (UniProtKB-060259), human KLK11 (UniProtKB-Q9UBX7), human KLK13 (UniProtKB-Q9UKR3), human KLK14 (UniProtKB-Q9P0G3), and human KLK15 (UniProtKB-Q9H2R5). Data demonstrating the utility of human KLK proteases and corresponding cleavage sites in the STASH Select system is provided in the Experimental section herein. Any of these sequences or functional variants thereof having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, or 99% or greater amino acid sequence identity to any one of these sequences, or proteolytic fragments thereof, may be employed.

In certain embodiments, the protease cleavage site is a protease cleavage site for a human protease selected from acrosin (ACR), AGBL carboxypeptidase 1 (AGBL1), AGBL carboxypeptidase 2 (AGBL2), AGBL carboxypeptidase 3 (AGBL3), AGBL carboxypeptidase 4 (AGBL4), AGBL carboxypeptidase 5 (AGBL5), ATP/GTP binding carboxypeptidase 1 (AGTPBP1), asparaginase and isoaspartyl peptidase 1 (ASRGL1), astacin like metalloendopeptidase (ASTL), ATP23 metallopeptidase and ATP synthase assembly factor homolog (ATP23), ataxin 3 (ATXN3), ataxin 3 like (ATXN3L), azurocidin 1 (AZU1), beta-secretase 1 (BACE1), beta-secretase 2 (BACE2), bone morphogenetic protein 1 (BMP1), BRCA1/BRCA2-containing complex subunit 3 (BRCC3), calpain 14 (CAPN14), calpain 3 (CAPN3), caspase recruitment domain family member 8 (CARD8), caspase 4 (CASP4), chymotrypsin like elastase 1 (CELA1), chymotrypsin like elastase 2A (CELA2A), chymotrypsin like elastase 2B (CELA2B), chymotrypsin like elastase 3A (CELA3A), chymotrypsin like elastase 3B (CELA3B), CUGBP Elav-like family member 3 (CELF3), CUGBP Elav-like family member 4 (CELF4), CUGBP Elav-like family member 5 (CELF5), CUGBP Elav-like family member 6 (CELF6), cell growth regulator with EF-hand domain 1 (CGREF1), charged multivesicular body protein 3 (CHMP3), CLN5 intracellular trafficking protein (CLN5), chymase 1 (CMA1), collectin subfamily member 11 (COLEC11), COP9 signalosome subunit 5 (COPS5), corin, serine peptidase (CORIN), carboxypeptidase A4 (CPA4), carboxypeptidase vitellogenic like (CPVL), cystatin SN (CST1), cystatin 11 (CST11), cystatin C (CST3), cystatin S (CST4), cystatin D (CST5), cystatin E/M (CST6), cystatin 8 (CST8), cystatin 9 (CST9), cystatin like 1 (CSTL1), chymotrypsinogen B2 (CTRB2), chymotrypsin like (CTRL), cathepsin L (CTSL), DNA damage inducible 1 homolog 2 (DDI2), DAP3 binding cell death enhancer 1 (DELE1), adipsin (DF), dickkopf WNT signaling pathway inhibitor 2 (DKK2), dickkopf WNT signaling pathway inhibitor 4 (DKK4), dipeptidase 1 (DPEP1), dipeptidyl peptidase 3 (DPP3), dipeptidyl peptidase 9 (DPP9), FAM111 trypsin like peptidase A (FAM111A), ficolin 1 (FCN1), ficolin 2 (FCN2), ficolin 3 (FCN3), G3BP stress granule assembly factor 1 (G3BP1), hepsin (HPN), HtrA serine peptidase 1 (HTRA1), insulin degrading enzyme (IDE), inner mitochondrial membrane peptidase subunit 2 (IMMP2L), jumonji domain containing 7 (JMJD7), Josephin domain containing 2 (JOSD2), kallikrein 1 (KLK1), kallikrein related peptidase 10 (KLK10), kallikrein related peptidase 11 (KLK11), kallikrein related peptidase 12 (KLK12), kallikrein related peptidase 13 (KLK13), kallikrein related peptidase 14 (KLK14), kallikrein related peptidase 15 (KLK15), kallikrein related peptidase 2 (KLK2), kallikrein related peptidase 3 (KLK3), kallikrein related peptidase 4 (KLK4), kallikrein related peptidase 5 (KLK5), kallikrein related peptidase 6 (KLK6), kallikrein related peptidase 7 (KLK7), kallikrein related peptidase 8 (KLK8), kallikrein related peptidase 9 (KLK9), kallikrein pseudogene 1 (KLKP1), lipocalin 2 (LCN2), legumain (LGMN), leishmanolysin like peptidase (LMLN), MAS1 proto-oncogene like, G protein-coupled receptor (MAS1L), MBL associated serine protease 1 (MASP1), MBL associated serine protease 2 (MASP2), mannose binding lectin 2 (MBL2), matrix metallopeptidase 10 (MMP10), matrix metallopeptidase 11 (MMP11), matrix metallopeptidase 13 (MMP13), matrix metallopeptidase 16 (MMP16), matrix metallopeptidase 2 (MMP2), napsin A aspartic peptidase (NAPSA), neurolysin (NLN), NLR family CARD domain containing 4 (NLRC4), NLR family pyrin domain containing 1 (NLRP1), aminopeptidase puromycin sensitive (NPEPPS), opiorphin prepropeptide (OPRPN), OTU deubiquitinase, ubiquitin aldehyde binding 2 (OTUB2), poly (ADP-ribose) polymerase family member 9 (PARP9), proprotein convertase subtilisin/kexin type 1 (PCSK1), proprotein convertase subtilisin/kexin type 1 inhibitor (PCSK1N), proprotein convertase subtilisin/kexin type 2 (PCSK2), proprotein convertase subtilisin/kexin type 4 (PCSK4), proprotein convertase subtilisin/kexin type 5 (PCSK5), proprotein convertase subtilisin/kexin type 6 (PCSK6), proprotein convertase subtilisin/kexin type 7 (PCSK7), proprotein convertase subtilisin/kexin type 9 (PCSK9), platelet derived growth factor C (PDGFC), pepsinogen A3 (PGA3), pepsinogen A4 (PGA4), pepsinogen A5 (PGA5), pyroglutamyl-peptidase I like (PGPEP1L), PTEN induced kinase 1 (PINK1), prolyl endopeptidase like (PREPL), parkin RBR E3 ubiquitin protein ligase (PRKN), serine protease gene group (PRSS), serine protease 2 (PRSS2), serine protease 21 (PRSS21), serine protease 22 (PRSS22), serine protease 23 (PRSS23), serine protease 27 (PRSS27), serine protease 33 (PRSS33), serine protease 46, pseudogene (PRSS46P), serine protease 55 (PRSS55), serine protease 8 (PRSS8), proteinase 3 (PRTN3), presenilin 2 (PSEN2), PYD and CARD domain containing (PYCARD), retinoic acid receptor responder 1 (RARRES1), ring finger and FYVE like domain containing E3 ubiquitin protein ligase (RFFL), rhomboid like 2 (RHBDL2), SEC11 homolog A, signal peptidase complex subunit (SEC11A), SEC11 homolog B, signal peptidase complex subunit (SEC11B), SEC11 homolog C, signal peptidase complex subunit (SEC11BC), SUMO peptidase family member, NEDD8 specific (SENP8), SET nuclear proto-oncogene (SET), synaptosome associated protein 25 (SNAP25), secreted phosphoprotein 2 (SPP2), small proline rich protein 3 (SPRR3), spleen associated tyrosine kinase (SYK), transcription factor EB (TFEB), transglutaminase 2 (TGM2), toll like receptor adaptor molecule 1 (TICAM1), tubulointerstitial nephritis antigen like 1 (TINAGL1), transmembrane serine protease 11D (TMPRSS11D), transmembrane serine protease 11E (TMPRSS11E), transmembrane serine protease 4 (TMPRSS4), transmembrane serine protease 5 (TMPRSS5), transmembrane serine protease 6 (TMPRSS6), transmembrane serine protease 7 (TMPRSS7), TNF receptor superfamily member 10a (TNFRSF10A), tryptase alpha/beta 1 (TPSAB1), tryptase beta 2 (TPSB2), tryptase delta 1 (TPSD1), tryptase gamma 1 (TPSG1), tryptase pseudogene 2 (TPSP2), tyrosylprotein sulfotransferase 1 (TPST1), tyrosylprotein sulfotransferase 2 (TPST2), tyrosylprotein sulfotransferase 2 pseudogene 1 (TPST2P1), thyrotropin releasing hormone degrading enzyme (TRHDE), thyroid hormone receptor interactor 4 (TRIP4), ubiquitin C-terminal hydrolase L1 (UCHL1), ubiquitin specific peptidase 27 X-linked (USP27X), vasohibin 2 (VASH2), valosin containing protein (VCP), and WAP four-disulfide core domain 1 (WFDC1).

In some embodiments, the protease is highly selective for the cleavage site. Additionally, the protease activity may be capable of inhibition by known small molecule inhibitors that are cell-permeable and not toxic to the cell or individual under study or treatment. For a discussion of proteases, see, e.g., V. Y. H. Hook, Proteolytic and cellular mechanisms in prohormone and proprotein processing, RG Landes Company, Austin, Tex., USA (1998); N. M. Hooper et al., Biochem. J. 321:265-279 (1997); Z. Werb, Cell 91:439-442 (1997); T. G. Wolfsberg et al., J. Cell Biol. 131:275-278 (1995); T. Berg et al., Biochem. J. 307:313-326 (1995); M. J. Smyth and J. A. Trapani, Immunology Today 16:202-206 (1995); R. V. Talanian et al., J. Biol. Chem. 272: 9677-9682 (1997); and N. A. Thornberry et al., J. Biol. Chem. 272:17907-17911 (1997), the disclosures of which are incorporated herein by reference in their entireties for all purposes. In some embodiments, the protease employed is a sequence-specific non-human protease for which FDA-approved pharmacological inhibitors are available.

Any of the proteases employed according to the methods of the present disclosure, including any of the proteases described above, may be provided by two or more (e.g., two) complementary fragments of the protease, where the two or more (e.g., two) complementary fragments form an active protease complex. A protease may be provided by two or more (e.g., two) complementary fragments of the protease, e.g., in order to increase the number of separate expression constructs required for cell surface expression of the selection marker.

### Membrane Association Domains, Hinge Domains and Dimerization Domains

Any of the cell selection system components of the present disclosure may comprise a membrane association domain. Non-limiting examples of membrane association domains include transmembrane domains. A transmembrane (Tm) domain may be derived either from a natural, synthetic, semi-synthetic, or recombinant source. In some embodiments, the Tm domain is derived from (e.g., includes at least the transmembrane region(s) or a functional portion thereof) of the alpha or beta chain of CD35, CD3ζ, CD3γ, CD3δ, CD4, CD5, CD8α, CD9, CD16, CD22, CD27, CD28, CD33, CD37, CD45, CD64, CD80, CD86, CD134, CD137, CD152, CD154, or PD-1. In certain embodiments, the transmembrane domain is a CD8α transmembrane domain. According to some embodiments, the transmembrane domain is a CD28 transmembrane domain. Non-limiting examples of transmembrane domains that may be included in one or more (e.g., each) of the cell selection system components are a transmembrane domain comprising 80% or greater, 85% or greater, 90% or greater, 95% or greater, or 100% amino acid sequence identity to a transmembrane domain comprising, consisting of, or present within, an amino acid sequence selected from WLRLLPFLGVLALLGYLAVRPFL (SEQ ID NO:42); VLWWSIAQTVILILTGIW (SEQ ID NO:43); LGPEWDLYLMTIIALLLGTVI (SEQ ID NO:44); YYASAFSMMLGLFIFSIVFL (SEQ ID NO:45); IAFLLACVATMIFMITKCCLF (SEQ ID NO:46); VIGFLLAVVLTVAFITF (SEQ ID NO:47); GLFLSAFLLLGLFKALGWAAV (SEQ ID NO:48); VGLVLAAILALLLAFYAFFYL (SEQ ID NO:49); TFCSTALLITALALVCTLLYL (SEQ ID NO:50); WYVWLAIFFAIIIFILILGWVLL (SEQ ID NO:51); WLWVVYILT VALPVFLVILFC (SEQ ID NO:52); IYIWAPLAGTCGVLLLSLVITLYC (SEQ ID NO:53); and FWVLVVVGGVLACYSLLVTVAFIIFWV (SEQ ID NO:54).

Any of the cell selection system components of the present disclosure may comprise a hinge domain, e.g., a CD8α hinge domain, a CD28 hinge domain, or the like.

Exemplary amino acid sequences of transmembrane domains and hinge domains that may be included in one or more (e.g., each) of the cell selection system components are provided herein.

Non-limiting examples of membrane association domains also include post-translational modifications that tether the cell selection system component to a membrane. That is, the cell selection system component may comprise a post-translationally added membrane-tethering domain. By “membrane-tethering domain” is meant a domain (e.g., moiety) capable of stably associating with a membrane (e.g., ER membrane) of the cell. Suitable membrane-tethering domains include, but are not limited to, post-translational modifications such as palmitoylation, myristoylation, prenylation, a glycosylphosphatidylinositol (GPI) anchor, and the like.

In some embodiments, when two or more cell selection system components comprise a membrane association domain (e.g., transmembrane domain), the membrane association domain of each component may be identical or substantially identical to each other.

In some embodiments, in order to increase the number of separate expression constructs required for cell surface expression of the selection marker, two or more cell selection system components each comprise a dimerization domain, where dimerization of the cell selection system components is required for cell surface expression of the selection marker. Examples of cell selection system configurations that employ one or more pairs of dimerization domains are described elsewhere. Non-limiting examples of dimerization domains that may be employed include domains comprising a coiled coil structure. When the dimerization domain comprises a coiled coil structure, in some embodiments, the dimerization domain comprises a leucine zipper domain.

### Genetic Modifications—Proteins of Interest and Gene Inactivations

The two or more separate expression constructs may each provide a genetic modification to the cells to which the two or more separate expression constructs are delivered. Non-limiting examples of genetic modifications include providing a region of the expression construct that encodes a protein of interest. Non-limiting examples of proteins of interest include a receptor, a ligand, a transcription factor, an antibody, a bispecific T-cell engager (BiTE), an enzyme, a cytokine, a chemokine, a toxin, a protein conferring resistance to cell exhaustion, and a suicide switch protein.

In some embodiments, a protein of interest further encoded by one or more expression constructs of the two or more separate expression constructs is a receptor. For example, one or more expression constructs of the two or more separate expression constructs may encode a receptor independently selected from a chimeric antigen receptor (CAR), a T cell receptor (TCR) such as a recombinant TCR, a chimeric cytokine receptor (CCR), a chimeric chemokine receptor, a synthetic notch receptor (synNotch), a Modular Extracellular Sensor Architecture (MESA) receptor, a Tango receptor, a ChaCha receptor, a generalized extracellular molecule sensor (GEMS) receptor, a growth factor receptor, a cytokine receptor, a chemokine receptor, a switch receptor, an adhesion molecule, an integrin, an inhibitory receptor, a stimulatory receptor, an immunoreceptor tyrosine-based activation motif (ITAM)-containing receptor, an immunoreceptor tyrosine-based inhibition motif (ITIM)-containing receptor, a hormone receptor, a receptor tyrosine kinase, an immune receptor such as CD28, CD80, ICOS, CTLA4, PD1, PD-L1, BTLA, HVEM, CD27, 4-1BB, 4-1BBL, OX40, OX40L, DR3, GITR, CD30, SLAM, CD2, 2B4, TIM1, TIM2, TIM3, TIGIT, CD226, CD160, LAG3, LAIR1, B7-1, B7-H1, and B7-H3, a type I cytokine receptor such as Interleukin-1 receptor, Interleukin-2 receptor, Interleukin-3 receptor, Interleukin-4 receptor, Interleukin-5 receptor, Interleukin-6 receptor, Interleukin-7 receptor, Interleukin-9 receptor, Interleukin-11 receptor, Interleukin-12 receptor, Interleukin-13 receptor, Interleukin-15 receptor, Interleukin-18 receptor, Interleukin-21 receptor, Interleukin-23 receptor, Interleukin-27 receptor, Erythropoietin receptor, GM-CSF receptor, G-CSF receptor, Growth hormone receptor, Prolactin receptor, Leptin receptor, Oncostatin M receptor, Leukemia inhibitory factor, a type II cytokine receptor such as interferon-alpha/beta receptor, interferon-gamma receptor, Interferon type Ill receptor, Interleukin-10 receptor, Interleukin-20 receptor, Interleukin-22 receptor, Interleukin-28 receptor, a receptor in the tumor necrosis factor receptor superfamily such as Tumor necrosis factor receptor 2 (1B), Tumor necrosis factor receptor 1, Lymphotoxin beta receptor, OX40, CD40, Fas receptor, Decoy receptor 3, CD27, CD30, 4-1BB, Decoy receptor 2, Decoy receptor 1, Death receptor 5, Death receptor 4, RANK, Osteoprotegerin, TWEAK receptor, TACI, BAFF receptor, Herpesvirus entry mediator, Nerve growth factor receptor, B-cell maturation antigen, Glucocorticoid-induced TNFR-related, TROY, Death receptor 6, Death receptor 3, Ectodysplasin A2 receptor, a chemokine receptor such as CCR1, CCR2, CCR3, CCR4, CCR5, CCR6, CCR7, CCR8, CCR9, CCR10, CXCR1, CXCR2, CXCR3, CXCR4, CXCR5, CXCR6, CX3CR1, XCR1, ACKR1, ACKR2, ACKR3, ACKR4, CCRL2, a receptor in the epidermal growth factor receptor (EGFR) family, a receptor in the fibroblast growth factor receptor (FGFR) family, a receptor in the vascular endothelial growth factor receptor (VEGFR) family, a receptor in the rearranged during transfection (RET) receptor family, a receptor in the Eph receptor family, a receptor that can induce cell differentiation (e.g., a Notch receptor), a cell adhesion molecule (CAM), an adhesion receptor such as integrin receptor, cadherin, selectin, and a receptor in the discoidin domain receptor (DDR) family, transforming growth factor beta receptor 1, and transforming growth factor beta receptor 2. In some embodiments, such a receptor is an immune cell receptor selected from a T cell receptor, a B cell receptor, a natural killer (NK) cell receptor, a macrophage receptor, a monocyte receptor, a neutrophil receptor, a dendritic cell receptor, a mast cell receptor, a basophil receptor, and an eosinophil receptor.

According to some embodiments, one or more expression constructs of the two or more separate expression constructs may encode a CAR. When two or more separate expression constructs encode a CAR, the CAR may be the same CAR, or the two or more separate expression constructs may encode two or more different CARs. In certain embodiments, when the protein of interest is a CAR, the extracellular binding domain of the CAR comprises a single chain antibody. The single-chain antibody may be a monoclonal single-chain antibody, a chimeric single-chain antibody, a humanized single-chain antibody, a fully human single-chain antibody, and/or the like. In one non-limiting example, the single chain antibody is a single chain variable fragment (scFv). Suitable CAR extracellular binding domains include those described in Labanieh et al. (2018) Nature Biomedical Engineering 2:377-391. In some embodiments, the extracellular binding domain of the CAR is a single-chain version (e.g., an scFv version) of an antibody approved by the United States Food and Drug Administration and/or the European Medicines Agency (EMA) for use as a therapeutic antibody, e.g., for inducing antibody-dependent cellular cytotoxicity (ADCC) of certain disease-associated cells in a patient, etc. Non-limiting examples of single-chain antibodies which may be employed when the protein of interest is a CAR include single-chain versions (e.g., scFv versions) of Adecatumumab, Ascrinvacumab, Cixutumumab, Conatumumab, Daratumumab, Drozitumab, Duligotumab, Durvalumab, Dusigitumab, Enfortumab, Enoticumab, Figitumumab, Ganitumab, Glembatumumab, Intetumumab, Ipilimumab, Iratumumab, Icrucumab, Lexatumumab, Lucatumumab, Mapatumumab, Narnatumab, Necitumumab, Nesvacumab, Ofatumumab, Olaratumab, Panitumumab, Patritumab, Pritumumab, Radretumab, Ramucirumab, Rilotumumab, Robatumumab, Seribantumab, Tarextumab, Teprotumumab, Tovetumab, Vantictumab, Vesencumab, Votumumab, Zalutumumab, Flanvotumab, Altumomab, Anatumomab, Arcitumomab, Bectumomab, Blinatumomab, Detumomab, Ibritumomab, Minretumomab, Mitumomab, Moxetumomab, Naptumomab, Nofetumomab, Pemtumomab, Pintumomab, Racotumomab, Satumomab, Solitomab, Taplitumomab, Tenatumomab, Tositumomab, Tremelimumab, Abagovomab, Igovomab, Oregovomab, Capromab, Edrecolomab, Nacolomab, Amatuximab, Bavituximab, Brentuximab, Cetuximab, Derlotuximab, Dinutuximab, Ensituximab, Futuximab, Girentuximab, Indatuximab, Isatuximab, Margetuximab, Rituximab, Siltuximab, Ublituximab, Ecromeximab, Abituzumab, Alemtuzumab, Bevacizumab, Bivatuzumab, Brontictuzumab, Cantuzumab, Cantuzumab, Citatuzumab, Clivatuzumab, Dacetuzumab, Demcizumab, Dalotuzumab, Denintuzumab, Elotuzumab, Emactuzumab, Emibetuzumab, Enoblituzumab, Etaracizumab, Farletuzumab, Ficlatuzumab, Gemtuzumab, Imgatuzumab, Inotuzumab, Labetuzumab, Lifastuzumab, Lintuzumab, Lorvotuzumab, Lumretuzumab, Matuzumab, Milatuzumab, Nimotuzumab, Obinutuzumab, Ocaratuzumab, Otlertuzumab, Onartuzumab, Oportuzumab, Parsatuzumab, Pertuzumab, Pinatuzumab, Polatuzumab, Sibrotuzumab, Simtuzumab, Tacatuzumab, Tigatuzumab, Trastuzumab, Tucotuzumab, Vandortuzumab, Vanucizumab, Veltuzumab, Vorsetuzumab, Sofituzumab, Catumaxomab, Ertumaxomab, Depatuxizumab, Ontuxizumab, Blontuvetmab, Tamtuvetmab, or an antigen-binding variant thereof. According to some embodiments, when the protein of interest is a CAR, the extracellular binding domain of the CAR specifically binds an antigen expressed on the surface of a cancer cell. For example, the extracellular binding domain may bind a cancer cell-surface antigen selected from B7-H3 (CD276), CD19, GD2, CD22, and HER2.

According to some embodiments, one or more expression constructs of the two or more separate expression constructs may encode an antibody. When two or more separate expression constructs encode an antibody, the antibody may be the same antibody, or the two or more separate expression constructs may encode two or more different antibodies. The term “antibody” (also used interchangeably with “immunoglobulin”) encompasses antibodies of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the antigen, including, but not limited to single chain Fv (scFv), Fab, (Fab′)₂, (scFv′)₂, and diabodies; chimeric antibodies; monoclonal antibodies, humanized antibodies, human antibodies; and fusion proteins comprising an antigen-binding portion of an antibody and a non-antibody protein.

Immunoglobulin polypeptides include the kappa and lambda light chains and the alpha, gamma (IgG1, IgG2, IgG3, IgG4), delta, epsilon and mu heavy chains or equivalents in other species. Full-length immunoglobulin “light chains” (usually of about 25 kDa or about 214 amino acids) comprise a variable region of about 110 amino acids at the NH₂-terminus and a kappa or lambda constant region at the COOH-terminus. Full-length immunoglobulin “heavy chains” (of about 150 kDa or about 446 amino acids), similarly comprise a variable region (of about 116 amino acids) and one of the aforementioned heavy chain constant regions, e.g., gamma (of about 330 amino acids).

An immunoglobulin light or heavy chain variable region (V_{L} and V_{H}, respectively) is composed of a “framework” region (FR) interrupted by three hypervariable regions, also called “complementarity determining regions” or “CDRs”. The extent of the framework region and CDRs have been defined (see, E. Kabat et al., Sequences of proteins of immunological interest, 4th ed. U.S. Dept. Health and Human Services, Public Health Services, Bethesda, MD (1987); and Lefranc et al. IMGT, the international ImMunoGeneTics information System®. Nucl. Acids Res., 2005, 33, D593-D597)). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen. All CDRs and framework provided by the present disclosure are defined according to Kabat, supra, unless otherwise indicated.

An “antibody” thus encompasses a protein having one or more polypeptides that can be genetically encodable, e.g., by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. In some embodiments, an antibody of the present disclosure is an IgG antibody, e.g., an IgG1 antibody, such as a human IgG1 antibody. In some embodiments, an antibody of the present disclosure comprises a human Fc domain.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies encompass intact immunoglobulins as well as a number of well characterized fragments which may be genetically encoded or produced by digestion with various peptidases. Thus, for example, pepsin digests an antibody below the disulfide linkages in the hinge region to produce F(ab)′₂, a dimer of Fab which itself is a light chain joined to VH-CHI by a disulfide bond. The F(ab)′₂ may be reduced under mild conditions to break the disulfide linkage in the hinge region thereby converting the (Fab′) 2 dimer into an Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein, also includes antibody fragments either produced by the modification of whole antibodies or synthesized de novo using recombinant DNA methodologies. In certain embodiments, an antibody of the present disclosure is selected from an IgG, Fv, single chain antibody, scFv, Fab, F (ab′) 2, and Fab′.

One or more of the two or more separate expression constructs may encode a protein of interest that finds use in the context of cell therapy (e.g., a cell-based cancer therapy), non-limiting examples of which include a therapy comprising administration of therapeutic immune cells such as T cells (e.g., CAR T cells, T cells that express an engineered T cell receptor (TCR), and the like), NK cells (e.g., CAR NK cells), macrophages (e.g., CAR macrophages), etc. Examples of such proteins that may be expressed by one or more of the two or more separate expression constructs include those described in Rodriguez-Garcia et al. (2020) Front Immunol. 11:1109; Martinez & Moon (2019) Front. Immunol. 10:128; Knochelmann et al. (2018) Front Immunol. 9:1740; and the like, the disclosures of which are incorporated herein in their entireties for all purposes.

In some embodiments, one or more of the two or more separate expression constructs express a protein independently selected from a protein that reduces immunogenicity of the engineered cells upon administration to an individual, a protein that confers upon the cells resistance to cell exhaustion upon administration to an individual (e.g., cJun, etc.), a protein that enhances the effectiveness of the cells in the tumor microenvironment (TME) for treatment of solid tumors (e.g., a switch receptor, a dominant negative receptor), an HLA-E protein, a CD47 protein, a homing protein (e.g., a chemokine receptor), a persistence promoting protein (e.g., a cytokine receptor), an autonomous control unit protein (e.g., a gene circuit protein, an oscillator protein, etc.), a protein that rewires the metabolism of the cells, logic gating proteins (e.g., SynNotch, iCAR), a suicide switch protein (e.g., EGFRt, iCASP9, etc.), and any other proteins useful in the context of cell therapy.

Non-limiting examples of genetic modifications also include inactivating (e.g., knocking out) one or more genes in the genome of the cell. Accordingly, in some embodiments, one or more of the two or more separate expression constructs are configured to site-specifically integrate into the genome of the cell in a manner that inactivates one or more target genes.

In some embodiments, one or more of the two or more separate expression constructs are configured to site-specifically integrate into the genome of the cell in a manner that inactivates one or more target genes, where such gene inactivation finds use in the context of cell therapy (e.g., a cell-based cancer therapy), non-limiting examples of which include a therapy comprising administration of therapeutic immune cells such as T cells (e.g., CAR T cells, T cells that express an engineered T cell receptor (TCR), and the like), NK cells (e.g., CAR NK cells), macrophages (e.g., CAR macrophages), etc. Examples of such gene inactivations include those described in Rodriguez-Garcia et al. (2020) Front Immunol. 11:1109; Martinez & Moon (2019) Front. Immunol. 10:128; Knochelmann et al. (2018) Front Immunol. 9:1740; and the like, the disclosures of which are incorporated herein in their entireties for all purposes.

In some embodiments, one or more of the two or more separate expression constructs are configured to site-specifically integrate into the genome of the cell in a manner that inactivates one or more target genes, where such gene inactivation reduces immunogenicity of the engineered cells upon administration to an individual (e.g., knockout of one or more T cell receptor genes, e.g., a TRAC knockout), confers upon the cells resistance to cell exhaustion upon administration to an individual, enhances the effectiveness of the cells in the tumor microenvironment (TME) for treatment of solid tumors, promotes persistence of the cells, and any other gene inactivation useful in the context of cell therapy.

### Selection Markers and Cell Selection

The first expression construct may encode a fusion protein comprising any convenient selection marker that enables selection of cells comprising the two or more separate expression constructs. In some embodiments, the selection marker is one that is already used for cell selection purposes for which there are existing reagents (e.g., antibodies, etc.) and devices for selecting cells exhibiting cell surface expression of the selection marker. For example, the selection marker may be one that is currently employed in magnetic-activated cell sorting (MACS) workflows, flow cytometry workflows (e.g., fluorescence-activated cell sorting (FACS) workflows), and the like.

The selection marker may be a protein tag. For example, the selection marker may be a Myc-tag, a His-tag, an HA-tag, a FLAG-tag, a Strep-tag, an NE-tag, an Xpress tag, an Avi-tag, a polyglutamate tag, a polyarginine tag, or the like.

In certain embodiments, the selection marker comprises a cluster of differentiation (CD) protein. A non-limiting example of a CD protein that finds use as a selection marker is CD34.

According to some embodiments, the selection marker comprises a truncated receptor comprising the extracellular domain of the receptor. Examples of the truncated receptors that find use as selection markers include a truncated epidermal growth factor receptor (EGFRt), a truncated nerve growth factor receptor (NGFRt), a truncated CD19 (CD19t), and a truncated CD20 (CD20t).

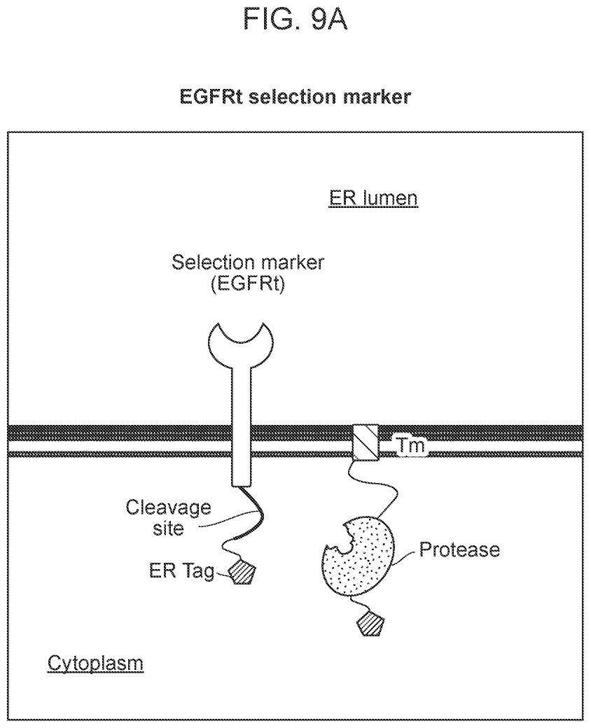

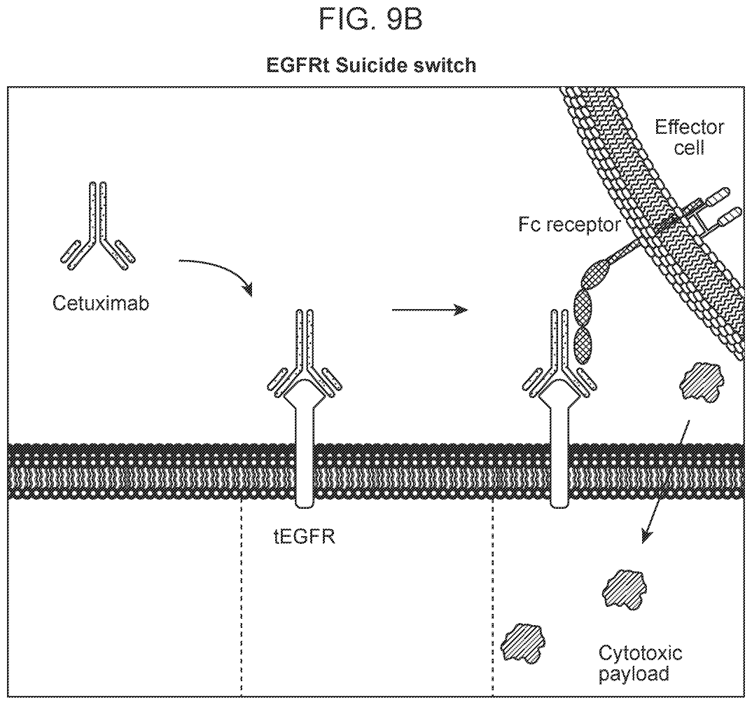

As will be appreciated with the benefit of the present disclosure, the selection marker may be chosen such that the selection marker provides a functionality in addition to facilitating selection of the cells comprising each of the two or more expression constructs. For example, the selection marker may further serve a useful function in the context of cell therapy, e.g., during a cell manufacturing process, or subsequent to administration of the cells to an individual in need thereof. In one non-limiting example, the selection marker may further serve as a suicide switch enabling ablation of the cells when the individual experiences excessive adverse side effects from the cell therapy. The use of a selection marker as a suicide switch is schematically illustrated in FIG. 9B. The suicide switch in that particular example is a truncated EGFR (EGFRt) which enables targeting of the cells using an anti-EGFR antibody such as Cetuximab.

Any convenient approach may be used to selecting for cells exhibiting cell surface expression of the selection marker. In certain embodiments, a magnetic-based cell selection approach is employed. By way of example, cells exhibiting cell surface expression of the selection marker may be selected (purified, enriched) by magnetic-activated cell sorting (MACS). MACS involves labeling cells exhibiting cell surface expression of a selection marker with magnetic beads, e.g., by combining the population of cells with magnetic beads coated with a moiety (antibody, lectin, enzyme, or the like) that binds the selection marker on the cell surface. The labeled cells may then be transferred to a column, where a magnetic field applied is applied and magnetizes the labeled cells to the walls of the column while non-labeled cells flow through the column. The magnetic field is then removed and the labeled cells (i.e., those exhibiting cell surface expression of the selection marker) may be retrieved from the column.

Also by way of example, cells exhibiting cell surface expression of the selection marker may be selected (purified, enriched) by flow cytometry, e.g., fluorescence-activated cell sorting (FACS). FACS involves labeling cells exhibiting cell surface expression of a selection marker with a fluorophore, e.g., by combining the population of cells with fluorophore-labeled antibodies that bind the selection marker on the cell surface. The fluorescently-labeled cells may then be separated from unlabeled cells using a fluorescence-activated cell sorter according to the manufacturer's instructions.

Further non-limiting examples of cell selection systems according to embodiments of the present disclosure will now be described.

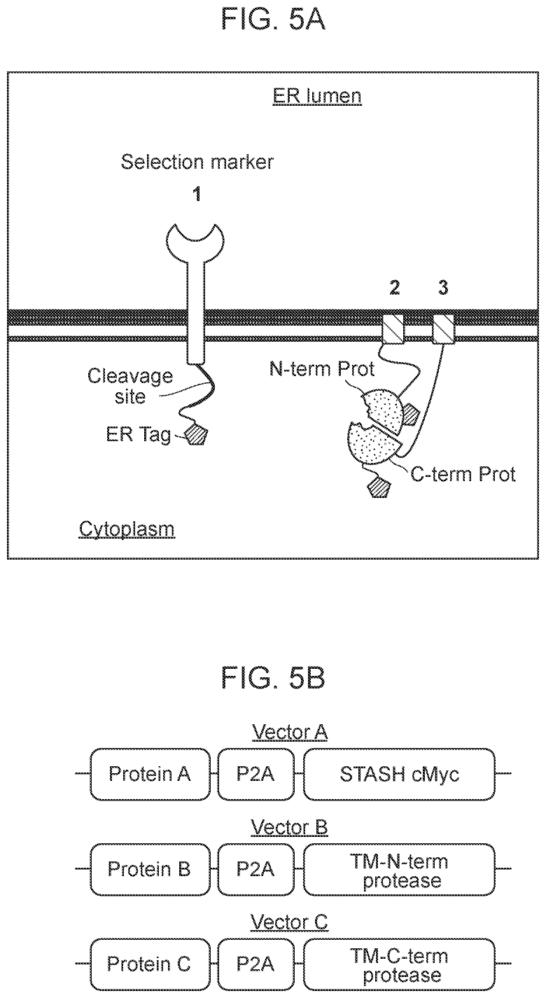

An example three-way cell selection system is schematically illustrated in FIG. 5A. In this example, an epitope-based selection marker (EGFRt, CD34, Myc tag, etc.) is fused to a protease cleavage site and an intracellular retention tag (e.g., an endoplasmic reticulum retention tag). Co-expression of a split protease (a protease comprising first and second complementary fragments that form an active protease complex), whereby the N-terminal domain of the protease is tethered to one transmembrane protein and the C-terminal domain is tethered to another transmembrane protein, results in reconstitution of an active protease complex. The active protease complex cleaves the selection marker at the protease cleavage site, which liberates the selection marker from the protein localization tag (here, an ER retention tag) and allows the selection marker to translocate to the surface of the cell. The surface-expressed selection tag can then be used as a selection handle to isolate cells expressing both the selection marker and the two protease domains (N-term protease and C-term protease).

FIG. 5B is a schematic depicting separate expression constructs which encode for three proteins of interest (protein A, protein B, and protein C) and the components of the selection system (stashed selection marker, N-term protease, and C-term protease) shown in FIG. 5A. A ribosome skipping site (here, P2A from porcine teschovirus) allows for bicistronic expression of a protein of interest and the STASH selection system components.

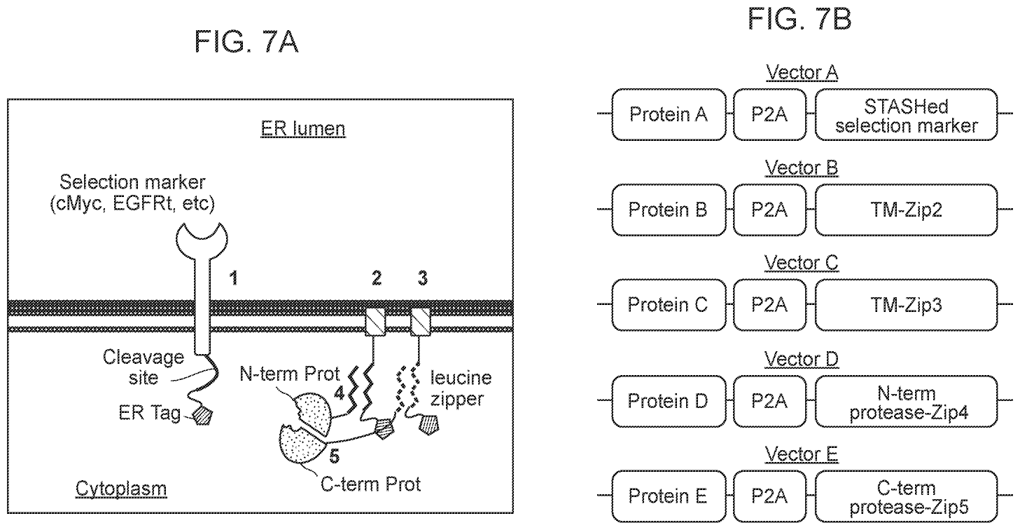

An example five-way cell selection system is schematically illustrated in FIG. 7A. The system is comprised of: 1) An epitope-based selection marker (EGFRt, CD34, Myc tag, etc.) fused to a protease cleavage site and an intracellular retention tag (e.g., endoplasmic reticulum retention tag); 2) a transmembrane domain fused to a leucine zipper (Zip2) and an ER retention tag; 3) another transmembrane domain fused to an orthogonal leucine zipper (Zip3), and an ER retention tag; 4) an N-term protease domain fused to a leucine zipper (Zip4) which binds Zip2 5) a C-term protease domain fused to a leucine zipper (Zip5) which binds Zip3. Binding events between Zip2+Zip4, Zip3+Zip5, and the two transmembrane domains result in reconstitution of an active protease complex. The active protease complex cleaves the selection marker at the protease cleavage site, which liberates the selection marker from the ER retention tag and allows the selection marker to translocate to the surface of the cell. The surface-expressed selection tag can then be used as a selection handle to isolate cells expressing the selection marker, the two protease domains (N-term protease and C-term protease), and the two transmembrane domains.

FIG. 7B is a schematic depicting separate expression constructs which encode for five proteins of interest (protein A, protein B, protein C, protein D, and protein E) and the components of the selection system (stashed selection marker, transmembrane-Zip2, transmembrane Zip3, N-term protease Zip4, and C-term protease Zip5) shown in FIG. 7A. A ribosome skipping site (here, P2A from porcine teschovirus) allows for bicistronic expression of a protein of interest and the STASH selection system components.

An example cell selection system in which a truncated epidermal growth factor receptor (EGFRt) is used as a selectable surface marker and a suicide switch is schematically illustrated in FIG. 9A. FIG. 9B is a schematic of the EGFRt suicide switch, whereby cells expressing EGFRt can be ablated by administration of an anti-EGFR antibody such as Cetuximab.

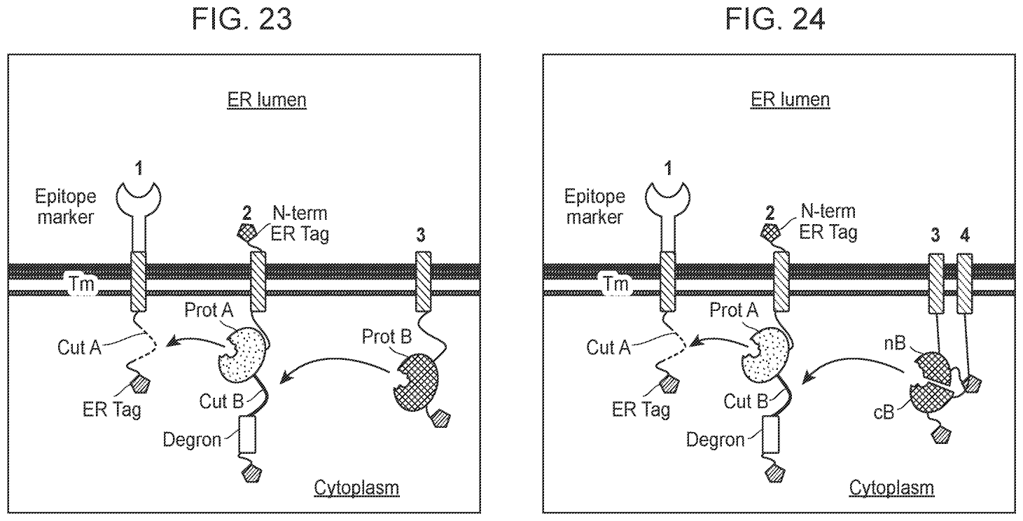

An example three-way cell selection system is schematically illustrated in FIG. 23. The first component (1) of the system is an epitope-based selection marker fused to a cleavage site A (Cut A) and a protein localization tag (here, an ER retention tag (“ER Tag”)). The second component (2) is comprised of an N-terminal ER retention Tag (N-term ER Tag), a transmembrane domain, protease A (Prot A), cleavage site B (Cut B), a degron which induces degradation of the protein, and an ER retention tag. The third component (3) is a transmembrane domain fused to protease B (Prot B) and an ER retention tag. Cut A and Cut B are cleavage sites for Prot A and Prot B, respectively. When all three components are present within the same cell, they associate at the ER membrane. Prot B of component 3 cleaves at Cut B, removing the degron from component 2 and allowing component 2 to be expressed at high levels. Component 2 in turn cleaves Cut A, which removes the ER Tag from the epitope marker and allows it to translocate to the cell surface. The surface-expressed selection tag can then be used as a selection handle to isolate cells expressing all three constructs.

As used herein, a “degron” is a sequence of amino acids which provides a degradation signal that directs a polypeptide to intracellular pathways for proteolytic degradation. The degron may promote degradation of an attached polypeptide through either the proteasome or autophagy-lysosome pathways. In some embodiments, the degron induces rapid degradation of the polypeptide. For a discussion of degrons and their function in protein degradation, see, e.g., Kanemaki et al. (2013) Pflugers Arch. 465 (3): 419-425, Erales et al. (2014) Biochim Biophys Acta 1843 (1): 216-221, Schrader et al. (2009) Nat. Chem. Biol. 5 (11): 815-822, Ravid et al. (2008) Nat. Rev. Mol. Cell. Biol. 9 (9): 679-690, Tasaki et al. (2007) Trends Biochem Sci. 32 (1 I): 520-528, Meinnel et al. (2006) Biol. Chem. 387 (7): 839-851, Kim et al. (2013) Autophagy 9 (7): 1100-1103, Varshavsky (2012) Methods Mol. Biol. 832:1-11, and Fayadat et al. (2003) Mol Biol Cell. 14 (3): 1268-1278; the disclosures of which are incorporated herein by reference in their entireties for all purposes.

According to some embodiments, the degron is one found in p53, HIF1alpha, ubiquitin, or a functional variant thereof. In certain embodiments, the degron includes portions of the HCV nonstructural proteins NS3 and NS4A. According to some embodiments, the degron comprises or consists of amino the acid sequence PITKIDTKYIMTCMSADLEVVTSTWVLVGGVLAALAAYCLST (the amino acid sequence of a degron from HCV genotype 1a; SEQ ID NO:55), or a functional variant thereof having 70% or greater, 75% or greater, 80% or greater, 85% or greater, 90% or greater, 95% or greater, or 99% or greater amino acid sequence identity to such an amino acid sequence, or a fragment thereof, such as a fragment having a length of from 30 to 41 amino acids, 32 to 41 amino acids, 34 to 41 amino acids, 36 to 41 amino acids, or 38 to 41 amino acids, wherein a functional variant of the degron is capable of promoting degradation of the polypeptide.

An example four-way cell selection system is schematically illustrated in FIG. 24. The first component (1) of the system is an epitope-based selection marker fused to a Cleavage site A (Cut A) and an ER retention tag (ER Tag). The second component (2) is comprised of an N-terminal ER retention Tag (N-term ER Tag), a transmembrane domain, Protease A (Prot A), cleavage site B (Cut B), a degron which induces degradation of the protein, and an ER retention tag. The third component (3) is a transmembrane domain fused to the n-terminal fragment of Protease B (nB) and an ER retention tag. The fourth component (4) is a transmembrane domain fused to the c-terminal fragment of Protease B (cB) and an ER retention tag. Cut A and Cut B are cleavage sites for Prot A and Prot B, respectively. When all four components are present within the same cell, they associate at the ER membrane. Protease B is reconstituted into an active form by association of the two protease fragments on components 3 and 4. The reconstituted Protease B cleaves at Cut B, removing the degron from component 2 and allowing component 2 to be expressed at high levels. Component 2 in turn cleaves Cut A, which removes the ER Tag from the epitope marker and allows it to translocate to the cell surface. The surface-expressed selection tag can then be used as a selection handle to isolate cells expressing all four constructs.

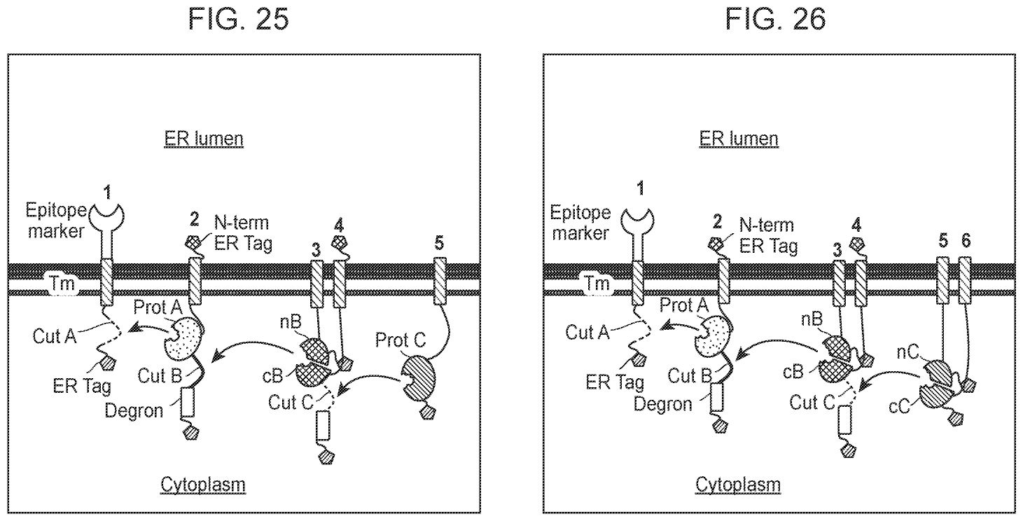

An example five-way cell selection system is schematically illustrated in FIG. 25. The first component (1) of the system is an epitope-based selection marker fused to a Cleavage site A (Cut A) and an ER retention tag (ER Tag). The second component (2) is comprised of an N-terminal ER retention Tag (N-term ER Tag), a transmembrane domain, Protease A (Prot A), cleavage site B (Cut B), a degron which induces degradation of the protein, and an ER retention tag. The third component (3) is a transmembrane domain fused to the n-terminal fragment of Protease B (nB) and an ER retention tag. The fourth component (4) is comprised of an N-terminal ER retention Tag, a transmembrane domain fused to the c-terminal fragment of Protease B (cB), cleavage site C (Cut C), a degron which induces degradation of the protein, and an ER retention tag. The fifth component (5) is a transmembrane domain fused to Protease C and an ER retention tag.

Cut A, Cut B, and Cut C are cleavage sites for Prot A, Prot B, and Prot C, respectively. When all five components are present within the same cell, they associate at the ER membrane. Prot C on component 5 cleaves at Cut C, which removes the degron from component 4 and allows it to be expressed at high levels. Protease B is reconstituted by association of components 3 and 4, which cleaves at Cut B, removing the degron from component 2 and allowing component 2 to be expressed at high levels. Prot A on component 2 in turn cleaves at Cut A, which removes the ER Tag from the epitope marker and allows it to translocate to the cell surface. The surface-expressed selection tag can then be used as a selection handle to isolate cells expressing all five constructs.

An example six-way cell selection system is schematically illustrated in FIG. 26. The first component (1) of the system is an epitope-based selection marker fused to a Cleavage site A (Cut A) and an ER retention tag (ER Tag). The second component (2) is comprised of a an N-terminal ER retention Tag (N-term ER Tag), a transmembrane domain, Protease A (Prot A), cleavage site B (Cut B), a degron which induces degradation of the protein, and an ER retention tag. The third component (3) is a transmembrane domain fused to the n-terminal fragment of Protease B (nB) and an ER retention tag. The fourth component (4) is comprised of an N-terminal ER retention Tag, a transmembrane domain fused to the c-terminal fragment of Protease B (cB), cleavage site C (Cut C), a degron which induces degradation of the protein, and an ER retention tag.

The fifth component (5) is a transmembrane domain fused to the N-terminal fragment of Protease C (nC), and ER retention tag. The sixth component (6) is a transmembrane domain fused to the C-terminal fragment of Protease C (cC), and ER retention tag. Cut A, Cut B, and Cut C are cleavage sites for Prot A, Prot B, and Prot C respectively. When all six components are present within the same cell, they associate at the ER membrane. Component 5 and 6 associate and reconstitute Prot C, which cleaves at Cut C, removing the degron from component 4 and allows it to be expressed at high levels. Protease B is reconstituted by association of components 3 and 4, which cleaves at Cut B, removing the degron from component 2 and allowing component 2 to be expressed at high levels. Prot A on component 2 in turn cleaves at Cut A, which removes the ER Tag from the epitope marker and allows it to translocate to the cell surface. The surface-expressed selection tag can then be used as a selection handle to isolate cells expressing all six constructs.

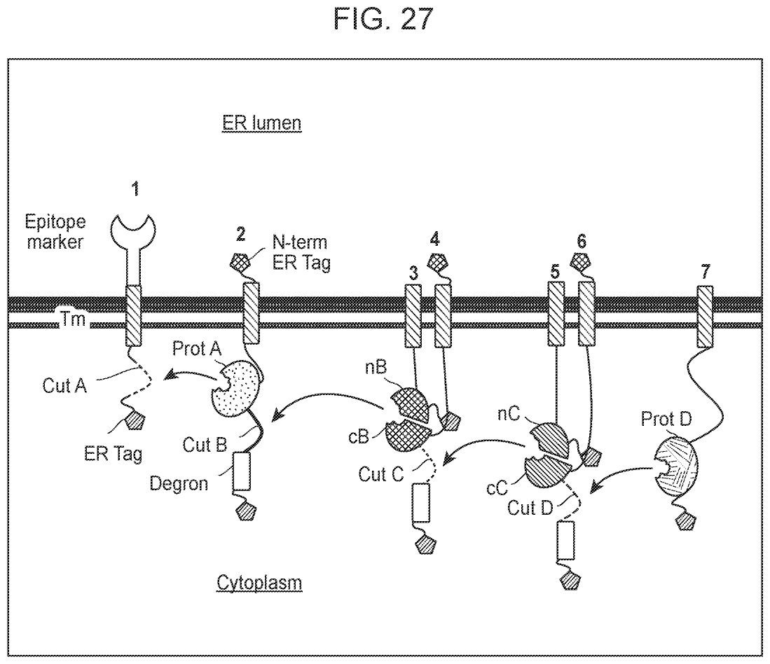

An example seven-way cell selection system is schematically illustrated in FIG. 27. The first component (1) of the system is an epitope-based selection marker fused to a Cleavage site A (Cut A) and an ER retention tag (ER Tag). The second component (2) is comprised of a an N-terminal ER retention Tag (N-term ER Tag), a transmembrane domain, Protease A (Prot A), cleavage site B (Cut B), a degron which induces degradation of the protein, and an ER retention tag. The third component (3) is a transmembrane domain fused to the n-terminal fragment of Protease B (nB) and an ER retention tag. The fourth component (4) is comprised of a an N-terminal ER retention Tag, a transmembrane domain fused to the C-terminal fragment of Protease B (cB), cleavage site C (Cut C), a degron which induces degradation of the protein, and an ER retention tag.

The fifth component (5) is a transmembrane domain fused to the N-terminal fragment of Protease C (nC), and ER retention tag. The sixth component (6) is comprised of an N-terminal ER retention Tag, a transmembrane domain fused to the C-terminal fragment of Protease C (cC), cleavage site D (Cut D), a degron which induces degradation of the protein, and an ER retention tag. The seventh component (7) is comprised of a transmembrane domain fused to Protease D (Prot D), and an ER retention tag. Cut A, Cut B, Cut C, and Cut D are cleavage sites for Prot A, Prot B, Prot C, and Prot D, respectively. When all seven components are present within the same cell, they associate at the ER membrane. Prot D on component 7 cleaves at Cut D, which removes the degron from component 6 and allows component 6 to be expressed at high levels. Component 5 and 6 associate and reconstitute Prot C, which cleaves at Cut C, removing the degron from component 4 and allows it to be expressed at high levels. Protease B is reconstituted by association of components 3 and 4, which cleaves at Cut B, removing the degron from component 2 and allowing component 2 to be expressed at high levels. Prot A on component 2 in turn cleaves at Cut A, which removes the ER Tag from the epitope marker and allows it to translocate to the cell surface. The surface-expressed selection tag can then be used as a selection handle to isolate cells expressing all seven constructs.

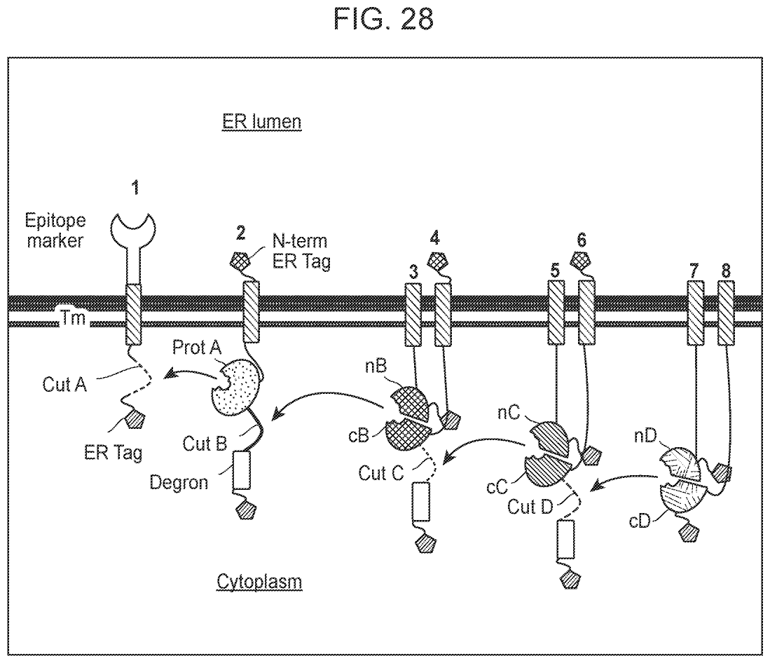

An example eight-way cell selection system is schematically illustrated in FIG. 28. The first component (1) of the system is an epitope-based selection marker fused to a Cleavage site A (Cut A) and an ER retention tag (ER Tag). The second component (2) is comprised of a an N-terminal ER retention Tag (N-term ER Tag), a transmembrane domain, Protease A (Prot A), cleavage site B (Cut B), a degron which induces degradation of the protein, and an ER retention tag. The third component (3) is a transmembrane domain fused to the n-terminal fragment of Protease B (nB) and an ER retention tag. The fourth component (4) is comprised of an N-terminal ER retention Tag, a transmembrane domain fused to the C-terminal fragment of Protease B (cB), cleavage site C (Cut C), a degron which induces degradation of the protein, and an ER retention tag.

The fifth component (5) is a transmembrane domain fused to the N-terminal fragment of Protease C (nC), and ER retention tag. The sixth component (6) is comprised of an N-terminal ER retention Tag, a transmembrane domain fused to the C-terminal fragment of Protease C (cC), cleavage site D (Cut D), a degron which induces degradation of the protein, and an ER retention tag.

The seventh component (7) is a transmembrane domain fused to the N-terminal fragment of Protease D (nD), and ER retention tag. The eighth component (8) is a transmembrane domain fused to the C-terminal fragment of Protease D (cD), and ER retention tag. Cut A, Cut B, Cut C, and Cut D are cleavage sites for Prot A, Prot B, Prot C, and Prot D, respectively. When all eight components are present within the same cell, they associate at the ER membrane. Prot D, which is reconstituted by association of components 7 and 8, cleaves at Cut D, which removes the degron from component 6 and allows component 6 to be expressed at high levels. Component 5 and 6 associate and reconstitute Prot C, which cleaves at Cut C, removing the degron from component 4 and allows it to be expressed at high levels. Protease B is reconstituted by association of components 3 and 4, which cleaves at Cut B, removing the degron from component 2 and allowing component 2 to be expressed at high levels. Prot A on component 2 in turn cleaves at Cut A, which removes the ER Tag from the epitope marker and allows it to translocate to the cell surface. The surface-expressed selection tag can then be used as a selection handle to isolate cells expressing all eight constructs.

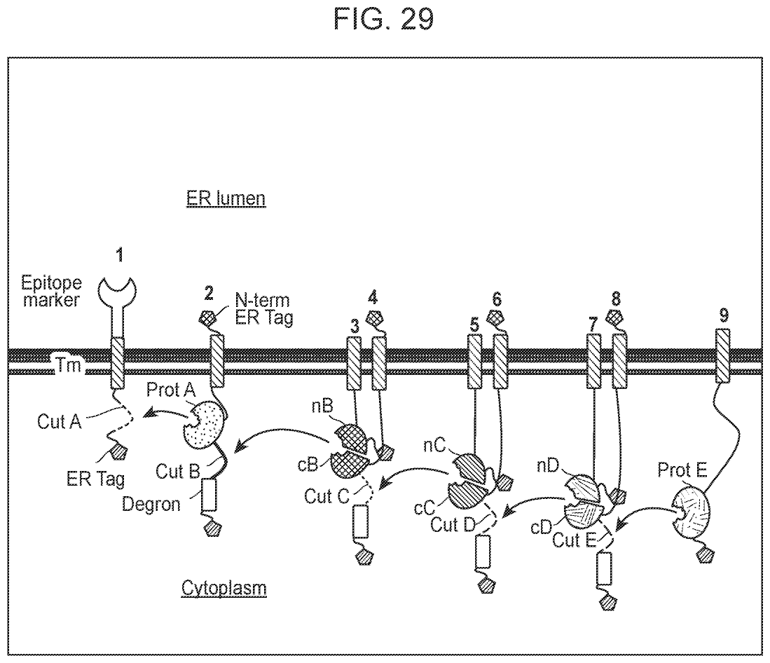

An example nine-way cell selection system is schematically illustrated in FIG. 29. The first component (1) of the system is an epitope-based selection marker fused to a Cleavage site A (Cut A) and an ER retention tag (ER Tag). The second component (2) is comprised of an N-terminal ER retention Tag (N-term ER Tag), a transmembrane domain, Protease A (Prot A), cleavage site B (Cut B), a degron which induces degradation of the protein, and an ER retention tag. The third component (3) is a transmembrane domain fused to the n-terminal fragment of Protease B (nB) and an ER retention tag. The fourth component (4) is comprised of an N-terminal ER retention Tag, a transmembrane domain fused to the C-terminal fragment of Protease B (cB), cleavage site C (Cut C), a degron which induces degradation of the protein, and an ER retention tag.

The fifth component (5) is a transmembrane domain fused to the N-terminal fragment of Protease C (nC), and ER retention tag. The sixth component (6) is comprised of an N-terminal ER retention Tag, a transmembrane domain fused to the C-terminal fragment of Protease C (cC), cleavage site D (Cut D), a degron which induces degradation of the protein, and an ER retention tag. The seventh component (7) is a transmembrane domain fused to the N-terminal fragment of Protease D (nD), and ER retention tag. The eighth component (8) is comprised of an N-terminal ER retention Tag, a transmembrane domain fused to the C-terminal fragment of Protease D (CD), cleavage site E (Cut E), a degron which induces degradation of the protein, and an ER retention tag. The ninth component (9) is comprised of a transmembrane domain fused to Protease E (Prot E), and an ER retention tag.

Cut A, Cut B, Cut C, Cut D, and Cut E are cleavage sites for Prot A, Prot B, Prot C, Prot D, and Prot E, respectively. When all nine components are present within the same cell, they associate at the ER membrane. Protease E on component 9 cleaves at Cut E, which removes the degron from component 8 and allows component 8 to be expressed at high levels. Prot D, which is reconstituted by association of components 7 and 8, cleaves at Cut D, which removes the degron from component 6 and allows component 6 to be expressed at high levels. Component 5 and 6 associate and reconstitute Prot C, which cleaves at Cut C, removing the degron from component 4 and allows it to be expressed at high levels. Protease B is reconstituted by association of components 3 and 4, which cleaves at Cut B, removing the degron from component 2 and allowing component 2 to be expressed at high levels. Prot A on component 2 in turn cleaves at Cut A, which removes the ER Tag from the epitope marker and allows it to translocate to the cell surface. The surface-expressed selection tag can then be used as a selection handle to isolate cells expressing all nine constructs.

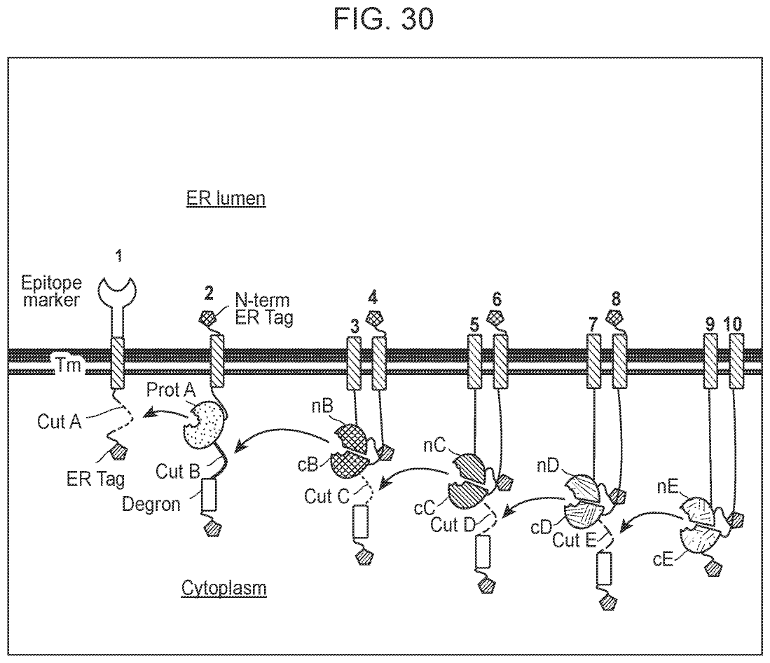

An example ten-way cell selection system is schematically illustrated in FIG. 30. The first component (1) of the system is an epitope-based selection marker fused to a Cleavage site A (Cut A) and an ER retention tag (ER Tag). The second component (2) is comprised of a N-terminal ER retention Tag (N-term ER Tag), a transmembrane domain, Protease A (Prot A), cleavage site B (Cut B), a degron which induces degradation of the protein, and an ER retention tag. The third component (3) is a transmembrane domain fused to the n-terminal fragment of Protease B (nB) and an ER retention tag. The fourth component (4) is comprised of a N-terminal ER retention Tag, a transmembrane domain fused to the C-terminal fragment of Protease B (cB), cleavage site C (Cut C), a degron which induces degradation of the protein, and an ER retention tag.

The fifth component (5) is a transmembrane domain fused to the N-terminal fragment of Protease C (nC), and ER retention tag. The sixth component (6) is comprised of an N-terminal ER retention Tag, a transmembrane domain fused to the C-terminal fragment of Protease C (cC), cleavage site D (Cut D), a degron which induces degradation of the protein, and an ER retention tag. The seventh component (7) is a transmembrane domain fused to the N-terminal fragment of Protease D (nD), and ER retention tag. The eighth component (8) is comprised of an N-terminal ER retention Tag, a transmembrane domain fused to the C-terminal fragment of Protease D (cD), cleavage site E (Cut E), a degron which induces degradation of the protein, and an ER retention tag.

The ninth component (9) is a transmembrane domain fused to the N-terminal fragment of Protease E (nE), and ER retention tag. The tenth component (10) is a transmembrane domain fused to the C-terminal fragment of Protease E (cE), and ER retention tag.

Cut A, Cut B, Cut C, Cut D, and Cut E are cleavage sites for Prot A, Prot B, Prot C, Prot D, and Prot E, respectively. When all ten components are present within the same cell, they associate at the ER membrane. Protease E, which is reconstituted by association of components 9 and 10, cleaves at Cut E, which removes the degron from component 8 and allows component 8 to be expressed at high levels. Prot D, which is reconstituted by association of components 7 and 8, cleaves at Cut D, which removes the degron from component 6 and allows component 6 to be expressed at high levels. Component 5 and 6 associate and reconstitute Prot C, which cleaves at Cut C, removing the degron from component 4 and allows it to be expressed at high levels. Protease B is reconstituted by association of components 3 and 4, which cleaves at Cut B, removing the degron from component 2 and allowing component 2 to be expressed at high levels. Prot A on component 2 in turn cleaves at Cut A, which removes the ER Tag from the epitope marker and allows it to translocate to the cell surface. The surface-expressed selection tag can then be used as a selection handle to isolate cells expressing all ten constructs.

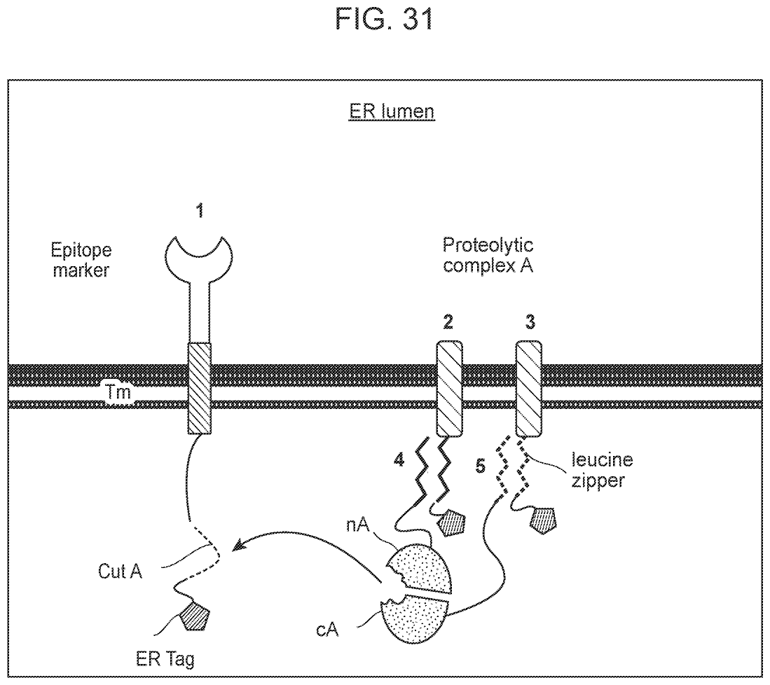

An example five-way cell selection system is schematically illustrated in FIG. 31. The first component (1) of the system is an epitope-based selection marker fused to a Cleavage site A (Cut A) and an ER retention tag (ER Tag). The second component (2) is comprised of a transmembrane domain, a leucine zipper (Zip2), and an ER retention tag. The third component (3) is comprised of a transmembrane domain, a leucine zipper (Zip3), and an ER retention tag. The fourth component (4) is comprised of a leucine zipper (Zip4), which is a cognate leucine zipper to Zip2, and the N-terminal fragment of Protease A (nA). The fifth component (5) is comprised of a leucine zipper (Zip5), which is a cognate leucine zipper to Zip3, and the C-terminal fragment of Protease A (cA).

Binding events between Zip2+Zip4, Zip3+Zip5, and the transmembrane domains result in reconstitution of the Proteolytic complex A and localization at the ER in close proximity to component 1. The Proteolytic complex A cleaves at Cut A, which removes the ER Tag from the epitope marker and allows it to translocate to the cell surface. The surface-expressed selection tag can then be used as a selection handle to isolate cells expressing all five constructs.

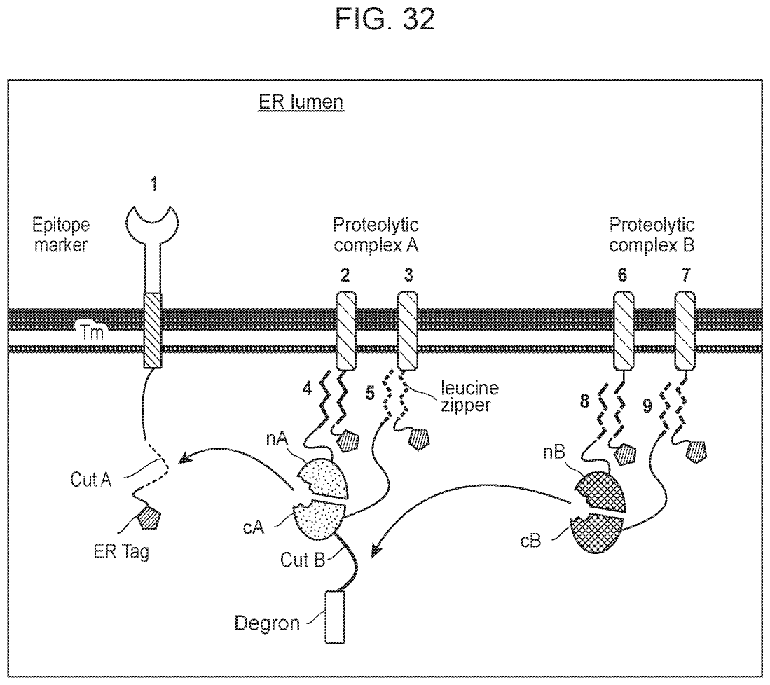

An example nine-way cell selection system is schematically illustrated in FIG. 32. The first component (1) of the system is an epitope-based selection marker fused to a Cleavage site A (Cut A) and an ER retention tag (ER Tag). The second component (2) is comprised of a transmembrane domain, a leucine zipper (Zip2), and an ER retention tag. The third component (3) is comprised of a transmembrane domain, a leucine zipper (Zip3), and an ER retention tag. The fourth component (4) is comprised of a leucine zipper (Zip4), which is a cognate leucine zipper to Zip2, and the N-terminal fragment of Protease A (nA). The fifth component (5) is comprised of a leucine zipper (Zip5), which is a cognate leucine zipper to Zip3, and the C-terminal fragment of Protease A (CA), cleavage site B (Cut B), and a degron which induces degradation of the protein. The sixth component (6) is comprised of a transmembrane domain, a leucine zipper (Zip6), and an ER retention tag. The seventh component (7) is comprised of a transmembrane domain, a leucine zipper (Zip7), and an ER retention tag. The eighth component (8) is comprised of a leucine zipper (Zip8), which is a cognate leucine zipper to Zip6, and the N-terminal fragment of Protease B (nB). The ninth component (9) is comprised of a leucine zipper (Zip9), which is a cognate leucine zipper to Zip7, and the C-terminal fragment of Protease B (cB).

Binding events between Zip6+Zip8, Zip7+Zip9, and the transmembrane domains result in reconstitution of the Proteolytic complex B and localization at the ER in close proximity to component Proteolytic complex A. Protease Complex B cleaves at Cut B, which removes the degron from component 5, allowing component 5 to be expressed at high levels.

Binding events between Zip2+Zip4, Zip3+Zip5, and the transmembrane domains result in reconstitution of the Proteolytic complex A and localization at the ER in close proximity to component 1. The Proteolytic complex A cleaves at Cut A, which removes the ER Tag from the epitope marker and allows it to translocate to the cell surface. The surface-expressed selection tag can then be used as a selection handle to isolate cells expressing all nine constructs.

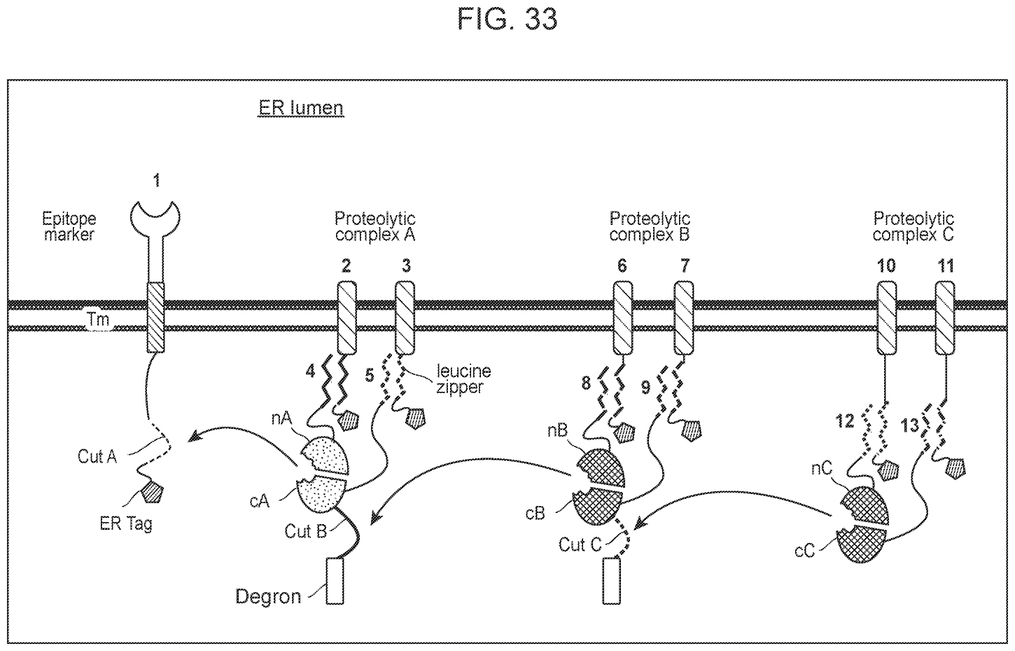

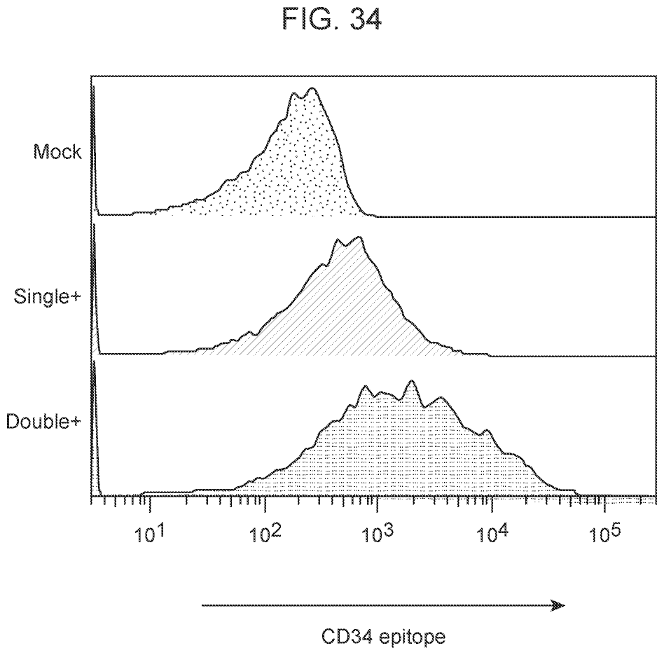

An example thirteen-way cell selection system is schematically illustrated in FIG. 33. The first component (1) of the system is an epitope-based selection marker fused to a Cleavage site A (Cut A) and an ER retention tag (ER Tag). The second component (2) is comprised of a transmembrane domain, a leucine zipper (Zip2), and an ER retention tag. The third component (3) is comprised of a transmembrane domain, a leucine zipper (Zip3), and an ER retention tag. The fourth component (4) is comprised of a leucine zipper (Zip4), which is a cognate leucine zipper to Zip2, and the N-terminal fragment of Protease A (nA). The fifth component (5) is comprised of a leucine zipper (Zip5), which is a cognate leucine zipper to Zip3, and the C-terminal fragment of Protease A (cA), cleavage site B (Cut B), and a degron which induces degradation of the protein. The sixth component (6) is comprised of a transmembrane domain, a leucine zipper (Zip6), and an ER retention tag. The seventh component (7) is comprised of a transmembrane domain, a leucine zipper (Zip7), and an ER retention tag. The eighth component (8) is comprised of a leucine zipper (Zip8), which is a cognate leucine zipper to Zip6, and the N-terminal fragment of Protease B (nB). The ninth component (9) is comprised of a leucine zipper (Zip9), which is a cognate leucine zipper to Zip7, and the C-terminal fragment of Protease B (cB), cleavage site C (Cut C), and a degron which induces degradation of the protein. The tenth component (10) is comprised of a transmembrane domain, a leucine zipper (Zip10), and an ER retention tag. The eleventh component (11) is comprised of a transmembrane domain, a leucine zipper (Zip11), and an ER retention tag. The twelfth component (12) is comprised of a leucine zipper (Zip12), which is a cognate leucine zipper to Zip10, and the N-terminal fragment of Protease C (nC). The thirteenth component (13) is comprised of a leucine zipper (Zip13), which is a cognate leucine zipper to Zip11, and the C-terminal fragment of Protease C (cC).

Binding events between Zip10+Zip12, Zip11+Zip13, and the transmembrane domains result in reconstitution of the Proteolytic complex C and localization at the ER in close proximity to Proteolytic complex B. Protease Complex C cleaves at Cut C, which removes the degron from component 9, allowing component 9 to be expressed at high levels.

Binding events between Zip6+Zip8, Zip7+Zip9, and the transmembrane domains result in reconstitution of the Proteolytic complex B and localization at the ER in close proximity to Proteolytic complex A. Protease Complex B cleaves at Cut B, which removes the degron from component 5, allowing component 5 to be expressed at high levels.

Binding events between Zip2+Zip4, Zip3+Zip5, and the transmembrane domains result in reconstitution of the Proteolytic complex A and localization at the ER in close proximity to component 1. The Proteolytic complex A cleaves at Cut A, which removes the ER Tag from the epitope marker and allows it to translocate to the cell surface. The surface-expressed selection tag can then be used as a selection handle to isolate cells expressing all thirteen constructs.

### Fusion Proteins

Also provided by the present disclosure are fusion proteins. In some embodiments, provided are any of the fusion proteins employed in the cell selection methods described above, e.g., any of the fusion proteins encoded by the first, second, etc. expression constructs described elsewhere herein, including any of the fusion proteins or equivalents thereof for which the amino acid sequences are provided herein, e.g., in the sequence table(s) herein. Also provided are nucleic acids encoding such fusion proteins and expression vectors comprising such nucleic acids. Cells comprising such fusion proteins, nucleic acids and/or expression vectors are also provided.

In certain embodiments, provided are fusion proteins comprising a protein fused to an ER localization tag, wherein the ER localization tag comprises 80% or greater, 85% or greater, 90% or greater, 95% or greater, or 100% amino acid sequence identity to an ER localization tag comprising, consisting of, or present within, an amino acid sequence selected from

(SEQ ID NO: 22)
PKKKQQKDSLINLKIQKENPKVVNEINIEDLCLTKAAYCRCWRSKTFPA
CDGSHNKHNELTGDNVGPLILKKKEV;
(SEQ ID NO: 23)
QMRHLKSFFEAKKLV;
(SEQ ID NO: 24)
AYRQRQHQDMPAPRPPGPRPAPPQQEGPPEQQPPQ;
(SEQ ID NO: 25)
HMKEKEKSD;
(SEQ ID NO: 26)
CFRKLAKTGKKKKRD;
(SEQ ID NO: 27)
KCCAYGYRKCLGKKGRVKKAHKSKTH;
(SEQ ID NO: 28)
YLSTCKDSKKKAE;
(SEQ ID NO: 29)
RLTTDVDPDLDQDED;
(SEQ ID NO: 30)
KYKSRRSFIDEKKMP;
(SEQ ID NO: 31)
MTGCCGCCCGCFGIIPLMSKCGKKSSYYTTFDNDVVIEQYRPKKSV;
(SEQ ID NO: 32)
NRSPRNRKPRRE;
(SEQ ID NO: 9)
LYKYKSRRSFIEEKKMP;
(SEQ ID NO: 33)
TKVLKGKKLSLPA;
(SEQ ID NO: 34)
KSNRHKDGFHRLRGHHDEYEDEIRMMSTGSKKSLLSHEFQDETDTEETL
YSSKH;
and
(SEQ ID NO: 35)
KCGKKSSYYTTFDNDVVIEQYRPKKSV.

According to some embodiments, provided are fusion proteins comprising a protein fused to an ER localization tag, where the ER localization tag comprises a transmembrane (Tm) domain, an intracellular domain (ICD), or both, of an ER localization tag of a polypeptide set forth in Table 1, or a variant Tm and/or ICD thereof which retains the ability to localize a polypeptide to the ER. By a “variant” Tm and/or ICD is meant a variant that comprises an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, or 99% or greater amino acid sequence identity to the parental/reference sequence, or a fragment thereof, where the variant retains the ability of the ER localization tag to localize a polypeptide to the ER.

In certain embodiments, provided are fusion proteins comprising a protein fused to an ER localization tag, wherein the ER localization tag comprises a Tm domain, an ICD, or both, of an ER localization tag of a human ER-resident protein, or a variant Tm and/or ICD thereof which retains the ability to localize a polypeptide to the ER. For example, as demonstrated in the Experimental section herein, aspects of the present disclosure include novel human ER localization tags that find use in localizing proteins to the ER. According to some embodiments, the human ER-resident protein is CDGSH iron sulfur domain 2 (CISD2). In certain embodiments, such an ER localization tag comprises the Tm domain, the ICD, or both, of the polypeptide set forth in SEQ ID NO:91, or a variant Tm and/or ICD thereof which retains the ability to localize a polypeptide to the ER. According to some embodiments, the human ER-resident protein is UDP glucuronosyltransferase family 2 member B17 (UGT2B17). In certain embodiments, such an ER localization tag comprises the Tm domain, the ICD, or both, of the polypeptide set forth in SEQ ID NO:95, or a variant Tm and/or ICD thereof which retains the ability to localize a polypeptide to the ER.

The fusion protein may be fused directly to the ER localization tag, or indirectly via one or more domains, e.g., other protein-encoding domain(s), linker(s), and/or the like. The fusion protein may further comprise a protease cleavage site, e.g., disposed between the protein and the ER localization tag. The fusion protein may further comprise a membrane association domain, e.g., any of the transmembrane domains described elsewhere herein. The fusion protein may further comprise a protein localization tag, e.g., any of the protein localization tags described elsewhere herein. Also provided are nucleic acids encoding such fusion proteins and expression vectors comprising such nucleic acids. Cells comprising such fusion proteins, nucleic acids and/or expression vectors are also provided. Methods of producing such fusion proteins are also provided. In some embodiments, such methods comprise culturing a cell comprising an expression vector encoding the fusion protein under conditions suitable for the cell to express the fusion protein, wherein the fusion protein is produced.

Also provided are fusion proteins comprising a protein fused to a transmembrane domain, wherein the transmembrane domain comprises 80% or greater, 85% or greater, 90% or greater, 95% or greater, or 100% amino acid sequence identity to a transmembrane domain comprising, consisting of, or present within, an amino acid sequence selected from

(SEQ ID NO: 42)
WLRLLPFLGVLALLGYLAVRPFL;
(SEQ ID NO: 43)
VLWWSIAQTVILILTGIW;
(SEQ ID NO: 44)
LGPEWDLYLMTIIALLLGTVI;
(SEQ ID NO: 45)
YYASAFSMMLGLFIFSIVFL;
(SEQ ID NO: 46)
IAFLLACVATMIFMITKCCLF;
(SEQ ID NO: 47)
VIGFLLAVVLTVAFITF;
(SEQ ID NO: 48)
GLFLSAFLLLGLFKALGWAAV;
(SEQ ID NO: 49)
VGLVLAAILALLLAFYAFFYL;
(SEQ ID NO: 50)
TFCSTALLITALALVCTLLYL;
(SEQ ID NO: 51)
WYVWLAIFFAIIIFILILGWVLL;
(SEQ ID NO: 52)
WLWVVYILT VALPVFLVILFC;
(SEQ ID NO: 53)
IYIWAPLAGTCGVLLLSLVITLYC;
and
(SEQ ID NO: 54)
FWVLVVVGGVLACYSLLVTVAFIIFWV.

The fusion protein may be fused directly to the transmembrane domain, or indirectly via one or more domains, e.g., other protein-encoding domain(s), linker(s), and/or the like. The fusion protein may further comprise a protease cleavage site. The fusion protein may further comprise a membrane association domain, e.g., any of the transmembrane domains described elsewhere herein. The fusion protein may further comprise a protein localization tag, e.g., any of the protein localization tags described elsewhere herein. Also provided are nucleic acids encoding such fusion proteins and expression vectors comprising such nucleic acids. Cells comprising such fusion proteins, nucleic acids and/or expression vectors are also provided. Methods of producing such fusion proteins are also provided. In some embodiments, such methods comprise culturing a cell comprising an expression vector encoding the fusion protein under conditions suitable for the cell to express the fusion protein, wherein the fusion protein is produced.

The amino acid sequences of exemplary cell selection system components are provided in Table 1 below. For each sequence, the domains as ordered from N- to C-terminus are listed in the left column. The sequence in the right column indicates the domains by alternating underlining. The present disclosure provides each of the proteins provided in Table 1, and each of the individual domains therein, as well as nucleic acids that encode such proteins and individual domains. Cells comprising such proteins and nucleic acids are also provided. As will be appreciated, the present disclosure also provides variants of any of the proteins and individual domains therein, where in some instances a variant protein or domain thereof comprises an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, or 99% or greater amino acid sequence identity to the parental/reference sequence, or a functional fragment thereof, where the variant retains the functionality (e.g., protease activity, cleavability by the protease, localization/retention (e.g., at the ER), selectability by a cell selection system, and/or the like) of the parental/reference sequence.

TABLE 1
Amino Acid and Nucleotide Sequences of Exemplary Cell Selection System
Components

| | | ID |
|---|---|---|
| Description: | Sequence: | NO: |
| # 415 Two-way protease | MVSKGEELIKENMHMKLYMEGTVDNHHFKCTSEGE | 56 |
| •mTagBFP2 | GKPYEGTQTMRIKVVEGGPLPFAFDILATSFLYGSKT | |
| •Linker | FINHTQGIPDFFKQSFPEGFTWERVTTYEDGGVLTAT | |
| •P2A | QDTSLQDGCLIYNVKIRGVNFTSNGPVMQKKTLGWE | |
| •TCRß Leader | AFTETLYPADGGLEGRNDMALKLVGGSHLIANAKTTY | |
| •RQR8 Tag | RSKKPAKNLKMPGVYYVDYRLERIKEANNETYVEQH | |
| •CD8α Hinge | EVAVARYCDLPSKLGHKLNNASATNFSLLKQAGDVE | |
| •CD8α Tm | ENPGPMGTSLLCWMALCLLGADHADACPYSNPSLC | |
| •CD8α intracellular linker | SGGGGSELPTQGTFSNVSTNVSPAKPTTTACPYSNP | |
| •NS4A cofactor domain | SLCSGGGGSPAPRPPTPAPTIASQPLSLRPEACRPA | |
| •linker | AGGAVHTRGLDFACDIYIWAPLAGTCGVLLLSLVITLY | |
| •HCV NS3 protease | CNHRNRRRVCKCPRPVVGSSGNSSGGSTGCVVIVG | |
| •NS3 helicase fragment | RIVLSGSGTSAPITAYAQQTRGLLGCIITSLTGRDKNQ | |
| •linker | VEGEVQIMSTATQTFLATCINGVCWAVYHGAGTRTIA | |
| •adenovirus E3-19K ER | SPKGPVIQMYTNVDQDLVGWPAPQGSRSLTPCTCG | |
| retention tag | SSDLYLVTRHADVIPVRRRGDGRGSLLSPRPISYLKG | |
| | SSGGPLLCPAGHAVGLFRAAVCTRGVAKAVDFIPVE | |
| | NLETTMRSPVFTDNSSPPAVTLTHAAASTGSSGGGG | |
| | GSGGLYKYKSRRSFIDEKKMP | |
| # 413 Two-way protease | MVSKGEELIKENMHMKLYMEGTVDNHHFKCTSEGE | 57 |
| •mTagBFP2 | GKPYEGTQTMRIKVVEGGPLPFAFDILATSFLYGSKT | |
| •Linker | FINHTQGIPDFFKQSFPEGFTWERVTTYEDGGVLTAT | |
| •P2A | QDTSLQDGCLIYNVKIRGVNFTSNGPVMQKKTLGWE | |
| •TCRß Leader | AFTETLYPADGGLEGRNDMALKLVGGSHLIANAKTTY | |
| •RQR8 Tag | RSKKPAKNLKMPGVYYVDYRLERIKEANNETYVEQH | |
| •CD8α Hinge | EVAVARYCDLPSKLGHKLNNASATNFSLLKQAGDVE | |
| •CD8α Tm | ENPGPMGTSLLCWMALCLLGADHADACPYSNPSLC | |
| •CD8α intracellular linker | SGGGGSELPTQGTFSNVSTNVSPAKPTTTACPYSNP | |
| •TEV protease | SLCSGGGGSPAPRPPTPAPTIASQPLSLRPEACRPA | |
| •linker | AGGAVHTRGLDFACDIYIWAPLAGTCGVLLLSLVITLY | |
| •adenovirus E3-19K ER | CNHRNRRRVCKCPRPVVGSSGNSSGGSGSTGSSG | |
| retention tag | GSGGSGSSGGSGESLFKGPRDYNPISSTICHLTNES | |
| | DGHTTSLYGIGFGPFIITNKHLFRRNNGTLLVQSLHGV | |
| | FKVKNTTTLQQHLIDGRDMIIIRMPKDFPPFPQKLKFR | |
| | EPQREERICLVTTNFQTKSMSSMVSDTSCTFPSSDGI | |
| | FWKHWIQTKDGQCGSPLVSTRDGFIVGIHSASNFTN | |
| | TNNYFTSVPKNFMELLTNQEAQQWVSGWRLNADSV | |
| | LWGGHKVFMVKPEEPFQPVKEATQLMNAAASTGSS | |
| | GGGGGSGGLYKYKSRRSFIDEKKMP | |
| # 414 Three-way protease | MVSKGEELIKENMHMKLYMEGTVDNHHFKCTSEGE | 58 |
| •mTagBFP2 | GKPYEGTQTMRIKVVEGGPLPFAFDILATSFLYGSKT | |
| •Linker | FINHTQGIPDFFKQSFPEGFTWERVTTYEDGGVLTAT | |
| •P2A | QDTSLQDGCLIYNVKIRGVNFTSNGPVMQKKTLGWE | |
| •TCRß Leader | AFTETLYPADGGLEGRNDMALKLVGGSHLIANAKTTY | |
| •RQR8 Tag | RSKKPAKNLKMPGVYYVDYRLERIKEANNETYVEQH | |
| •CD8α Hinge | EVAVARYCDLPSKLGHKLNNASATNFSLLKQAGDVE | |
| •CD8α Tm | ENPGPMGTSLLCWMALCLLGADHADACPYSNPSLC | |
| •CD8α intracellular linker | SGGGGSELPTQGTFSNVSTNVSPAKPTTTACPYSNP | |
| •nTEV protease | SLCSGGGGSPAPRPPTPAPTIASQPLSLRPEACRPA | |
| •linker | AGGAVHTRGLDFACDIYIWAPLAGTCGVLLLSLVITLY | |
| •adenovirus E3-19K ER | CNHRNRRRVCKCPRPVVGSSGNSSGGSGSTGSSG | |
| retention tag | GSGGSGSSGGSGESLFKGPRDYNPISSTICHLTNES | |
| | DGHTTSLYGIGFGPFIITNKHLFRRNNGTLLVQSLHGV | |
| | FKVKNTTTLQQHLIDGRDMIIIRMPKDFPPFPQKLKFR | |
| | EPQREERICLVTTNFQTAAASTGSSGGGGGSGGLYK | |
| | YKSRRSFIDEKKMP | |
| #416 Three-way protease | MVSKGEEVIKEFMRFKVRMEGSMNGHEFEIEGEGE | 59 |
| •tdTomato | GRPYEGTQTAKLKVTKGGPLPFAWDILSPQFMYGSK | |
| •Linker | AYVKHPADIPDYKKLSFPEGFKWERVMNFEDGGLVT | |
| •P2A | VTQDSSLQDGTLIYKVKMRGTNFPPDGPVMQKKTM | |
| •TCRß Leader | GWEASTERLYPRDGVLKGEIHQALKLKDGGHYLVEF | |
| •RQR8 Tag | KTIYMAKKPVQLPGYYYVDTKLDITSHNEDYTIVEQYE | |
| •CD8α Hinge | RSEGRHHLFLGHGTGSTGSGSSGTASSEDNNMAVIK | |
| •CD8α Tm | EFMRFKVRMEGSMNGHEFEIEGEGEGRPYEGTQTA | |
| •CD8α intracellular linker | KLKVTKGGPLPFAWDILSPQFMYGSKAYVKHPADIPD | |
| •cTEV protease | YKKLSFPEGFKWERVMNFEDGGLVTVTQDSSLQDG | |
| •linker | TLIYKVKMRGTNFPPDGPVMQKKTMGWEASTERLYP | |
| •adenovirus E3-19K ER | RDGVLKGEIHQALKLKDGGHYLVEFKTIYMAKKPVQL | |
| retention tag | PGYYYVDTKLDITSHNEDYTIVEQYERSEGRHHLFLY | |
| | GMDELYKNAGNSSIGATNFSLLKQAGDVEENPGPM | |
| | GTSLLCWMALCLLGADHADACPYSNPSLCSGGGGS | |
| | ELPTQGTFSNVSTNVSPAKPTTTACPYSNPSLCSGG | |
| | GGSPAPRPPTPAPTIASQPLSLRPEACRPAAGGAVH | |
| | TRGLDFACDIYIWAPLAGTCGVLLLSLVITLYCNHRNR | |
| | RRVCKCPRPVVGSSGNSSGGSGSTGSSGGSGGSG | |
| | SSGGSKSMSSMVSDTSCTFPSSDGIFWKHWIQTKD | |
| | GQCGSPLVSTRDGFIVGIHSASNFTNTNNYFTSVPKN | |
| | FMELLTNQEAQQWVSGWRLNADSVLWGGHKVFMV | |
| | KPEEPFQPVKEATQLMNAAASTGSSGGGGGSGGLY | |
| | KYKSRRSFIDEKKMP | |
| # 446 Three-way protease | MVSKGEELIKENMHMKLYMEGTVDNHHFKCTSEGE | 60 |
| •mTagBFP2 | GKPYEGTQTMRIKVVEGGPLPFAFDILATSFLYGSKT | |
| •Linker | FINHTQGIPDFFKQSFPEGFTWERVTTYEDGGVLTAT | |
| •P2A | QDTSLQDGCLIYNVKIRGVNFTSNGPVMQKKTLGWE | |
| •TCRβ Leader | AFTETLYPADGGLEGRNDMALKLVGGSHLIANAKTTY | |
| •RQR8 Tag | RSKKPAKNLKMPGVYYVDYRLERIKEANNETYVEQH | |
| •CD28 Hinge | EVAVARYCDLPSKLGHKLNNASATNFSLLKQAGDVE | |
| •CD28 Tm | ENPGPMGTSLLCWMALCLLGADHADACPYSNPSLC | |
| •CD28 intracellular linker | SGGGGSELPTQGTFSNVSTNVSPAKPTTTACPYSNP | |
| •nTEV protease | SLCSGGGGSIEVMYPPPYLDNEKSNGTIIHVKGKHLC | |
| •linker | PSPLFPGPSKPFWVLVVVGGVLACYSLLVTVAFIIFW | |
| •adenovirus E3-19K ER | VGSSGGSSGSSGGGSSGSGSSGNSSGGSGSTGSS | |
| retention tag | GGSGGSGSSGGSGESLFKGPRDYNPISSTICHLTNE | |
| | SDGHTTSLYGIGFGPFIITNKHLFRRNNGTLLVQSLHG | |
| | VFKVKNTTTLQQHLIDGRDMIIIRMPKDFPPFPQKLKE | |
| | REPQREERICLVTTNFQTAAASTGSSGGGGGSGGLY | |
| | KYKSRRSFIDEKKMP | |
| # 447 Three-way protease | MVSKGEEVIKEFMRFKVRMEGSMNGHEFEIEGEGE | 61 |
| •tdTomato | GRPYEGTQTAKLKVTKGGPLPFAWDILSPQFMYGSK | |
| •Linker | AYVKHPADIPDYKKLSFPEGFKWERVMNFEDGGLVT | |
| •P2A | VTQDSSLQDGTLIYKVKMRGTNFPPDGPVMQKKTM | |
| •TCRß Leader | GWEASTERLYPRDGVLKGEIHQALKLKDGGHYLVEF | |
| •RQR8 Tag | KTIYMAKKPVQLPGYYYVDTKLDITSHNEDYTIVEQYE | |
| •CD28 Hinge | RSEGRHHLFLGHGTGSTGSGSSGTASSEDNNMAVIK | |
| •CD28 Tm | EFMRFKVRMEGSMNGHEFEIEGEGEGRPYEGTQTA | |
| •CD28 intracellular linker | KLKVTKGGPLPFAWDILSPQFMYGSKAYVKHPADIPD | |
| •cTEV protease | YKKLSFPEGFKWERVMNFEDGGLVTVTQDSSLQDG | |
| •linker | TLIYKVKMRGTNFPPDGPVMQKKTMGWEASTERLYP | |
| •adenovirus E3-19K ER | RDGVLKGEIHQALKLKDGGHYLVEFKTIYMAKKPVQL | |
| retention tag | PGYYYVDTKLDITSHNEDYTIVEQYERSEGRHHLFLY | |
| | GMDELYKNAGNSSIGATNFSLLKQAGDVEENPGPM | |
| | GTSLLCWMALCLLGADHADACPYSNPSLCSGGGGS | |
| | ELPTQGTFSNVSTNVSPAKPTTTACPYSNPSLCSGG | |
| | GGSIEVMYPPPYLDNEKSNGTIIHVKGKHLCPSPLFP | |
| | GPSKPFWVLVVVGGVLACYSLLVTVAFIIFWVGSSGG | |
| | SSGSSGGGSSGSGSSGNSSGGSGSTGSSGGSGGS | |
| | GSSGGSKSMSSMVSDTSCTFPSSDGIFWKHWIQTK | |
| | DGQCGSPLVSTRDGFIVGIHSASNFTNTNNYFTSVPK | |
| | NFMELLTNQEAQQWVSGWRLNADSVLWGGHKVFM | |
| | VKPEEPFQPVKEATQLMNAAASTGSSGGGGGSGGL | |
| | YKYKSRRSFIDEKKMP | |
| # 575 Three-way protease | MLLLVTSLLLCELPHPAFLLIPDIQMTQTTSSLSASLG | 62 |
| •GM-CSFR Leader | DRVTISCRASQDISKYLNWYQQKPDGTVKLLIYHTSR | |
| •CD19 scFv | LHSGVPSRFSGSGSGTDYSLTISNLEQEDIATYFCQQ | |
| •Linker | GNTLPYTFGGGTKLEITGSTSGSGKPGSGEGSTKGE | |
| •CD8α Hinge | VKLQESGPGLVAPSQSLSVTCTVSGVSLPDYGVSWI | |
| •CD8α Tm 41BB | RQPPRKGLEWLGVIWGSETTYYNSALKSRLTIIKDNS | |
| •CD3Z Linker | KSQVFLKMNSLQTDDTAIYYCAKHYYYGGSYAMDY | |
| •P2A | WGQGTSVTVSSAAATTTPAPRPPTPAPTIASQPLSLR | |
| •TCRß Leader | PEACRPAAGGAVHTRGLDFACDIYIWAPLAGTCGVLL | |
| •RQR8 Tag | LSLVITLYCKRGRKKLLYIFKQPFMRPVQTTQEEDGC | |
| •CD28 Hinge | SCRFPEEEEGGCELRVKFSRSADAPAYKQGQNQLY | |
| •CD28 Tm | NELNLGRREEYDVLDKRRGRDPEMGGKPRRKNPQE | |
| •CD28 intracellular linker | GLYNELQKDKMAEAYSEIGMKGERRRGKGHDGLYQ | |
| •nTEV protease | GLSTATKDTYDALHMQALPPRNASATNFSLLKQAGD | |
| •linker | VEENPGPMGTSLLCWMALCLLGADHADACPYSNPS | |
| •adenovirus E3-19K ER | LCSGGGGSELPTQGTFSNVSTNVSPAKPTTTACPYS | |
| retention tag | NPSLCSGGGGSIEVMYPPPYLDNEKSNGTIIHVKGKH | |
| | LCPSPLFPGPSKPFWVLVVVGGVLACYSLLVTVAFIIF | |
| | WVGSSGGSSGSSGGGSSGSGSSGNSSGGSGSTGS | |
| | SGGSGGSGSSGGSGESLFKGPRDYNPISSTICHLTN | |
| | ESDGHTTSLYGIGFGPFIITNKHLFRRNNGTLLVQSLH | |
| | GVFKVKNTTTLQQHLIDGRDMIIIRMPKDFPPFPQKLK | |
| | FREPQREERICLVTTNFQTAAASTGSSGGGGGSGGL | |
| | YKYKSRRSFIDEKKMP | |
| # 576 Three-way protease | MARSVTLVFLVLVSLTGLYAADIQMTQSPSSLSASVG | 63 |
| •B2M Leader | DRVTITCRASQDVNTAVAWYQQKPGKAPKLLIYSASF | |
| •HER2 scFv | LESGVPSRFSGSRSGTDFTLTISSLQPEDFATYYCQQ | |
| •Linker | HYTTPPTFGQGTKVEIKGSTSGSGKPGSGEGSGEVQ | |
| •CD8α Hinge | LVESGGGLVQPGGSLRLSCAASGFNIKDTYIHWVRQ | |
| •CD8α Tm | APGKGLEWVARIYPTNGYTRYADSVKGRFTISADTSK | |
| •41BB | NTAYLQMNSLRAEDTAVYYCSRWGGDGFYAMDVW | |
| •CD3ζ | GQGTLVTVSSAAATTTPAPRPPTPAPTIASQPLSLRP | |
| •Linker | EACRPAAGGAVHTRGLDFACDIYIWAPLAGTCGVLLL | |
| •P2A | SLVITLYCKRGRKKLLYIFKQPFMRPVQTTQEEDGCS | |
| •TCRß Leader | CRFPEEEEGGCELRVKFSRSADAPAYKQGQNQLYN | |
| •RQR8 Tag | ELNLGRREEYDVLDKRRGRDPEMGGKPRRKNPQEG | |
| •CD28 Hinge | LYNELQKDKMAEAYSEIGMKGERRRGKGHDGLYQG | |
| •CD28 Tm | LSTATKDTYDALHMQALPPRSIGATNESLLKQAGDVE | |
| •CD28 intracellular linker | ENPGPMGTSLLCWMALCLLGADHADACPYSNPSLC | |
| •cTEV protease | SGGGGSELPTQGTFSNVSTNVSPAKPTTTACPYSNP | |
| •linker | SLCSGGGGSIEVMYPPPYLDNEKSNGTIIHVKGKHLC | |
| •adenovirus E3-19K ER | PSPLFPGPSKPFWVLVVVGGVLACYSLLVTVAFIIFW | |
| retention tag | VGSSGGSSGSSGGGSSGSGSSGNSSGGSGSTGSS | |
| | GGSGGSGSSGGSKSMSSMVSDTSCTFPSSDGIFWK | |
| | HWIQTKDGQCGSPLVSTRDGFIVGIHSASNFTNTNNY | |
| | FTSVPKNFMELLTNQEAQQWVSGWRLNADSVLWG | |
| | GHKVFMVKPEEPFQPVKEATQLMNAAASTGSSGGG | |
| | GGSGGLYKYKSRRSFIDEKKMP | |
| # 534 Two-way protease | MVSKGEELIKENMHMKLYMEGTVDNHHFKCTSEGE | 64 |
| •mTagBFP2 | GKPYEGTQTMRIKVVEGGPLPFAFDILATSFLYGSKT | |
| •Linker | FINHTQGIPDFFKQSFPEGFTWERVTTYEDGGVLTAT | |
| •P2A | QDTSLQDGCLIYNVKIRGVNFTSNGPVMQKKTLGWE | |
| •Linker | AFTETLYPADGGLEGRNDMALKLVGGSHLIANAKTTY | |
| •TCRβ Leader | RSKKPAKNLKMPGVYYVDYRLERIKEANNETYVEQH | |
| •Linker | EVAVARYCDLPSKLGHKLNNASATNFSLLKQAGDVE | |
| •RQR8 Tag | ENPGPSIGMGTSLLCWMALCLLGADHADGNSACPY | |
| •CISD2 Tm | SNPSLCSGGGGSELPTQGTFSNVSTNVSPAKPTTTA | |
| •Linker | CPYSNPSLCSGGGGSGGSWLRLLPFLGVLALLGYLA | |
| •TEV protease | VRPFLAAAGSSGGSSGGSGSTGSSGGSGGSGSSG | |
| •linker | GSGESLFKGPRDYNPISSTICHLTNESDGHTTSLYGI | |
| •adenovirus E3-19K ER | GFGPFIITNKHLFRRNNGTLLVQSLHGVFKVKNTTTL | |
| retention tag | QQHLIDGRDMIIIRMPKDFPPFPQKLKFREPQREERIC | |
| | LVTTNFQTKSMSSMVSDTSCTFPSSDGIFWKHWIQT | |
| | KDGQCGSPLVSTRDGFIVGIHSASNFTNTNNYFTSVP | |
| | KNFMELLTNQEAQQWVSGWRLNADSVLWGGHKVF | |
| | MVKPEEPFQPVKEATQLMNGSSGGGGGSGGNASP | |
| | KKKQQKDSLINLKIQKENPKVVNEINIEDLCLTKAAYC | |
| | RCWRSKTFPACDGSHNKHNELTGDNVGPLILKKKEV | |
| # 531 Two-way protease | MVSKGEELIKENMHMKLYMEGTVDNHHFKCTSEGE | 65 |
| •mTagBFP2 | GKPYEGTQTMRIKVVEGGPLPFAFDILATSFLYGSKT | |
| •Linker | FINHTQGIPDFFKQSFPEGFTWERVTTYEDGGVLTAT | |
| •P2A | QDTSLQDGCLIYNVKIRGVNFTSNGPVMQKKTLGWE | |
| •TCRß Leader | AFTETLYPADGGLEGRNDMALKLVGGSHLIANAKTTY | |
| •RQR8 Tag | RSKKPAKNLKMPGVYYVDYRLERIKEANNETYVEQH | |
| •CD8α Hinge | EVAVARYCDLPSKLGHKLNNASATNFSLLKQAGDVE | |
| •CD8α Tm | ENPGPMGTSLLCWMALCLLGADHADACPYSNPSLC | |
| •CD8α intracellular linker | SGGGGSELPTQGTFSNVSTNVSPAKPTTTACPYSNP | |
| •Human enterokinase light | SLCSGGGGSPAPRPPTPAPTIASQPLSLRPEACRPA | |
| chain | AGGAVHTRGLDFACDIYIWAPLAGTCGVLLLSLVITLY | |
| •linker | CNHRNRRRVCKCPRPVVGSSGNSSGGSGSTGSSG | |
| •adenovirus E3-19K ER | GSGGSGSSGGSIVGGSNAKEGAWPWVVGLYYGGR | |
| retention tag | LLCGASLVSSDWLVSAAHCVYGRNLEPSKWTAILGL | |
| | HMKSNLTSPQTVPRLIDEIVINPHYNRRRKDNDIAMM | |
| | HLEFKVNYTDYIQPICLPEENQVFPPGRNCSIAGWGT | |
| | VVYQGTTANILQEADVPLLSNERCQQQMPEYNITEN | |
| | MICAGYEEGGIDSCQGDSGGPLMCQENNRWFLAGV | |
| | TSFGYKCALPNRPGVYARVSRFTEWIQSFLHAAAST | |
| | GSSGGGGGSGGLYKYKSRRSFIDEKKMP | |
| # 405 STASHed myc with HCV NS3 | MLLLVTSLLLCELPHPAFLLIPGGSEQKLISEEDLTTTP | 66 |
| cleavage site | APRPPTPAPTIASQPLSLRPEACRPAAGGAVHTRGL | |
| •GM-CSFR Leader | DFACDIYIWAPLAGTCGVLLLSLVITLYCAAAGSVSKG | |
| •Linker | EEDNMASLPATHELHIFGSINGVDFDMVGQGTGNPN | |
| •Myc Tag | DGYEELNLKSTKGDLQFSPWILVPHIGYGFHQYLPYP | |
| •CD8α Hinge | DGMSPFQAAMVDGSGYQVHRTMQFEDGASLTVNY | |
| •CD8α Tm | RYTYEGSHIKGEAQVKGTGFPADGPVMTNSLTAAD | |
| •Linker | WCRSKKTYPNDKTIISTFKWSYTTGNGKRYRSTART | |
| •mNeonGreen | TYTFAKPMAANYLKNQPMYVFRKTELKHSKTELNFK | |
| •Linker | EWQKAFTDVMGMDELYKSIGGGSGGSDEMEECSQ | |
| •HCV NS3 4a4b cleavage site | HGSTGGSGGSLYKYKSRRSFIDEKKMP | |
| •Linker | | |
| •adenovirus E3-19K ER | | |
| retention tag | | |
| # 408 STASHed myc with TEV | MLLLVTSLLLCELPHPAFLLIPGGSEQKLISEEDLTTTP | 67 |
| cleavage site | APRPPTPAPTIASQPLSLRPEACRPAAGGAVHTRGL | |
| •GM-CSFR Leader | DFACDIYIWAPLAGTCGVLLLSLVITLYCAAAGSVSKG | |
| •Linker | EEDNMASLPATHELHIFGSINGVDFDMVGQGTGNPN | |
| •Myc Tag | DGYEELNLKSTKGDLQFSPWILVPHIGYGFHQYLPYP | |
| •CD8α Hinge | DGMSPFQAAMVDGSGYQVHRTMQFEDGASLTVNY | |
| •CD8α Tm | RYTYEGSHIKGEAQVKGTGFPADGPVMTNSLTAAD | |
| •Linker | WCRSKKTYPNDKTIISTFKWSYTTGNGKRYRSTART | |
| •mNeonGreen | TYTFAKPMAANYLKNQPMYVFRKTELKHSKTELNEK | |
| •Linker | EWQKAFTDVMGMDELYKSIGGGSGGSTENLYFQSG | |
| •TEV cleavage site | STGGSGGSLYKYKSRRSFIDEKKMP | |
| •Linker | | |
| •adenovirus E3-19K ER | | |
| retention tag | | |
| # 409 STASHed NGFRt with TEV | MGAGATGRAMDGPRLLLLLLLGVSLGGAKEACPTGL | 68 |
| cleavage site | YTHSGECCKACNLGEGVAQPCGANQTVCEPCLDSV | |
| •NGFRt | TFSDVVSATEPCKPCTECVGLQSMSAPCVEADDAVC | |
| •Linker | RCAYGYYQDETTGRCEACRVCEAGSGLVFSCQDKQ | |
| •mNeonGreen | NTVCEECPDGTYSDEANHVDPCLPCTVCEDTERQLR | |
| •Linker | ECTRWADAECEEIPGRWITRSTPPEGSDSTAPSTQE | |
| •TEV cleavage site | PEAPPEQDLIASTVAGVVTTVMGSSQPVVTRGTTDN | |
| •Linker | LIPVYCSILAAVVVGLVAYIAFKRAAAGSVSKGEEDNM | |
| •adenovirus E3-19K ER | ASLPATHELHIFGSINGVDFDMVGQGTGNPNDGYEE | |
| retention tag | LNLKSTKGDLQFSPWILVPHIGYGFHQYLPYPDGMSP | |
| | FQAAMVDGSGYQVHRTMQFEDGASLTVNYRYTYEG | |
| | SHIKGEAQVKGTGFPADGPVMTNSLTAADWCRSKKT | |
| | YPNDKTIISTFKWSYTTGNGKRYRSTARTTYTFAKPM | |
| | AANYLKNQPMYVFRKTELKHSKTELNFKEWQKAFTD | |
| | VMGMDELYKSIGGGSGGSTENLYFQSGSTGGSGGS | |
| | LYKYKSRRSFIDEKKMP | |
| # 407 STASHed NGFRt with HCV NS3 | MGAGATGRAMDGPRLLLLLLLGVSLGGAKEACPTGL | 69 |
| cleavage site | YTHSGECCKACNLGEGVAQPCGANQTVCEPCLDSV | |
| •NGFRt | TFSDVVSATEPCKPCTECVGLQSMSAPCVEADDAVC | |
| •Linker | RCAYGYYQDETTGRCEACRVCEAGSGLVFSCQDKQ | |
| •mNeonGreen | NTVCEECPDGTYSDEANHVDPCLPCTVCEDTERQLR | |
| •Linker | ECTRWADAECEEIPGRWITRSTPPEGSDSTAPSTQE | |
| •HCV NS3 4a4b cleavage site | PEAPPEQDLIASTVAGVVTTVMGSSQPVVTRGTTDN | |
| •Linker | LIPVYCSILAAVVVGLVAYIAFKRAAAGSVSKGEEDNM | |
| •adenovirus E3-19K ER | ASLPATHELHIFGSINGVDFDMVGQGTGNPNDGYEE | |
| retention tag | LNLKSTKGDLQFSPWILVPHIGYGFHQYLPYPDGMSP | |
| | FQAAMVDGSGYQVHRTMQFEDGASLTVNYRYTYEG | |
| | SHIKGEAQVKGTGFPADGPVMTNSLTAADWCRSKKT | |
| | YPNDKTIISTFKWSYTTGNGKRYRSTARTTYTFAKPM | |
| | AANYLKNQPMYVFRKTELKHSKTELNFKEWQKAFTD | |
| | VMGMDELYKSIGGGSGGSDEMEECSQHGSTGGSG | |
| | GSLYKYKSRRSFIDEKKMP | |
| # 437 STASHed RQR8 with CD34 | MLLLVTSLLLCELPHPAFLLIPGGSACPYSNPSLCSG | 70 |
| epitope and TEV cleavage site | GGGSELPTQGTFSNVSTNVSPAKPTTTACPYSNPSL | |
| •GM-CSFR Leader | CSGGGGSPAPRPPTPAPTIASQPLSLRPEACRPAAG | |
| •Linker | GAVHTRGLDFACDIYIWAPLAGTCGVLLLSLVITLYCA | |
| •RQR8 | AAGSVSKGEEDNMASLPATHELHIFGSINGVDFDMV | |
| •CD8α Hinge | GQGTGNPNDGYEELNLKSTKGDLQFSPWILVPHIGY | |
| •CD8α Tm | GFHQYLPYPDGMSPFQAAMVDGSGYQVHRTMQFE | |
| •Linker | DGASLTVNYRYTYEGSHIKGEAQVKGTGFPADGPVM | |
| •mNeonGreen | TNSLTAADWCRSKKTYPNDKTIISTFKWSYTTGNGKR | |
| •Linker | YRSTARTTYTFAKPMAANYLKNQPMYVFRKTELKHS | |
| •TEV cleavage site | KTELNFKEWQKAFTDVMGMDELYKSIGGGSGGSTE | |
| •Linker | NLYFQSGSTGGSGGSLYKYKSRRSFIDEKKMP | |
| •adenovirus E3-19K ER | | |
| retention tag | | |
| # 438 STASHed NGFR ECD on CD8 | MGAGATGRAMDGPRLLLLLLLGVSLGGAKEACPTGL | 71 |
| H/Tm with TEV cleavage site | YTHSGECCKACNLGEGVAQPCGANQTVCEPCLDSV | |
| •NGFR ECD | TFSDVVSATEPCKPCTECVGLQSMSAPCVEADDAVC | |
| •Linker | RCAYGYYQDETTGRCEACRVCEAGSGLVFSCQDKQ | |
| •Myc Tag | NTVCEECPDGTYSDEANHVDPCLPCTVCEDTERQLR | |
| •CD8α Hinge | ECTRWADAECEEIPGRWITRSTPPEGSDSTAPSTQE | |
| •CD8α Tm | PEAPPEQDLIASTVAGVVTTVMGSSQPVVTRGTTDN | |
| •Linker | GGSEQKLISEEDLTTTPAPRPPTPAPTIASQPLSLRPE | |
| •mNeonGreen | ACRPAAGGAVHTRGLDFACDIYIWAPLAGTCGVLLLS | |
| •Linker | LVITLYCAAAGSVSKGEEDNMASLPATHELHIFGSING | |
| •TEV cleavage site | VDFDMVGQGTGNPNDGYEELNLKSTKGDLQFSPWIL | |
| •Linker | VPHIGYGFHQYLPYPDGMSPFQAAMVDGSGYQVHR | |
| •adenovirus E3-19K ER | TMQFEDGASLTVNYRYTYEGSHIKGEAQVKGTGFPA | |
| retention tag | DGPVMTNSLTAADWCRSKKTYPNDKTIISTFKWSYTT | |
| | GNGKRYRSTARTTYTFAKPMAANYLKNQPMYVFRKT | |
| | ELKHSKTELNFKEWQKAFTDVMGMDELYKSIGGGSG | |
| | GSTENLYFQSGSTGGSGGSLYKYKSRRSFIDEKKMP | |
| # 448 STASHed myc on CD28 H/Tm | MLLLVTSLLLCELPHPAFLLIPGGSEQKLISEEDLIEVM | 72 |
| with TEV cleavage site | YPPPYLDNEKSNGTIIHVKGKHLCPSPLFPGPSKPFW | |
| •GM-CSFR Leader | VLVVVGGVLACYSLLVTVAFIIFWVAAAGSVSKGEED | |
| •Linker | NMASLPATHELHIFGSINGVDFDMVGQGTGNPNDGY | |
| •Myc Tag | EELNLKSTKGDLQFSPWILVPHIGYGFHQYLPYPDGM | |
| •CD28 Hinge | SPFQAAMVDGSGYQVHRTMQFEDGASLTVNYRYTY | |
| •CD28 Tm | EGSHIKGEAQVKGTGFPADGPVMTNSLTAADWCRS | |
| •Linker | KKTYPNDKTIISTFKWSYTTGNGKRYRSTARTTYTFA | |
| •mNeonGreen | KPMAANYLKNQPMYVFRKTELKHSKTELNFKEWQKA | |
| •Linker | FTDVMGMDELYKSIGGGSGGSTENLYFQSGSTGGS | |
| •TEV cleavage site | GGSLYKYKSRRSFIDEKKMP | |
| •Linker | | |
| •adenovirus E3-19K ER | | |
| retention tag | | |
| # 449 STASHed RQR8 with CD34 | MLLLVTSLLLCELPHPAFLLIPGGSACPYSNPSLCSG | 73 |
| epitope on CD28 H/Tm and TEV | GGGSELPTQGTFSNVSTNVSPAKPTTTACPYSNPSL | |
| cleavage site | CSGGGGSIEVMYPPPYLDNEKSNGTIIHVKGKHLCPS | |
| •GM-CSFR Leader | PLFPGPSKPFWVLVVVGGVLACYSLLVTVAFIIFWVA | |
| •Linker | AAGSVSKGEEDNMASLPATHELHIFGSINGVDFDMV | |
| •RQR8 | GQGTGNPNDGYEELNLKSTKGDLQFSPWILVPHIGY | |
| •CD28 Hinge | GFHQYLPYPDGMSPFQAAMVDGSGYQVHRTMQFE | |
| •CD28 Tm | DGASLTVNYRYTYEGSHIKGEAQVKGTGFPADGPVM | |
| •Linker | TNSLTAADWCRSKKTYPNDKTIISTFKWSYTTGNGKR | |
| •mNeonGreen | YRSTARTTYTFAKPMAANYLKNQPMYVFRKTELKHS | |
| •Linker | KTELNFKEWQKAFTDVMGMDELYKSIGGGSGGSTE | |
| •TEV cleavage site | NLYFQSGSTGGSGGSLYKYKSRRSFIDEKKMP | |
| •Linker | | |
| •adenovirus E3-19K ER | | |
| retention tag | | |
| # 450 STASHed NGFR ECD on CD28 | MGAGATGRAMDGPRLLLLLLLGVSLGGAKEACPTGL | 74 |
| H/Tm with TEV cleavage site | YTHSGECCKACNLGEGVAQPCGANQTVCEPCLDSV | |
| •NGFR ECD | TFSDVVSATEPCKPCTECVGLQSMSAPCVEADDAVC | |
| •Linker | RCAYGYYQDETTGRCEACRVCEAGSGLVFSCQDKQ | |
| •Myc Tag | NTVCEECPDGTYSDEANHVDPCLPCTVCEDTERQLR | |
| •CD28 Hinge | ECTRWADAECEEIPGRWITRSTPPEGSDSTAPSTQE | |
| •CD28 Tm | PEAPPEQDLIASTVAGVVTTVMGSSQPVVTRGTTDN | |
| •Linker | GGSEQKLISEEDLIEVMYPPPYLDNEKSNGTIIHVKGK | |
| •mNeonGreen | HLCPSPLFPGPSKPFWVLVVVGGVLACYSLLVTVAFII | |
| •Linker | FWVAAAGSVSKGEEDNMASLPATHELHIFGSINGVD | |
| •TEV cleavage site | FDMVGQGTGNPNDGYEELNLKSTKGDLQFSPWILVP | |
| •Linker | HIGYGFHQYLPYPDGMSPFQAAMVDGSGYQVHRTM | |
| E3-19K ER | QFEDGASLTVNYRYTYEGSHIKGEAQVKGTGFPADG | |
| •adenovirus | PVMTNSLTAADWCRSKKTYPNDKTIISTFKWSYTTGN | |
| retention tag | GKRYRSTARTTYTFAKPMAANYLKNQPMYVFRKTEL | |
| | KHSKTELNFKEWQKAFTDVMGMDELYKSIGGGSGG | |
| | STENLYFQSGSTGGSGGSLYKYKSRRSFIDEKKMP | |
| # 451 Protease recruiting Tm | MLLLVTSLLLCELPHPAFLLIPGGSDYKDDDDKGGTT | 75 |
| protein with SYNZIP 1 leucine | TPAPRPPTPAPTIASQPLSLRPEACRPAAGGAVHTR | |
| zipper | GLDFACDIYIWAPLAGTCGVLLLSLVITLYCKGGGGS | |
| •GM-CSFR Leader | GGAAAGSNLVAQLENEVASLENENETLKKKNLHKKD | |
| •Linker | LIAYLEKEIANLRKKIEEGGSGSSGSTGGSGGSLYKY | |
| •FLAG Tag | KSRRSFIDEKKMP | |
| •Linker | | |
| •CD8α Hinge | | |
| •CD8α Tm | | |
| •Linker | | |
| •SYNZIP1 | | |
| •Linker | | |
| •adenovirus E3-19K ER | | |
| retention tag | | |
| # 452 Protease recruiting Tm | MLLLVTSLLLCELPHPAFLLIPGGSYPYDVPDYAGGTT | 76 |
| protein with SYNZIP 3 leucine | TPAPRPPTPAPTIASQPLSLRPEACRPAAGGAVHTR | |
| zipper | GLDFACDIYIWAPLAGTCGVLLLSLVITLYCKGGGGS | |
| •GM-CSFR Leader | GGAAAGSNEVTTLENDAAFIENENAYLEKEIARLRKE | |
| •Linker | KAALRNRLAHKKGGSGSSGSTGGSGGSLYKYKSRR | |
| •HA Tag | SFIDEKKMP | |
| •Linker | | |
| •CD8α Hinge | | |
| •CD8α Tm | | |
| •Linker | | |
| •SYNZIP3 | | |
| •Linker | | |
| •adenovirus E3-19K ER | | |
| retention tag | | |
| # 453 mTagBFP2 with SYNZIP2 AND | MVSKGEELIKENMHMKLYMEGTVDNHHFKCTSEGE | 77 |
| nTEV | GKPYEGTQTMRIKVVEGGPLPFAFDILATSFLYGSKT | |
| •mTagBFP2 | FINHTQGIPDFFKQSFPEGFTWERVTTYEDGGVLTAT | |
| •Linker | QDTSLQDGCLIYNVKIRGVNFTSNGPVMQKKTLGWE | |
| •P2A | AFTETLYPADGGLEGRNDMALKLVGGSHLIANAKTTY | |
| •Linker | RSKKPAKNLKMPGVYYVDYRLERIKEANNETYVEQH | |
| •SYNZIP2 | EVAVARYCDLPSKLGHKLNNASATNFSLLKQAGDVE | |
| •Linker | ENPGPGGSARNAYLRKKIARLKKDNLQLERDEQNLE | |
| •nTEV protease | KIIANLRDEIARLENEVASHEQGSTGSSGGSGGSGSS | |
| •linker | GGSGESLFKGPRDYNPISSTICHLTNESDGHTTSLYG | |
| | IGFGPFIITNKHLFRRNNGTLLVQSLHGVFKVKNTTTL | |
| | QQHLIDGRDMIIIRMPKDFPPFPQKLKFREPQREERIC | |
| | LVTTNFQTAAA | |
| # 455 tdTomato with SYNZIP4 AND | MVSKGEEVIKEFMRFKVRMEGSMNGHEFEIEGEGE | 78 |
| cTEV | GRPYEGTQTAKLKVTKGGPLPFAWDILSPQFMYGSK | |
| •tdTomato | AYVKHPADIPDYKKLSFPEGFKWERVMNFEDGGLVT | |
| •Linker | VTQDSSLQDGTLIYKVKMRGTNFPPDGPVMQKKTM | |
| •P2A | GWEASTERLYPRDGVLKGEIHQALKLKDGGHYLVEF | |
| •Linker | KTIYMAKKPVQLPGYYYVDTKLDITSHNEDYTIVEQYE | |
| •SYNZIP4 | RSEGRHHLFLGHGTGSTGSGSSGTASSEDNNMAVIK | |
| •Linker | EFMRFKVRMEGSMNGHEFEIEGEGEGRPYEGTQTA | |
| •cTEV protease | KLKVTKGGPLPFAWDILSPQFMYGSKAYVKHPADIPD | |
| •linker | YKKLSFPEGFKWERVMNFEDGGLVTVTQDSSLQDG | |
| | TLIYKVKMRGTNFPPDGPVMQKKTMGWEASTERLYP | |
| | RDGVLKGEIHQALKLKDGGHYLVEFKTIYMAKKPVQL | |
| | PGYYYVDTKLDITSHNEDYTIVEQYERSEGRHHLFLY | |
| | GMDELYKNAGNSGGGATNFSLLKQAGDVEENPGPG | |
| | GSQKVAELKNRVAVKLNRNEQLKNKVEELKNRNAYL | |
| | KNELATLENEVARLENDVAEGSTGSSGGSGGSGSS | |
| | GGSKSMSSMVSDTSCTFPSSDGIFWKHWIQTKDGQ | |
| | CGSPLVSTRDGFIVGIHSASNFTNTNNYFTSVPKNFM | |
| | ELLTNQEAQQWVSGWRLNADSVLWGGHKVFMVKP | |
| | EEPFQPVKEATQLMNAAA | |
| # 464 STASHed EGFRt with | MLLLVTSLLLCELPHPAFLLIPRKVCNGIGIGEFKDSLS | 79 |
| mNeonGreen and TEV cleavage site | INATNIKHFKNCTSISGDLHILPVAFRGDSFTHTPPLDP | |
| •GM-CSFR Leader | QELDILKTVKEITGFLLIQAWPENRTDLHAFENLEIIRG | |
| •EGFR ECD | RTKQHGQFSLAVVSLNITSLGLRSLKEISDGDVIISGN | |
| •EGFR Tm | KNLCYANTINWKKLFGTSGQKTKIISNRGENSCKATG | |
| •Linker | QVCHALCSPEGCWGPEPRDCVSCRNVSRGRECVD | |
| •mNeonGreen | KCNLLEGEPREFVENSECIQCHPECLPQAMNITCTG | |
| •Linker | RGPDNCIQCAHYIDGPHCVKTCPAGVMGENNTLVW | |
| •TEV cleavage site | KYADAGHVCHLCHPNCTYGCTGPGLEGCPTNGPKIP | |
| •Linker | SIATGMVGALLLLLVVALGIGLFMAAAGSVSKGEEDN | |
| •adenovirus E3-19K ER | MASLPATHELHIFGSINGVDFDMVGQGTGNPNDGYE | |
| retention tag | ELNLKSTKGDLQFSPWILVPHIGYGFHQYLPYPDGMS | |
| | PFQAAMVDGSGYQVHRTMQFEDGASLTVNYRYTYE | |
| | GSHIKGEAQVKGTGFPADGPVMTNSLTAADWCRSK | |
| | KTYPNDKTIISTFKWSYTTGNGKRYRSTARTTYTFAK | |
| | PMAANYLKNQPMYVFRKTELKHSKTELNFKEWQKAF | |
| | TDVMGMDELYKSIGGGSGGSTENLYFQSGSTGGSG | |
| | GSLYKYKSRRSFIDEKKMP | |
| # 467 STASHed EGFR ECD on CD8 | MLLLVTSLLLCELPHPAFLLIPRKVCNGIGIGEFKDSLS | 80 |
| H/Tm with TEV cleavage site | INATNIKHFKNCTSISGDLHILPVAFRGDSFTHTPPLDP | |
| •GM-CSFR Leader | QELDILKTVKEITGFLLIQAWPENRTDLHAFENLEIIRG | |
| •EGFR ECD | RTKQHGQFSLAVVSLNITSLGLRSLKEISDGDVIISGN | |
| •Linker | KNLCYANTINWKKLFGTSGQKTKIISNRGENSCKATG | |
| •Myc Tag | QVCHALCSPEGCWGPEPRDCVSCRNVSRGRECVD | |
| •CD8α Hinge | KCNLLEGEPREFVENSECIQCHPECLPQAMNITCTG | |
| •CD8α Tm | RGPDNCIQCAHYIDGPHCVKTCPAGVMGENNTLVW | |
| •Linker | KYADAGHVCHLCHPNCTYGCTGPGLEGCPTNGPKIP | |
| •TEV cleavage site | SGGSEQKLISEEDLTTTPAPRPPTPAPTIASQPLSLRP | |
| •Linker | EACRPAAGGAVHTRGLDFACDIYIWAPLAGTCGVLLL | |
| •adenovirus E3-19K ER | SLVITLYCAAAGSIGGGSGGSTENLYFQSGSTGGSG | |
| retention tag | GSLYKYKSRRSFIDEKKMP | |
| # 487 mNeonGreen-P2A-EGFR ECD | MVSKGEEDNMASLPATHELHIFGSINGVDFDMVGQG | 81 |
| with CD8 H/Tm and adenovirus | TGNPNDGYEELNLKSTKGDLQFSPWILVPHIGYGFH | |
| E3-19K ER variant 1 | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVFRKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •Myc Tag | RTDLHAFENLEIIRGRTKQHGQFSLAVVSLNITSLGLR | |
| •CD8α Hinge | SLKEISDGDVIISGNKNLCYANTINWKKLFGTSGQKTK | |
| •CD8α TM | IISNRGENSCKATGQVCHALCSPEGCWGPEPRDCVS | |
| •Linker | CRNVSRGRECVDKCNLLEGEPREFVENSECIQCHPE | |
| •TEV cleavage site | CLPQAMNITCTGRGPDNCIQCAHYIDGPHCVKTCPA | |
| •Linker | GVMGENNTLVWKYADAGHVCHLCHPNCTYGCTGP | |
| •adenovirus E3-19K ER variant | GLEGCPTNGPKIPSGGSEQKLISEEDLTTTPAPRPPT | |
| 1 | PAPTIASQPLSLRPEACRPAAGGAVHTRGLDFACDIYI | |
| | WAPLAGTCGVLLLSLVITLYCAAAGGTENLYFQSGST | |
| | LYKYKSRRSFIEEKKMP | |
| # 488 mNeonGreen-P2A-EGFR ECD | MVSKGEEDNMASLPATHELHIFGSINGVDFDMVGQG | 82 |
| with CD8 H/Tm and adenovirus | TGNPNDGYEELNLKSTKGDLQFSPWILVPHIGYGFH | |
| E3-19K ER variant 2 | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVERKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •Myc Tag | RTDLHAFENLEIIRGRTKQHGQFSLAVVSLNITSLGLR | |
| •CD8α Hinge | SLKEISDGDVIISGNKNLCYANTINWKKLFGTSGQKTK | |
| •CD8α TM | IISNRGENSCKATGQVCHALCSPEGCWGPEPRDCVS | |
| •Linker | CRNVSRGRECVDKCNLLEGEPREFVENSECIQCHPE | |
| •TEV cleavage site | CLPQAMNITCTGRGPDNCIQCAHYIDGPHCVKTCPA | |
| •Linker | GVMGENNTLVWKYADAGHVCHLCHPNCTYGCTGP | |
| •adenovirus E3-19K ER variant | GLEGCPTNGPKIPSGGSEQKLISEEDLTTTPAPRPPT | |
| 2 | PAPTIASQPLSLRPEACRPAAGGAVHTRGLDFACDIYI | |
| | WAPLAGTCGVLLLSLVITLYCAAAGGTENLYFQSGST | |
| | LYKYKSRRSFIEEKKYN | |
| # 489 mNeonGreen-P2A-EGFR ECD | MVSKGEEDNMASLPATHELHIFGSINGVDFDMVGQG | 83 |
| with CD8 H/Tm and adenovirus | TGNPNDGYEELNLKSTKGDLQFSPWILVPHIGYGFH | |
| E3-19K ER variant 3 | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVERKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •Myc Tag | RTDLHAFENLEIIRGRTKQHGQFSLAVVSLNITSLGLR | |
| •CD8α Hinge | SLKEISDGDVIISGNKNLCYANTINWKKLFGTSGQKTK | |
| •CD8α TM | IISNRGENSCKATGQVCHALCSPEGCWGPEPRDCVS | |
| •Linker | CRNVSRGRECVDKCNLLEGEPREFVENSECIQCHPE | |
| •TEV cleavage site | CLPQAMNITCTGRGPDNCIQCAHYIDGPHCVKTCPA | |
| •Linker | GVMGENNTLVWKYADAGHVCHLCHPNCTYGCTGP | |
| •adenovirus E3-19K ER variant | GLEGCPTNGPKIPSGGSEQKLISEEDLTTTPAPRPPT | |
| 3 | PAPTIASQPLSLRPEACRPAAGGAVHTRGLDFACDIYI | |
| | WAPLAGTCGVLLLSLVITLYCAAAGGTENLYFQSGST | |
| | LYKYKSRRSFIEEKKYL | |
| # 490 mNeonGreen-P2A-EGFR ECD | MVSKGEEDNMASLPATHELHIFGSINGVDFDMVGQG | 84 |
| with CD8 H/Tm and adenovirus | TGNPNDGYEELNLKSTKGDLQFSPWILVPHIGYGFH | |
| E3-19K ER variant 4 | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVERKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •Myc Tag | RTDLHAFENLEIIRGRTKQHGQFSLAVVSLNITSLGLR | |
| •CD8α Hinge | SLKEISDGDVIISGNKNLCYANTINWKKLFGTSGQKTK | |
| •CD8α TM | IISNRGENSCKATGQVCHALCSPEGCWGPEPRDCVS | |
| •Linker | CRNVSRGRECVDKCNLLEGEPREFVENSECIQCHPE | |
| •TEV cleavage site | CLPQAMNITCTGRGPDNCIQCAHYIDGPHCVKTCPA | |
| •Linker | GVMGENNTLVWKYADAGHVCHLCHPNCTYGCTGP | |
| •adenovirus E3-19K ER variant | GLEGCPTNGPKIPSGGSEQKLISEEDLTTTPAPRPPT | |
| 4 | PAPTIASQPLSLRPEACRPAAGGAVHTRGLDFACDIYI | |
| | WAPLAGTCGVLLLSLVITLYCAAAGGTENLYFQSGST | |
| | LYKYKSRRSFIEEFLKKTN | |
| # 491 mNeonGreen-P2A-EGFR ECD | MVSKGEEDNMASLPATHELHIFGSINGVDFDMVGQG | 85 |
| with CD8 H/Tm and retention | TGNPNDGYEELNLKSTKGDLQFSPWILVPHIGYGFH | |
| variant KDELR2 ICD | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVFRKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •Myc Tag | RTDLHAFENLEIIRGRTKQHGQFSLAVVSLNITSLGLR | |
| •CD8α Hinge | SLKEISDGDVIISGNKNLCYANTINWKKLFGTSGQKTK | |
| •CD8α TM | IISNRGENSCKATGQVCHALCSPEGCWGPEPRDCVS | |
| •Linker | CRNVSRGRECVDKCNLLEGEPREFVENSECIQCHPE | |
| •TEV cleavage site | CLPQAMNITCTGRGPDNCIQCAHYIDGPHCVKTCPA | |
| •Linker | GVMGENNTLVWKYADAGHVCHLCHPNCTYGCTGP | |
| •KDELR2 ICD | GLEGCPTNGPKIPSGGSEQKLISEEDLTTTPAPRPPT | |
| | PAPTIASQPLSLRPEACRPAAGGAVHTRGLDFACDIYI | |
| | WAPLAGTCGVLLLSLVITLYCAAAGGTENLYFQSGST | |
| | TKVLKGKKLSLPA | |
| # 492 mNeonGreen-P2A-EGFR ECD | MVSKGEEDNMASLPATHELHIFGSINGVDFDMVGQG | 86 |
| with CD8 H/Tm and retention | TGNPNDGYEELNLKSTKGDLQFSPWILVPHIGYGFH | |
| variant carboxypeptidase D ICD | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVFRKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •Myc Tag | RTDLHAFENLEIIRGRTKQHGQFSLAVVSLNITSLGLR | |
| •CD8α Hinge | SLKEISDGDVIISGNKNLCYANTINWKKLFGTSGQKTK | |
| •CD8α TM | IISNRGENSCKATGQVCHALCSPEGCWGPEPRDCVS | |
| •Linker | CRNVSRGRECVDKCNLLEGEPREFVENSECIQCHPE | |
| •TEV cleavage site | CLPQAMNITCTGRGPDNCIQCAHYIDGPHCVKTCPA | |
| •Linker | GVMGENNTLVWKYADAGHVCHLCHPNCTYGCTGP | |
| •carboxypeptidase D ICD | GLEGCPTNGPKIPSGGSEQKLISEEDLTTTPAPRPPT | |
| | PAPTIASQPLSLRPEACRPAAGGAVHTRGLDFACDIYI | |
| | WAPLAGTCGVLLLSLVITLYCAAAGGTENLYFQSGST | |
| | KSNRHKDGFHRLRGHHDEYEDEIRMMSTGSKKSLLS | |
| | HEFQDETDTEETLYSSKH | |
| # 493 mNeonGreen-P2A-EGFR ECD | MVSKGEEDNMASLPATHELHIFGSINGVDFDMVGQG | 87 |
| with CD8 H/Tm with degron and | TGNPNDGYEELNLKSTKGDLQFSPWILVPHIGYGFH | |
| adenovirus E3-19K ER retention | QYLPYPDGMSPFQAAMVDGSGYQVHRTMQFEDGA | |
| tag | SLTVNYRYTYEGSHIKGEAQVKGTGFPADGPVMTNS | |
| •mNeonGreen | LTAADWCRSKKTYPNDKTIISTFKWSYTTGNGKRYR | |
| •Linker | STARTTYTFAKPMAANYLKNQPMYVERKTELKHSKT | |
| •P2A | ELNFKEWQKAFTDVMGMDELYKNASGATNFSLLKQ | |
| •Linker | AGDVEENPGPIASMLLLVTSLLLCELPHPAFLLIPRKV | |
| •GM-CSFR Leader | CNGIGIGEFKDSLSINATNIKHFKNCTSISGDLHILPVA | |
| •EGFR ECD | FRGDSFTHTPPLDPQELDILKTVKEITGFLLIQAWPEN | |
| •Linker | RTDLHAFENLEIIRGRTKQHGQFSLAVVSLNITSLGLR | |
| •Myc Tag | SLKEISDGDVIISGNKNLCYANTINWKKLFGTSGQKTK | |
| •CD8α Hinge | IISNRGENSCKATGQVCHALCSPEGCWGPEPRDCVS | |
| •CD8α TM | CRNVSRGRECVDKCNLLEGEPREFVENSECIQCHPE | |
| •Linker | CLPQAMNITCTGRGPDNCIQCAHYIDGPHCVKTCPA | |
| •TEV cleavage site | GVMGENNTLVWKYADAGHVCHLCHPNCTYGCTGP | |
| •Linker | GLEGCPTNGPKIPSGGSEQKLISEEDLTTTPAPRPPT | |
| •Degron | PAPTIASQPLSLRPEACRPAAGGAVHTRGLDFACDIYI | |
| •adenovirus E3-19K ER | WAPLAGTCGVLLLSLVITLYCAAAGGTENLYFQSGST | |
| retention tag | PITKIDTKYIMTCMSADLEVVTSTWVLVGGVLAALAAY | |
| | CLSTLYKYKSRRSFIEEKKMP | |
| # 494 mNeonGreen-P2A-EGFR ECD | MVSKGEEDNMASLPATHELHIFGSINGVDFDMVGQG | 88 |
| with CD8 H/Tm and retention | TGNPNDGYEELNLKSTKGDLQFSPWILVPHIGYGFH | |
| variant Coronavirus IBV S | QYLPYPDGMSPFQAAMVDGSGYQVHRTMQFEDGA | |
| protein ER motif | SLTVNYRYTYEGSHIKGEAQVKGTGFPADGPVMTNS | |
| •mNeonGreen | LTAADWCRSKKTYPNDKTIISTFKWSYTTGNGKRYR | |
| •Linker | STARTTYTFAKPMAANYLKNQPMYVERKTELKHSKT | |
| •P2A | ELNFKEWQKAFTDVMGMDELYKNASGATNFSLLKQ | |
| •Linker | AGDVEENPGPIASMLLLVTSLLLCELPHPAFLLIPRKV | |
| •GM-CSFR Leader | CNGIGIGEFKDSLSINATNIKHFKNCTSISGDLHILPVA | |
| •EGFR ECD | FRGDSFTHTPPLDPQELDILKTVKEITGFLLIQAWPEN | |
| •Linker | RTDLHAFENLEIIRGRTKQHGQFSLAVVSLNITSLGLR | |
| •Myc Tag | SLKEISDGDVIISGNKNLCYANTINWKKLFGTSGQKTK | |
| •CD8α Hinge | IISNRGENSCKATGQVCHALCSPEGCWGPEPRDCVS | |
| •CD8α TM | CRNVSRGRECVDKCNLLEGEPREFVENSECIQCHPE | |
| •Linker | CLPQAMNITCTGRGPDNCIQCAHYIDGPHCVKTCPA | |
| •TEV cleavage site | GVMGENNTLVWKYADAGHVCHLCHPNCTYGCTGP | |
| •Linker | GLEGCPTNGPKIPSGGSEQKLISEEDLTTTPAPRPPT | |
| •Coronavirus IBV S protein ER | PAPTIASQPLSLRPEACRPAAGGAVHTRGLDFACDIYI | |
| motif | WAPLAGTCGVLLLSLVITLYCAAAGGTENLYFQSGST | |
| | KCGKKSSYYTTFDNDVVIEQYRPKKSV | |
| # 495 mNeonGreen-P2A-EGFR ECD | MVSKGEEDNMASLPATHELHIFGSINGVDFDMVGQG | 89 |
| with CD8 H/Tm and retention | TGNPNDGYEELNLKSTKGDLQFSPWILVPHIGYGFH | |
| variant HCV NS3 Helix | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVFRKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •Myc Tag | RTDLHAFENLEIIRGRTKQHGQFSLAVVSLNITSLGLR | |
| •CD8α Hinge | SLKEISDGDVIISGNKNLCYANTINWKKLFGTSGQKTK | |
| •CD8α TM | IISNRGENSCKATGQVCHALCSPEGCWGPEPRDCVS | |
| •Linker | CRNVSRGRECVDKCNLLEGEPREFVENSECIQCHPE | |
| •HCV NS3 Helix | CLPQAMNITCTGRGPDNCIQCAHYIDGPHCVKTCPA | |
| •Linker | GVMGENNTLVWKYADAGHVCHLCHPNCTYGCTGP | |
| •TEV cleavage site | GLEGCPTNGPKIPSGGSEQKLISEEDLTTTPAPRPPT | |
| •Linker | PAPTIASQPLSLRPEACRPAAGGAVHTRGLDFACDIYI | |
| •adenovirus E3-19K ER | WAPLAGTCGVLLLSLVITLYCAAATRGLLGCIITSLTG | |
| retention tag | RGGGSGGSTENLYFQSGSTLYKYKSRRSFIDEKKMP | |
| # 496 mNeonGreen-P2A-EGFR ECD | MVSKGEEDNMASLPATHELHIFGSINGVDFDMVGQG | 90 |
| with CD8 H/Tm and retention | TGNPNDGYEELNLKSTKGDLQFSPWILVPHIGYGFH | |
| variant HCV NS3 helicase domain | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVFRKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •Myc Tag | RTDLHAFENLEIIRGRTKQHGQFSLAVVSLNITSLGLR | |
| •CD8α Hinge | SLKEISDGDVIISGNKNLCYANTINWKKLFGTSGQKTK | |
| •CD8α TM | IISNRGENSCKATGQVCHALCSPEGCWGPEPRDCVS | |
| •Linker | CRNVSRGRECVDKCNLLEGEPREFVENSECIQCHPE | |
| •HCV NS3 Helix | CLPQAMNITCTGRGPDNCIQCAHYIDGPHCVKTCPA | |
| •Linker | GVMGENNTLVWKYADAGHVCHLCHPNCTYGCTGP | |
| •TEV cleavage site | GLEGCPTNGPKIPSGGSEQKLISEEDLTTTPAPRPPT | |
| •Linker | PAPTIASQPLSLRPEACRPAAGGAVHTRGLDFACDIYI | |
| •HCV NS3 helicase domain | WAPLAGTCGVLLLSLVITLYCAAATRGLLGCIITSLTG | |
| •Linker | RGGGSGGSTENLYFQSGSTGGSNSSPPAVTLTHGG | |
| •adenovirus E3-19K ER | SLYKYKSRRSFIDEKKMP | |
| retention tag | | |
| # 497 mNeonGreen-P2A-EGFR ECD | MVSKGEEDNMASLPATHELHIFGSINGVDFDMVGQG | 91 |
| with CISD2 Tm, TEV cleavage | TGNPNDGYEELNLKSTKGDLQFSPWILVPHIGYGFH | |
| site, and CISD2 ICD | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVERKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •CISD2 Tm | RTDLHAFENLEIIRGRTKQHGQFSLAVVSLNITSLGLR | |
| •Linker | SLKEISDGDVIISGNKNLCYANTINWKKLFGTSGQKTK | |
| •TEV cleavage site | IISNRGENSCKATGQVCHALCSPEGCWGPEPRDCVS | |
| •Linker | CRNVSRGRECVDKCNLLEGEPREFVENSECIQCHPE | |
| •CISD2 ICD | CLPQAMNITCTGRGPDNCIQCAHYIDGPHCVKTCPA | |
| | GVMGENNTLVWKYADAGHVCHLCHPNCTYGCTGP | |
| | GLEGCPTNGPKIPSGGSWLRLLPFLGVLALLGYLAVR | |
| | PFLAAAGGTENLYFQSGSTPKKKQQKDSLINLKIQKE | |
| | NPKVVNEINIEDLCLTKAAYCRCWRSKTFPACDGSH | |
| | NKHNELTGDNVGPLILKKKEV | |
| # 498 mNeonGreen-P2A-EGFR ECD | MVSKGEEDNMASLPATHELHIFGSINGVDFDMVGQG | 92 |
| with TMED4 Tm, TEV cleavage | TGNPNDGYEELNLKSTKGDLQFSPWILVPHIGYGFH | |
| site, and TMED4 ICD | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVERKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •TMED4 Tm | RTDLHAFENLEIIRGRTKQHGQFSLAVVSLNITSLGLR | |
| •Linker | SLKEISDGDVIISGNKNLCYANTINWKKLFGTSGQKTK | |
| •TEV cleavage site | IISNRGENSCKATGQVCHALCSPEGCWGPEPRDCVS | |
| •Linker | CRNVSRGRECVDKCNLLEGEPREFVENSECIQCHPE | |
| •TMED4 ICD | CLPQAMNITCTGRGPDNCIQCAHYIDGPHCVKTCPA | |
| | GVMGENNTLVWKYADAGHVCHLCHPNCTYGCTGP | |
| | GLEGCPTNGPKIPSGGSVLWWSIAQTVILILTGIWAAA | |
| | GGTENLYFQSGSTQMRHLKSFFEAKKLV | |
| # 499 mNeonGreen-P2A-EGFR ECD | MVSKGEEDNMASLPATHELHIFGSINGVDFDMVGQG | 93 |
| with SEL1L Tm, TEV cleavage | TGNPNDGYEELNLKSTKGDLQFSPWILVPHIGYGFH | |
| site, and SEL1L ICD | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVERKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •SEL1L Tm | RTDLHAFENLEIIRGRTKQHGQFSLAVVSLNITSLGLR | |
| •Linker | SLKEISDGDVIISGNKNLCYANTINWKKLFGTSGQKTK | |
| •TEV cleavage site | IISNRGENSCKATGQVCHALCSPEGCWGPEPRDCVS | |
| •Linker | CRNVSRGRECVDKCNLLEGEPREFVENSECIQCHPE | |
| •SEL1L ICD | CLPQAMNITCTGRGPDNCIQCAHYIDGPHCVKTCPA | |
| | GVMGENNTLVWKYADAGHVCHLCHPNCTYGCTGP | |
| | GLEGCPTNGPKIPSGGSLGPEWDLYLMTIIALLLGTVI | |
| | AAAGGTENLYFQSGSTAYRQRQHQDMPAPRPPGPR | |
| | PAPPQQEGPPEQQPPQ | |
| # 500 mNeonGreen-P2A-EGFR ECD | MVSKGEEDNMASLPATHELHIFGSINGVDFDMVGQG | 94 |
| with DDOST Tm, TEV cleavage | TGNPNDGYEELNLKSTKGDLQFSPWILVPHIGYGFH | |
| site, and DDOST ICD | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVERKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •DDOST Tm | RTDLHAFENLEIIRGRTKQHGQFSLAVVSLNITSLGLR | |
| •Linker | SLKEISDGDVIISGNKNLCYANTINWKKLFGTSGQKTK | |
| •TEV cleavage site | IISNRGENSCKATGQVCHALCSPEGCWGPEPRDCVS | |
| •Linker | CRNVSRGRECVDKCNLLEGEPREFVENSECIQCHPE | |
| •DDOST ICD | CLPQAMNITCTGRGPDNCIQCAHYIDGPHCVKTCPA | |
| | GVMGENNTLVWKYADAGHVCHLCHPNCTYGCTGP | |
| | GLEGCPTNGPKIPSGGSYYASAFSMMLGLFIFSIVFLA | |
| | AAGGTENLYFQSGSTHMKEKEKSD | |
| # 501 mNeonGreen-P2A-EGFR ECD | MVSKGEEDNMASLPATHELHIFGSINGVDFDMVGQG | 95 |
| with UGT2B17 Tm, TEV cleavage | TGNPNDGYEELNLKSTKGDLQFSPWILVPHIGYGFH | |
| site, and UGT2B17 ICD | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVFRKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •UGT2B17 Tm | RTDLHAFENLEIIRGRTKQHGQFSLAVVSLNITSLGLR | |
| •Linker | SLKEISDGDVIISGNKNLCYANTINWKKLFGTSGQKTK | |
| •TEV cleavage site | IISNRGENSCKATGQVCHALCSPEGCWGPEPRDCVS | |
| •Linker | CRNVSRGRECVDKCNLLEGEPREFVENSECIQCHPE | |
| •UGT2B17 ICD | CLPQAMNITCTGRGPDNCIQCAHYIDGPHCVKTCPA | |
| | GVMGENNTLVWKYADAGHVCHLCHPNCTYGCTGP | |
| | GLEGCPTNGPKIPSGGSIAFLLACVATMIFMITKCCLF | |
| | AAAGGTENLYFQSGSTCFRKLAKTGKKKKRD | |
| # 502 mNeonGreen-P2A-EGFR ECD | MVSKGEEDNMASLPATHELHIFGSINGVDFDMVGQG | 96 |
| with UGT1A1 Tm, TEV cleavage | TGNPNDGYEELNLKSTKGDLQFSPWILVPHIGYGFH | |
| site, and UGT1A1 ICD | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVFRKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •UGT1A1 Tm | RTDLHAFENLEIIRGRTKQHGQFSLAVVSLNITSLGLR | |
| •Linker | SLKEISDGDVIISGNKNLCYANTINWKKLFGTSGQKTK | |
| •TEV cleavage site | IISNRGENSCKATGQVCHALCSPEGCWGPEPRDCVS | |
| •Linker | CRNVSRGRECVDKCNLLEGEPREFVENSECIQCHPE | |
| •UGT1A1 ICD | CLPQAMNITCTGRGPDNCIQCAHYIDGPHCVKTCPA | |
| | GVMGENNTLVWKYADAGHVCHLCHPNCTYGCTGP | |
| | GLEGCPTNGPKIPSGGSVIGFLLAVVLTVAFITFAAAG | |
| | GTENLYFQSGSTKCCAYGYRKCLGKKGRVKKAHKSKTH | |
| # 503 mNeonGreen-P2A-EGFR ECD | MVSKGEEDNMASLPATHELHIFGSINGVDFDMVGQG | 97 |
| with TAPBP Tm, TEV cleavage | TGNPNDGYEELNLKSTKGDLQFSPWILVPHIGYGFH | |
| site, and TAPBP ICD | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVFRKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •TAPBP Tm | RTDLHAFENLEIIRGRTKQHGQFSLAVVSLNITSLGLR | |
| •Linker | SLKEISDGDVIISGNKNLCYANTINWKKLFGTSGQKTK | |
| •TEV cleavage site | IISNRGENSCKATGQVCHALCSPEGCWGPEPRDCVS | |
| •Linker | CRNVSRGRECVDKCNLLEGEPREFVENSECIQCHPE | |
| •TAPBP ICD | CLPQAMNITCTGRGPDNCIQCAHYIDGPHCVKTCPA | |
| | GVMGENNTLVWKYADAGHVCHLCHPNCTYGCTGP | |
| | GLEGCPTNGPKIPSGGSGLFLSAFLLLGLFKALGWAA | |
| | VAAAGGTENLYFQSGSTYLSTCKDSKKKAE | |
| # 505 mNeonGreen-P2A-EGFR ECD | MVSKGEEDNMASLPATHELHIFGSINGVDFDMVGQG | 98 |
| with TRIQK Tm, TEV cleavage | TGNPNDGYEELNLKSTKGDLQFSPWILVPHIGYGFH | |
| site, and TRIQK ICD | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVFRKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •TRIQK Tm | RTDLHAFENLEIIRGRTKQHGQFSLAVVSLNITSLGLR | |
| •Linker | SLKEISDGDVIISGNKNLCYANTINWKKLFGTSGQKTK | |
| •TEV cleavage site | IISNRGENSCKATGQVCHALCSPEGCWGPEPRDCVS | |
| •Linker | CRNVSRGRECVDKCNLLEGEPREFVENSECIQCHPE | |
| •TRIQK ICD | CLPQAMNITCTGRGPDNCIQCAHYIDGPHCVKTCPA | |
| | GVMGENNTLVWKYADAGHVCHLCHPNCTYGCTGP | |
| | GLEGCPTNGPKIPSGGSVGLVLAAILALLLAFYAFFYL | |
| | AAAGGTENLYFQSGSTRLTTDVDPDLDQDED | |
| # 506 mNeonGreen-P2A-EGFR ECD | MVSKGEEDNMASLPATHELHIFGSINGVDFDMVGQG | 99 |
| with mastadenovirus E3 19K Tm, | TGNPNDGYEELNLKSTKGDLQFSPWILVPHIGYGFH | |
| TEV cleavage site, and | QYLPYPDGMSPFQAAMVDGSGYQVHRTMQFEDGA | |
| mastadenovirus E3 19K ICD | SLTVNYRYTYEGSHIKGEAQVKGTGFPADGPVMTNS | |
| •mNeonGreen | LTAADWCRSKKTYPNDKTIISTFKWSYTTGNGKRYR | |
| •Linker | STARTTYTFAKPMAANYLKNQPMYVFRKTELKHSKT | |
| •P2A | ELNFKEWQKAFTDVMGMDELYKNASGATNFSLLKQ | |
| •Linker | AGDVEENPGPIASMLLLVTSLLLCELPHPAFLLIPRKV | |
| •GM-CSFR Leader | CNGIGIGEFKDSLSINATNIKHFKNCTSISGDLHILPVA | |
| •EGFR ECD | FRGDSFTHTPPLDPQELDILKTVKEITGFLLIQAWPEN | |
| •Linker | RTDLHAFENLEIIRGRTKQHGQFSLAVVSLNITSLGLR | |
| •mastadenovirus E3 19K Tm | SLKEISDGDVIISGNKNLCYANTINWKKLFGTSGQKTK | |
| •Linker | IISNRGENSCKATGQVCHALCSPEGCWGPEPRDCVS | |
| •TEV cleavage site | CRNVSRGRECVDKCNLLEGEPREFVENSECIQCHPE | |
| •Linker | CLPQAMNITCTGRGPDNCIQCAHYIDGPHCVKTCPA | |
| •mastadenovirus E3 19K ICD | GVMGENNTLVWKYADAGHVCHLCHPNCTYGCTGP | |
| | GLEGCPTNGPKIPSGGSTFCSTALLITALALVCTLLYL | |
| | AAAGGTENLYFQSGSTKYKSRRSFIDEKKMP | |
| # 507 mNeonGreen-P2A-EGFR ECD | MVSKGEEDNMASLPATHELHIFGSINGVDFDMVGQG | 100 |
| with IBV S protein Tm, TEV | TGNPNDGYEELNLKSTKGDLQFSPWILVPHIGYGFH | |
| cleavage site, and IBV S | QYLPYPDGMSPFQAAMVDGSGYQVHRTMQFEDGA | |
| protein ICD | SLTVNYRYTYEGSHIKGEAQVKGTGFPADGPVMTNS | |
| •mNeonGreen | LTAADWCRSKKTYPNDKTIISTFKWSYTTGNGKRYR | |
| •Linker | STARTTYTFAKPMAANYLKNQPMYVFRKTELKHSKT | |
| •P2A | ELNFKEWQKAFTDVMGMDELYKNASGATNFSLLKQ | |
| •Linker | AGDVEENPGPIASMLLLVTSLLLCELPHPAFLLIPRKV | |
| •GM-CSFR Leader | CNGIGIGEFKDSLSINATNIKHFKNCTSISGDLHILPVA | |
| •EGFR ECD | FRGDSFTHTPPLDPQELDILKTVKEITGFLLIQAWPEN | |
| •Linker | RTDLHAFENLEIIRGRTKQHGQFSLAVVSLNITSLGLR | |
| •IBV S protein Tm | SLKEISDGDVIISGNKNLCYANTINWKKLFGTSGQKTK | |
| •Linker | IISNRGENSCKATGQVCHALCSPEGCWGPEPRDCVS | |
| •TEV cleavage site | CRNVSRGRECVDKCNLLEGEPREFVENSECIQCHPE | |
| •Linker | CLPQAMNITCTGRGPDNCIQCAHYIDGPHCVKTCPA | |
| •IBV S protein ICD | GVMGENNTLVWKYADAGHVCHLCHPNCTYGCTGP | |
| | GLEGCPTNGPKIPSGGSWYVWLAIFFAIIIFILILGWVL | |
| | LAAAGGTENLYFQSGSTMTGCCGCCCGCFGIIPLMS | |
| | KCGKKSSYYTTFDNDVVIEQYRPKKSV | |
| # 508 mNeonGreen-P2A-EGFR ECD | MVSKGEEDNMASLPATHELHIFGSINGVDFDMVGQG | 101 |
| with calnexin Tm, TEV cleavage | TGNPNDGYEELNLKSTKGDLQFSPWILVPHIGYGFH | |
| site, and calnexin ICD | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVFRKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •Calnexin Tm | RTDLHAFENLEIIRGRTKQHGQFSLAVVSLNITSLGLR | |
| •Linker | SLKEISDGDVIISGNKNLCYANTINWKKLFGTSGQKTK | |
| •TEV cleavage site | IISNRGENSCKATGQVCHALCSPEGCWGPEPRDCVS | |
| •Linker | CRNVSRGRECVDKCNLLEGEPREFVENSECIQCHPE | |
| •Calnexin ICD | CLPQAMNITCTGRGPDNCIQCAHYIDGPHCVKTCPA | |
| | GVMGENNTLVWKYADAGHVCHLCHPNCTYGCTGP | |
| | GLEGCPTNGPKIPSGGSWLWVVYILTVALPVFLVILFC | |
| | AAAGGTENLYFQSGSTNRSPRNRKPRRE | |
| 532 mNeonGreen-P2A-EGFR ECD | MVSKGEEDNMASLPATHELHIFGSINGVDFDMVGQG | 102 |
| with CISD2 Tm, human enterokinase | TGNPNDGYEELNLKSTKGDLQFSPWILVPHIGYGFH | |
| cleavage site 1, and CISD2 ICD | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVFRKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •CISD2 Tm | RTDLHAFENLEIIRGRTKQHGQFSLAVVSLNITSLGLR | |
| •Linker | SLKEISDGDVIISGNKNLCYANTINWKKLFGTSGQKTK | |
| •human enterokinase cleavage | IISNRGENSCKATGQVCHALCSPEGCWGPEPRDCVS | |
| •site 1 | CRNVSRGRECVDKCNLLEGEPREFVENSECIQCHPE | |
| •Linker | CLPQAMNITCTGRGPDNCIQCAHYIDGPHCVKTCPA | |
| •CISD2 ICD | GVMGENNTLVWKYADAGHVCHLCHPNCTYGCTGP | |
| | GLEGCPTNGPKIPSGGSWLRLLPFLGVLALLGYLAVR | |
| | PFLAAAGGTDDDDKGSTPKKKQQKDSLINLKIQKENP | |
| | KVVNEINIEDLCLTKAAYCRCWRSKTFPACDGSHNK | |
| | HNELTGDNVGPLILKKKEV | |
| 533 mNeonGreen-P2A-EGFR ECD | MVSKGEEDNMASLPATHELHIFGSINGVDFDMVGQG | 103 |
| with CISD2 Tm, human enterokinase | TGNPNDGYEELNLKSTKGDLQFSPWILVPHIGYGFH | |
| cleavage site 2, and CISD2 ICD | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVFRKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •CISD2 Tm | RTDLHAFENLEIIRGRTKQHGQFSLAVVSLNITSLGLR | |
| •Linker | SLKEISDGDVIISGNKNLCYANTINWKKLFGTSGQKTK | |
| •human enterokinase cleavage | IISNRGENSCKATGQVCHALCSPEGCWGPEPRDCVS | |
| site 2 | CRNVSRGRECVDKCNLLEGEPREFVENSECIQCHPE | |
| •Linker | CLPQAMNITCTGRGPDNCIQCAHYIDGPHCVKTCPA | |
| •CISD2 ICD | GVMGENNTLVWKYADAGHVCHLCHPNCTYGCTGP | |
| | GLEGCPTNGPKIPSGGSWLRLLPFLGVLALLGYLAVR | |
| | PFLAAAGGTDDDDRGSTPKKKQQKDSLINLKIQKENP | |
| | KVVNEINIEDLCLTKAAYCRCWRSKTFPACDGSHNK | |
| | HNELTGDNVGPLILKKKEV | |
| # 564 mNeonGreen-P2A-EGFR ECD | MVSKGEEDNMASLPATHELHIFGSINGVDFDMVGQG | 104 |
| with CD28 H/Tm with degron and | TGNPNDGYEELNLKSTKGDLQFSPWILVPHIGYGFH | |
| adenovirus E3-19K ER retention | QYLPYPDGMSPFQAAMVDGSGYQVHRTMQFEDGA | |
| tag | SLTVNYRYTYEGSHIKGEAQVKGTGFPADGPVMTNS | |
| •mNeonGreen | LTAADWCRSKKTYPNDKTIISTFKWSYTTGNGKRYR | |
| •Linker | STARTTYTFAKPMAANYLKNQPMYVFRKTELKHSKT | |
| •P2A | ELNFKEWQKAFTDVMGMDELYKNASGATNFSLLKQ | |
| •Linker | AGDVEENPGPIASMLLLVTSLLLCELPHPAFLLIPRKV | |
| •GM-CSFR Leader | CNGIGIGEFKDSLSINATNIKHFKNCTSISGDLHILPVA | |
| •EGFR ECD | FRGDSFTHTPPLDPQELDILKTVKEITGFLLIQAWPEN | |
| •Linker | RTDLHAFENLEIIRGRTKQHGQFSLAVVSLNITSLGLR | |
| •CD28 Hinge | SLKEISDGDVIISGNKNLCYANTINWKKLFGTSGQKTK | |
| •CD28 TM | IISNRGENSCKATGQVCHALCSPEGCWGPEPRDCVS | |
| •Linker | CRNVSRGRECVDKCNLLEGEPREFVENSECIQCHPE | |
| •TEV cleavage site | CLPQAMNITCTGRGPDNCIQCAHYIDGPHCVKTCPA | |
| •Linker | GVMGENNTLVWKYADAGHVCHLCHPNCTYGCTGP | |
| •Degron | GLEGCPTNGPKIPSGGSIEVMYPPPYLDNEKSNGTII | |
| •adenovirus E3-19K ER | HVKGKHLCPSPLFPGPSKPFWVLVVVGGVLACYSLL | |
| retention tag | VTVAFIIFWVAAAGGTENLYFQSGSTPITKIDTKYIMTC | |
| | MSADLEVVTSTWVLVGGVLAALAAYCLSTLYKYKSR | |
| | RSFIEEKKMP | |
| # 565 mNeonGreen-P2A-EGFR ECD | MVSKGEEDNMASLPATHELHIFGSINGVDFDMVGQG | 105 |
| with CD28 H/Tm, TEV cleavage | TGNPNDGYEELNLKSTKGDLQFSPWILVPHIGYGFH | |
| site, and CISD2 ICD | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVFRKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •CD28 hinge | RTDLHAFENLEIIRGRTKQHGQFSLAVVSLNITSLGLR | |
| •CD28 Tm | SLKEISDGDVIISGNKNLCYANTINWKKLFGTSGQKTK | |
| •Linker | IISNRGENSCKATGQVCHALCSPEGCWGPEPRDCVS | |
| •TEV cleavage site | CRNVSRGRECVDKCNLLEGEPREFVENSECIQCHPE | |
| •Linker | CLPQAMNITCTGRGPDNCIQCAHYIDGPHCVKTCPA | |
| •CISD2 ICD | GVMGENNTLVWKYADAGHVCHLCHPNCTYGCTGP | |
| | GLEGCPTNGPKIPSGGSIEVMYPPPYLDNEKSNGTII | |
| | HVKGKHLCPSPLFPGPSKPFWVLVVVGGVLACYSLL | |
| | VTVAFIIFWVAAAGGTENLYFQSGSTPKKKQQKDSLI | |
| | NLKIQKENPKVVNEINIEDLCLTKAAYCRCWRSKTFP | |
| | ACDGSHNKHNELTGDNVGPLILKKKEV | |
| # 566 mNeonGreen-P2A-EGFR ECD | MVSKGEEDNMASLPATHELHIFGSINGVDFDMVGQG | 106 |
| with CD28 H/Tm, TEV cleavage | TGNPNDGYEELNLKSTKGDLQFSPWILVPHIGYGFH | |
| site, and UGT2B17 ICD | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVFRKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •CD28 Hinge | RTDLHAFENLEIIRGRTKQHGQFSLAVVSLNITSLGLR | |
| •CD28 Tm | SLKEISDGDVIISGNKNLCYANTINWKKLFGTSGQKTK | |
| •Linker | IISNRGENSCKATGQVCHALCSPEGCWGPEPRDCVS | |
| •TEV cleavage site | CRNVSRGRECVDKCNLLEGEPREFVENSECIQCHPE | |
| •Linker | CLPQAMNITCTGRGPDNCIQCAHYIDGPHCVKTCPA | |
| •UGT2B17 ICD | GVMGENNTLVWKYADAGHVCHLCHPNCTYGCTGP | |
| | GLEGCPTNGPKIPSGGSIEVMYPPPYLDNEKSNGTII | |
| | HVKGKHLCPSPLFPGPSKPFWVLVVVGGVLACYSLL | |
| | VTVAFIIFWVAAAGGTENLYFQSGSTCFRKLAKTGKK | |
| | KKRD | |
| # 567 mNeonGreen-P2A-EGFR ECD | MVSKGEEDNMASLPATHELHIFGSINGVDFDMVGQG | 107 |
| with CD28 H/Tm, TEV cleavage | TGNPNDGYEELNLKSTKGDLQFSPWILVPHIGYGFH | |
| site, and IBV S protein ICD | QYLPYPDGMSPFQAAMVDGSGYQVHRTMQFEDGA | |
| •mNeonGreen | SLTVNYRYTYEGSHIKGEAQVKGTGFPADGPVMTNS | |
| •Linker | LTAADWCRSKKTYPNDKTIISTFKWSYTTGNGKRYR | |
| •P2A | STARTTYTFAKPMAANYLKNQPMYVFRKTELKHSKT | |
| •Linker | ELNFKEWQKAFTDVMGMDELYKNASGATNFSLLKQ | |
| •GM-CSFR Leader | AGDVEENPGPIASMLLLVTSLLLCELPHPAFLLIPRKV | |
| •EGFR ECD | CNGIGIGEFKDSLSINATNIKHFKNCTSISGDLHILPVA | |
| •Linker | FRGDSFTHTPPLDPQELDILKTVKEITGFLLIQAWPEN | |
| •CD28 Hinge | RTDLHAFENLEIIRGRTKQHGQFSLAVVSLNITSLGLR | |
| •CD28 Tm | SLKEISDGDVIISGNKNLCYANTINWKKLFGTSGQKTK | |
| •Linker | IISNRGENSCKATGQVCHALCSPEGCWGPEPRDCVS | |
| •TEV cleavage site | CRNVSRGRECVDKCNLLEGEPREFVENSECIQCHPE | |
| •Linker | CLPQAMNITCTGRGPDNCIQCAHYIDGPHCVKTCPA | |
| •IBV S protein ICD | GVMGENNTLVWKYADAGHVCHLCHPNCTYGCTGP | |
| | GLEGCPTNGPKIPSGGSIEVMYPPPYLDNEKSNGTII | |
| | HVKGKHLCPSPLFPGPSKPFWVLVVVGGVLACYSLL | |
| | VTVAFIIFWVAAAGGTENLYFQSGSTMTGCCGCCCG | |
| | CFGIIPLMSKCGKKSSYYTTFDNDVVIEQYRPKKSV | |
| # 568 mNeonGreen-P2A-EGFR ECD | MVSKGEEDNMASLPATHELHIFGSINGVDFDMVGQG | 108 |
| with CD8 H/Tm with PuroR, TEV | TGNPNDGYEELNLKSTKGDLQFSPWILVPHIGYGFH | |
| cleavage site, degron and | QYLPYPDGMSPFQAAMVDGSGYQVHRTMQFEDGA | |
| adenovirus E3-19K ER retention | SLTVNYRYTYEGSHIKGEAQVKGTGFPADGPVMTNS | |
| tag | LTAADWCRSKKTYPNDKTIISTFKWSYTTGNGKRYR | |
| •mNeonGreen | STARTTYTFAKPMAANYLKNQPMYVFRKTELKHSKT | |
| •Linker | ELNFKEWQKAFTDVMGMDELYKNASGATNFSLLKQ | |
| •P2A | AGDVEENPGPIASMLLLVTSLLLCELPHPAFLLIPRKV | |
| •Linker | CNGIGIGEFKDSLSINATNIKHFKNCTSISGDLHILPVA | |
| •GM-CSFR Leader | FRGDSFTHTPPLDPQELDILKTVKEITGFLLIQAWPEN | |
| •EGFR ECD | RTDLHAFENLEIIRGRTKQHGQFSLAVVSLNITSLGLR | |
| •Linker | SLKEISDGDVIISGNKNLCYANTINWKKLFGTSGQKTK | |
| •Myc Tag | IISNRGENSCKATGQVCHALCSPEGCWGPEPRDCVS | |
| •CD8α Hinge | CRNVSRGRECVDKCNLLEGEPREFVENSECIQCHPE | |
| •CD8α TM | CLPQAMNITCTGRGPDNCIQCAHYIDGPHCVKTCPA | |
| •Linker | GVMGENNTLVWKYADAGHVCHLCHPNCTYGCTGP | |
| •PuroR | GLEGCPTNGPKIPSGGSEQKLISEEDLTTTPAPRPPT | |
| •linker | PAPTIASQPLSLRPEACRPAAGGAVHTRGLDFACDIYI | |
| •TEV cleavage site | WAPLAGTCGVLLLSLVITLYCAAGAGGSGGTEYKPTV | |
| •Linker | RLATRDDVPRAVRTLAAAFADYPATRHTVDPDRHIER | |
| •Degron | VTELQELFLTRVGLDIGKVWVADDGAAVAVWTTPES | |
| •adenovirus E3-19K ER | VEAGAVFAEIGPRMAELSGSRLAAQQQMEGLLAPHR | |
| retention tag | PKEPAWFLATVGVSPDHQGKGLGSAVVLPGVEAAE | |
| | RAGVPAFLETSAPRNLPFYERLGFTVTADVEVPEGP | |
| | RTWCMTRKPGAAAAGGTENLYFQSGSTPITKIDTKYI | |
| | MTCMSADLEVVTSTWVLVGGVLAALAAYCLSTLYKY | |
| | KSRRSFIEEKKMP | |
| # 569 mNeonGreen-P2A-EGFR ECD | MVSKGEEDNMASLPATHELHIFGSINGVDFDMVGQG | 109 |
| with CD8 H/Tm with PuroR, dual | TGNPNDGYEELNLKSTKGDLQFSPWILVPHIGYGFH | |
| TEV cleavage sites, degron and | QYLPYPDGMSPFQAAMVDGSGYQVHRTMQFEDGA | |
| adenovirus E3-19K ER retention | SLTVNYRYTYEGSHIKGEAQVKGTGFPADGPVMTNS | |
| tag | LTAADWCRSKKTYPNDKTIISTFKWSYTTGNGKRYR | |
| •mNeonGreen | STARTTYTFAKPMAANYLKNQPMYVFRKTELKHSKT | |
| •Linker | ELNFKEWQKAFTDVMGMDELYKNASGATNFSLLKQ | |
| •P2A | AGDVEENPGPIASMLLLVTSLLLCELPHPAFLLIPRKV | |
| •Linker | CNGIGIGEFKDSLSINATNIKHFKNCTSISGDLHILPVA | |
| •GM-CSFR Leader | FRGDSFTHTPPLDPQELDILKTVKEITGFLLIQAWPEN | |
| •EGFR ECD | RTDLHAFENLEIIRGRTKQHGQFSLAVVSLNITSLGLR | |
| •Linker | SLKEISDGDVIISGNKNLCYANTINWKKLFGTSGQKTK | |
| •Myc Tag | IISNRGENSCKATGQVCHALCSPEGCWGPEPRDCVS | |
| •CD8α Hinge | CRNVSRGRECVDKCNLLEGEPREFVENSECIQCHPE | |
| •CD8α TM | CLPQAMNITCTGRGPDNCIQCAHYIDGPHCVKTCPA | |
| •Linker | GVMGENNTLVWKYADAGHVCHLCHPNCTYGCTGP | |
| •TEV cleavage site 2 | GLEGCPTNGPKIPSGGSEQKLISEEDLTTTPAPRPPT | |
| •PuroR | PAPTIASQPLSLRPEACRPAAGGAVHTRGLDFACDIYI | |
| •linker | WAPLAGTCGVLLLSLVITLYCAAGAGGSGGENLYFQ | |
| •TEV cleavage site | FTEYKPTVRLATRDDVPRAVRTLAAAFADYPATRHTV | |
| •Linker | DPDRHIERVTELQELFLTRVGLDIGKVWVADDGAAVA | |
| •Degron | VWTTPESVEAGAVFAEIGPRMAELSGSRLAAQQQM | |
| •adenovirus E3-19K ER | EGLLAPHRPKEPAWFLATVGVSPDHQGKGLGSAVVL | |
| retention tag | PGVEAAERAGVPAFLETSAPRNLPFYERLGFTVTAD | |
| | VEVPEGPRTWCMTRKPGAAAAGGTENLYFQSGSTPI | |
| | TKIDTKYIMTCMSADLEVVTSTWVLVGGVLAALAAYC | |
| | LSTLYKYKSRRSFIEEKKMP | |
| # 577 CD22.BBz-P2A-EGFR ECD | MLLLVTSLLLCELPHPAFLLIPQVQLQQSGPGLVKPS | 110 |
| with CISD2 Tm, TEV cleavage | QTLSLTCAISGDSVSSNSAAWNWIRQSPSRGLEWLG | |
| site, and CISD2 ICD | RTYYRSKWYNDYAVSVKSRITINPDTSKNQFSLQLNS | |
| •Leader sequence | VTPEDTAVYYCAREVTGDLEDAFDIWGQGTMVTVSS | |
| •CD22 scFv | GGGGSDIQMTQSPSSLSASVGDRVTITCRASQTIWS | |
| •Linker | YLNWYQQRPGKAPNLLIYAASSLQSGVPSRFSGRGS | |
| •CD8α H/Tm | GTDFTLTISSLQAEDFATYYCQQSYSIPQTFGQGTKL | |
| •4-1 BB | EIKAAATTTPAPRPPTPAPTIASQPLSLRPEACRPAAG | |
| •CD3z | GAVHTRGLDFACDIYIWAPLAGTCGVLLLSLVITLYCK | |
| •Linker | RGRKKLLYIFKQPFMRPVQTTQEEDGCSCRFPEEEE | |
| •P2A | GGCELRVKFSRSADAPAYKQGQNQLYNELNLGRRE | |
| •Linker | EYDVLDKRRGRDPEMGGKPRRKNPQEGLYNELQKD | |
| •GM-CSFR Leader | KMAEAYSEIGMKGERRRGKGHDGLYQGLSTATKDT | |
| •EGFR ECD | YDALHMQALPPRNASGATNFSLLKQAGDVEENPGPI | |
| •Linker | ASMLLLVTSLLLCELPHPAFLLIPRKVCNGIGIGEFKDS | |
| •CISD2 Tm | LSINATNIKHFKNCTSISGDLHILPVAFRGDSFTHTPPL | |
| •Linker | DPQELDILKTVKEITGFLLIQAWPENRTDLHAFENLEII | |
| •TEV cleavage site | RGRTKQHGQFSLAVVSLNITSLGLRSLKEISDGDVIIS | |
| •Linker | GNKNLCYANTINWKKLFGTSGQKTKIISNRGENSCKA | |
| •CISD2 ICD | TGQVCHALCSPEGCWGPEPRDCVSCRNVSRGREC | |
| | VDKCNLLEGEPREFVENSECIQCHPECLPQAMNITCT | |
| | GRGPDNCIQCAHYIDGPHCVKTCPAGVMGENNTLV | |
| | WKYADAGHVCHLCHPNCTYGCTGPGLEGCPTNGPK | |
| | IPSGGSWLRLLPFLGVLALLGYLAVRPFLAAAGGTEN | |
| | LYFQSGSTPKKKQQKDSLINLKIQKENPKVVNEINIED | |
| | LCLTKAAYCRCWRSKTFPACDGSHNKHNELTGDNV | |
| | GPLILKKKEV | |
| # 578 CD22.BBz-P2A-EGFR ECD | MLLLVTSLLLCELPHPAFLLIPQVQLQQSGPGLVKPS | 111 |
| with IBV S protein Tm, TEV | QTLSLTCAISGDSVSSNSAAWNWIRQSPSRGLEWLG | |
| cleavage site, and IBV S | RTYYRSKWYNDYAVSVKSRITINPDTSKNQFSLQLNS | |
| protein ICD | VTPEDTAVYYCAREVTGDLEDAFDIWGQGTMVTVSS | |
| •Leader sequence | GGGGSDIQMTQSPSSLSASVGDRVTITCRASQTIWS | |
| •CD22 scFv | YLNWYQQRPGKAPNLLIYAASSLQSGVPSRFSGRGS | |
| •Linker | GTDFTLTISSLQAEDFATYYCQQSYSIPQTFGQGTKL | |
| •CD8α H/Tm | EIKAAATTTPAPRPPTPAPTIASQPLSLRPEACRPAAG | |
| •4-1BB | GAVHTRGLDFACDIYIWAPLAGTCGVLLLSLVITLYCK | |
| •CD3z | RGRKKLLYIFKQPFMRPVQTTQEEDGCSCRFPEEEE | |
| •Linker | GGCELRVKFSRSADAPAYKQGQNQLYNELNLGRRE | |
| •P2A | EYDVLDKRRGRDPEMGGKPRRKNPQEGLYNELQKD | |
| •Linker | KMAEAYSEIGMKGERRRGKGHDGLYQGLSTATKDT | |
| •GM-CSFR Leader | YDALHMQALPPRNASGATNFSLLKQAGDVEENPGPI | |
| •EGFR ECD | ASMLLLVTSLLLCELPHPAFLLIPRKVCNGIGIGEFKDS | |
| •Linker | LSINATNIKHFKNCTSISGDLHILPVAFRGDSFTHTPPL | |
| •IBV S protein Tm | DPQELDILKTVKEITGFLLIQAWPENRTDLHAFENLEII | |
| •Linker | RGRTKQHGQFSLAVVSLNITSLGLRSLKEISDGDVIIS | |
| •TEV cleavage site | GNKNLCYANTINWKKLFGTSGQKTKIISNRGENSCKA | |
| •Linker | TGQVCHALCSPEGCWGPEPRDCVSCRNVSRGREC | |
| •IBV S protein ICD | VDKCNLLEGEPREFVENSECIQCHPECLPQAMNITCT | |
| | GRGPDNCIQCAHYIDGPHCVKTCPAGVMGENNTLV | |
| | WKYADAGHVCHLCHPNCTYGCTGPGLEGCPTNGPK | |
| | IPSGGSWYVWLAIFFAIIIFILILGWVLLAAAGGTENLYF | |
| | QSGSTMTGCCGCCCGCFGIIPLMSKCGKKSSYYTTF | |
| | DNDVVIEQYRPKKSV | |
| # 579 cJun-P2A-EGFR ECD with | MTAKMETTFYDDALNASFLPSESGPYGYSNPKILKQS | 112 |
| CISD2 Tm, TEV cleavage site, | MTLNLADPVGSLKPHLRAKNSDLLTSPDVGLLKLASP | |
| and CISD2 ICD | ELERLIIQSSNGHITTTPTPTQFLCPKNVTDEQEGFAE | |
| •cJun | GFVRALAELHSQNTLPSVTSAAQPVNGAGMVAPAVA | |
| •Linker | SVAGGSGSGGFSASLHSEPPVYANLSNFNPGALSSG | |
| •P2A | GGAPSYGAAGLAFPAQPQQQQQPPHHLPQQMPVQ | |
| •Linker | HPRLQALKEEPQTVPEMPGETPPLSPIDMESQERIKA | |
| •GM-CSFR Leader | ERKRMRNRIAASKCRKRKLERIARLEEKVKTLKAQNS | |
| •EGFR ECD | ELASTANMLREQVAQLKQKVMNHVNSGCQLMLTQQ | |
| •Linker | LQTFNASGATNFSLLKQAGDVEENPGPIASMLLLVTS | |
| •CISD2 Tm | LLLCELPHPAFLLIPRKVCNGIGIGEFKDSLSINATNIK | |
| •Linker | HFKNCTSISGDLHILPVAFRGDSFTHTPPLDPQELDIL | |
| •TEV cleavage site | KTVKEITGFLLIQAWPENRTDLHAFENLEIIRGRTKQH | |
| •Linker | GQFSLAVVSLNITSLGLRSLKEISDGDVIISGNKNLCY | |
| •CISD2 ICD | ANTINWKKLFGTSGQKTKIISNRGENSCKATGQVCHA | |
| | LCSPEGCWGPEPRDCVSCRNVSRGRECVDKCNLLE | |
| | GEPREFVENSECIQCHPECLPQAMNITCTGRGPDNC | |
| | IQCAHYIDGPHCVKTCPAGVMGENNTLVWKYADAGH | |
| | VCHLCHPNCTYGCTGPGLEGCPTNGPKIPSGGSWL | |
| | RLLPFLGVLALLGYLAVRPFLAAAGGTENLYFQSGST | |
| | PKKKQQKDSLINLKIQKENPKVVNEINIEDLCLTKAAY | |
| | CRCWRSKTFPACDGSHNKHNELTGDNVGPLILKKKE | |
| | V | |
| # 580 cJun-P2A-EGFR ECD with IBV | MTAKMETTFYDDALNASFLPSESGPYGYSNPKILKQS | 113 |
| S protein Tm, TEV cleavage site, | MTLNLADPVGSLKPHLRAKNSDLLTSPDVGLLKLASP | |
| and IBV S protein ICD | ELERLIIQSSNGHITTTPTPTQFLCPKNVTDEQEGFAE | |
| •cJun | GFVRALAELHSQNTLPSVTSAAQPVNGAGMVAPAVA | |
| •Linker | SVAGGSGSGGFSASLHSEPPVYANLSNFNPGALSSG | |
| •P2A | GGAPSYGAAGLAFPAQPQQQQQPPHHLPQQMPVQ | |
| •Linker | HPRLQALKEEPQTVPEMPGETPPLSPIDMESQERIKA | |
| •GM-CSFR Leader | ERKRMRNRIAASKCRKRKLERIARLEEKVKTLKAQNS | |
| •EGFR ECD | ELASTANMLREQVAQLKQKVMNHVNSGCQLMLTQQ | |
| •Linker | LQTFNASGATNFSLLKQAGDVEENPGPIASMLLLVTS | |
| •IBV S protein Tm | LLLCELPHPAFLLIPRKVCNGIGIGEFKDSLSINATNIK | |
| •Linker | HFKNCTSISGDLHILPVAFRGDSFTHTPPLDPQELDIL | |
| •TEV cleavage site | KTVKEITGFLLIQAWPENRTDLHAFENLEIIRGRTKQH | |
| •Linker | GQFSLAVVSLNITSLGLRSLKEISDGDVIISGNKNLCY | |
| •IBV S protein ICD | ANTINWKKLFGTSGQKTKIISNRGENSCKATGQVCHA | |
| | LCSPEGCWGPEPRDCVSCRNVSRGRECVDKCNLLE | |
| | GEPREFVENSECIQCHPECLPQAMNITCTGRGPDNC | |
| | IQCAHYIDGPHCVKTCPAGVMGENNTLVWKYADAGH | |
| | VCHLCHPNCTYGCTGPGLEGCPTNGPKIPSGGSWY | |
| | VWLAIFFAIIIFILILGWVLLAAAGGTENLYFQSGSTMT | |
| | GCCGCCCGCFGIIPLMSKCGKKSSYYTTFDNDVVIEQ | |
| | YRPKKSV | |
| # 797 Minimized Two-way TEV | MVSKGEELIKENMHMKLYMEGTVDNHHFKCTSEGE | 114 |
| protease with UGT2B17 Tm and | GKPYEGTQTMRIKVVEGGPLPFAFDILATSFLYGSKT | |
| ICD | FINHTQGIPDFFKQSFPEGFTWERVTTYEDGGVLTAT | |
| •mTagBFP2 | QDTSLQDGCLIYNVKIRGVNFTSNGPVMQKKTLGWE | |
| •Linker | AFTETLYPADGGLEGRNDMALKLVGGSHLIANAKTTY | |
| •P2A | RSKKPAKNLKMPGVYYVDYRLERIKEANNETYVEQH | |
| •TCRβ Leader | EVAVARYCDLPSKLGHKLNNASATNFSLLKQAGDVE | |
| •Linker | ENPGPMGTSLLCWMALCLLGADHADGGSIAFLLACV | |
| •UGT2B17 Tm | ATMIFMITKCCLFGNSGSSGGSGGSGSSGGSGESLF | |
| •Intracellular linker | KGPRDYNPISSTICHLTNESDGHTTSLYGIGFGPFIITN | |
| •TEV protease | KHLFRRNNGTLLVQSLHGVFKVKNTTTLQQHLIDGRD | |
| •Linker | MIIIRMPKDFPPFPQKLKFREPQREERICLVTTNFQTK | |
| •UGT2B17 ICD | SMSSMVSDTSCTFPSSDGIFWKHWIQTKDGQCGSP | |
| | LVSTRDGFIVGIHSASNFTNTNNYFTSVPKNFMELLT | |
| | NQEAQQWVSGWRLNADSVLWGGHKVFMVKPEEPF | |
| | QPVKEATQLMNGSTCFRKLAKTGKKKKRD | |
| # 821 Two-way protease using | MVSKGEELIKENMHMKLYMEGTVDNHHFKCTSEGE | 115 |
| human protease KLK3 | GKPYEGTQTMRIKVVEGGPLPFAFDILATSFLYGSKT | |
| •mTagBFP2 | FINHTQGIPDFFKQSFPEGFTWERVTTYEDGGVLTAT | |
| •Linker | QDTSLQDGCLIYNVKIRGVNFTSNGPVMQKKTLGWE | |
| •P2A | AFTETLYPADGGLEGRNDMALKLVGGSHLIANAKTTY | |
| •TCRβ Leader | RSKKPAKNLKMPGVYYVDYRLERIKEANNETYVEQH | |
| •RQR8 Tag | EVAVARYCDLPSKLGHKLNNASATNFSLLKQAGDVE | |
| •CD8α Hinge | ENPGPMGTSLLCWMALCLLGADHADACPYSNPSLC | |
| •CD8α Tm | SGGGGSELPTQGTFSNVSTNVSPAKPTTTACPYSNP | |
| •CD8α intracellular linker | SLCSGGGGSPAPRPPTPAPTIASQPLSLRPEACRPA | |
| •KLK3 protease | AGGAVHTRGLDFACDIYIWAPLAGTCGVLLLSLVITLY | |
| •linker | CNHRNRRRVCKCPRPVVGSSGNSSGGSGSTGSSG | |
| •adenovirus E3-19K ER | GSGGSGSSGGSIVGGWECEKHSQPWQVLVASRGR | |
| retention tag | AVCGGVLVHPQWVLTAAHCIRNKSVILLGRHSLFHPE | |
| | DTGQVFQVSHSFPHPLYDMSLLKNRFLRPGDDSSHD | |
| | LMLLRLSEPAELTDAVKVMDLPTQEPALGTTCYASG | |
| | WGSIEPEEFLTPKKLQCVDLHVISNDVCAQVHPQKVT | |
| | KFMLCAGRWTGGKSTCSGDSGGPLVCNGVLQGITS | |
| | WGSEPCALPERPSLYTKVVHYRKWIKDTIVANPAAAS | |
| | TGSSGGGGGSGGLYKYKSRRSFIDEKKMP | |
| # 822 Two-way protease using | MVSKGEELIKENMHMKLYMEGTVDNHHFKCTSEGE | 116 |
| human protease KLK4 | GKPYEGTQTMRIKVVEGGPLPFAFDILATSFLYGSKT | |
| •mTagBFP2 | FINHTQGIPDFFKQSFPEGFTWERVTTYEDGGVLTAT | |
| •Linker | QDTSLQDGCLIYNVKIRGVNFTSNGPVMQKKTLGWE | |
| •P2A | AFTETLYPADGGLEGRNDMALKLVGGSHLIANAKTTY | |
| •TCRß Leader | RSKKPAKNLKMPGVYYVDYRLERIKEANNETYVEQH | |
| •RQR8 Tag | EVAVARYCDLPSKLGHKLNNASATNFSLLKQAGDVE | |
| •CD8α Hinge | ENPGPMGTSLLCWMALCLLGADHADACPYSNPSLC | |
| •CD8α Tm | SGGGGSELPTQGTFSNVSTNVSPAKPTTTACPYSNP | |
| •CD8α intracellular linker | SLCSGGGGSPAPRPPTPAPTIASQPLSLRPEACRPA | |
| •KLK4 protease | AGGAVHTRGLDFACDIYIWAPLAGTCGVLLLSLVITLY | |
| •linker | CNHRNRRRVCKCPRPVVGSSGNSSGGSGSTGSSG | |
| •adenovirus E3-19K ER | GSGGSGSSGGSIINGEDCSPHSQPWQAALVMENEL | |
| retention tag | FCSGVLVHPQWVLSAAHCFQNSYTIGLGLHSLEADQ | |
| | EPGSQMVEASLSVRHPEYNRPLLANDLMLIKLDESVS | |
| | ESDTIRSISIASQCPTAGNSCLVSGWGLLANGRMPTV | |
| | LQCVNVSVVSEEVCSKLYDPLYHPSMFCAGGGHDQ | |
| | KDSCNGDSGGPLICNGYLQGLVSFGKAPCGQVGVP | |
| | GVYTNLCKFTEWIEKTVQASAAASTGSSGGGGGSG | |
| | GLYKYKSRRSFIDEKKMP | |
| # 823 Two-way protease using | MVSKGEELIKENMHMKLYMEGTVDNHHFKCTSEGE | 117 |
| human protease KLK6 | GKPYEGTQTMRIKVVEGGPLPFAFDILATSFLYGSKT | |
| •mTagBFP2 | FINHTQGIPDFFKQSFPEGFTWERVTTYEDGGVLTAT | |
| •Linker | QDTSLQDGCLIYNVKIRGVNFTSNGPVMQKKTLGWE | |
| •P2A | AFTETLYPADGGLEGRNDMALKLVGGSHLIANAKTTY | |
| •TCRβ Leader | RSKKPAKNLKMPGVYYVDYRLERIKEANNETYVEQH | |
| •RQR8 Tag | EVAVARYCDLPSKLGHKLNNASATNFSLLKQAGDVE | |
| •CD8α Hinge | ENPGPMGTSLLCWMALCLLGADHADACPYSNPSLC | |
| •CD8α Tm | SGGGGSELPTQGTFSNVSTNVSPAKPTTTACPYSNP | |
| •CD8α intracellular linker | SLCSGGGGSPAPRPPTPAPTIASQPLSLRPEACRPA | |
| •KLK6 protease | AGGAVHTRGLDFACDIYIWAPLAGTCGVLLLSLVITLY | |
| •linker | CNHRNRRRVCKCPRPVVGSSGNSSGGSGSTGSSG | |
| •adenovirus E3-19K ER | GSGGSGSSGGSLVHGGPCDKTSHPYQAALYTSGHL | |
| retention tag | LCGGVLIHPLWVLTAAHCKKPNLQVFLGKHNLRQRE | |
| | SSQEQSSVVRAVIHPDYDAASHDQDIMLLRLARPAKL | |
| | SELIQPLPLERDCSANTTSCHILGWGKTADGDFPDTI | |
| | QCAYIHLVSREECEHAYPGQITQNMLCAGDEKYGKD | |
| | SCQGDSGGPLVCGDHLRGLVSWGNIPCGSKEKPGV | |
| | YTNVCRYTNWIQKTIQAKAAASTGSSGGGGGSGGLY | |
| | KYKSRRSFIDEKKMP | |
| # 824 Two-way protease using | MVSKGEELIKENMHMKLYMEGTVDNHHFKCTSEGE | 118 |
| human protease KLK8 | GKPYEGTQTMRIKVVEGGPLPFAFDILATSFLYGSKT | |
| •mTagBFP2 | FINHTQGIPDFFKQSFPEGFTWERVTTYEDGGVLTAT | |
| •Linker | QDTSLQDGCLIYNVKIRGVNFTSNGPVMQKKTLGWE | |
| •P2A | AFTETLYPADGGLEGRNDMALKLVGGSHLIANAKTTY | |
| •TCRβ Leader | RSKKPAKNLKMPGVYYVDYRLERIKEANNETYVEQH | |
| •RQR8 Tag | EVAVARYCDLPSKLGHKLNNASATNFSLLKQAGDVE | |
| •CD8α Hinge | ENPGPMGTSLLCWMALCLLGADHADACPYSNPSLC | |
| •CD8α Tm | SGGGGSELPTQGTFSNVSTNVSPAKPTTTACPYSNP | |
| •CD8α intracellular linker | SLCSGGGGSPAPRPPTPAPTIASQPLSLRPEACRPA | |
| •KLK8 protease | AGGAVHTRGLDFACDIYIWAPLAGTCGVLLLSLVITLY | |
| •linker | CNHRNRRRVCKCPRPVVGSSGNSSGGSGSTGSSG | |
| •adenovirus E3-19K ER | GSGGSGSSGGSVLGGHECQPHSQPWQAALFQGQQ | |
| retention tag | LLCGGVLVGGNWVLTAAHCKKPKYTVRLGDHSLQN | |
| | KDGPEQEIPVVQSIPHPCYNSSDVEDHNHDLMLLQL | |
| | RDQASLGSKVKPISLADHCTQPGQKCTVSGWGTVTS | |
| | PRENFPDTLNCAEVKIFPQKKCEDAYPGQITDGMVC | |
| | AGSSKGADTCQGDSGGPLVCDGALQGITSWGSDPC | |
| | GRSDKPGVYTNICRYLDWIKKIIGSKGAAASTGSSGG | |
| | GGGSGGLYKYKSRRSFIDEKKMP | |
| # 825 Two-way protease using | MVSKGEELIKENMHMKLYMEGTVDNHHFKCTSEGE | 119 |
| human protease KLK11 | GKPYEGTQTMRIKVVEGGPLPFAFDILATSFLYGSKT | |
| •mTagBFP2 | FINHTQGIPDFFKQSFPEGFTWERVTTYEDGGVLTAT | |
| •Linker | QDTSLQDGCLIYNVKIRGVNFTSNGPVMQKKTLGWE | |
| •P2A | AFTETLYPADGGLEGRNDMALKLVGGSHLIANAKTTY | |
| •TCRβ Leader | RSKKPAKNLKMPGVYYVDYRLERIKEANNETYVEQH | |
| •RQR8 Tag | EVAVARYCDLPSKLGHKLNNASATNFSLLKQAGDVE | |
| •CD8α Hinge | ENPGPMGTSLLCWMALCLLGADHADACPYSNPSLC | |
| •CD8α Tm | SGGGGSELPTQGTFSNVSTNVSPAKPTTTACPYSNP | |
| •CD8α intracellular linker | SLCSGGGGSPAPRPPTPAPTIASQPLSLRPEACRPA | |
| •KLK11 protease | AGGAVHTRGLDFACDIYIWAPLAGTCGVLLLSLVITLY | |
| •linker | CNHRNRRRVCKCPRPVVGSSGNSSGGSGSTGSSG | |
| •adenovirus E3-19K ER | GSGGSGSSGGSIIKGFECKPHSQPWQAALFEKTRLL | |
| retention tag | CGATLIAPRWLLTAAHCLKPRYIVHLGQHNLQKEEGC | |
| | EQTRTATESFPHPGFNNSLPNKDHRNDIMLVKMASP | |
| | VSITWAVRPLTLSSRCVTAGTSCLISGWGSTSSPQLR | |
| | LPHTLRCANITIIEHQKCENAYPGNITDTMVCASVQEG | |
| | GKDSCQGDSGGPLVCNQSLQGIISWGQDPCAITRKP | |
| | GVYTKVCKYVDWIQETMKNNAAASTGSSGGGGGSG | |
| | GLYKYKSRRSFIDEKKMP | |
| # 826 Two-way protease using | MVSKGEELIKENMHMKLYMEGTVDNHHFKCTSEGE | 120 |
| human protease KLK13 | GKPYEGTQTMRIKVVEGGPLPFAFDILATSFLYGSKT | |
| •mTagBFP2 | FINHTQGIPDFFKQSFPEGFTWERVTTYEDGGVLTAT | |
| •Linker | QDTSLQDGCLIYNVKIRGVNFTSNGPVMQKKTLGWE | |
| •P2A | AFTETLYPADGGLEGRNDMALKLVGGSHLIANAKTTY | |
| •TCRβ Leader | RSKKPAKNLKMPGVYYVDYRLERIKEANNETYVEQH | |
| •RQR8 Tag | EVAVARYCDLPSKLGHKLNNASATNFSLLKQAGDVE | |
| •CD8α Hinge | ENPGPMGTSLLCWMALCLLGADHADACPYSNPSLC | |
| •CD8α Tm | SGGGGSELPTQGTFSNVSTNVSPAKPTTTACPYSNP | |
| •CD8α intracellular linker | SLCSGGGGSPAPRPPTPAPTIASQPLSLRPEACRPA | |
| •KLK13 protease | AGGAVHTRGLDFACDIYIWAPLAGTCGVLLLSLVITLY | |
| •linker | CNHRNRRRVCKCPRPVVGSSGNSSGGSGSTGSSG | |
| •adenovirus E3-19K ER | GSGGSGSSGGSGGVSQESSKVLNTNGTSGFLPGGY | |
| retention tag | TCFPHSQPWQAALLVQGRLLCGGVLVHPKWVLTAA | |
| | HCLKEGLKVYLGKHALGRVEAGEQVREVVHSIPHPE | |
| | YRRSPTHLNHDHDIMLLELQSPVQLTGYIQTLPLSHN | |
| | NRLTPGTTCRVSGWGTTTSPQVNYPKTLQCANIQLR | |
| | SDEECRQVYPGKITDNMLCAGTKEGGKDSCEGDSG | |
| | GPLVCNRTLYGIVSWGDFPCGQPDRPGVYTRVSRY | |
| | VLWIRETIRKYETQQQKWLKGPQAAASTGSSGGGG | |
| | GSGGLYKYKSRRSFIDEKKMP | |
| # 827 Two-way protease using | MVSKGEELIKENMHMKLYMEGTVDNHHFKCTSEGE | 121 |
| human protease KLK14 | GKPYEGTQTMRIKVVEGGPLPFAFDILATSFLYGSKT | |
| •mTagBFP2 | FINHTQGIPDFFKQSFPEGFTWERVTTYEDGGVLTAT | |
| •Linker | QDTSLQDGCLIYNVKIRGVNFTSNGPVMQKKTLGWE | |
| •P2A | AFTETLYPADGGLEGRNDMALKLVGGSHLIANAKTTY | |
| •TCRβ Leader | RSKKPAKNLKMPGVYYVDYRLERIKEANNETYVEQH | |
| •RQR8 Tag | EVAVARYCDLPSKLGHKLNNASATNFSLLKQAGDVE | |
| •CD8α Hinge | ENPGPMGTSLLCWMALCLLGADHADACPYSNPSLC | |
| •CD8α Tm | SGGGGSELPTQGTFSNVSTNVSPAKPTTTACPYSNP | |
| •CD8α intracellular linker | SLCSGGGGSPAPRPPTPAPTIASQPLSLRPEACRPA | |
| •KLK14 protease | AGGAVHTRGLDFACDIYIWAPLAGTCGVLLLSLVITLY | |
| •linker | CNHRNRRRVCKCPRPVVGSSGNSSGGSGSTGSSG | |
| •adenovirus E3-19K ER | GSGGSGSSGGSIIGGHTCTRSSQPWQAALLAGPRR | |
| retention tag | RFLCGGALLSGQWVITAAHCGRPILQVALGKHNLRR | |
| | WEATQQVLRVVRQVTHPNYNSRTHDNDLMLLQLQQ | |
| | PARIGRAVRPIEVTQACASPGTSCRVSGWGTISSPIA | |
| | RYPASLQCVNINISPDEVCQKAYPRTITPGMVCAGVP | |
| | QGGKDSCQGDSGGPLVCRGQLQGLVSWGMERCAL | |
| | PGYPGVYTNLCKYRSWIEETMRDKAAASTGSSGGG | |
| | GGSGGLYKYKSRRSFIDEKKMP | |
| # 828 Two-way protease using | MVSKGEELIKENMHMKLYMEGTVDNHHFKCTSEGE | 122 |
| human protease KLK15 | GKPYEGTQTMRIKVVEGGPLPFAFDILATSFLYGSKT | |
| •mTagBFP2 | FINHTQGIPDFFKQSFPEGFTWERVTTYEDGGVLTAT | |
| •Linker | QDTSLQDGCLIYNVKIRGVNFTSNGPVMQKKTLGWE | |
| •P2A | AFTETLYPADGGLEGRNDMALKLVGGSHLIANAKTTY | |
| •TCRß Leader | RSKKPAKNLKMPGVYYVDYRLERIKEANNETYVEQH | |
| •RQR8 Tag | EVAVARYCDLPSKLGHKLNNASATNFSLLKQAGDVE | |
| •CD8α Hinge | ENPGPMGTSLLCWMALCLLGADHADACPYSNPSLC | |
| •CD8α Tm | SGGGGSELPTQGTFSNVSTNVSPAKPTTTACPYSNP | |
| •CD8α intracellular linker | SLCSGGGGSPAPRPPTPAPTIASQPLSLRPEACRPA | |
| •KLK15 protease | AGGAVHTRGLDFACDIYIWAPLAGTCGVLLLSLVITLY | |
| •linker | CNHRNRRRVCKCPRPVVGSSGNSSGGSGSTGSSG | |
| •adenovirus E3-19K ER | GSGGSGSSGGSLLEGDECAPHSQPWQVALYERGR | |
| retention tag | FNCGASLISPHWVLSAAHCQSRFMRVRLGEHNLRKR | |
| | DGPEQLRTTSRVIPHPRYEARSHRNDIMLLRLVQPAR | |
| | LNPQVRPAVLPTRCPHPGEACVVSGWGLVSHNEPG | |
| | TAGSPRSQVSLPDTLHCANISIISDTSCDKSYPGRLTN | |
| | TMVCAGAEGRGAESCEGDSGGPLVCGGILQGIVSW | |
| | GDVPCDNTTKPGVYTKVCHYLEWIRETMKRNAAAST | |
| | GSSGGGGGSGGLYKYKSRRSFIDEKKMP | |
| # 776 AAV6 vector for EGFR STASH | CCTATTAAATAAAAGAATAAGCAGTATTATTAAGTA | 123 |
| 501 TRAC knock-in (nucleotide | GCCCTGCATTTCAGGTTTCCTTGAGTGGCAGGCCA | |
| sequence) | GGCCTGGCCGTGAACGTTCACTGAAATCATGGCCT | |
| •TRAC left homology arm | CTTGGCCAAGATTGATAGCTTGTGCCTGTCCCTGA | |
| •Linker | GTCCCAGTCCATCACGAGCAGCTGGTTTCTAAGAT | |
| •EF1a promoter | GCTATTTCCCGTATAAAGCATGAGACCGTGACTTG | |
| •Linker | CCAGCCCCACAGAGCCCCGCCCTTGTCCATCACT | |
| •EGFR STASH variant 501 | GGCATCTGGACTCCAGCCTGGGTTGGGGCAAAGA | |
| •linker | GGGAAATGAGATCATGTCCTAACCCTGATCCTCTT | |
| •bGH Poly(A) signal | GTCCCACAGATATCCAGAACCCTGACCCTGCCGTG | |
| •TRAC right homology arm | TACCAGCTGAGAGACTCTAAATCCAGTGACAAGTC | |
| | TGTCTGCCTATTCACCAACGCGTCTTAGAAGGATC | |
| | TGCGATCGCTCCGGTGCCCGTCAGTGGGCAGAGC | |
| | GCACATCGCCCACAGTCCCCGAGAAGTTGGGGGG | |
| | AGGGGTCGGCAATTGAACGGGTGCCTAGAGAAGG | |
| | TGGCGCGGGGTAAACTGGGAAAGTGATGTCGTGT | |
| | ACTGGCTCCGCCTTTTTCCCGAGGGTGGGGGAGA | |
| | ACCGTATATAAGTGCAGTAGTCGCCGTGAACGTTC | |
| | TTTTTCGCAACGGGTTTGCCGCCAGAACACAGCTG | |
| | AAGCTTCGAGGGGCTCGCATCTCTCCTTCACGCG | |
| | CCCGCCGCCCTACCTGAGGCCGCCATCCACGCCG | |
| | GTTGAGTCGCGTTCTGCCGCCTCCCGCCTGTGGT | |
| | GCCTCCTGAACTGCGTCCGCCGTCTAGGTAAGTTT | |
| | AAAGCTCAGGTCGAGACCGGGCCTTTGTCCGGCG | |
| | CTCCCTTGGAGCCTACCTAGACTCAGCCGGCTCTC | |
| | CACGCTTTGCCTGACCCTGCTTGCTCAACTCTACG | |
| | TCTTTGTTTCGTTTTCTGTTCTGCGCCGTTACAGAT | |
| | CCAAGCTGTGACCGGCGCCTAatcgcgagcGCCGCC | |
| | ACcATGCTTCTCCTTGTAACGTCTCTCCTTTTGTGC | |
| | GAACTTCCCCATCCGGCCTTCCTTTTGATACCCCG | |
| | CAAGGTGTGTAACGGAATTGGGATTGGTGAATTCA | |
| | AGGATTCACTTAGTATAAATGCCACAAACATTAAGC | |
| | ATTTTAAGAACTGTACGTCCATTTCAGGAGACCTG | |
| | CATATTCTTCCCGTAGCTTTCCGGGGCGATTCATT | |
| | CACCCACACACCGCCACTTGATCCACAAGAACTTG | |
| | ACATCCTGAAAACGGTTAAGGAGATAACAGGATTC | |
| | CTCCTGATACAGGCCTGGCCCGAGAATAGAACCG | |
| | ACTTGCACGCCTTTGAAAATTTGGAGATAATTCGG | |
| | GGTCGGACTAAGCAACATGGACAATTTTCACTGGC | |
| | GGTAGTTTCTTTGAATATTACGAGCCTCGGCCTTA | |
| | GATCTCTCAAGGAGATCTCAGACGGCGACGTTATA | |
| | ATATCTGGGAACAAGAACCTGTGCTACGCTAACAC | |
| | AATCAATTGGAAAAAGCTGTTCGGCACGTCTGGAC | |
| | AAAAGACAAAGATAATTTCAAATCGAGGCGAAAATA | |
| | GCTGCAAGGCTACGGGACAGGTTTGTCACGCCCT | |
| | CTGTAGCCCAGAGGGCTGTTGGGGACCCGAGCCA | |
| | AGAGATTGCGTCTCATGTCGGAATGTGTCCCGAGG | |
| | CCGAGAATGTGTCGATAAATGCAATCTTCTGGAGG | |
| | GAGAACCACGGGAATTCGTTGAAAACAGTGAGTGC | |
| | ATTCAATGTCACCCGGAGTGCCTTCCGCAAGCGAT | |
| | GAATATTACATGTACAGGCCGGGGTCCCGATAATT | |
| | GCATCCAGTGTGCTCATTATATTGACGGACCACAC | |
| | TGTGTAAAGACATGCCCTGCCGGCGTTATGGGTGA | |
| | AAACAATACGCTGGTCTGGAAGTATGCAGACGCAG | |
| | GACATGTTTGTCACCTGTGCCATCCTAACTGCACG | |
| | TATGGCTGTACAGGACCGGGTCTGGAAGGCTGCC | |
| | CTACGAATGGTCCCAAAATACCATCAggaggatccATA | |
| | GCCTTCCTCCTCGCaTGCGTCGCTACCATGATCTT | |
| | CATGATAACTAAATGCTGTCTCTTCGCGGCCGCAg | |
| | gtGGTACAgagaaccTCTATTTTCAGTCAgggtcgacaTG | |
| | TTTCAGGAAACTGGCGAAGACAGGTAAGAAAAAAA | |
| | AAAGAGACtaatgatgagtataccctgcaggTtaattagct | |
| | aacaattgCACAGTAAGAATTCTAACTAGAGCTCGCTGATCA | |
| | GCCTCGACTGTGCCTTCTAGTTGCCAGCCATCTGT | |
| | TGTTTGCCCCTCCCCCGTGCCTTCCTTGACCCTGG | |
| | AAGGTGCCACTCCCACTGTCCTTTCCTAATAAAAT | |
| | GAGGAAATTGCATCGCATTGTCTGAGTAGGTGTCA | |
| | TTCTATTCTGGGGGGTGGGGTGGGGCAGGACAGC | |
| | AAGGGGGAGGATTGGGAAGAGAATAGCAGGCATG | |
| | CTGGGGAGCGGCCGCGTTAAgttaacATTTTGATTCT | |
| | CAAACAAATGTGTCACAAAGTAAGGATTCTGATGT | |
| | GTATATCACAGACAAAACTGTGCTAGACATGAGGT | |
| | CTATGGACTTCAAGAGCAACAGTGCTGTGGCCTGG | |
| | AGCAACAAATCTGACTTTGCATGTGCAAACGCCTT | |
| | CAACAACAGCATTATTCCAGAAGACACCTTCTTCC | |
| | CCAGCCCAGGTAAGGGCAGCTTTGGTGCCTTCGC | |
| | AGGCTGTTTCCTTGCTTCAGGAATGGCCAGGTTCT | |
| | GCCCAGAGCTCTGGTCAATGATGTCTAAAACTCCT | |
| | CTGATTGGTGGTCTCGGCCTTATCCATTGCCACCA | |
| | AAACCCTCTTTTTACTAAGAAACAGTGAGCCTTGTT | |
| | CTGGCAGTCCAGAGAATGACACGGGAAAAAAGCA | |
| | GATG | |
| EGFR Truncation | RKVCNGIGIGEFKDSLSINATNIKHFKNCTSISGDLHIL | 124 |
| # 789 EGFR Domain III only | PVAFRGDSFTHTPPLDPQELDILKTVKEITGFLLIQAW | |
| •EGFR Domain III | PENRTDLHAFENLEIIRGRTKQHGQFSLAVVSLNITSL | |
| | GLRSLKEISDGDVIISGNKNLCYANTINWKKLFGTSGQ | |
| | KTKIISNRGENSCKATGQ | |
| EGFR Truncation | RKVCNGIGIGEFKDSLSINATNIKHFKNCTSISGDLHIL | 125 |
| # 790 EGFR Domain III + Domain | PVAFRGDSFTHTPPLDPQELDILKTVKEITGFLLIQAW | |
| IV₅₀₁ | PENRTDLHAFENLEIIRGRTKQHGQFSLAVVSLNITSL | |
| •EGFR Domain III | GLRSLKEISDGDVIISGNKNLCYANTINWKKLFGTSGQ | |
| •EGFR Domain IV501 | KTKIISNRGENSCKATGQVCHALCSPEGCWGPEPRD | |
| | CVS | |
| EGFR Truncation | RKVCNGIGIGEFKDSLSINATNIKHFKNCTSISGDLHIL | 126 |
| #791 EGFR Domain III + Domain | PVAFRGDSFTHTPPLDPQELDILKTVKEITGFLLIQAW | |
| IV ₅₁₄ | PENRTDLHAFENLEIIRGRTKQHGQFSLAVVSLNITSL | |
| •EGFR Domain III | GLRSLKEISDGDVIISGNKNLCYANTINWKKLFGTSGQ | |
| •EGFR Domain IV514 | KTKIISNRGENSCKATGQVCHALCSPEGCWGPEPRD | |
| | CVSCRNVSRGRECVDK | |
| EGFR Truncation | RKVCNGIGIGEFKDSLSINATNIKHFKNCTSISGDLHIL | 127 |
| # 792 EGFR Domain III + Domain | PVAFRGDSFTHTPPLDPQELDILKTVKEITGFLLIQAW | |
| IV₅₂₄ | PENRTDLHAFENLEIIRGRTKQHGQFSLAVVSLNITSL | |
| •EGFR Domain III | GLRSLKEISDGDVIISGNKNLCYANTINWKKLFGTSGQ | |
| •EGFR Domain IV524 | KTKIISNRGENSCKATGQVCHALCSPEGCWGPEPRD | |
| | CVSCRNVSRGRECVDKCNLLEGEPRE | |
| EGFR Truncation | RKVCNGIGIGEFKDSLSINATNIKHFKNCTSISGDLHIL | 128 |
| # 793 EGFR Domain III + Domain | PVAFRGDSFTHTPPLDPQELDILKTVKEITGFLLIQAW | |
| IV₅₂₉ | PENRTDLHAFENLEIIRGRTKQHGQFSLAVVSLNITSL | |
| •EGFR Domain III | GLRSLKEISDGDVIISGNKNLCYANTINWKKLFGTSGQ | |
| •EGFR Domain IV529 | KTKIISNRGENSCKATGQVCHALCSPEGCWGPEPRD | |
| | CVSCRNVSRGRECVDKCNLLEGEPREFVENS | |
| EGFR Truncation | RKVCNGIGIGEFKDSLSINATNIKHFKNCTSISGDLHIL | 129 |
| # 794 EGFR Domain III + Domain | PVAFRGDSFTHTPPLDPQELDILKTVKEITGFLLIQAW | |
| IV₅₃₃ | PENRTDLHAFENLEIIRGRTKQHGQFSLAVVSLNITSL | |
| •EGFR Domain III | GLRSLKEISDGDVIISGNKNLCYANTINWKKLFGTSGQ | |
| •EGFR Domain IV533 | KTKIISNRGENSCKATGQVCHALCSPEGCWGPEPRD | |
| | CVSCRNVSRGRECVDKCNLLEGEPREFVENSECIQ | |
| Nucleotide sequence of CRISPR | GGGAATCAAAATCGGTGAAT | 130 |
| guide for TRAC knock-in | | |
| experiments | | |

### Cells, Compositions and Methods of Use

### Cells

Also provided by the present disclosure are cells. According to some embodiments, provided is a cell comprising two or more separate expression constructs, wherein the two or more separate expression constructs comprise a first expression construct that encodes a fusion protein comprising a selection marker, a protein localization tag, and a protease cleavage site disposed between the selection marker and the protein localization tag. The two or more separate expression constructs further comprise a second expression construct that encodes a protein required for cell surface expression of the selection marker. The first and/or second expression construct may further encode a protein of interest, e.g., any of the proteins of interest described elsewhere herein. In certain embodiments, the first and/or second expression construct is site-specifically integrated into the genome of the cell. The site-specific integration may result in the inactivation of one or more target genes in the genome of the cell.

The present disclosure also provides cells or progeny thereof selected according to the cell selection methods of the present disclosure.

Cells of the present disclosure may be autologous/autogeneic (“self”) or non-autologous (“non-self,” e.g., allogeneic, syngeneic or xenogeneic). “Autologous” as used herein, refers to cells derived from the same individual to which they are subsequently administered. “Allogeneic” as used herein refers to cells of the same species that differ genetically from the cell in comparison. “Syngeneic,” as used herein, refers to cells of a different individual that are genetically identical to the cell in comparison.

In some embodiments, the cells are T cells obtained from a mammal. In some embodiments, the mammal is a primate. In some embodiments, the primate is a human.

T cells may be obtained from a number of sources including, but not limited to, peripheral blood, peripheral blood mononuclear cells, bone marrow, lymph node tissue, cord blood, thymus tissue, tissue from a site of infection, ascites, pleural effusion, spleen tissue, and tumors. In certain embodiments, T cells can be obtained from a unit of blood collected from an individual using any number of known techniques such as sedimentation, e.g., FICOLL™ separation.

In some embodiments, an isolated or purified population of T cells is used. In some embodiments, T_{CTL} and T_{H} lymphocytes are purified from PBMCs. In some embodiments, the T_{CTL} and T_{H} lymphocytes are sorted into naïve (T_{N}), memory (T_{MEM}), stem cell memory (T_{SCM}), central memory (T_{CM}), effector memory (T_{EM}), and effector (T_{EFF}) T cell subpopulations either before or after activation, expansion, and/or genetic modification. Suitable approaches for such sorting are known and include, e.g., magnetic-activated cell sorting (MACS), where TN are CD45RA⁺ CD62L⁺ CD95⁻; TSCM are CD45RA⁺ CD62L⁺ CD95⁺; TCM are CD45RO⁺ CD62L⁺ CD95⁺; and TEM are CD45RO⁺ CD62L⁻ CD95⁺. An exemplary approach for such sorting is described in Wang et al. (2016) Blood 127 (24): 2980-90.

A specific subpopulation of T cells expressing one or more of the following markers: CD3, CD4, CD8, CD28, CD45RA, CD45RO, CD62, CD127, and HLA-DR can be further isolated by positive or negative selection techniques. In some embodiments, a specific subpopulation of T cells, expressing one or more of the markers selected from the group consisting of CD62L, CCR7, CD28, CD27, CD122, CD127, CD197; or CD38 or CD62L, CD127, CD197, and CD38, is further isolated by positive or negative selection techniques. In some embodiments, the manufactured T cell compositions do not express one or more of the following markers: CD57, CD244, CD 160, PD-1, CTLA4, TIM3, and LAG3. In some embodiments, the manufactured T cell compositions do not substantially express one or more of the following markers: CD57, CD244, CD 160, PD-1, CTLA4, TIM3, and LAG3.

In order to achieve therapeutically effective doses of T cell compositions, the T cells may be subjected to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety for all purposes. In some embodiments, T cells are activated and expanded for about 1 to 21 days, e.g., about 5 to 21 days. In some embodiments, T cells are activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to introduction of a nucleic acid (e.g., expression vector) encoding the polypeptide into the T cells.

In some embodiments, T cells are activated and expanded for about 6 hours, about 12 hours, about 18 hours or about 24 hours prior to introduction of a nucleic acid (e.g., expression vector) encoding the cell surface receptor the into the T cells. In some embodiments, T cells are activated at the same time that a nucleic acid (e.g., an expression vector) encoding the cell surface receptor is introduced into the T cells.

In some embodiments, conditions appropriate for T cell culture include an appropriate media (e.g., Minimal Essential Media or RPMI Media 1640 or, X-vivo 15, (Lonza)) and one or more factors necessary for proliferation and viability including, but not limited to serum (e.g., fetal bovine or human serum), interleukin-2 (IL-2), insulin, IFN-γ, IL-4, IL-7, IL-21, GM-CSF, IL-10, IL-12, IL-15, TGFβ, and TNF-α or any other additives suitable for the growth of cells known to the skilled artisan. Further illustrative examples of cell culture media include, but are not limited to RPMI 1640, Clicks, AEVI-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells.

### Compositions

Also provided by the present disclosure are compositions. According to some embodiments, provided are compositions comprising any of the cells of the present disclosure or progeny thereof, e.g., cells selected according to the methods of the present disclosure, etc.

Such compositions may comprise the cells present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl) piperazine-N′-(2-ethanesulfonic acid) (HEPES), 2-(N-Morpholino) ethanesulfonic acid (MES), 2-(N-Morpholino) ethanesulfonic acid sodium salt (MES), 3-(N-Morpholino) propanesulfonic acid (MOPS), N-tris [Hydroxymethyl]methyl-3-aminopropanesulfonic acid (TAPS), etc.), a solubilizing agent, a detergent (e.g., a non-ionic detergent such as Tween-20, etc.), a nuclease inhibitor, glycerol, a chelating agent, and the like may be present in such compositions. In certain embodiments, the liquid medium is a cell culture medium. Non-limiting examples of cell culture media include Minimal Essential Media, DMEM, a-MEM, RPMI Media, Clicks, F-12, X-Vivo 15, X-Vivo 20, Optimizer, and the like.

In certain embodiments, provided are pharmaceutical compositions comprising cells or progeny thereof selected according to the methods of the present disclosure. The pharmaceutical compositions may comprise such cells and a pharmaceutically acceptable carrier. The pharmaceutical compositions generally include a therapeutically effective amount of the cells. By “therapeutically effective amount” is meant a number of cells sufficient to produce a desired result, e.g., an amount sufficient to effect beneficial or desired therapeutic (including preventative) results, such as a reduction in a symptom of a disease (e.g., cancer) or disorder associated, e.g., with the target cell or a population thereof (e.g., cancer cells), as compared to a control. An effective amount can be administered in one or more administrations. A therapeutically effective amount may vary according to factors such as the disease state, age, sex, and weight of the individual, and the ability of the cells to elicit a desired response in the individual. A therapeutically effective amount is also one in which any toxic or detrimental effects of the cells are outweighed by the therapeutically beneficial effects. The term “therapeutically effective amount” includes an amount that is effective to “treat” an individual, e.g., a patient. When a therapeutic amount is indicated, the precise amount of the compositions contemplated in particular embodiments, to be administered, can be determined by a physician in view of the specification and with consideration of individual differences in age, weight, tumor size, extent of infection or metastasis, and condition of the patient (individual). In some embodiments, a pharmaceutical composition of the present disclosure includes from 1×10⁶ to 5×10¹⁰ of the cells of the present disclosure.

The cells of the present disclosure can be incorporated into a variety of formulations for therapeutic administration. More particularly, the cells of the present disclosure can be formulated into pharmaceutical compositions by combination with appropriate, pharmaceutically acceptable excipients or diluents.

Formulations of the cells suitable for administration to a patient (e.g., suitable for human administration) are generally sterile and may further be free of detectable pyrogens or other contaminants contraindicated for administration to a patient according to a selected route of administration.

The cells may be formulated for parenteral (e.g., intravenous, intra-arterial, intraosseous, intramuscular, intracerebral, intracerebroventricular, intrathecal, subcutaneous, etc.) administration, or any other suitable route of administration.

Pharmaceutical compositions that include the cells of the present disclosure may be prepared by mixing the cells having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

An aqueous formulation of the cells may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included in the formulation to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20TM) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

In some embodiments, the pharmaceutical composition comprises cells of the present disclosure, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Methods of Use

Also provided by the present disclosure are methods of using the cells and compositions of the present disclosure. In certain embodiments, the methods comprise administering a therapeutically effective amount of any of the pharmaceutical compositions of the present disclosure to an individual in need thereof.

In some embodiments, the individual in need thereof has cancer, and one or more of the two or more separate expression constructs encode a receptor (e.g., a CAR, a TCR, and/or the like) that binds to a molecule on the surface of the cancer cells. The pharmaceutical composition typically includes a therapeutically effective amount of such cells as described above. The cells may be any cells capable of effecting the desired therapy. In some embodiments, the cells are immune cells. Non-limiting examples of immune cells which may be administered include T cells, B cells, natural killer (NK) cells, macrophages, monocytes, neutrophils, dendritic cells, mast cells, basophils, and eosinophils. In certain embodiments, the cells are T cells. According to some embodiments, the cells are T cells and a protein of interest expressed by one or more of the two or more expression constructs is a CAR, such that the cells are CAR T cells. In certain embodiments, the cells are stem cells, e.g., embryonic stem cells or adult stem cells. In some embodiments, the pharmaceutical composition is an autologous composition produced by a method including removing cells from the individual and introducing into the removed cells or progeny thereof the desired two or more expression constructs, followed by selection of such cells based on cell surface expression of the selection marker.

In certain embodiments, the individual in need thereof has a cell proliferative disorder. By “cell proliferative disorder” is meant a disorder wherein unwanted cell proliferation of one or more subset(s) of cells in a multicellular organism occurs, resulting in harm, for example, pain or decreased life expectancy to the organism. Cell proliferative disorders include, but are not limited to, cancer, pre-cancer, benign tumors, blood vessel proliferative disorders (e.g., arthritis, restenosis, and the like), fibrotic disorders (e.g., hepatic cirrhosis, atherosclerosis, and the like), psoriasis, epidermic and dermoid cysts, lipomas, adenomas, capillary and cutaneous hemangiomas, lymphangiomas, nevi lesions, teratomas, nephromas, myofibromatosis, osteoplastic tumors, dysplastic masses, mesangial cell proliferative disorders, and the like.

In some embodiments, the individual has cancer. The subject methods may be employed for the treatment of a large variety of cancers. “Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. Examples of cancers that may be treated according to the methods of the present disclosure include, but are not limited to, carcinoma, lymphoma, blastoma, and sarcoma. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bile duct cancer, bladder cancer, hepatoma, breast cancer, colon cancer, colorectal cancer, endometrial or uterine carcinoma, salivary gland carcinoma, kidney cancer, prostate cancer, vulval cancer, thyroid cancer, hepatic carcinoma, various types of head and neck cancer, and the like. In certain embodiments, the individual has a cancer selected from a solid tumor, recurrent glioblastoma multiforme (GBM), non-small cell lung cancer, metastatic melanoma, melanoma, peritoneal cancer, epithelial ovarian cancer, glioblastoma multiforme (GBM), metastatic colorectal cancer, colorectal cancer, pancreatic ductal adenocarcinoma, squamous cell carcinoma, esophageal cancer, gastric cancer, neuroblastoma, fallopian tube cancer, bladder cancer, metastatic breast cancer, pancreatic cancer, soft tissue sarcoma, recurrent head and neck cancer squamous cell carcinoma, head and neck cancer, anaplastic astrocytoma, malignant pleural mesothelioma, breast cancer, squamous non-small cell lung cancer, rhabdomyosarcoma, metastatic renal cell carcinoma, basal cell carcinoma (basal cell epithelioma), and gliosarcoma. In certain aspects, the individual has a cancer selected from melanoma, Hodgkin lymphoma, renal cell carcinoma (RCC), bladder cancer, non-small cell lung cancer (NSCLC), and head and neck squamous cell carcinoma (HNSCC).

### Kits

Also provided by the present disclosure are kits. In certain embodiments, provided are kits that include any reagents that find use in practicing the methods of the present disclosure. By way of example, in certain embodiments, provided are kits that comprise a first expression construct that encodes a fusion protein comprising a selection marker, a protein localization tag, and a protease cleavage site disposed between the selection marker and the protein localization tag, and a second expression construct that encodes a protein required for cell surface expression of the selection marker. The first and/or second expression constructs may further comprise a cloning site for a nucleic acid encoding a protein of interest. In certain embodiments, the first and/or second expression constructs further encode one or more proteins of interest, e.g., any of the proteins of interest described elsewhere herein.

The kits of the present disclosure may further include any other reagents useful for practicing the methods of the present disclosure, such as transfection/transduction reagents useful for introducing the expression constructs into cells of interest, e.g., immune cells (e.g., T cells) or other cells of interest.

Components of the kits may be present in separate containers, or multiple components may be present in a single container. For example, the first and second expression constructs may be provided in separate containers or the same container. A suitable container includes a single tube (e.g., vial), one or more wells of a plate (e.g., a 96-well plate, a 384-well plate, etc.), or the like.

The kits of the present disclosure may further comprise instructions for contacting a population of cells with the two or more expression constructs under conditions in which the two or more expression constructs are delivered to cells of the population of cells. The kits of the present disclosure may further comprise instructions for selecting for cells exhibiting cell surface expression of the selection marker.

The instructions of the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging), etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, the means for obtaining the instructions is recorded on a suitable substrate.

Notwithstanding the appended claims, the present disclosure is also defined by the following embodiments:

1. A method of selecting for cells that comprise two or more separate expression constructs, the method comprising:
contacting a population of cells with two or more separate expression constructs under conditions in which the two or more expression constructs are delivered to cells of the population of cells, wherein the two or more separate expression constructs comprise:
   a first expression construct that encodes a fusion protein comprising a selection marker, a protein localization tag, and a protease cleavage site disposed between the selection marker and the protein localization tag; and
   a second expression construct that encodes a protein required for cell surface expression of the selection marker; and
selecting for cells exhibiting cell surface expression of the selection marker.

2. The method according to embodiment 1, wherein the first expression construct further encodes a protein of interest.

3. The method according to embodiment 1 or embodiment 2, wherein the first expression construct site-specifically integrates into the genome of the cell.

4. The method according to embodiment 3, wherein site-specific integration of the first expression construct into the genome of the cell inactivates a target gene within the genome of the cell.

5. The method according to any one of embodiments 1 to 4, wherein the second expression construct further encodes a protein of interest.

6. The method according to any one of embodiments 1 to 5, wherein the second expression construct site-specifically integrates into the genome of the cell.

7. The method according to embodiment 6, wherein site-specific integration of the second expression construct into the genome of the cell inactivates a target gene within the genome of the cell.

8. The method according to any one of embodiments 1 to 7, wherein the protein localization tag is selected from the group consisting of: an endoplasmic reticulum (ER) localization tag, a Golgi apparatus (Golgi) localization tag, a lysosome localization tag, a plasma membrane localization tag, a mitochondria localization tag, a peroxisome localization tag, a cytosolic localization tag, and a nuclear localization tag.

9. The method according to any one of embodiments 1 to 7, wherein the protein localization tag is an ER localization tag.

10. The method according to embodiment 9, wherein the ER localization tag comprises the amino acid sequence KKMP.

11. The method according to embodiment 9, wherein the ER localization tag comprises 85% or greater, 90% or greater, or 100% amino acid sequence identity to an ER localization tag comprising, consisting of, or present within, an amino acid sequence selected from the group consisting of: LYKYKSRRSFIDEKKMP (SEQ ID NO:1); AEKDEL (SEQ ID NO:2); EQKLISEEDLKDEL (SEQ ID NO:3); GGGGSGGGGSKDEL (SEQ ID NO:4); GGGGSGGGGSGGGGSGGGGSKDEL (SEQ ID NO:5); GGGGSGGGGSGGGGSGGGGSAEKDEL (SEQ ID NO:6); KYKSRRSFIEEKKMP (SEQ ID NO: 7); LKYKSRRSFIEEKKMP (SEQ ID NO:8); LYKYKSRRSFIEEKKMP (SEQ ID NO:9); LYCKYKSRRSFIEEKKMP (SEQ ID NO:10); LYCNKYKSRRSFIEEKKMP (SEQ ID NO:11); LYCNKYKSRRSFIDEKKMP (SEQ ID NO:12); LYEQKLISEEDLKYKSRRSFIEEKKMP (SEQ ID NO: 13); LYCYPYDVPDYAKYKSRRSFIEEKKMP (SEQ ID NO:14); LYKKLETFKKTN (SEQ ID NO: 15); LYEQKLISEEDLKKLETFKKTN (SEQ ID NO:16); LYYQRL (SEQ ID NO:17); LYEQKLISEEDLYQRL (SEQ ID NO:18); LYKRKIIAFALEGKRSKVTRRPKASDYQRL (SEQ ID NO: 19); LYRNIKCD (SEQ ID NO:20); and LYEQKLISEEDLRNIKCD (SEQ ID NO:21).

12. The method according to embodiment 9, wherein the ER localization tag comprises 85% or greater, 90% or greater, 95% or greater, or 100% amino acid sequence identity to an ER localization tag comprising, consisting of, or present within, an amino acid sequence selected from the group consisting of:

(SEQ ID NO: 22)
PKKKQQKDSLINLKIQKENPKVVNEINIEDLCLTKAAYCRCWRSKTFPA
CDGSHNKHNELTGDNVGPLILKKKEV;
(SEQ ID NO: 23)
QMRHLKSFFEAKKLV;
(SEQ ID NO: 24)
AYRQRQHQDMPAPRPPGPRPAPPQQEGPPEQQPPQ;
(SEQ ID NO: 25)
HMKEKEKSD;
(SEQ ID NO: 26)
CFRKLAKTGKKKKRD;
(SEQ ID NO: 27)
KCCAYGYRKCLGKKGRVKKAHKSKTH;
(SEQ ID NO: 28)
YLSTCKDSKKKAE;
(SEQ ID NO: 29)
RLTTDVDPDLDQDED;
(SEQ ID NO: 30)
KYKSRRSFIDEKKMP;
(SEQ ID NO: 31)
MTGCCGCCCGCFGIIPLMSKCGKKSSYYTTFDNDVVIEQYRPKKSV;
(SEQ ID NO: 32)
NRSPRNRKPRRE;
(SEQ ID NO: 9)
LYKYKSRRSFIEEKKMP;
(SEQ ID NO: 33)
TKVLKGKKLSLPA;
(SEQ ID NO: 34)
KSNRHKDGFHRLRGHHDEYEDEIRMMSTGSKKSLLSHEFQDETDTEETL
YSSKH;
and
(SEQ ID NO: 35)
KCGKKSSYYTTFDNDVVIEQYRPKKSV.

13. The method according to embodiment 11 or embodiment 12, wherein the C-terminus of the ER localization tag comprises the four C-terminal residues of one of the sequences recited in embodiment 11 or embodiment 12.

14. The method according to embodiment 9, wherein the ER localization tag comprises a transmembrane (Tm) domain, an intracellular domain (ICD), or both, of an ER localization tag of a polypeptide set forth in Table 1, or a variant Tm and/or ICD thereof which retains the ability to localize a polypeptide to the ER.

15. The method according to embodiment 9, wherein the ER localization tag comprises a Tm domain, an ICD, or both, of an ER localization tag of a human ER-resident protein, or a variant Tm and/or ICD thereof which retains the ability to localize a polypeptide to the ER.

16. The method according to embodiment 15, wherein the human ER-resident protein is CDGSH iron sulfur domain 2 (CISD2).

17. The method according to embodiment 16, wherein the ER localization tag comprises the Tm domain, the ICD, or both, of the polypeptide set forth in SEQ ID NO:91, or a variant Tm and/or ICD thereof which retains the ability to localize a polypeptide to the ER.

18. The method according to embodiment 15, wherein the human ER-resident protein is UDP glucuronosyltransferase family 2 member B17 (UGT2B17).

19. The method according to embodiment 18, wherein the ER localization tag comprises the Tm domain, the ICD, or both, of the polypeptide set forth in SEQ ID NO:95, or a variant Tm and/or ICD thereof which retains the ability to localize a polypeptide to the ER.

20. The method according to any one of embodiments 1 to 7, wherein the protein localization tag is a Golgi localization tag.

21. The method according to embodiment 20, wherein the wherein the Golgi localization tag comprises the amino acid sequence YQRL (SEQ ID NO:36).

22. The method according to any one of embodiments 1 to 7, wherein the protein localization tag is a lysosome localization tag.

23. The method according to embodiment 22, wherein the lysosome localization tag comprises the amino acid sequence KFERQ (SEQ ID NO:37). 24. The method according to any one of embodiments 1 to 23, wherein the protease cleavage site is a viral protease cleavage site.

25. The method according to embodiment 24, wherein the viral protease cleavage site is a cleavage site for a potyviral family protease.

26. The method according to embodiment 25, wherein the potyviral family protease is Tobacco Etch Virus (TEV) protease, plum pox virus protease (PPVp), soybean mosaic virus protease (SbMVp), sunflower mild mosaic virus protease (SuMMVp), tobacco vein mottling virus protease (TVMVp), or West Nile virus protease (WNVp).

27. The method according to embodiment 25, wherein the viral protease cleavage site is a TEV protease cleavage site.

28. The method according to embodiment 24, wherein the viral protease cleavage site is for a viral protease derived from hepatitis C virus (HCV) nonstructural protein 3 (NS3).

29. The method according to embodiment 28, wherein the viral protease cleavage site is for a viral protease that further comprises a cofactor polypeptide derived from HCV nonstructural protein 4A (NS4A).

30. The method according to embodiment 28 or embodiment 29, wherein the viral protease cleavage site is selected from the group consisting of: an NS4A/4B junction cleavage site, an NS3/NS4A junction cleavage site, an NS4A/NS4B junction cleavage site, an NS4B/NS5A junction cleavage site, an NS5A/NS5B junction cleavage site, and variants thereof cleavable by the viral protease.

31. The method according to any one of embodiments 1 to 23, wherein the protease cleavage site is a human protease cleavage site.

32. The method according to embodiment 31, wherein the human protease cleavage site is a cleavage site for a human protease selected from the group consisting of: a human kallikrein (KLK) protease, human enterokinase protease, human thrombin, a human matrix metalloprotease (MMP), human urokinase-type plasminogen activator receptor (uPAR), human plasmin, and human cathepsin.

33. The method according to embodiment 32, wherein the human kallikrein protease is selected from the group consisting of: human KLK3, human KLK4, human KLK6, human KLK8, human KLK11, human KLK13, human KLK14, and human KLK15.

34. The method according to any one of embodiments 1 to 33, wherein the protein required for cell surface expression of the selection marker is a protease, wherein the protease cleavage site is a cleavage site for the protease.

35. The method according to embodiment 34, wherein the protease is fused to a protein localization tag that localizes the protease to the same cellular compartment as the fusion protein comprising the selection marker.

36. The method according to embodiment 35, wherein the protease is fused to a protein localization tag having the same amino acid sequence as that of the protein localization tag of the fusion protein comprising the selection marker.

37. The method according to embodiment 35 or embodiment 36, wherein the protease is fused to a membrane association domain.

38. The method according to embodiment 37, wherein the membrane association domain is a transmembrane domain.

39. The method according to embodiment 38, wherein the transmembrane domain is a CD8α transmembrane domain.

40. The method according to embodiment 38, wherein the transmembrane domain is a CD28 transmembrane domain.

41. The method according to embodiment 38, wherein the transmembrane domain comprises 80% or greater, 85% or greater, 90% or greater, 95% or greater, or 100% amino acid sequence identity to a transmembrane domain comprising, consisting of, or present within, an amino acid sequence selected from the group consisting of:

(SEQ ID NO: 42)
WLRLLPFLGVLALLGYLAVRPFL;
(SEQ ID NO: 43)
VLWWSIAQTVILILTGIW;
(SEQ ID NO: 44)
LGPEWDLYLMTIIALLLGTVI;
(SEQ ID NO: 45)
YYASAFSMMLGLFIFSIVFL;
(SEQ ID NO: 46)
IAFLLACVATMIFMITKCCLF;
(SEQ ID NO: 47)
VIGFLLAVVLTVAFITF;
(SEQ ID NO: 48)
GLFLSAFLLLGLFKALGWAAV;
(SEQ ID NO: 49)
VGLVLAAILALLLAFYAFFYL;
(SEQ ID NO: 50)
TFCSTALLITALALVCTLLYL;
(SEQ ID NO: 51)
WYVWLAIFFAIIIFILILGWVLL;
(SEQ ID NO: 52)
WLWVVYILT VALPVFLVILFC;
(SEQ ID NO: 53)
IYIWAPLAGTCGVLLLSLVITLYC;
and
(SEQ ID NO: 54)
FWVLVVVGGVLACYSLLVTVAFIIFWV.

42. The method according to embodiment 38 or embodiment 41, wherein the protease is fused to a hinge domain.

43. The method according to embodiment 42, wherein the hinge domain is a CD8α hinge domain.

44. The method according to embodiment 34, wherein the protease is fused to a dimerization domain.

45. The method according to embodiment 44, wherein the method comprises contacting the population of cells with a third expression construct that encodes a fusion protein comprising a membrane association domain, a dimerization domain that dimerizes with the dimerization domain fused to the protease, and a protein localization tag that localizes the dimerization domain to the same cellular compartment as the fusion protein comprising the selection marker.

46. The method according to embodiment 45, wherein the third expression construct further encodes a protein of interest.

47. The method according to embodiment 45 or embodiment 46, wherein the first expression construct site-specifically integrates into the genome of the cell.

48. The method according to embodiment 47, wherein site-specific integration of the first expression construct into the genome of the cell inactivates a target gene within the genome of the cell.

49. The method according to any one of embodiments 1 to 33, wherein the protein required for cell surface expression of the selection marker is a first complementary fragment of a protease, wherein the protease cleavage site is a cleavage site for the protease.

50. The method according to embodiment 49, wherein the two or more expression constructs comprise a third expression construct that encodes a second complementary fragment of the protease, wherein the first and second complementary fragments form an active protease complex.

51. The method according to embodiment 50, wherein the third expression construct further encodes a protein of interest.

52. The method according to embodiment 50 or embodiment 51, wherein the first expression construct site-specifically integrates into the genome of the cell.

53. The method according to embodiment 52, wherein site-specific integration of the first expression construct into the genome of the cell inactivates a target gene within the genome of the cell.

54. The method according to any one of embodiments 50 to 53, wherein the first and second complementary fragments are each fused to a protein localization tag that localizes the first and second complementary fragments to the same cellular compartment as the fusion protein comprising the selection marker.

55. The method according to embodiment 54, wherein the first and second complementary fragments are each fused to a protein localization tag having the same amino acid sequence as that of the protein localization tag of the fusion protein comprising the selection marker.

56. The method according to any one of embodiments 50 to 55, wherein the first and second complementary fragments are each fused to a membrane association domain. 57. The method according to embodiment 56, wherein the membrane association domain is transmembrane domain.

58. The method according to embodiment 57, wherein the transmembrane domain is as defined in any one of embodiments 39 to 41.

59. The method according to embodiment 50, wherein the first and second complementary fragments are each fused to a dimerization domain.

60. The method according to embodiment 59, wherein the two or more expression constructs comprise:
a fourth expression construct that encodes a fusion protein comprising a membrane association domain, a dimerization domain that dimerizes with the dimerization domain fused to the first complementary fragment, and a protein localization tag that localizes the dimerization domain to the same cellular compartment as the fusion protein comprising the selection marker; and
a fifth expression construct that encodes a fusion protein comprising a membrane association domain, a dimerization domain that dimerizes with the dimerization domain fused to the second complementary fragment, and a protein localization tag that localizes the dimerization domain to the same cellular compartment as the fusion protein comprising the selection marker.

61. The method according to embodiment 60, wherein the fourth expression construct further encodes a protein of interest.

62. The method according to embodiment 60 or embodiment 61, wherein the fourth expression construct site-specifically integrates into the genome of the cell.

63. The method according to embodiment 62, wherein site-specific integration of the fourth expression construct into the genome of the cell inactivates a target gene within the genome of the cell.

64. The method according to any one of embodiments 60 to 63, wherein the fifth expression construct further encodes a protein of interest.

65. The method according to any one of embodiments 60 to 64, wherein the fifth expression construct site-specifically integrates into the genome of the cell.

66. The method according to embodiment 65, wherein site-specific integration of the fourth expression construct into the genome of the cell inactivates a target gene within the genome of the cell.

67. The method according to any one of embodiments 60 to 66, wherein the membrane association domain of the fusion protein encoded by each of the fourth and fifth expression constructs is, independently, a transmembrane domain as defined in any one of embodiments 39 to 41.

68. The method according to any one of embodiments 44, 45, or 59 to 67, wherein the dimerization domain comprises a coiled coil structure.

69. The method according to embodiment 68, wherein the dimerization domain comprises a leucine zipper domain.

70. The method according to any one of embodiments 2 to 69, wherein a protein of interest further encoded by one or more expression constructs of the two or more separate expression constructs is independently selected from the group consisting of: a receptor, a ligand, a transcription factor, an antibody, a bispecific T-cell engager (BiTE), an enzyme, a cytokine, a chemokine, a toxin, a protein conferring resistance to cell exhaustion, and a suicide switch protein.

71. The method according to embodiment 70, wherein a protein of interest further encoded by one or more expression constructs of the two or more separate expression constructs is a receptor.

72. The method according to embodiment 71, wherein the receptor is a chimeric antigen receptor (CAR), a T cell receptor (TCR), a synthetic Notch (SynNotch) receptor, a Modular Extracellular Sensor Architecture (MESA) receptor, a Tango receptor, a ChaCha receptor, a generalized extracellular molecule sensor (GEMS) receptor, a cytokine receptor, a chemokine receptor, a switch receptor, an adhesion molecule, an integrin, an inhibitory receptor, a stimulatory receptor, an immunoreceptor tyrosine-based activation motif (ITAM)-containing receptor, or an immunoreceptor tyrosine-based inhibition motif (ITIM)-containing receptor.

73. The method according to embodiment 72, wherein the receptor is a CAR.

74. The method according to any one of embodiments 1 to 73, wherein the selection marker comprises a protein tag.

75. The method according to embodiment 74, wherein the protein tag is selected from the group consisting of: a Myc-tag, a His-tag, an HA-tag, a FLAG-tag, a Strep-tag, an NE-tag, an Xpress tag, an Avi-tag, a polyglutamate tag, and a polyarginine tag.

76. The method according to any one of embodiments 1 to 75, wherein the selection marker comprises a cluster of differentiation (CD) protein.

77. The method according to embodiment 76, wherein the CD protein is CD34.

78. The method according to any one of embodiments 1 to 75, wherein the selection marker comprises a truncated receptor comprising the extracellular domain of the receptor.

79. The method according to embodiment 78, wherein the truncated receptor is truncated epidermal growth factor receptor (EGFRt), a truncated nerve growth factor receptor (NGFRt), a truncated CD19 (CD19t), or a truncated CD20 (CD20t).

80. The method according to any one of embodiments 1 to 79, wherein the selection marker is fused to a membrane association domain.

81. The method according to embodiment 80, wherein the membrane association domain is a transmembrane domain as defined in any one of embodiments 39 to 41.

82. The method according to any one of embodiments 1 to 81, wherein the fusion protein encoded by the first expression construct further comprises a degron, wherein the protease cleavage site disposed between the selection marker and the degron.

83. The method according to any one of embodiments 1 to 82, wherein the fusion protein encoded by the first expression construct further comprises a domain that confers antibiotic resistance.

84. The method according to embodiment 83, wherein the domain that confers antibiotic resistance is disposed between the selection marker and the protease cleavage site.

85. The method according to embodiment 83 or embodiment 84, wherein the domain that confers antibiotic resistance confers puromycin resistance.

86. The method according to embodiment 85, wherein the domain that confers puromycin resistance comprises a puromycin-N-acetyltransferase (PuroR).

87. The method according to any one of embodiments 1 to 86, wherein the selecting comprises magnetic-activated cell sorting (MACS).

88. The method according to any one of embodiments 1 to 86, wherein the selecting comprises flow cytometry.

89. The method according to embodiment 88, wherein the flow cytometry comprises fluorescence-activated cell sorting (FACS).

90. The method according to any one of embodiments 1 to 89, wherein the population of cells is a population of mammalian cells.

91. The method according to embodiment 90, wherein the mammalian cells comprise immune cells.

92. The method according to embodiment 91, wherein the immune cells comprise T cells, B cells, natural killer (NK) cells, macrophages, monocytes, neutrophils, dendritic cells, mast cells, basophils, eosinophils, and any combination thereof.

93. The method according to embodiment 91, wherein the immune cells comprise T cells.

94. The method according to embodiment 93, wherein the T cells comprise naive T cells (T_{N}), cytotoxic T cells (T_{CTL}), memory T cells (T_{MEM}), T memory stem cells (T_{SCM}), central memory T cells (T_{CM}), effector memory T cells (T_{EM}), tissue resident memory T cells (T_{RM}), effector T cells (T_{EFF}), regulatory T cells (T_{REGs}), helper T cells, CD4+ T cells, CD8+ T cells, virus-specific T cells, alpha beta T cells (T_{αβ}), gamma delta T cells (T_{γδ}), and any combination thereof.

95. The method according to embodiment 90, wherein the mammalian cells comprise stem cells.

6. The method according to embodiment 95, wherein the stem cells comprise embryonic stem (ES) cells, adult stem cells, hematopoietic stem cells (HSCs), induced pluripotent stem cells (iPSCs), mesenchymal stem cells (MSCs), neural stem cells (NSCs), or any combination thereof.

97. A cell comprising two or more separate expression constructs, wherein the two or more separate expression constructs comprise:
a first expression construct that encodes a fusion protein comprising a selection marker, a protein localization tag, and a protease cleavage site disposed between the selection marker and the protein localization tag; and
a second expression construct that encodes a protein required for cell surface expression of the selection marker.

98. The cell of embodiment 97, wherein the first expression construct further encodes a protein of interest.

99. The cell of embodiment 97 or embodiment 98, wherein the first expression construct is site-specifically integrated into the genome of the cell.

100. The cell of embodiment 99, wherein a target gene within the genome of the cell is inactivated as a result of the site-specific integration of the first expression construct.

101. The cell of any one of embodiments 97 to 100, wherein the second expression construct further encodes a protein of interest.

102. The cell of any one of embodiments 97 to 101, wherein the second expression construct is site-specifically integrated into the genome of the cell.

103. The cell of embodiment 102, wherein a target gene within the genome of the cell is inactivated as a result of the site-specific integration of the second expression construct.

104. The cell of any one of embodiments 97 to 103, wherein the cell is a mammalian cell.

105. The cell of embodiment 104, wherein the mammalian cell is a human cell.

106. The cell of embodiment 104 or embodiment 105, wherein the cell is an immune cell.

107. The cell of embodiment 106, wherein the immune cell is a T cell, a B cell, a natural killer (NK) cell, a macrophage, a monocyte, a neutrophil, a dendritic cell, a mast cell, a basophil, or an eosinophil.

108. The cell of embodiment 106, wherein the immune cell is a T cell.

109. The cell of embodiment 108, wherein the T cell is a naive T cell (T_{N}), a cytotoxic T cell (T_{CTL}), a memory T cell (T_{MEM}), a T memory stem cell (T_{SCM}), a central memory T cell (T_{CM}), an effector memory T cell (T_{EM}), a tissue resident memory T cell (T_{RM}), an effector T cell (T_{EFF}), a regulatory T cell (T_{REGs}), a helper T cell, a CD4+ T cell, a CD8+ T cell, a virus-specific T cell, an alpha beta T cell (T_{αβ}), or a gamma delta T cell (T_{γδ}).

110. The cell of embodiment 104 or embodiment 105, wherein the cell is a stem cell.

111. The cell of embodiment 110, wherein the stem cell is an embryonic stem (ES) cell, an adult stem cell, a hematopoietic stem cell (HSC), an induced pluripotent stem cell (iPSC), a mesenchymal stem cell (MSC), or a neural stem cell (NSC).

112. A kit comprising two or more separate expression constructs, wherein the two or more separate expression constructs comprise:
a first expression construct that encodes a fusion protein comprising a selection marker, a protein localization tag, and a protease cleavage site disposed between the selection marker and the protein localization tag; and
a second expression construct that encodes a protein required for cell surface expression of the selection marker.

113. The kit of embodiment 112, wherein the first expression construct further encodes a protein of interest.

114. The kit of embodiment 112, wherein the first expression construct comprises a cloning site for a nucleic acid encoding a protein of interest.

115. The kit of any one of embodiments 112 to 114, wherein the second expression construct further encodes a protein of interest.

116. The kit of any one of embodiments 112 to 114, wherein the second expression construct comprises a cloning site for a nucleic acid encoding a protein of interest.

117. The kit of any one of embodiments 112 to 116, further comprising instructions for contacting a population of cells with the two or more expression constructs under conditions in which the two or more expression constructs are delivered to cells of the population of cells.

118. The kit of any one of embodiments 112 to 117, further comprising instructions for selecting for cells exhibiting cell surface expression of the selection marker.

119. The cell of any one of embodiments 97 to 118, wherein the protein localization tag is as defined in any one of embodiments 8 to 23.

120. The cell or kit of any one of embodiments 97 to 119, wherein the protease cleavage site is as defined in any one of embodiments 24 to 33.

121. The cell or kit of any one of embodiments 97 to 120, wherein the protein required for cell surface expression of the selection marker is a protease, wherein the protease cleavage site is a cleavage site for the protease.

122. The cell or kit of embodiment 121, wherein the protease is fused to a protein localization tag that localizes the protease to the same cellular compartment as the fusion protein comprising the selection marker.

123. The cell or kit of embodiment 122, wherein the protease is fused to a protein localization tag having the same amino acid sequence as that of the protein localization tag of the fusion protein comprising the selection marker.

124. The cell or kit of embodiment 122 or embodiment 123, wherein the protease is fused to a membrane association domain.

125. The cell or kit of embodiment 124, wherein the membrane association domain is a transmembrane domain as defined in any one of embodiments 39 to 41.

126. The cell or kit of embodiment 121, wherein the protease is fused to a dimerization domain.

127. The cell or kit of embodiment 126, comprising a third expression construct that encodes a fusion protein comprising a transmembrane domain, a dimerization domain that dimerizes with the dimerization domain fused to the protease, and a protein localization tag that localizes the dimerization domain to the same cellular compartment as the fusion protein comprising the selection marker.

128. The cell or kit of any one of embodiments 97 to 120, wherein the protein required for cell surface expression of the selection marker is a first complementary fragment of a protease, wherein the protease cleavage site is a cleavage site for the protease.

129. The cell or kit of embodiment 128, comprising a third expression construct that encodes a second complementary fragment of the protease, wherein the first and second complementary fragments form an active protease complex.

130. The cell or kit of embodiment 129, wherein the third expression construct further encodes a protein of interest.

131. The cell or kit of embodiment 129 or embodiment 130, wherein the first and second complementary fragments are each fused to a protein localization tag that localizes the protease to the same cellular compartment as the fusion protein comprising the selection marker.

132. The cell or kit of embodiment 131, wherein the first and second complementary fragments are each fused to a protein localization tag having the same amino acid sequence as that of the protein localization tag of the fusion protein comprising the selection marker.

133. The cell or kit of any one of embodiments 129 to 132, wherein the first and second complementary fragments are each fused to a membrane association domain.

134. The cell or kit of embodiment 133, wherein the membrane association domain is a transmembrane domain.

135. The cell or kit of embodiment 134, wherein the transmembrane domain is as defined in any one of embodiments 39 to 41.

136. The cell or kit of any one of embodiments 129 to 132, wherein the first and second complementary fragments are each fused to a dimerization domain.

137. The cell or kit of embodiment 136, comprising:
a fourth expression construct that encodes a fusion protein comprising a membrane association domain, a dimerization domain that dimerizes with the dimerization domain fused to the first complementary fragment, and a protein localization tag that localizes the dimerization domain to the same cellular compartment as the fusion protein comprising the selection marker; and
a fifth expression construct that encodes a fusion protein comprising a membrane association domain, a dimerization domain that dimerizes with the dimerization domain fused to the second complementary fragment, and a protein localization tag that localizes the dimerization domain to the same cellular compartment as the fusion protein comprising the selection marker.

138. The cell or kit of embodiment 137, wherein the fourth expression construct further encodes a protein of interest.

139. The cell or kit of embodiment 137 or embodiment 138, wherein the fifth expression construct further encodes a protein of interest.

140. The cell or kit of any one of embodiments 137 to 139, wherein the membrane association domain of the fusion protein encoded by each of the fourth and fifth expression constructs is, independently, a transmembrane domain as defined in any one of embodiments 39 to 41.

141. The cell or kit of embodiment 126, 127, or 136 to 140, wherein the dimerization domain comprises a coiled coil structure.

142. The cell or kit of embodiment 141, wherein the dimerization domain comprises a leucine zipper domain.

143. The cell or kit of any one of embodiments 97 to 142, wherein a protein of interest further encoded by one or more expression constructs of the two or more separate expression constructs is independently selected from the group consisting of: a receptor, a ligand, a transcription factor, an antibody, a bispecific T-cell engager (BiTE), an enzyme, a cytokine, a chemokine, a toxin, a protein conferring resistance to cell exhaustion, and a suicide switch protein.

144. The cell or kit of embodiment 143, wherein a protein of interest further encoded by one or more expression constructs of the two or more separate expression constructs is a receptor.

145. The cell or kit of embodiment 144, wherein the receptor is a chimeric antigen receptor (CAR), a T cell receptor (TCR), a synthetic Notch (SynNotch) receptor, a Modular Extracellular Sensor Architecture (MESA) receptor, a Tango receptor, a ChaCha receptor, a generalized extracellular molecule sensor (GEMS) receptor, a cytokine receptor, a chemokine receptor, a switch receptor, an adhesion molecule, an integrin, an inhibitory receptor, a stimulatory receptor, an immunoreceptor tyrosine-based activation motif (ITAM)-containing receptor, or an immunoreceptor tyrosine-based inhibition motif (ITIM)-containing receptor.

146. The cell or kit of embodiment 144, wherein the receptor is a CAR.

147. The cell or kit of any one of embodiments 97 to 146, wherein the selection marker is as defined in any one of embodiments 74 to 81.

148. The cell or kit of any one of embodiments 97 to 147, wherein the fusion protein encoded by the first expression construct further comprises a degron, wherein the protease cleavage site disposed between the selection marker and the degron.

149. The cell or kit of any one of embodiments 97 to 148, wherein the fusion protein encoded by the first expression construct further comprises a domain that confers antibiotic resistance.

150. The cell or kit of embodiment 149, wherein the domain that confers antibiotic resistance is disposed between the selection marker and the protease cleavage site.

151. The cell or kit of embodiment 149 or embodiment 150, wherein the domain that confers antibiotic resistance confers puromycin resistance.

152. The cell or kit of embodiment 151, wherein the domain that confers puromycin resistance comprises a puromycin-N-acetyltransferase (PuroR).

153. A composition comprising cells or progeny thereof selected according to the method of any one of embodiments 1 to 96 present in a liquid medium.

154. A composition comprising the cell of any one of embodiments 97 to 111 or 119 to 152 present in a liquid medium.

155. The composition of embodiment 153 or embodiment 154, wherein the liquid medium is a cell culture medium.

156. The composition of embodiment 153 or embodiment 154, wherein the liquid medium is suitable for administration of the composition to an individual in need thereof.

157. The composition of embodiment 156 formulated for parenteral administration to the individual.

158. A method comprising administering a therapeutically effective amount of the composition of embodiment 156 or embodiment 157 to an individual in need thereof.

159. A fusion protein comprising a protein fused to an ER localization tag, wherein the ER localization tag comprises 80% or greater, 85% or greater, 90% or greater, 95% or greater, or 100% amino acid sequence identity to an ER localization tag comprising, consisting of, or present within, an amino acid sequence selected from the group consisting of:

(SEQ ID NO: 22)
PKKKQQKDSLINLKIQKENPKVVNEINIEDLCLTKAAYCRCWRSKTFPA
CDGSHNKHNELTGDNVGPLILKKKEV;
(SEQ ID NO: 23)
QMRHLKSFFEAKKLV;
(SEQ ID NO: 24)
AYRQRQHQDMPAPRPPGPRPAPPQQEGPPEQQPPQ;
(SEQ ID NO: 25)
HMKEKEKSD;
(SEQ ID NO: 26)
CFRKLAKTGKKKKRD;
(SEQ ID NO: 27)
KCCAYGYRKCLGKKGRVKKAHKSKTH;
(SEQ ID NO: 28)
YLSTCKDSKKKAE;
(SEQ ID NO: 29)
RLTTDVDPDLDQDED;
(SEQ ID NO: 30)
KYKSRRSFIDEKKMP;
(SEQ ID NO: 31)
MTGCCGCCCGCFGIIPLMSKCGKKSSYYTTFDNDVVIEQYRPKKSV;
(SEQ ID NO: 32)
NRSPRNRKPRRE;
(SEQ ID NO: 9)
LYKYKSRRSFIEEKKMP;
(SEQ ID NO: 33)
TKVLKGKKLSLPA;
(SEQ ID NO: 34)
KSNRHKDGFHRLRGHHDEYEDEIRMMSTGSKKSLLSHEFQDETDTEETL
YSSKH;
and
(SEQ ID NO: 35)
KCGKKSSYYTTFDNDVVIEQYRPKKSV.

160. The fusion protein of embodiment 159, wherein the C-terminus of the ER localization tag comprises the four C-terminal residues of one of the sequences recited in embodiment 159.

161. A fusion protein comprising a protein fused to an ER localization tag, wherein the ER localization tag comprises a Tm domain, an ICD, or both, of an ER localization tag of a polypeptide set forth in Table 1, or a variant Tm and/or ICD thereof which retains the ability to localize a polypeptide to the ER.

162. A fusion protein comprising a protein fused to an ER localization tag, wherein the ER localization tag comprises a Tm domain, an ICD, or both, of an ER localization tag of a human ER-resident protein, or a variant Tm and/or ICD thereof which retains the ability to localize a polypeptide to the ER.

163. The fusion protein of embodiment 162, wherein the human ER-resident protein is CISD2.

164. The fusion protein of embodiment 163, wherein the ER localization tag comprises the Tm domain, the ICD, or both, of the polypeptide set forth in SEQ ID NO:91, or a variant Tm and/or ICD thereof which retains the ability to localize a polypeptide to the ER.

165. The fusion protein of embodiment 162, wherein the human ER-resident protein is UGT2B17.

166. The fusion protein of embodiment 165, wherein the ER localization tag comprises the Tm domain, the ICD, or both, of the polypeptide set forth in SEQ ID NO:95, or a variant Tm and/or ICD thereof which retains the ability to localize a polypeptide to the ER.

167. The fusion protein of embodiment 159, wherein the protein is fused directly to the ER localization tag.

168. The fusion protein of embodiment 159, wherein the protein is fused indirectly to the ER localization tag.

169. The fusion protein of any one of embodiments 159 to 168, further comprising a protease cleavage site.

170. The fusion protein of embodiment 169, wherein the protease cleavage site is disposed between the protein and the ER localization tag.

171. The fusion protein of embodiment 169 or embodiment 170, wherein the protease cleavage site is as defined in any one of embodiments 24 to 33.

172. The fusion protein of any one of embodiments 159 to 171, further comprising a transmembrane domain.

173. The fusion protein of embodiment 172, wherein the transmembrane domain is as defined in any one of embodiments 39 to 41.

174. A fusion protein comprising a protein fused to a transmembrane domain, wherein the transmembrane domain comprises 80% or greater, 85% or greater, 90% or greater, 95% or greater, or 100% amino acid sequence identity to a transmembrane domain comprising, consisting of, or present within, an amino acid sequence selected from the group consisting of:

(SEQ ID NO: 42)
WLRLLPFLGVLALLGYLAVRPFL;
(SEQ ID NO: 43)
VLWWSIAQTVILILTGIW;
(SEQ ID NO: 44)
LGPEWDLYLMTIIALLLGTVI;
(SEQ ID NO: 45)
YYASAFSMMLGLFIFSIVFL;
(SEQ ID NO: 46)
IAFLLACVATMIFMITKCCLF;
(SEQ ID NO: 47)
VIGFLLAVVLTVAFITF;
(SEQ ID NO: 48)
GLFLSAFLLLGLFKALGWAAV;
(SEQ ID NO: 49)
VGLVLAAILALLLAFYAFFYL;
(SEQ ID NO: 50)
TFCSTALLITALALVCTLLYL;
(SEQ ID NO: 51)
WYVWLAIFFAIIIFILILGWVLL;
(SEQ ID NO: 52)
WLWVVYILT VALPVFLVILFC;
(SEQ ID NO: 53)
IYIWAPLAGTCGVLLLSLVITLYC;
and
(SEQ ID NO: 54)
FWVLVVVGGVLACYSLLVTVAFIIFWV.

175. The fusion protein of embodiment 174, wherein the protein is fused directly to the transmembrane domain.

176. The fusion protein of embodiment 174, wherein the protein is fused indirectly to the transmembrane domain.

177. The fusion protein of any one of embodiments 174 to 176, further comprising a protease cleavage site.

178. The fusion protein of embodiment 177, wherein the protease cleavage site is as defined in any one of embodiments 24 to 33.

179. The fusion protein of any one of embodiments 174 to 177, further comprising a protein localization tag.

180. The fusion protein of embodiment 179, wherein the protein localization tag is as defined in any one of embodiments 8 to 23.

181. The fusion protein of any one of embodiments 159 to 180, wherein the protein is a receptor, a ligand, a transcription factor, an antibody, a bispecific T-cell engager (BiTE), an enzyme, a cytokine, a chemokine, a toxin, a protein conferring resistance to cell exhaustion, and a suicide switch protein.

182. The fusion protein of any one of embodiments 159 to 180, wherein the protein is a receptor selected from the group consisting of: a chimeric antigen receptor (CAR), a T cell receptor (TCR), a synthetic Notch (SynNotch) receptor, a Modular Extracellular Sensor Architecture (MESA) receptor, a Tango receptor, a ChaCha receptor, a generalized extracellular molecule sensor (GEMS) receptor, a cytokine receptor, a chemokine receptor, a switch receptor, an adhesion molecule, an integrin, an inhibitory receptor, a stimulatory receptor, an immunoreceptor tyrosine-based activation motif (ITAM)-containing receptor, and an immunoreceptor tyrosine-based inhibition motif (ITIM)-containing receptor.

183. The fusion protein of embodiment 182, wherein the receptor is a CAR.

184. The fusion protein of any one of embodiments 159 to 176, wherein the protein is a selection marker.

185. A nucleic acid that encodes the fusion protein of any one of embodiments 159 to 184.

186. An expression construct comprising the nucleic acid of embodiment 185.

187. A cell comprising the nucleic acid of embodiment 185 or the expression construct of embodiment 186.

188. A method of producing the fusion protein of any one of embodiments 159 to 184, comprising culturing the cell of embodiment 186 or embodiment 187 under conditions suitable for the cell to express the fusion protein, wherein the fusion protein is produced.

The following examples are offered by way of illustration and not by way of limitation.

### EXPERIMENTAL

Selection of Cells with Multiple Genetic Modifications Using a Single Selection Marker

Described herein are cell selection systems according to embodiments of the present disclosure. The systems are sometimes referred to herein as “STASH selection systems”, “STASH select”, etc. by virtue of the selection marker being “stashed” intracellularly in the absence of the desired combination of expression constructs being present in the cell. According to the selection systems, one of the expression constructs encodes a fusion protein comprising the selection marker, a protein localization tag, and a protease cleavage site disposed between the selection marker and the protein localization tag. In the absence of one or more additional expression constructs which provide a protease capable of cleaving the protease cleavage site, the selection marker remains localized to (i.e., retained or “stashed” at) the intracellular location (e.g., organelle) determined by the particular protein localization tag employed. When the one or more additional expression constructs are present in the cell, thereby providing a protease capable of cleaving the protease cleavage site, the selection marker is cleaved from the protein localization tag and traffics to the surface of the cell, such that the cell comprising the desired multiple genetic modifications exhibits cell surface expression of the selection marker. Non-limiting examples and data providing proof-of-concept of cell selection systems according to embodiments of the present disclosure will now be described.

High Surface Expression of a Myc Tag Selection Marker in Cells that are Double Positive for Separate Expression Constructs A and B—NS3 Protease

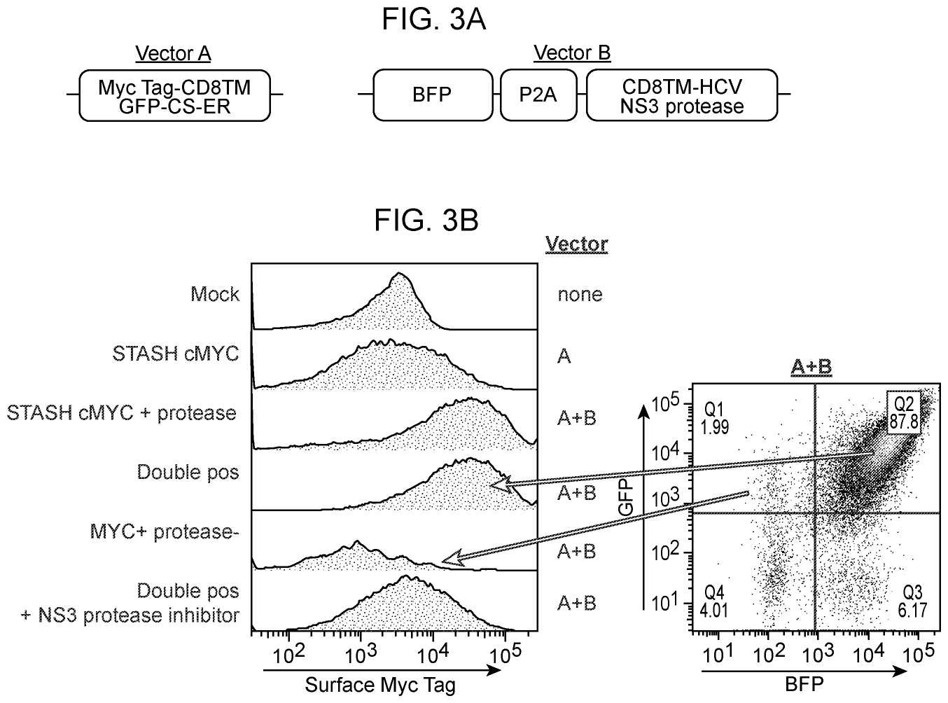

Shown in FIG. 3A is a schematic of an exemplary embodiment of the STASH select system. Expression construct A encodes a Myc-tag fused to the N terminus of a fusion protein comprising a CD8a hinge, CD8 transmembrane, GFP, HCV NS3 cleavage site, and ER retention tag. Expression construct B is comprised of BFP-P2A-membrane tethered HCV NS3 protease fused to an ER retention tag.

FIG. 3B is a series of flow cytometry histograms of surface Myc staining on primary human T cells that were retrovirally transduced with expression construct A and expression construct B (from FIG. 3A). As can be seen in the data, only cells engineered with expression constructs A and B have high surface expression of the Myc tag. Cells which have been exposed to expression construct A and expression construct B result in four populations of cells: non transduced (BFP−GFP−), single transduced expression construct A (BFP−GFP+), single transduced expression construct B (BFP+GFP−) and double transduced (BFP+GFP+). Only the double transduced population of cells have high surface expression of the Myc selection marker, whereas the single transduced expression construct A (BFP−GFP+) population has negligible levels of Myc staining. Addition of the NS3 protease inhibitor grazoprevir results in a dramatic reduction in surface expression of the Myc Tag, which demonstrates that the surface expression of the Myc tag is dependent on the proteolytic activity of NS3 protease.

High Surface Expression of a Myc Tag Selection Marker in Cells that are Double Positive for Separate Expression Constructs A and B-TEV Protease Variant

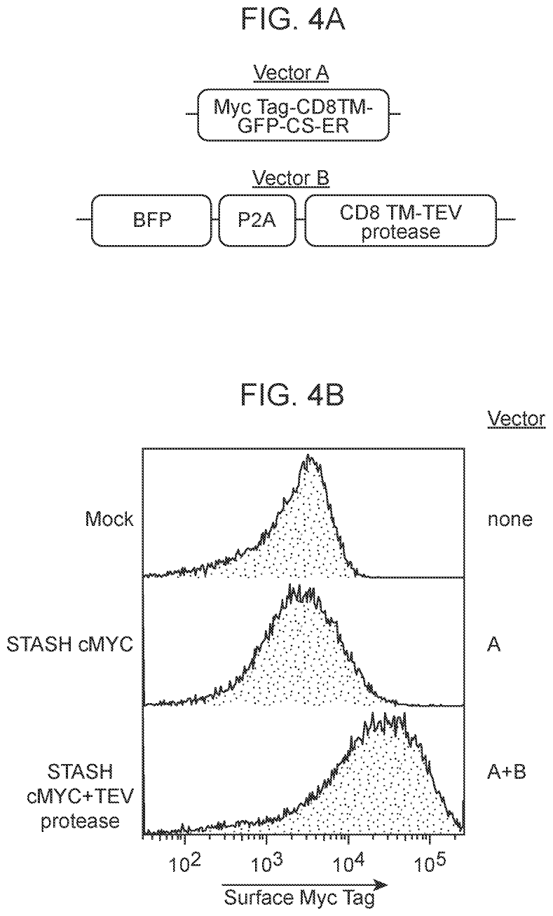

FIG. 4A is a schematic of an exemplary embodiment of the STASH select system. Expression construct A encodes a Myc-tag fused to the N terminus of a fusion protein comprising a CD8a hinge, CD8 transmembrane, GFP, TEV cleavage site, and ER retention tag. Expression construct B is comprised of BFP-P2A-membrane tethered TEV protease fused to an ER retention tag.

FIG. 4B is a series of flow cytometry histograms of surface Myc staining on primary human T cells that were retroviral transduced with expression construct A and expression construct B (from FIG. 4A). As can be seen in the data, only cells engineered with expression constructs A and B have high surface expression of the Myc tag.

High Surface Expression of a Myc Tag Selection Marker in Cells that are Triple Positive for Separate Expression Constructs A, B and C

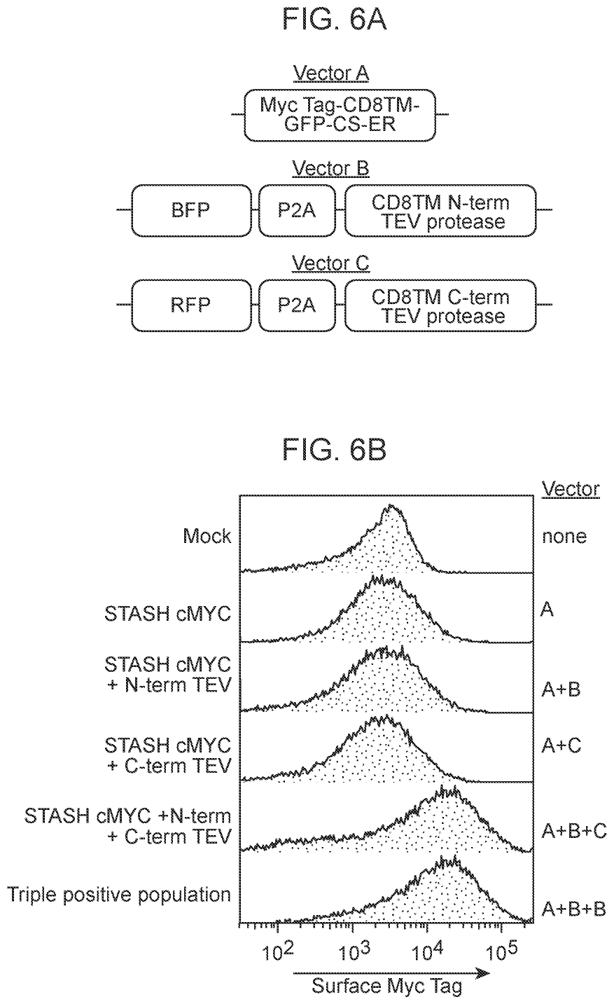

FIG. 6A is a schematic of an exemplary embodiment of the three-way STASH select system. Expression construct A encodes a Myc-tag fused to the N terminus of a fusion protein comprising a CD8a hinge, CD8a transmembrane, GFP, TEV cleavage site, and ER retention tag. Expression construct B is comprised of BFP-P2A-CD8a hinge-CD8a N-term TEV protease domain fused to an ER retention tag. Expression construct C is comprised of RFP-P2A-CD8a hinge-CD8a N-term TEV protease domain fused to an ER retention tag.

FIG. 6B is a series of flow cytometry histograms of surface Myc staining on primary human T cells that were retrovirally transduced with expression construct A, expression construct B, and expression construct C (from FIG. 6A). As can be seen in the data, only cells engineered with expression construct A, expression construct B, and expression construct C have high surface expression of the Myc tag. Cells which are only positive for expression construct A and expression construct B or expression construct A and expression construct C have minimal surface expression of the Myc tag selection marker. These data demonstrate that the three-way STASH Select system can be used to identify and enrich cells which are triple positive from cells that are non-transduced, single positive, or double positive.

High Surface Expression of a Myc Tag Selection Marker in Cells that are Quintuple Positive for Separate Expression Constructs A, B, C, D and E

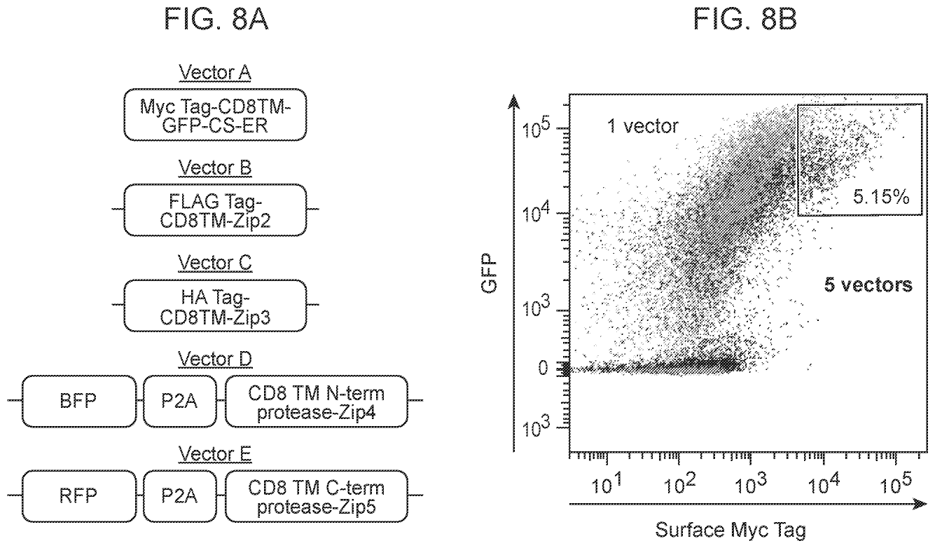

FIG. 8A is a schematic of an exemplary embodiment of the five-way STASH select system. Expression construct A encodes a Myc-tag fused to the N terminus of a fusion protein comprising a CD8a hinge, CD8 transmembrane, GFP, TEV cleavage site, and ER retention tag. Expression construct B is comprised of a FLAG Tag tethered to a CD8a hinge, CD8a transmembrane domain, a leucine zipper domain (Zip2), and an ER retention tag. Expression construct C is comprised of a HA Tag tethered to a CD8a hinge, CD8a transmembrane domain, a leucine zipper domain (Zip3), and an ER retention tag. Expression construct D is comprised of BFP-P2A-cytosolic N-terminal TEV protease domain fused to leucine zipper (Zip4). Expression construct E is comprised of RFP-P2A-cytosolic C-terminal TEV protease domain fused to leucine zipper (Zip5).

FIG. 8B is a series of flow cytometry dot plot of surface Myc staining on primary human T cells that were retrovirally transduced with expression construct A, expression construct B, expression construct C, expression construct D, and expression construct E (from FIG. 8A). As can be seen in the data, cells engineered with expression construct A, expression construct B, expression construct C, expression construct D, and expression construct E have a quintuple positive population of cells with high surface expression of the Myc tag. Cells which are only positive for expression construct A do not contain this population of high Myc tag surface expression. These data demonstrate that the five-way STASH Select system can be used to identify and enrich cells which are quintuple positive.

### Assessment and Development of Various ER Localization Tags

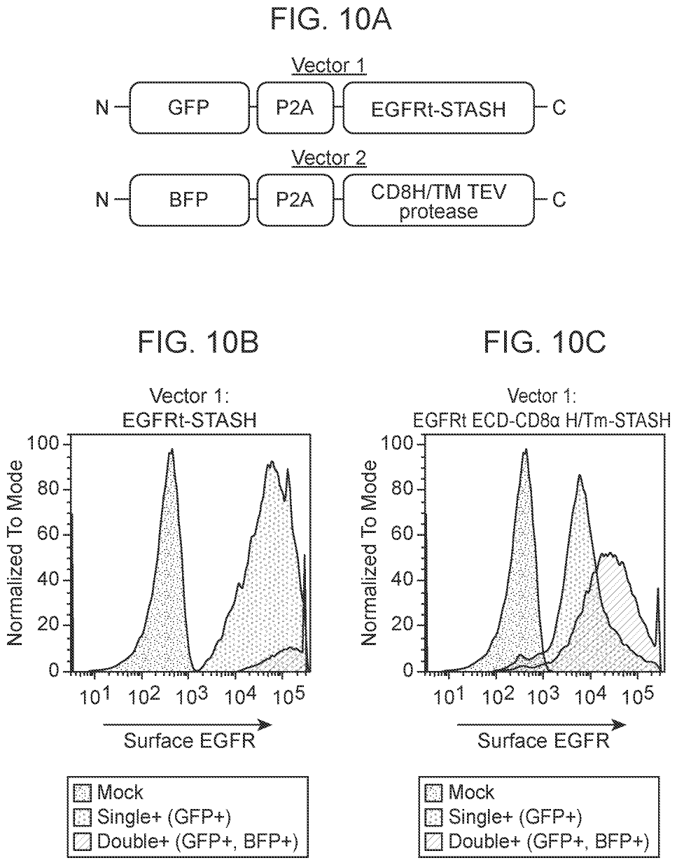

FIG. 10A is a schematic of a two-way STASH Select system with EGFRt as the STASHed surface marker. The first expression construct encodes GFP, a P2A ribosome skip sequence, EGFRt, a TEV cleavage site, and an E3-19K protein ER retention tag (LYKYKSRRSFIDEKKMP; SEQ ID NO:1). Expression construct 2 encodes BFP, a P2A ribosome skip sequence, a CD8 H/Tm, TEV protease, and an E3-19K protein ER retention tag (LYKYKSRRSFIDEKKMP; SEQ ID NO: 1).

FIG. 10B is a series of flow cytometry histograms of surface EGFR on primary human T cells that were retrovirally transduced with expression construct 1 and expression construct 2. As can be seen in the data, cells that are positive for GFP but not BFP (Single+) have high residual surface EGFR expression, which suggests that the E3-19K protein ER retention tag (LYKYKSRRSFIDEKKMP; SEQ ID NO:1) is a STASH tag that results in sub-optimal intracellular retention.

FIG. 10C is a series of flow cytometry histograms of surface EGFR on primary human T cells that were retrovirally transduced with expression construct 2 and a modified expression construct 1, whereby the EGFRt extracellular domain (ECD) was fused to a CD8α hinge and transmembrane domain (CD8α H/Tm), a TEV cleavage site, and an E3-19K protein ER retention tag (LYKYKSRRSFIDEKKMP; SEQ ID NO:1). As can be seen in the data, cells that are positive for GFP but not BFP (Single+) have high residual surface EGFR expression, which suggests that the E3-19K protein ER retention tag (LYKYKSRRSFIDEKKMP; SEQ ID NO:1) is a STASH tag that results in sub-optimal intracellular retention even when the EGFRt ECD is fused to a CD8α H/Tm.

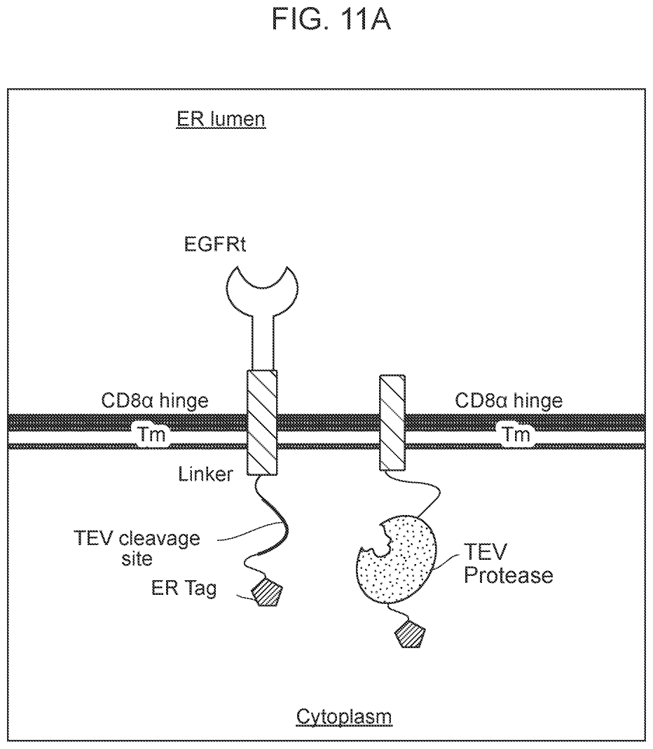

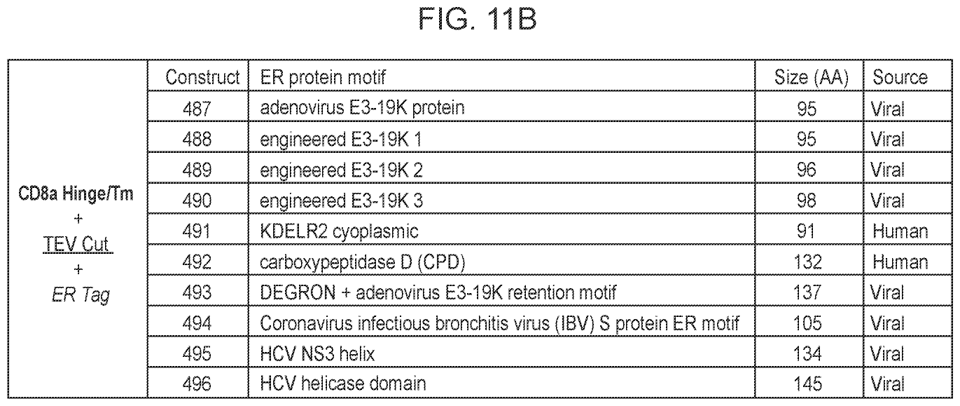

EGFRt fusion proteins with various ER tags (set 1) were then produced. FIG. 11A is a schematic of a two-way STASH Select system with EGFRt as the STASHed surface marker, whereby the extracellular domain (ECD) of EGFRt is fused to a CD8α hinge and transmembrane domain (CD8α H/Tm), a TEV cleavage site, and an ER retention tag.

FIG. 11B is a table of EGFRt-STASH variants comprising EGFRt ECD fused to CD8α H/Tm, a TEV cleavage site, and the indicate ER retention tag variant.

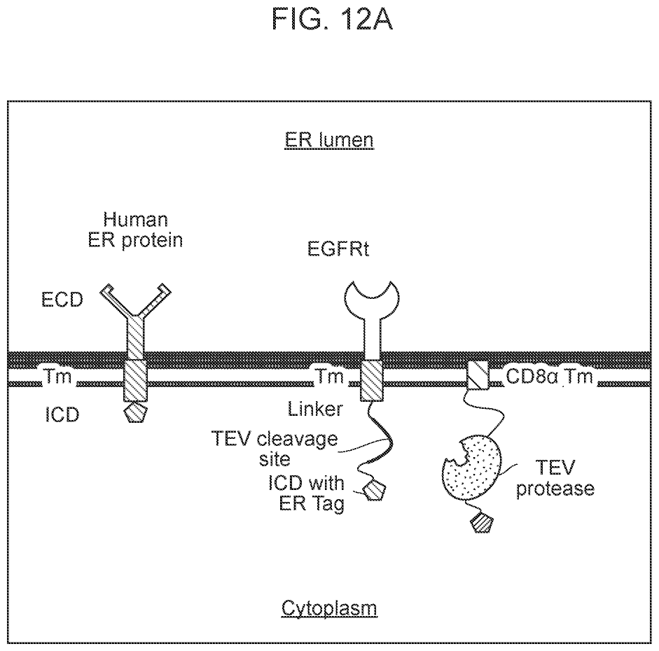

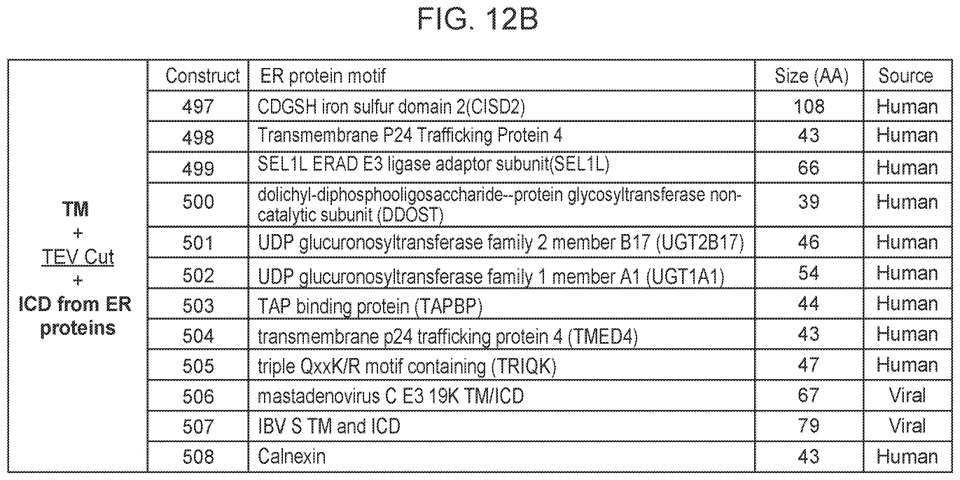

Additional EGFRt fusion proteins with various ER tags (set 2) were then produced. FIG. 12A is a schematic of a two-way STASH Select system with EGFRt as the STASHed surface marker, whereby the extracellular domain (ECD) of EGFRt is fused to a transmembrane domain (Tm) and intracellular domain (ICD) of an ER-resident membrane protein separated by a linker containing a TEV cleavage site.

FIG. 12B is table of EGFRt-STASH variants comprising EGFRt ECD fused to the Tm and ICD of the indicated ER-resident membrane protein, separated by a linker containing a TEV cleavage site.

### Development of High-Performance Stash Constructs

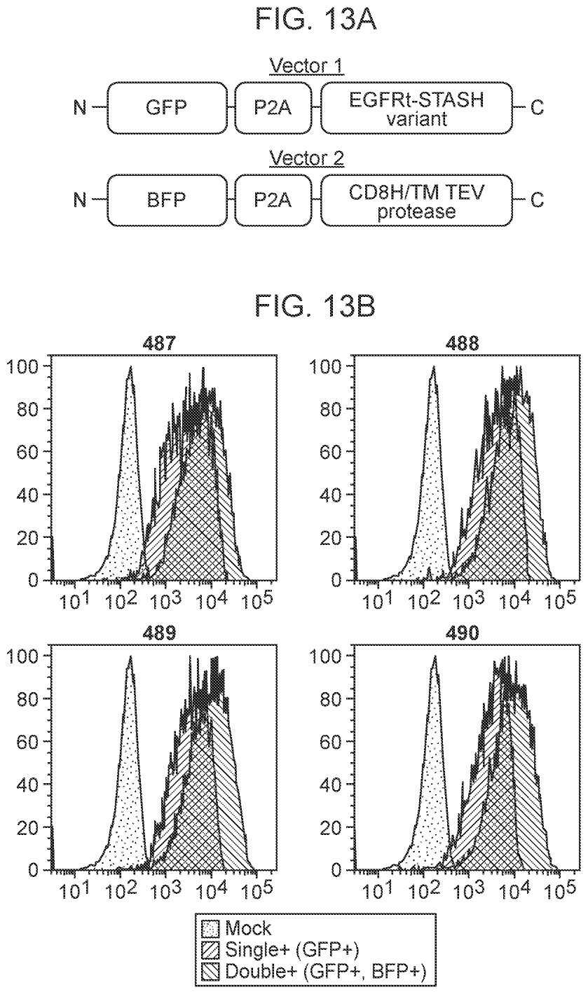

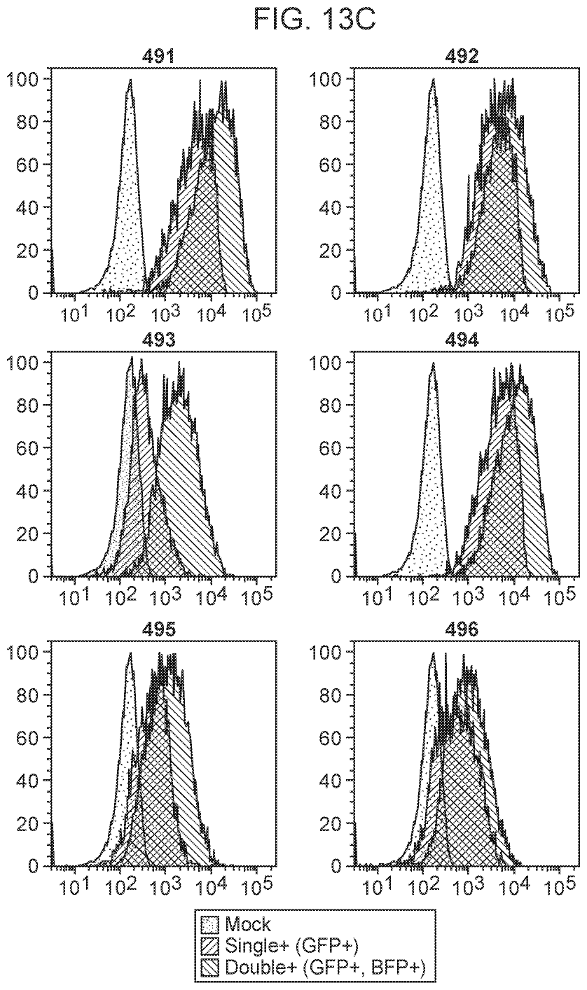

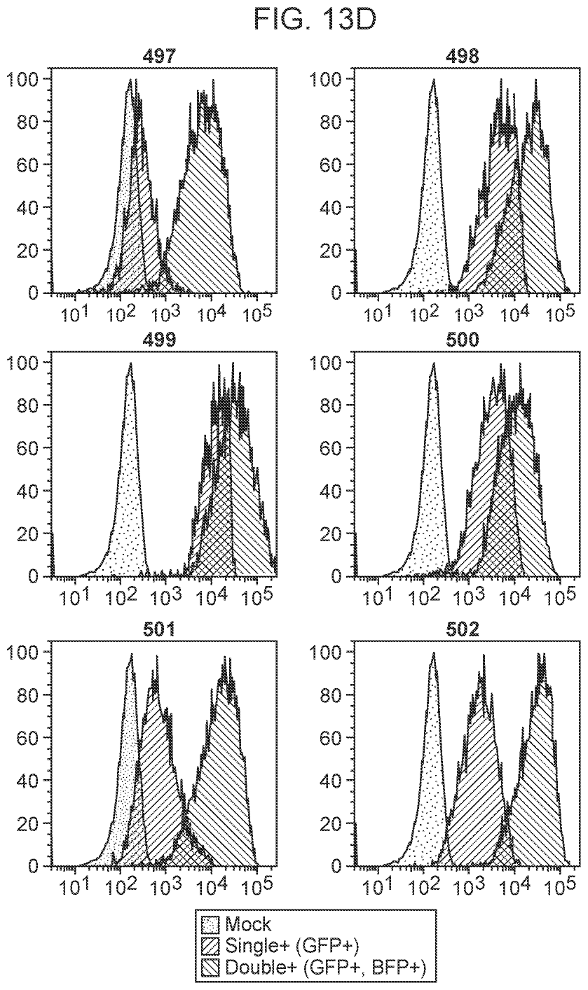

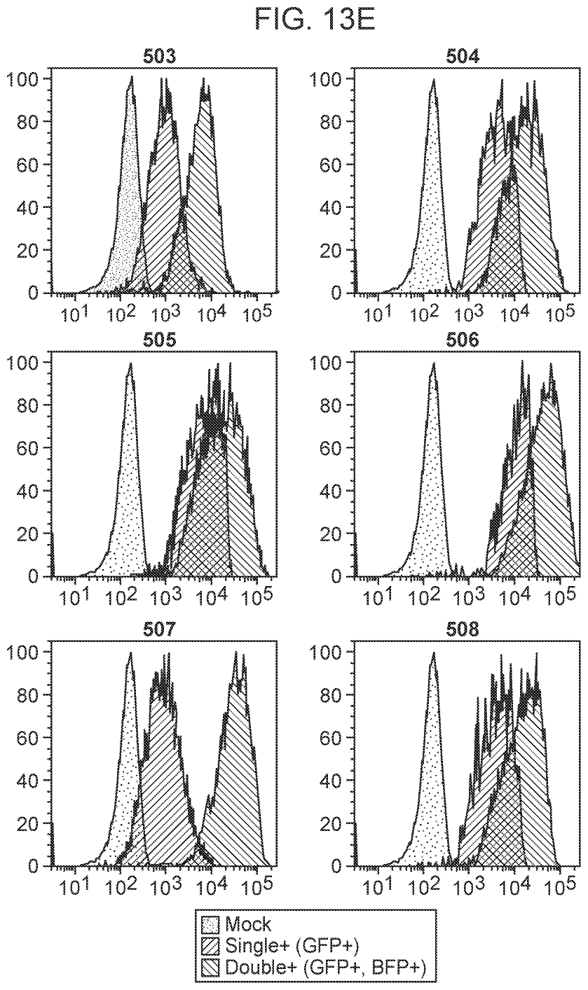

FIG. 13A is a schematic of a two-way STASH Select system with an EGFRt-STASH variant as the STASHed surface marker. The first expression construct encodes GFP, a P2A ribosome skip sequence, a EGFRt STASH variant. Expression construct 2 encodes BFP, a P2A ribosome skip sequence, a CD8 H/Tm, TEV protease, and an E3-19K protein ER retention tag (LYKYKSRRSFIDEKKMP; SEQ ID NO:1).

FIG. 13B a series of flow cytometry histograms of surface EGFRt on primary human T cells that were retrovirally transduced with expression construct 1 and expression construct 2. The number above each flow plot indicates the ER Tag variant used. Mock negative control cells, cells that are singly positive for GFP, and cells which are doubly positive for GFP and BFP are indicated. As can be seen in the data, several ER Tag variants show differential surface EGFRt expression levels between the single and double positive populations, which allow for selective purification of the double positive population via surface expressed EGFRt. Several of the high-performing ER Tag variants are novel sequences derived from human proteins, which is preferable for clinical applications in humans due to reduced risk of immunogenicity.

### MACS-Based Enrichment of EGFRt-STASH Select Variants

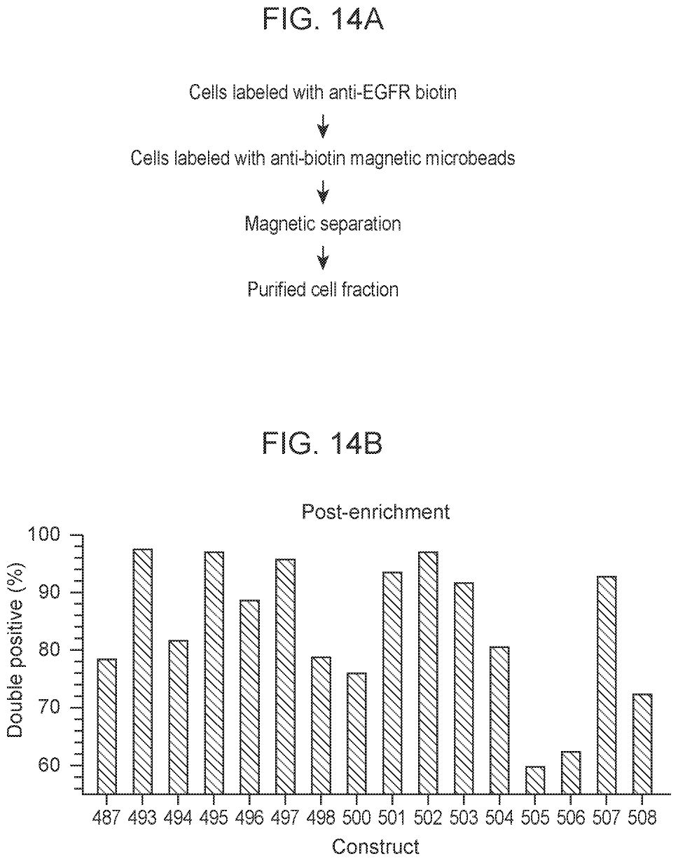

FIG. 14A is a schematic of the workflow for EGFR-based purification using magnetic activated cell sorting (MACS). Cells are labeled with anti-EGFR-biotin, which only binds surface expressed EGFR. Cells are washed then labeled with anti-biotin microbeads. Cells are applied to a magnetic separation column, the column is washed to remove the negative cell fraction, then the purified cell fraction is eluted off the column.

FIG. 14B is a plot of the percentage of double positive (BFP+GFP+) from the purified cell fraction after EGFR MACS selection (post-enrichment) of samples shown in FIG. 13. These data demonstrate that several of the EGFRt-STASH variants can be used to isolate highly pure double positive populations using a single selection marker.

Two-Way STASH Select Using EGFRt with Novel ER Tags

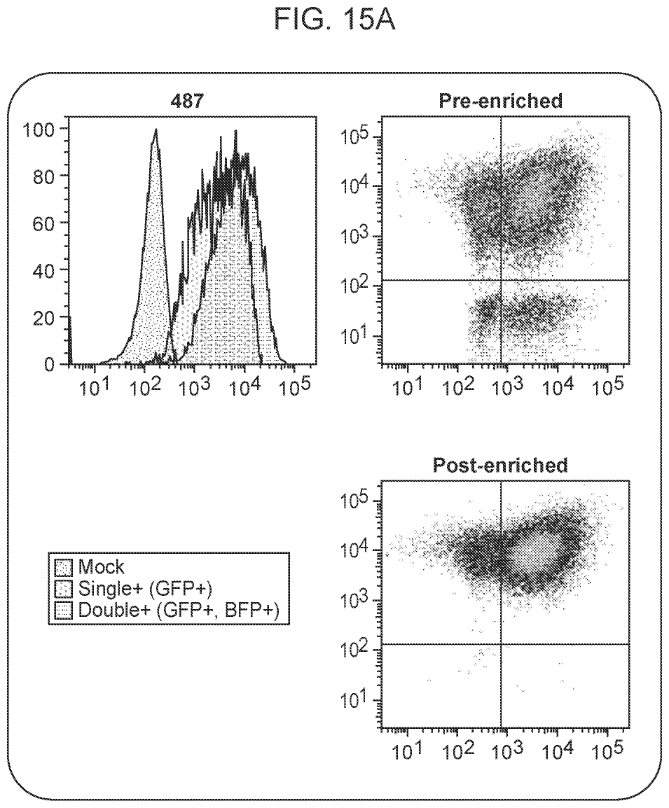

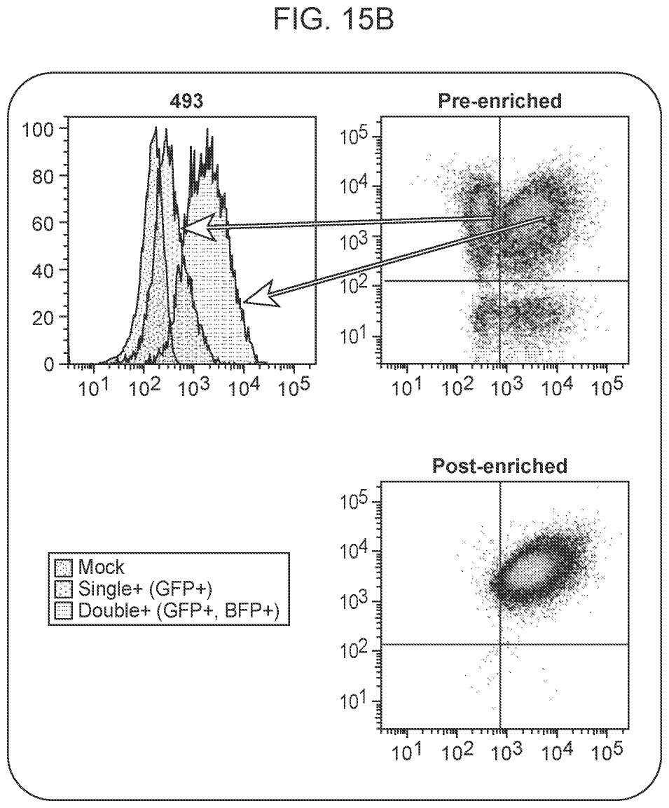

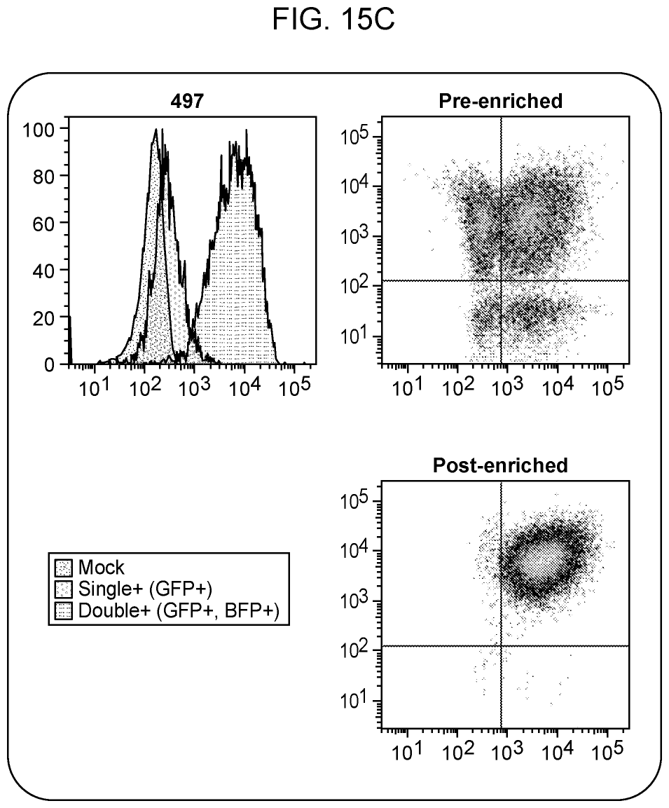

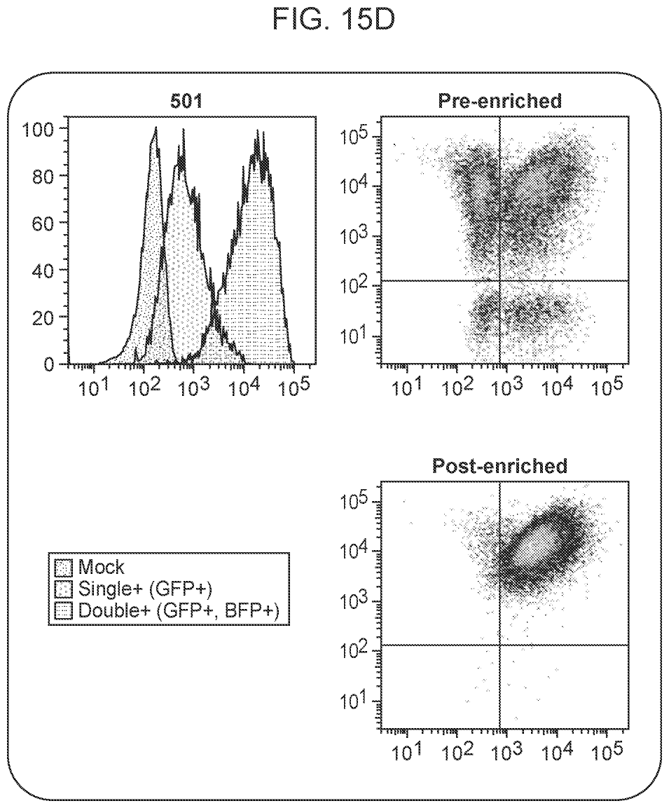

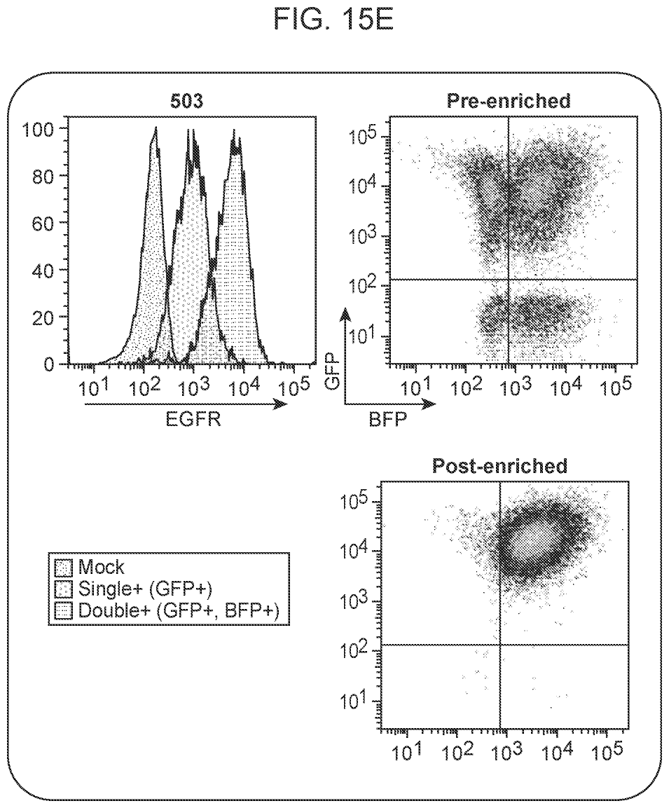

FIG. 15 is a series of flow plots showing BFP, GFP and surface EGFR expression on primary human T cells for five EGFRt STASH variants pre and post-enrichment by EGFR MACS selection. For each variant (variant indicated by the number above the histogram plot), a histogram of surface EGFR expression is shown for single+ and double+ populations, along with dot plots showing BFP and GFP expression pre and post-enrichment. Mock negative control cells, cells that are singly positive for GFP, and cells which are doubly positive for GFP and BFP are indicated. As can be seen in the data, variants 493, 497, 501, and 503 have large differential surface EGFR expression between single and double positive populations, which results in a high degree of purity of double populations after EGFR MACS selection, whereas construct 487, which has limited differential surface EGFR expression results in an impure population after MACS selection, as indicated by the relatively large single+ fraction (GFP+BFP−) in the post-enrichment sample.

### Two-Way STASH Select Using EGFRt-STASH Variant 497 at Low Initial Double Positive Cell Fractions

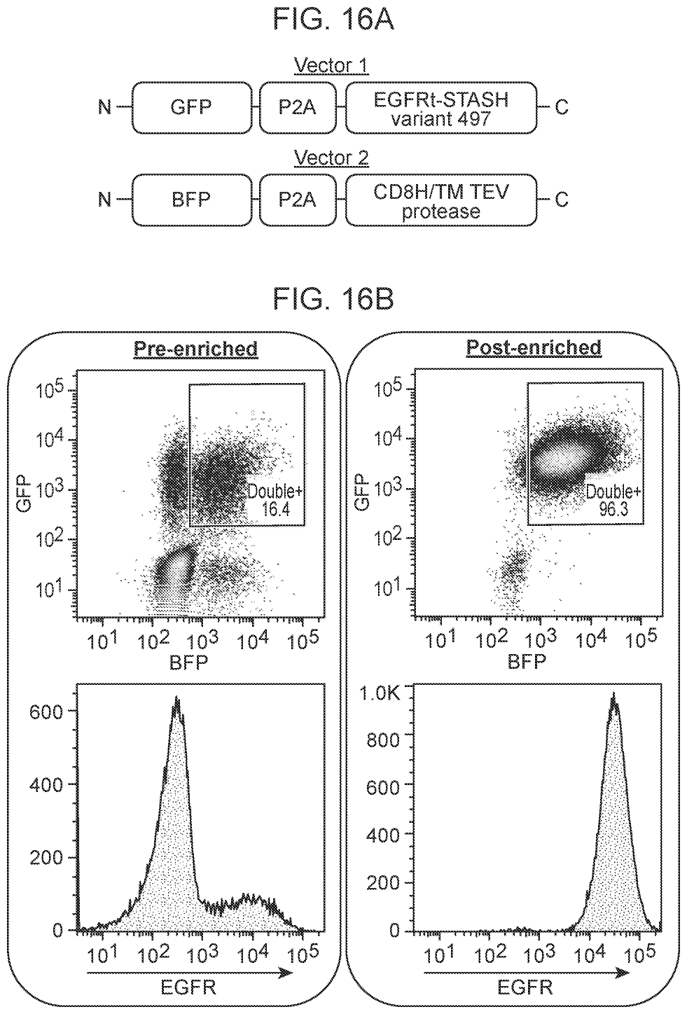

FIG. 16A is a schematic of a two-way STASH Select system with EGFRt-STASH variant 497 as the STASHed surface marker. The first expression construct encodes GFP, a P2A ribosome skip sequence, a EGFRt STASH variant 497. Expression construct 2 encodes BFP, a P2A ribosome skip sequence, a CD8 H/Tm, TEV protease, and an E3-19K protein ER retention tag (LYKYKSRRSFIDEKKMP; SEQ ID NO:1).

FIG. 16B is a series of flow plots showing BFP, GFP, and surface EGFR expression for EGFRt STASH variant 497 pre- and post-enrichment by EGFR MACS selection. As can be seen in the data, variant 497 results in a high degree of purity of double populations after EGFR MACS selection (96.3%), even when starting from low initial double positive populations (16.4%).

Two-way STASH Select using EGFRt-STASH variant 493 at low initial double positive cell fractions

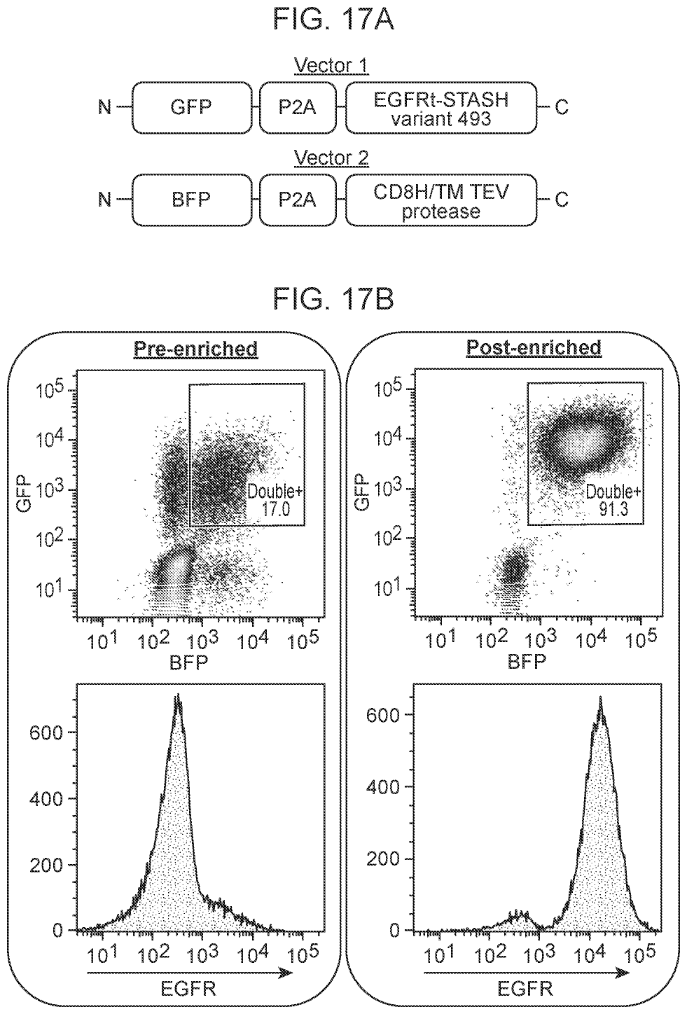

FIG. 17A is a schematic of a two-way STASH Select system with EGFRt-STASH variant 493 as the STASHed surface marker. The first expression construct encodes GFP, a P2A ribosome skip sequence, a EGFRt STASH variant 493. Expression construct 2 encodes BFP, a P2A ribosome skip sequence, a CD8 H/Tm, TEV protease, and an E3-19K protein ER retention tag (LYKYKSRRSFIDEKKMP; SEQ ID NO:1).

FIG. 17B is a series of flow plots showing BFP, GFP, and surface EGFR expression for EGFRt STASH variant 493 pre- and post-enrichment by EGFR MACS selection. As can be seen in the data, variant 493 results in a high degree of purity of double populations after EGFR MACS selection (91.3%), even when starting from low initial double positive populations (17.0%)

### Two-Way STASH Select Using EGFRt-STASH Variant 491 at Low Initial Double Positive Cell Fractions

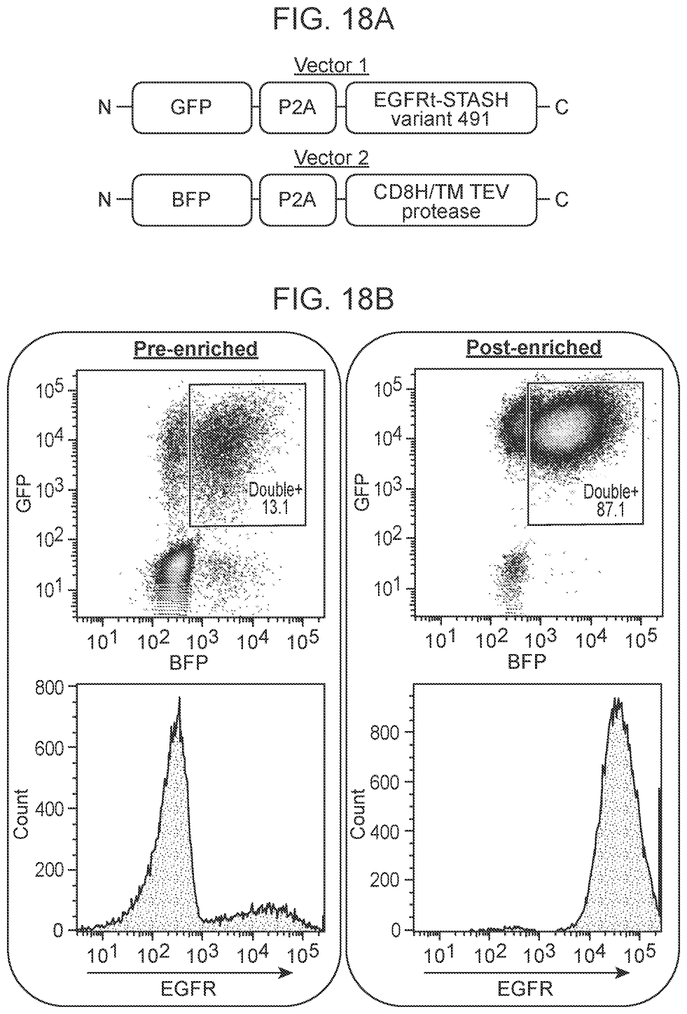

FIG. 18A is a schematic of a two-way STASH Select system with EGFRt-STASH variant 491 as the STASHed surface marker. The first expression construct encodes GFP, a P2A ribosome skip sequence, a EGFRt STASH variant 491. Expression construct 2 encodes BFP, a P2A ribosome skip sequence, a CD8 H/Tm, TEV protease, and an E3-19K protein ER retention tag (LYKYKSRRSFIDEKKMP; SEQ ID NO:1).

FIG. 18B is a series of flow plots showing BFP, GFP, and surface EGFR expression for EGFRt STASH variant 491 pre- and post-enrichment by EGFR MACS selection. As can be seen in the data, variant 491 results in a high degree of purity of double populations after EGFR MACS selection (87.1%), even when starting from low initial double positive populations (13.1%)

### Two-Way STASH Select Using EGFRt-STASH Variant 501 at Low Initial Double Positive Cell Fractions

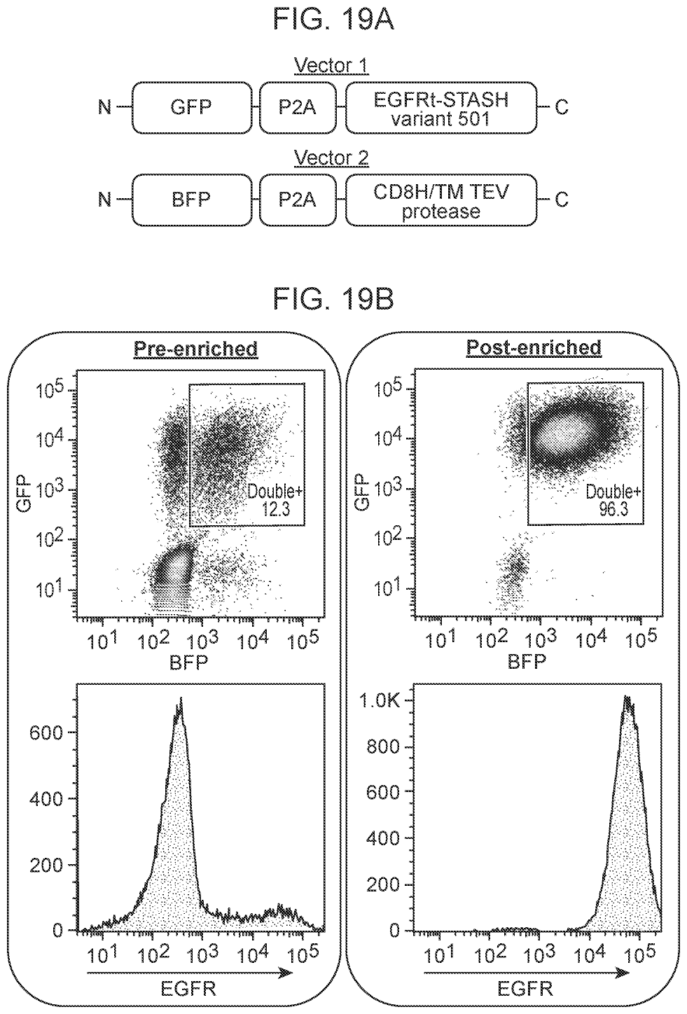

FIG. 19A is a schematic of a two-way STASH Select system with EGFRt-STASH variant 501 as the STASHed surface marker. The first expression construct encodes GFP, a P2A ribosome skip sequence, a EGFRt STASH variant 501. Expression construct 2 encodes BFP, a P2A ribosome skip sequence, a CD8 H/Tm, TEV protease, and an E3-19K protein ER retention tag (LYKYKSRRSFIDEKKMP; SEQ ID NO:1).

FIG. 19B is a series of flow plots showing BFP, GFP, and surface EGFR expression for EGFRt STASH variant 501 pre- and post-enrichment by EGFR MACS selection. As can be seen in the data, variant 501 results in a high degree of purity of double populations after EGFR MACS selection (96.3%), even when starting from low initial double positive populations (12.3%)

### Three-Way STASH Select of CD22, CD19 and HER2 CAR-T Cells Using EGFRt-STASH Variant 497

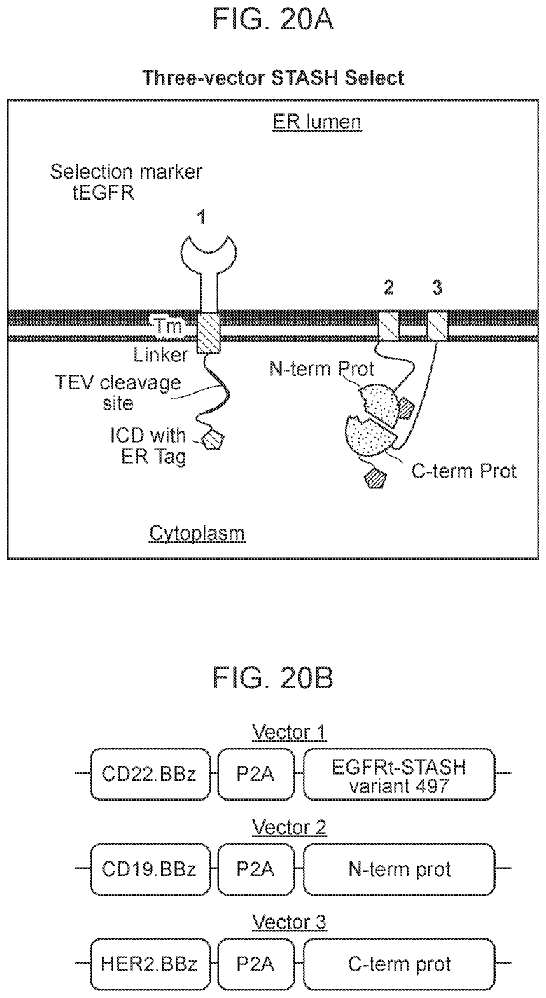

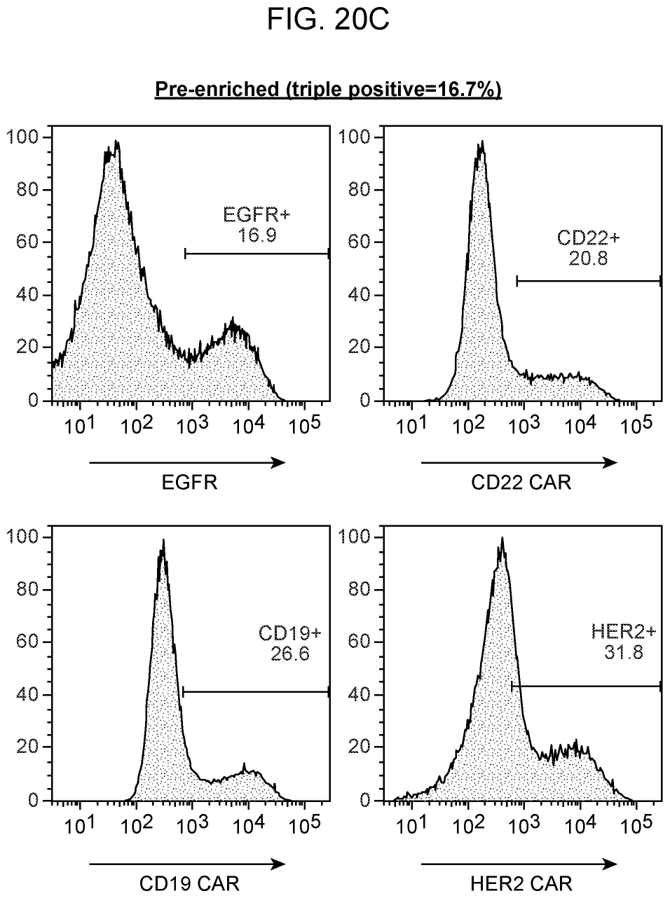

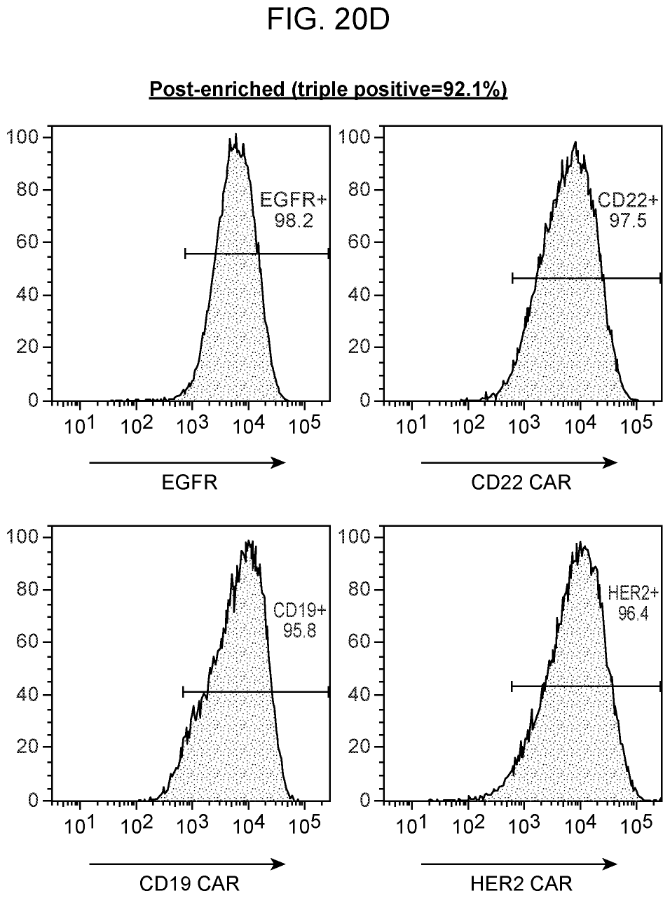

FIG. 20A is a schematic of the three-way STASH selection system. An epitope-based selection marker (e.g., EGFRt) is fused to a protease cleavage site and an intracellular retention tag (e.g. endoplasmic reticulum retention tag). Co-expression of a split protease, whereby the N-terminal domain of the protease is tethered to one transmembrane protein and the C-terminal domain is tethered to another transmembrane protein, results in reconstitution of an active protease complex. The active protease complex cleaves the selection marker at the protease cleavage site, which liberates the selection marker from the ER retention tag and allows the selection marker to translocate to the surface of the cell. The surface-expressed selection tag can then be used as a selection handle to isolate cells expressing both the selection marker and the two protease domains (N-term protease and C-term protease).

FIG. 20B is a schematic depicting expression constructs which encode for three proteins of interest (CD22, CD19, and HER2.BBz CAR) and the components of the STASH selection system (EGFRt-STASH variant 497, N-term protease, and C-term protease). A ribosome skipping site (P2A from porcine teschovirus) allows for bicistronic expression of the proteins of interest and the STASH selection system components.

FIG. 20C is a series of flow plot histograms showing surface expression of EGFR, CD22, CD19, and HER2.BBz CAR-T cells. Primary human T cells were activated at Day 0 with CD3/CD28 activation beads. At Day 2, they were exposed to viral expression construct 1 for 24 hours. At Day 3, the T cells were exposed to a 1:1 mixture of viral expression construct 2 and viral expression construct 3. On Day 7, T cells were harvested, purified by EGFR MACS, stained for the indicated surface markers, and analyzed by flow cytometry. As can be seen in the data, three way STASH Select allows for isolation of a highly pure population of tri-specific CAR-T cells that were transduced with three separate viral expression constructs. The isolation was accomplished using a single a single EGFR MACS selection.

STASH Select Variant 493 with a Degron Domain Allows for Antibiotic Selection

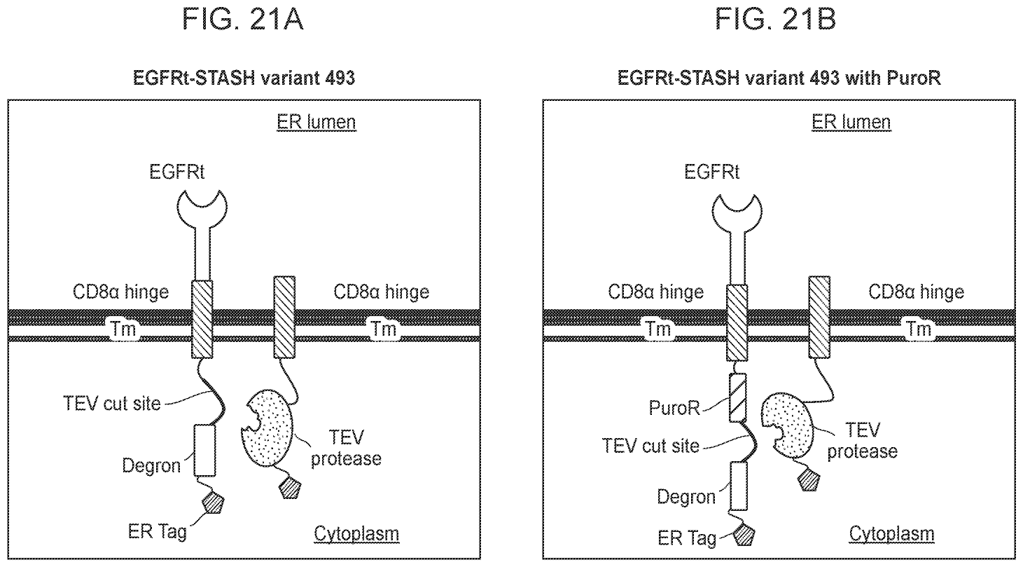

FIG. 21A is a schematic of the two-way STASH Selection system using EGFRt-STASH variant 493, which is comprised of the extracellular domain (ECD) of EGFRt fused to CD8α hinge and transmembrane domains, a TEV cleavage site, a degron, and an ER retention tag. The ER retention tag and degron reduce surface expression of EGFRt-STASH, in the absence of protease, by retaining EGFRt intracellularly and marking the protein for degradation. Co-expression of TEV protease, which is tethered to a CD8a transmembrane protein, results in cleavage of the selection marker at the TEV cleavage site, which liberates the selection marker from the ER retention tag and degron and allows the selection marker to translocate to the surface of the cell at high expression levels. The surface-expressed selection tag can then be used as a selection handle to isolate cells expressing both the STASHed selection marker and protease component.

FIG. 21B is a schematic of the two-way STASH Selection system using EGFRt-STASH variant 493, which is comprised of the extracellular domain (ECD) of EGFRt fused to CD8α hinge and transmembrane domains, a puromycin resistance gene (PuroR, puromycin-N-acetyltransferase), a TEV cleavage site, a degron, and an ER retention tag. The ER retention tag and degron reduce expression of EGFRt-STASH, in the absence of protease, by retaining EGFRt intracellularly and marking the protein for degradation. Co-expression of TEV protease, which is tethered to a CD8a transmembrane protein, results in cleavage of the selection marker at the TEV cleavage site, which liberates the selection marker from the ER retention tag and degron and allows the selection marker to translocate to the surface of the cell at high expression levels. The surface-expressed selection tag can then be used as a selection handle to isolate cells expressing both the STASHed selection marker and protease component by puromycin antibiotic selection or by MACS using the surface expressed EGFRt.

### Chemical-Based Selection of Double Positive Cells Using a Single Antibiotic Selection Marker

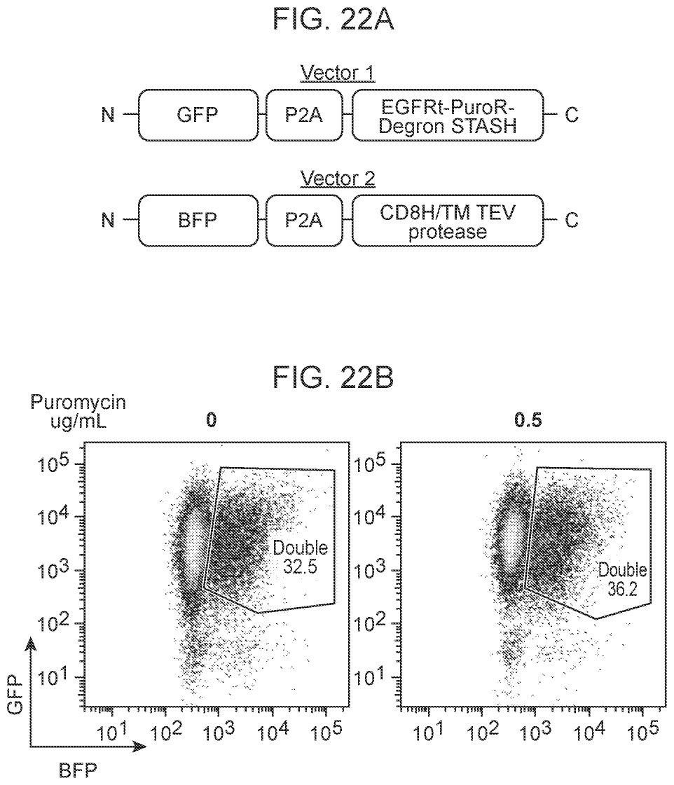

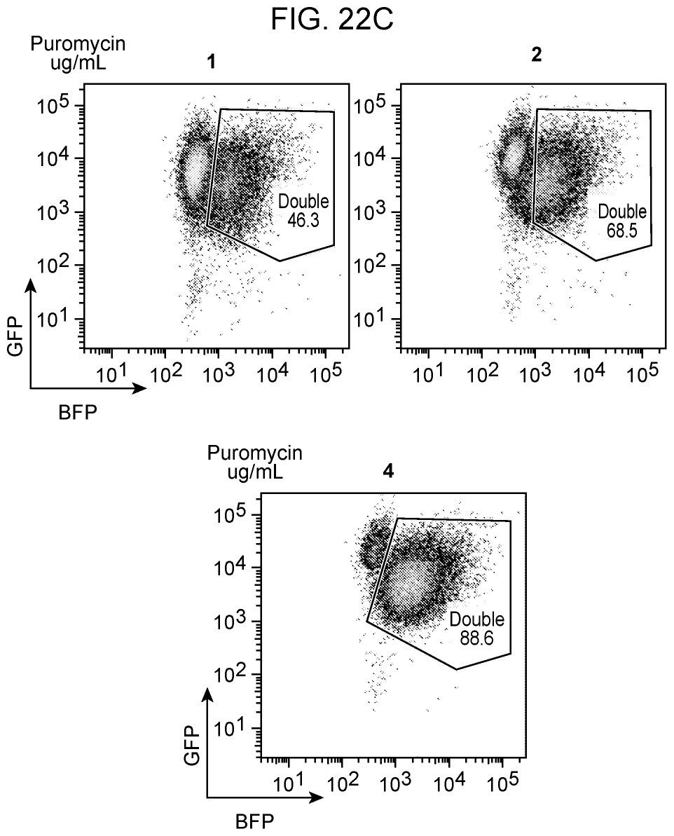

FIG. 22A is a schematic of a two-way STASH Select system with EGFRt-STASH variant 493 with an integrated puromycin resistance gene as the STASHed selection marker. The first expression construct encodes GFP, a P2A ribosome skip sequence, the extracellular domain (ECD) of EGFRt fused to CD8α hinge and transmembrane domains, a puromycin resistance gene (PuroR, puromycin-N-acetyltransferase), a TEV cleavage site, a degron, and an ER retention tag. Expression construct 2 encodes BFP, a P2A ribosome skip sequence, a CD8 H/Tm, TEV protease, and an E3-19K protein ER retention tag (LYKYKSRRSFIDEKKMP; SEQ ID NO:1).

FIG. 22B is a series of flow plots of primary human T cells transduced with a mixture of expression construct 1 and expression construct 2 shown in FIG. 22A, demonstrating BFP and GFP expression after 96 hours of puromycin exposure at the indicated puromycin concentrations. As can be seen in the data, cells that are double positive for expression construct 1 and 2 (BFP+ and GFP+) are progressively enriched with increasing concentrations of puromycin. These data demonstrate that this variant of STASH Select allows for purification of doubly positive cells using a single chemical selection marker.

Two-Way STASH Select with CD34 as the Epitope Marker

FIG. 34 is a series of flow cytometry histograms of surface CD34 staining using the QBEnd/10 antibody on primary human T cells). As can be seen in the data, only double positive cells display high surface expression of the C34 epitope.

Two-Way STASH Select with TEV Protease Bearing a CISD2 ER Retention Tag

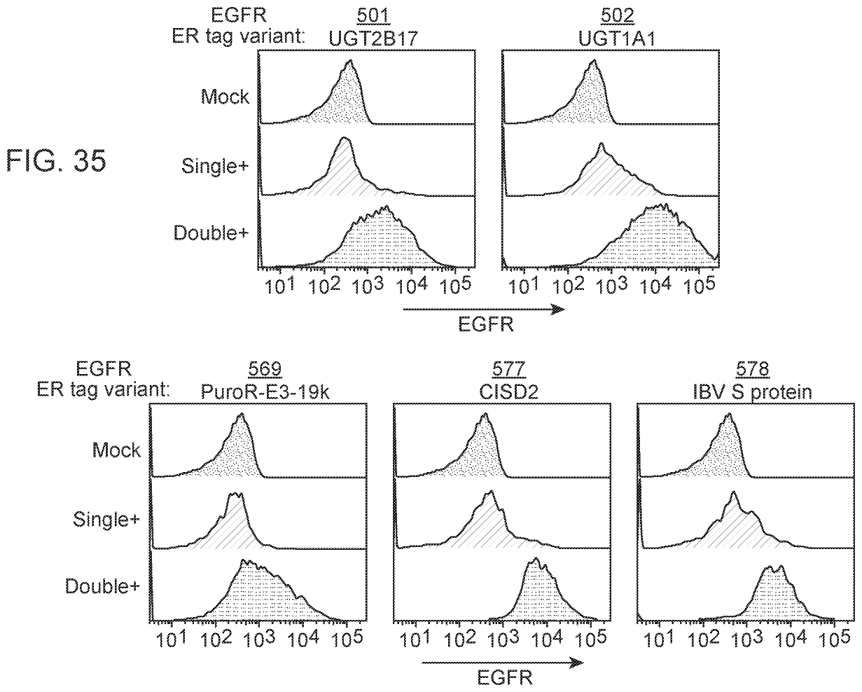

FIG. 35 is a series of flow cytometry histograms showing surface EGFR expression on primary human T cells transduced with a EGFRt-STASH variant and a TEV protease bearing a CISD2 ER retention tag. The specific EGFRt-STASH variant is indicated above each plot. These data demonstrate that TEV protease with a CISD2 ER retention tag is functional in the ER STASH Select system and displays compatibility with several EGFRt-STASH variants.

Three-Way STASH Select with Various Tm Combinations for EGFRt-STASH Variants with CISD2 Retention Signals

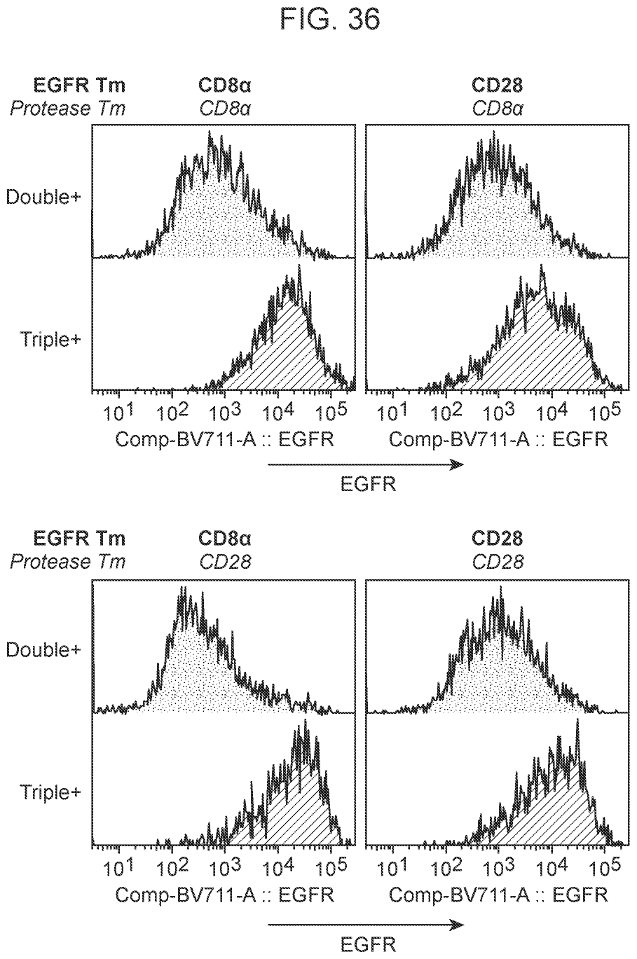

FIG. 36 is a series of flow cytometry histograms showing surface EGFR expression on primary human T cells transduced with the three-way STASH Select system using a EGFRt-STASH variant bearing a CD8α or CD28 Tm domain and a CISD2 ER retention signal. The cells were cotransduced with split TEV variants fused to CD8α or CD28 transmembrane (Tm) domains. These data demonstrate that both CD8α and CD28 Tm domains are compatible in the three-way STASH Select using split TEV protease and an EGFRt-STASH with a CISD2 ER retention signal.

Three-Way STASH Select with Various Tm Combinations for EGFRt-STASH Variants with IBV S Protein ER Tag

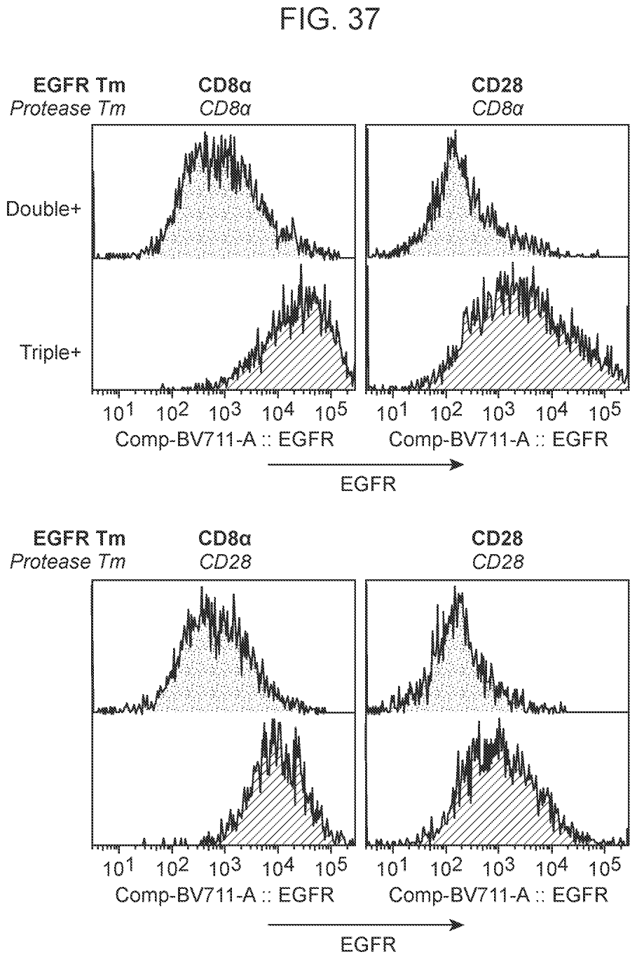

FIG. 37 is a series of flow cytometry histograms showing surface EGFR expression on primary human T cells transduced with the three-way STASH Select system using a EGFRt-STASH variant bearing a CD8α or CD28 Tm domain and an IBV S protein retention signal. The cells were cotransduced with split TEV variants fused to CD8α or CD28 transmembrane (Tm) domains. These data demonstrate that both CD8α and CD28 Tm domains are compatible in the three-way STASH Select using split TEV protease and an EGFRt-STASH with an IBV S protein ER retention signal.

Three-Way STASH Select with Various Tm Combinations for EGFRt-STASH Variants with a Degron Fused ER Tag

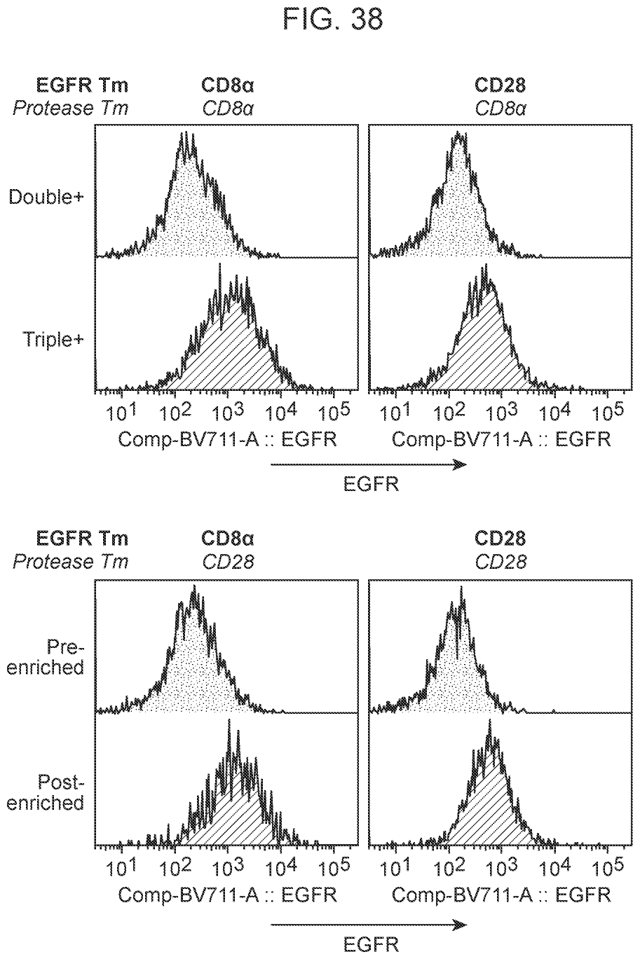

FIG. 38 is a series of flow cytometry histograms showing surface EGFR expression on primary human T cells transduced with the three-way STASH Select system using a EGFRt-STASH variant bearing a CD8α or CD28 Tm domain and a degron fused to the adenovirus E3-19K retention signal. The cells were cotransduced with split TEV variants fused to CD8α or CD28 transmembrane (Tm) domains. These data demonstrate that both CD8 and CD28 Tm domains are compatible in the three-way STASH Select using split TEV protease and an EGFRt-STASH with a degron fused to the adenovirus E3-19K retention signal.

Three-Way STASH Select of cJun, CD19, and HER2 CAR-T Cells Using EGFRt-STASH Variant 507

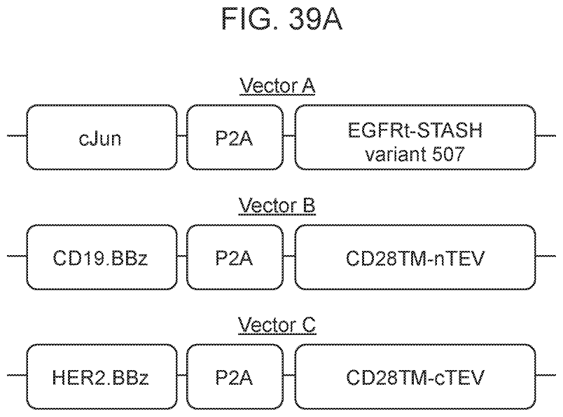

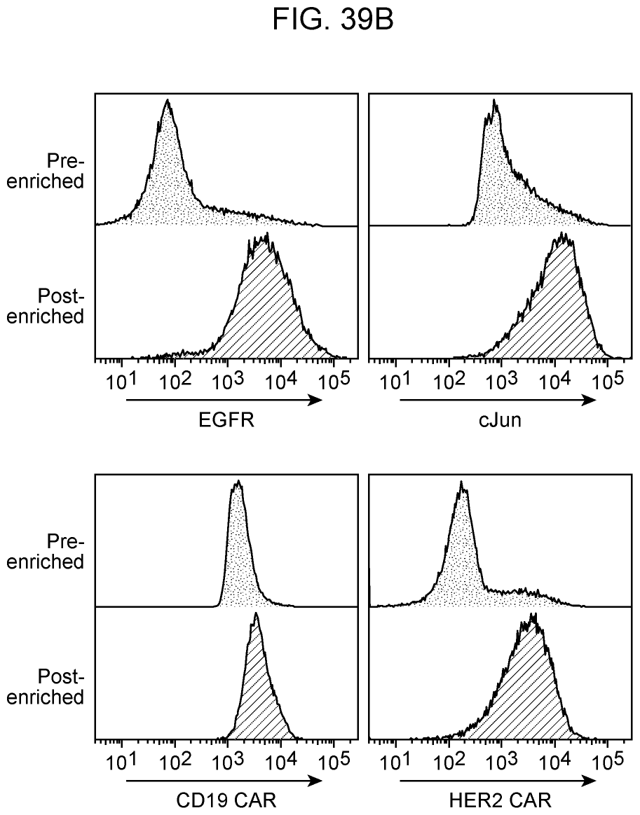

FIG. 39A is a schematic of the three-way STASH selection expression constructs used in this experiment. Expression construct A encode the transcription factor cJun, which renders T cells exhaustion resistant, and a bicistronically expressed EGFRt-STASH variant 507. Expression construct 2 encode a CD19.BBz CAR and a bicistronically expressed N-terminal fragment of split TEV fused to a CD28 hinge an Tm domain. Expression construct 3 encode a HER2.BBz CAR and a bicistronically expressed C-terminal fragment of split TEV fused to a CD28 hinge an Tm domain.

FIG. 39B is a series of flow plot histograms showing surface expression of EGFR, cJun, CD19.BBz, and HER2.BBz CAR. Primary human T cells were activated at Day 0 with CD3/CD28 activation beads. At Day 2, they were exposed to viral expression construct 1 for 24 hours. At Day 3, the T cells were exposed to a 1:1 mixture of viral expression construct 2 and viral expression construct 3. On Day 7, T cells were harvested, purified by EGFR MACS, stained for the indicated surface markers, and analyzed by flow cytometry. As can be seen in the data, three way STASH Select allows for isolation of a highly pure population of bi-specific CAR-T cells (CD19 and HER2 CAR+) expressing the transcription factor cJun. The isolation was accomplished using a single a single EGFR MACS selection. These data demonstrate that the STASH Select system can be used to purify cells expressing three proteins of interest, one of which is a transcription and two of which are receptors, from three expression constructs.

### Anti-EGFR-Biotin Antibody Titration for MACS Using EGFR STASH 497

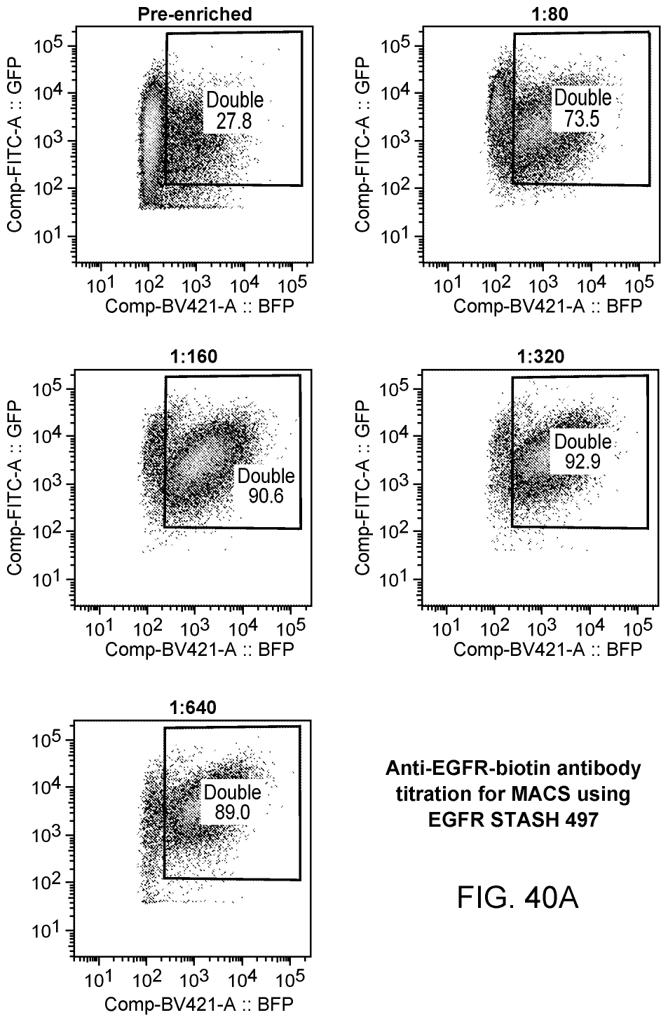

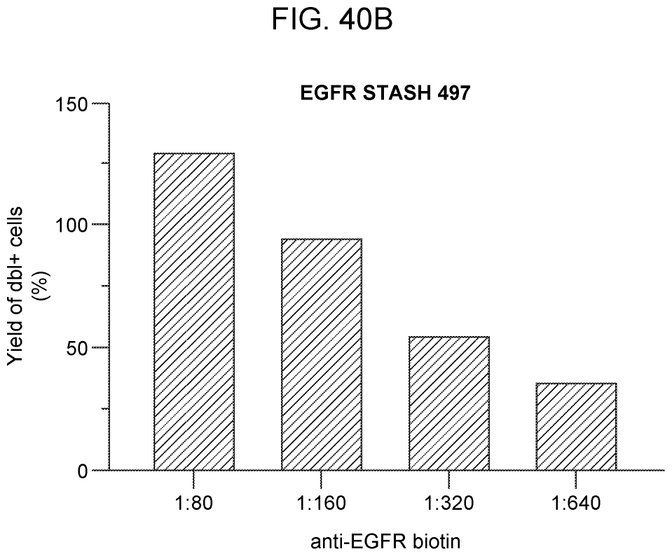

FIG. 40A is a series of flow plots of primary human T cells transduced with a mixture of vector 1 and vector 2 shown in FIG. 16A, demonstrating BFP and GFP expression after staining with anti-EGFR-biotin at the dilution indicated above the flow plot and MACS selection. As can be seen in the data, the concentration of anti-EGFR-biotin antibody during the MACS procedure influences purity of the selected product.

FIG. 40B is a bar plot showing the yield of double positive cells after MACS selection for the samples shown in FIG. 40A.

### Anti-EGFR-Biotin Antibody Titration for MACS Using EGFR STASH 501

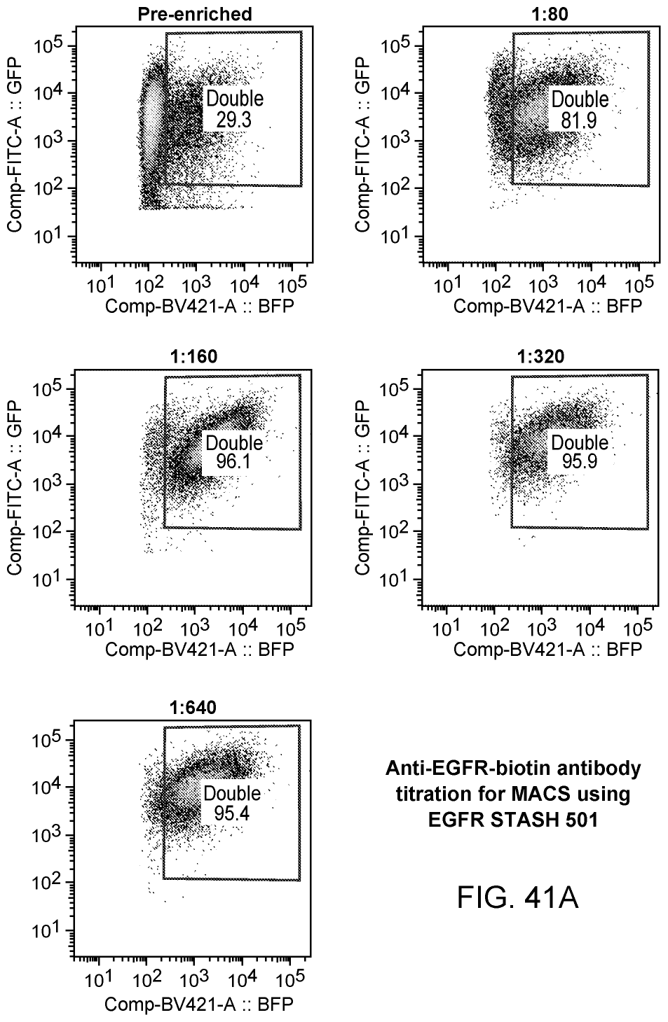

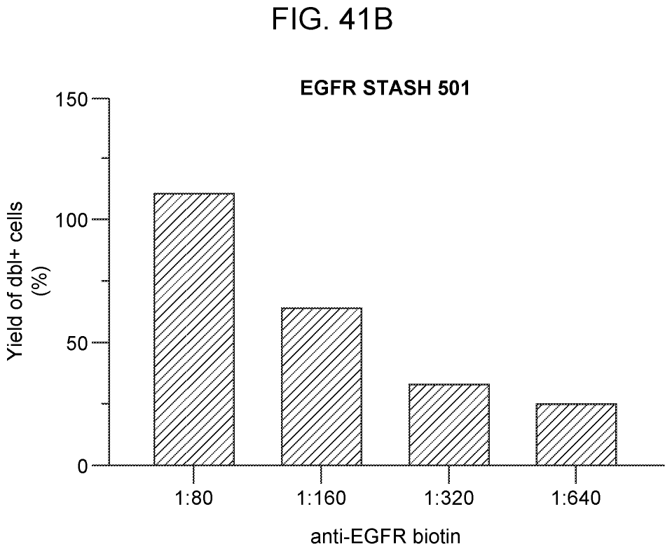

FIG. 41A is a series of flow plots of primary human T cells transduced with a mixture of vector 1 and vector 2 shown in FIG. 19A, demonstrating BFP and GFP expression after staining with anti-EGFR-biotin at the dilution indicated above the flow plot and MACS selection. As can be seen in the data, the concentration of anti-EGFR-biotin antibody during the MACS procedure influences purity of the selected product.

FIG. 41B is a bar plot showing the yield of double positive cells after MACS selection for the samples shown in FIG. 41A.

### Viral Supernatant Dilutions of EGFR STASH 501 and TEV Protease 413: BFP and GFP Expression

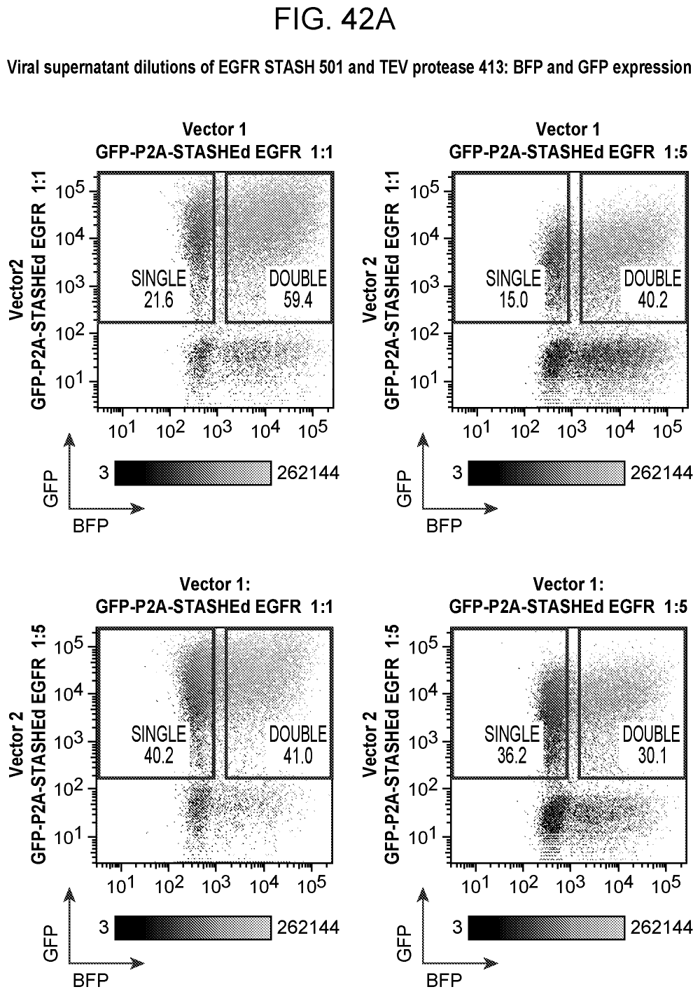

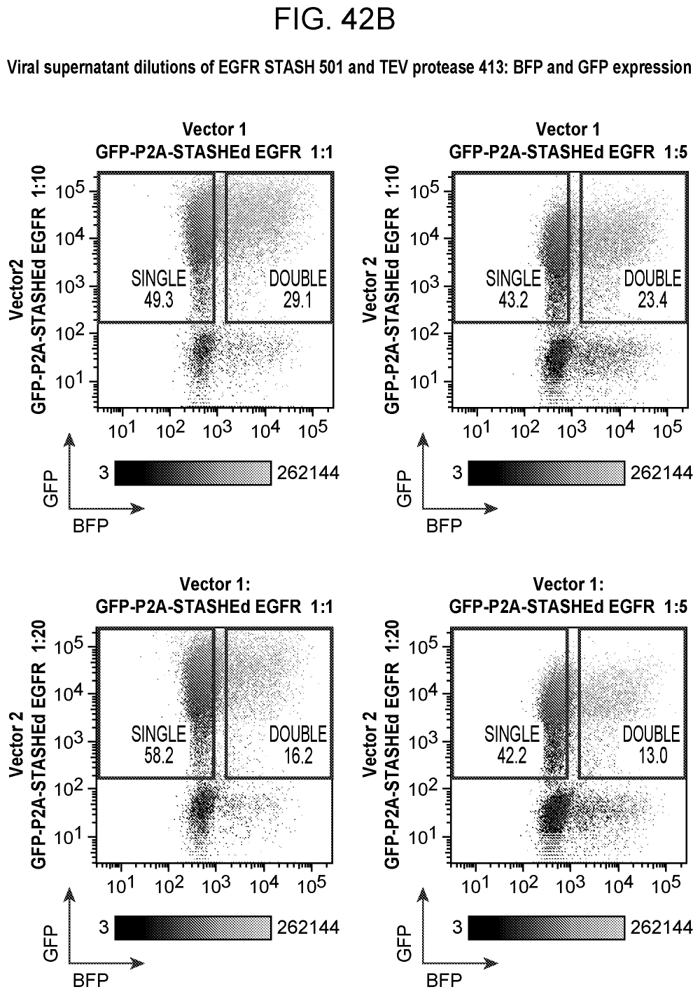

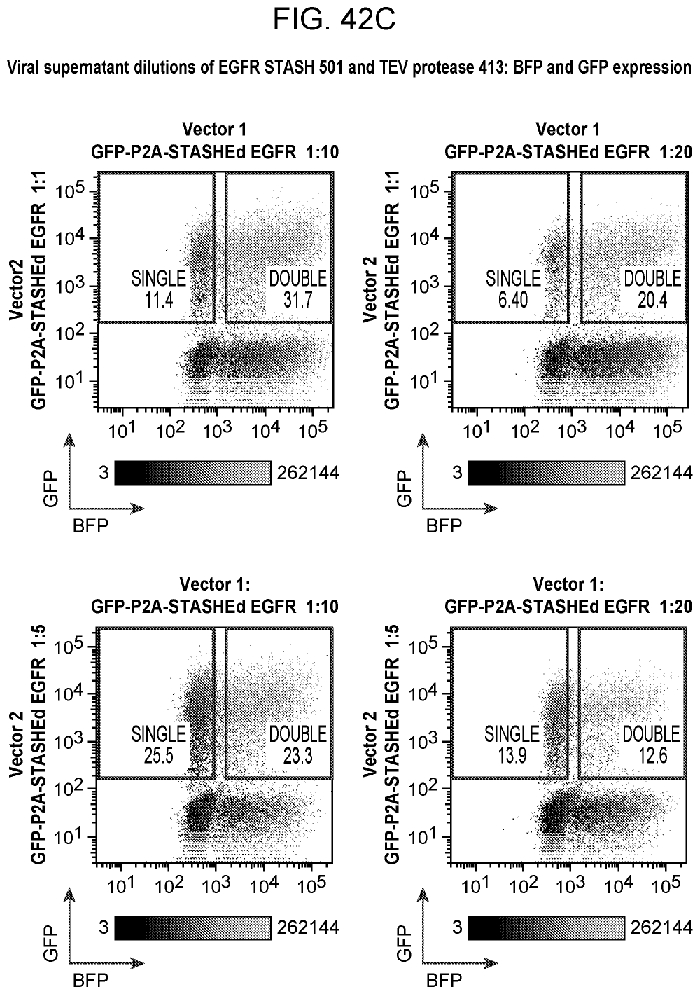

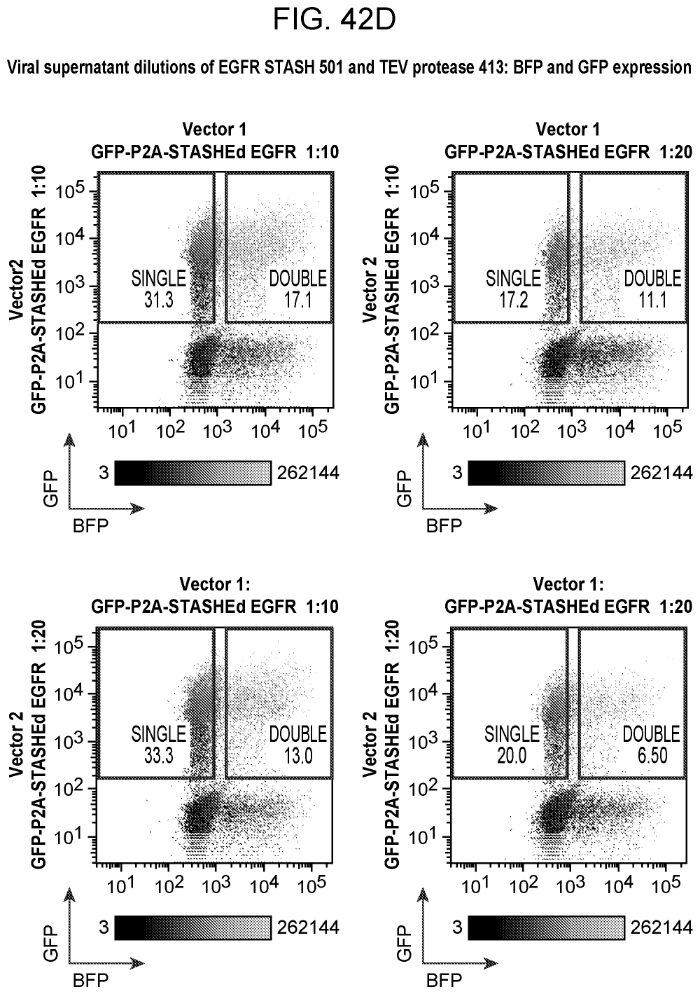

FIG. 42 is a series of flow plots demonstrating BFP and GFP expression in primary human T cells transduced with a mixture of vector 1 and vector 2 shown in FIG. 19A. The viral supernatant dilution for each vector is indicated above and to the side of the flow plots. The intensity of the color scale indicates EGFR expression.

### Viral Supernatant Dilutions of EGFR STASH 501 and TEV Protease 413: Surface EGFR Expression

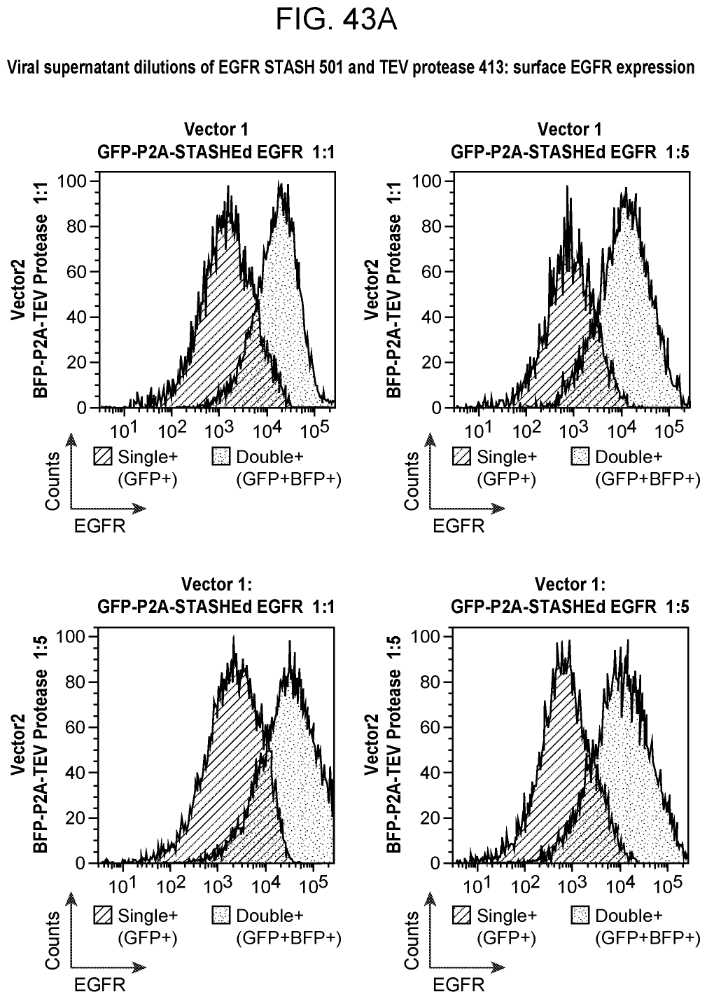

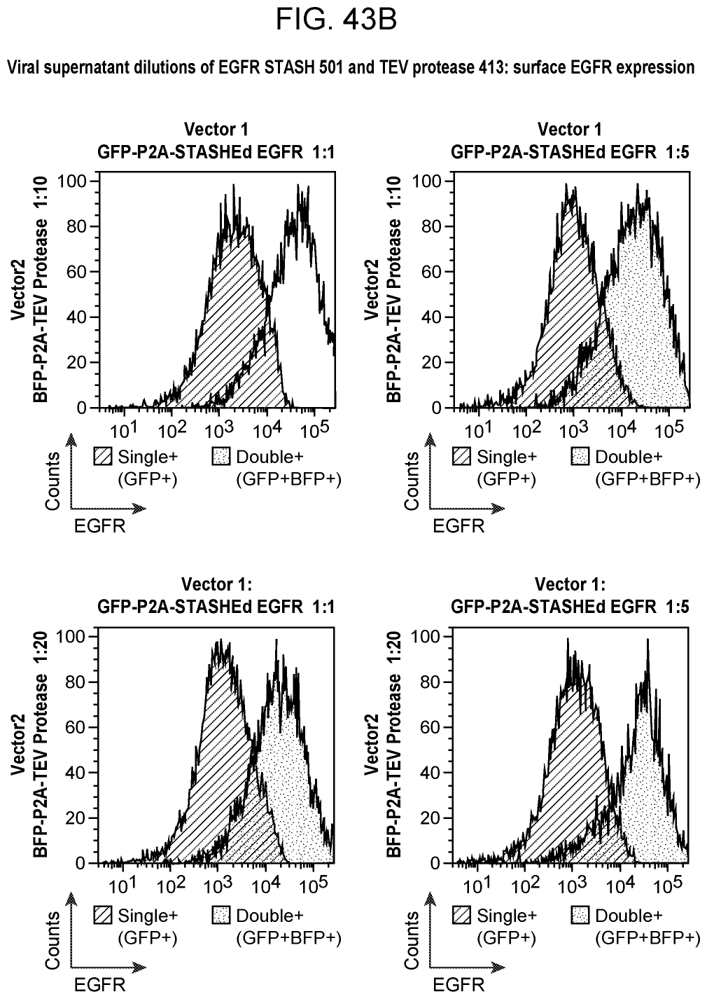

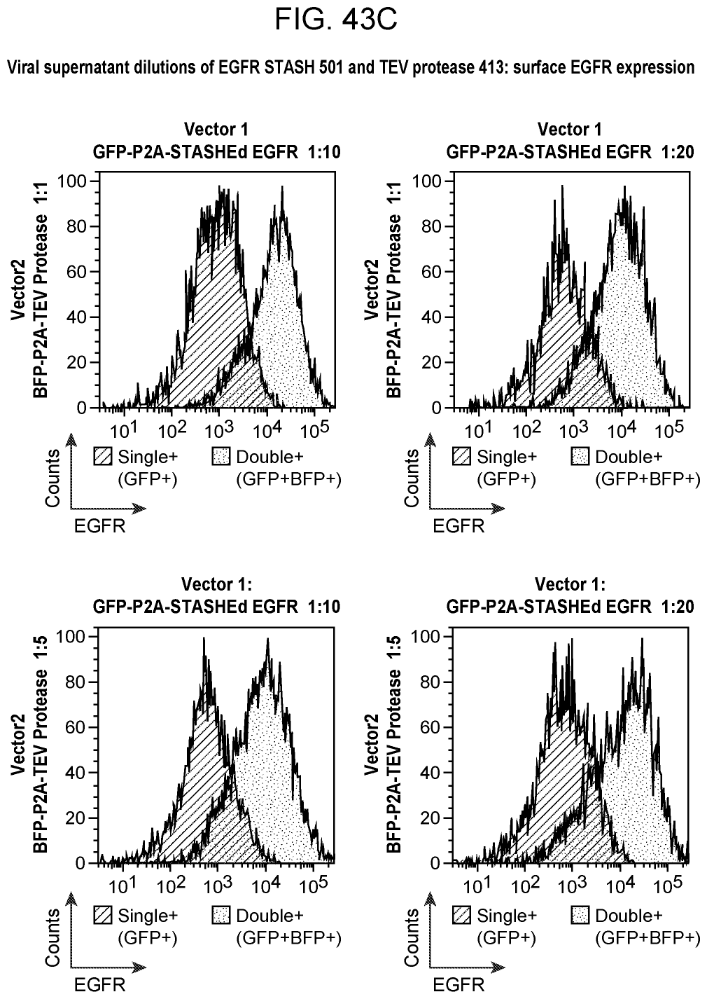

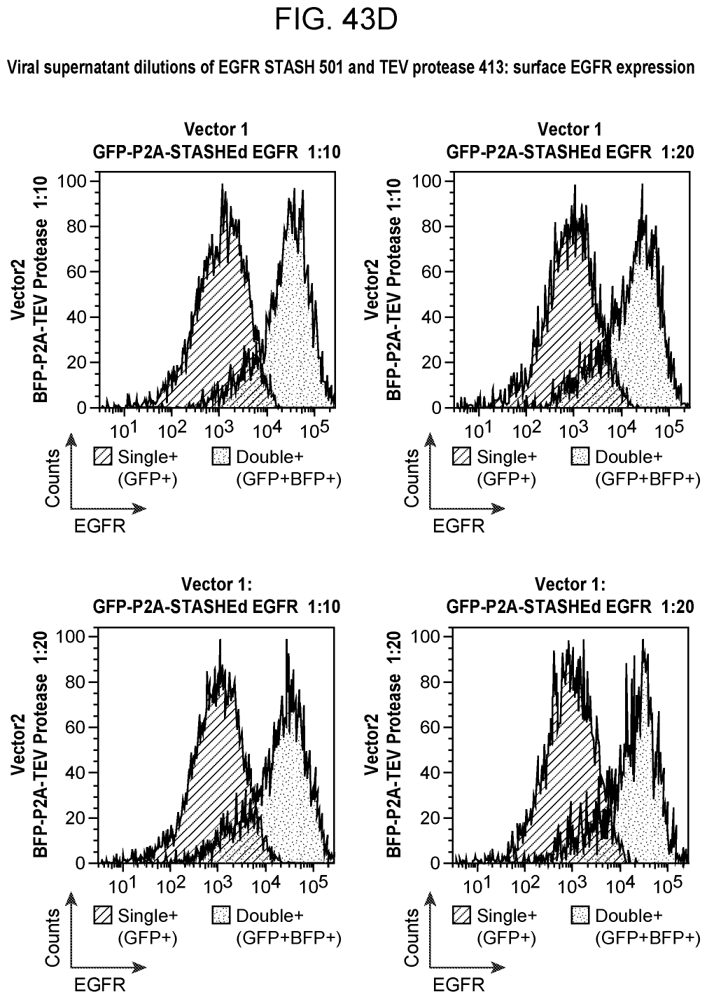

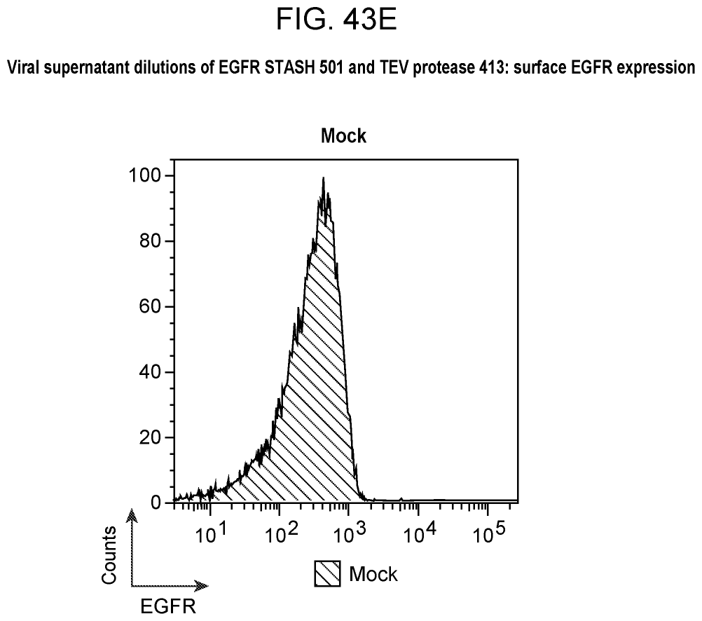

FIG. 43 is a series of flow plots demonstrating surface EGFR expression in primary human T cells transduced with a mixture of vector 1 and vector 2 shown in FIG. 19A. The viral supernatant dilution for each vector is indicated above and to the side of the flow plots. Mock untransduced T cells serve as a negative control. As can be seen in the data, a high degree of differential surface EGFR expression between single and double positive populations was achieved at all combinations of viral supernatant dilutions.

Surface EGFR Expression with Protease 797, a Minimized Protease Construct with a 501 ER Retention Tag

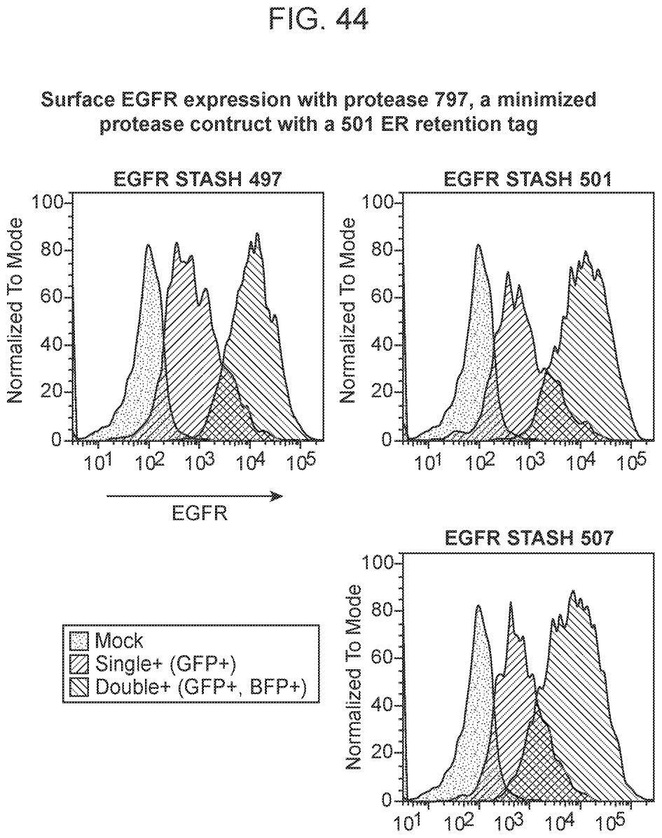

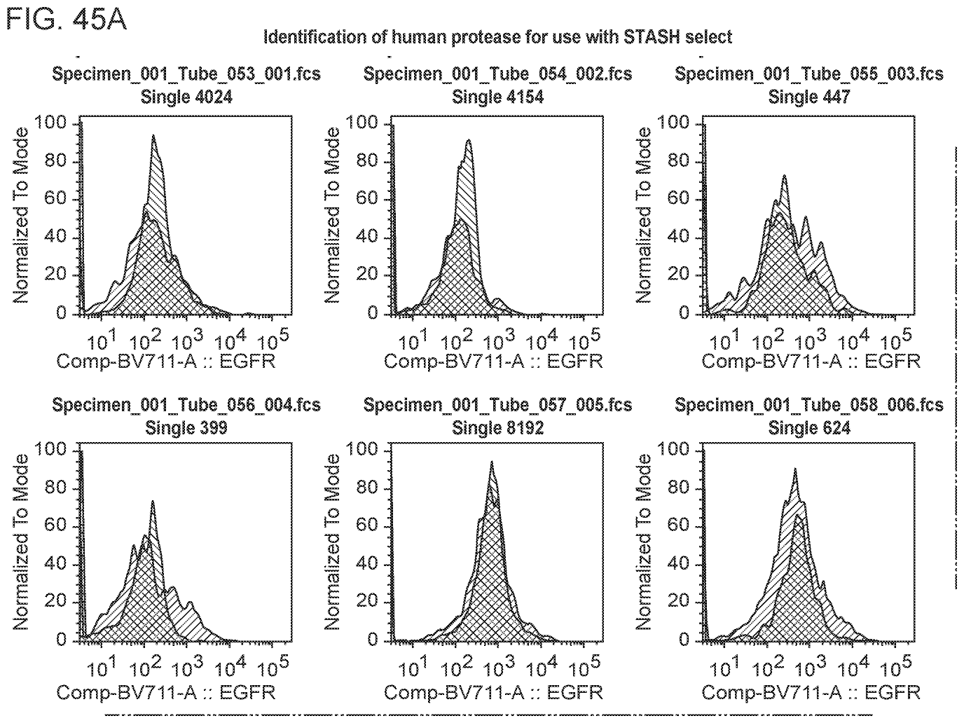

FIG. 44 is a series of flow plots demonstrating surface EGFR expression in primary human T cells transduced with the EGFR STASH variant indicated above each flow plot and a minimized TEV protease construct 797 comprised of BFP-P2A-UGT2B17 membrane tethered TEV protease fused to the variant 501 ER retention tag. As can be seen in the data, a high degree of differential surface EGFR expression between single and double positive populations was achieved using the TEV protease construct 797.

Identification of a Human Protease for Use with STASH Select

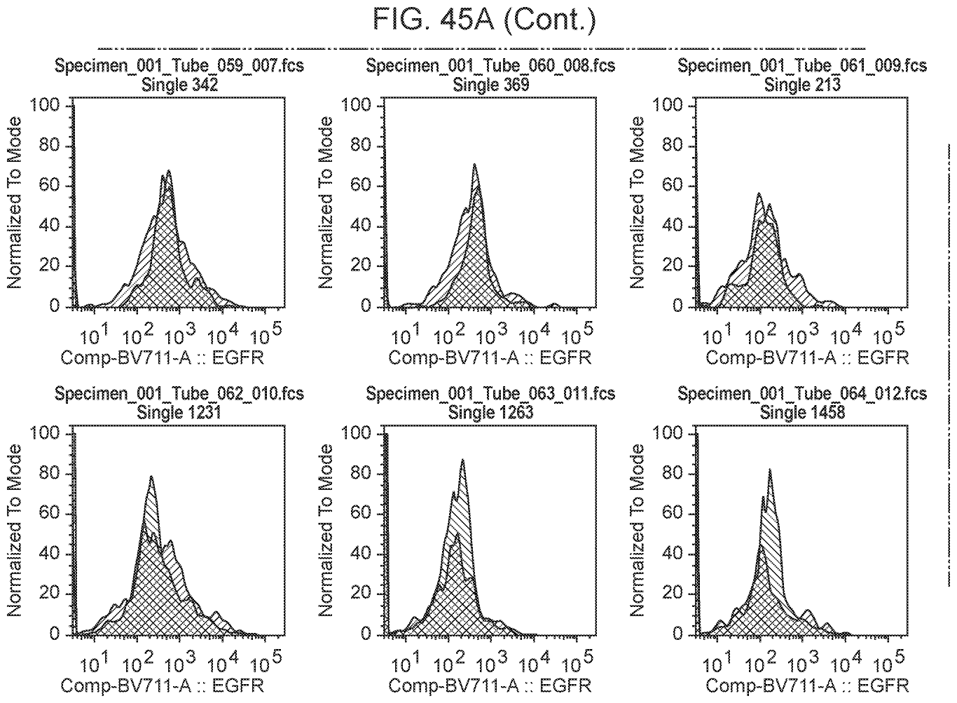

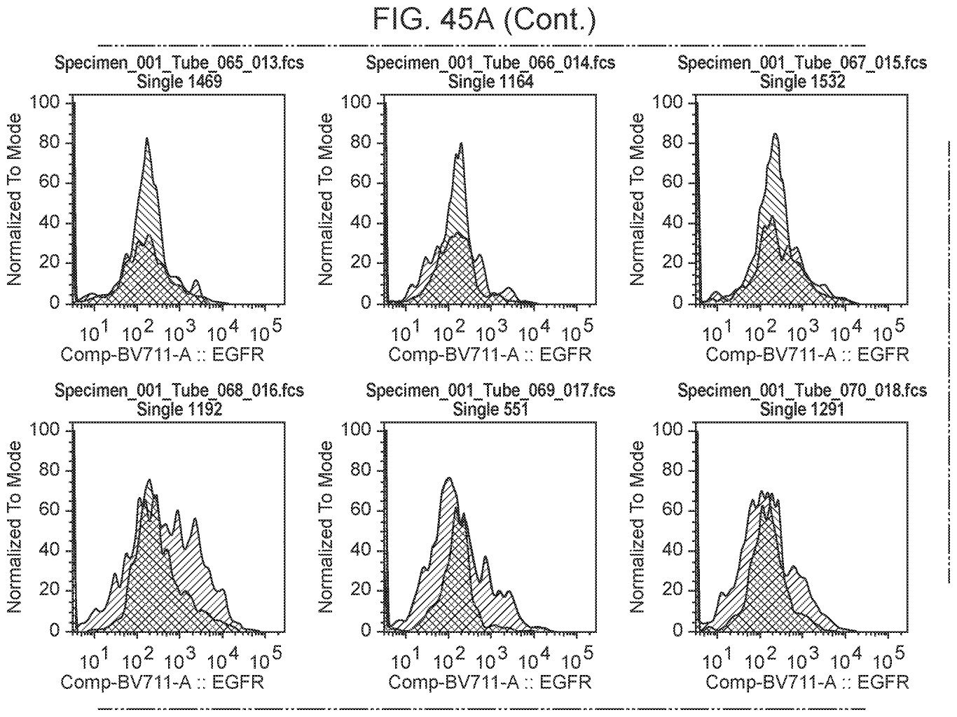

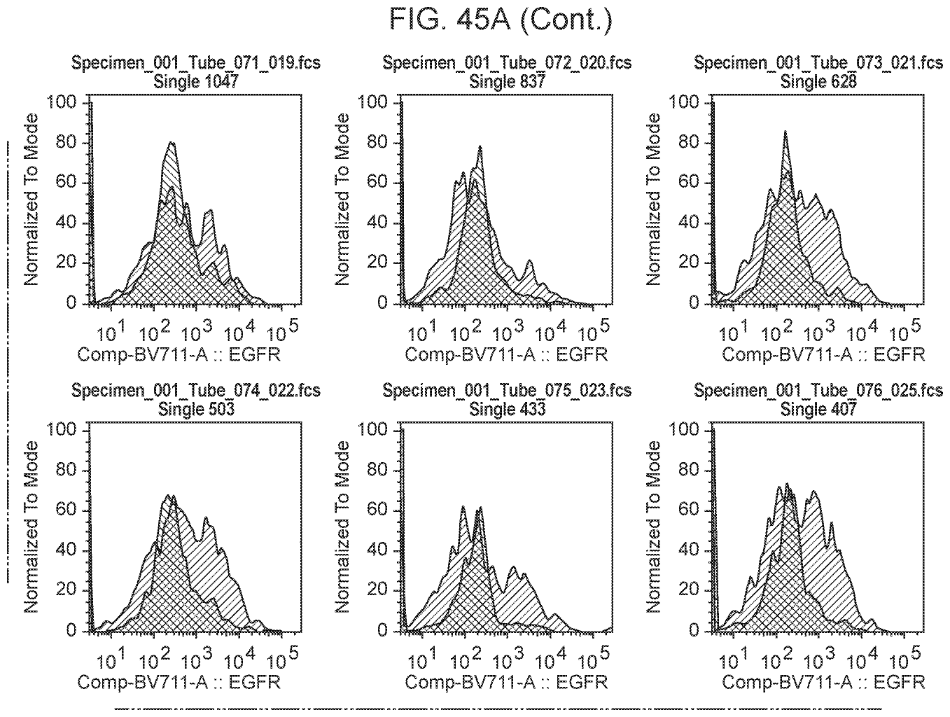

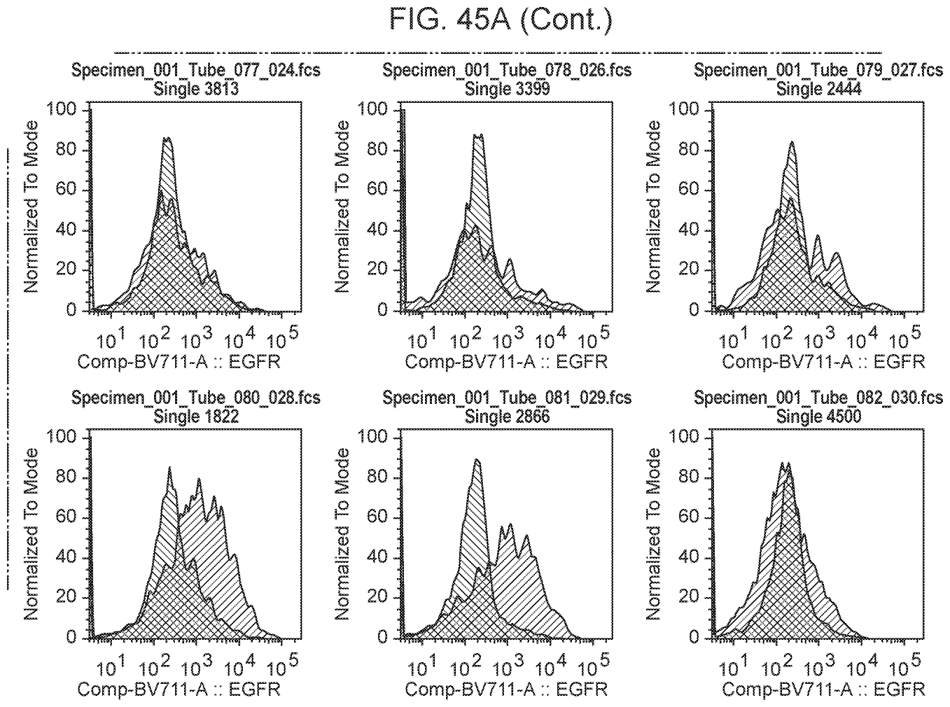

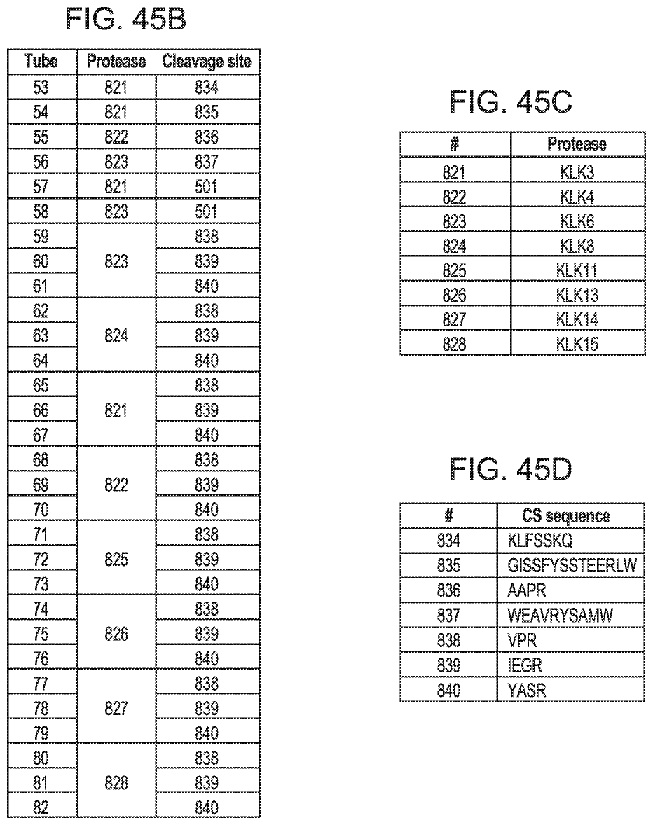

FIG. 45A is a series of flow plots demonstrating surface EGFR expression in primary human T cells co-transduced with two vectors. The first vector is a modified version of EGFR STASH 501 containing a human protease cleavage site instead of a TEV protease cleavage site. The second vector contains a human protease which is cognate for the human cleavage site. As can be seen in the data, several human protease and protease cleavage site combinations result in a high degree of differential surface EGFR expression between single and double positive populations.

FIG. 45B is a table indicating the constructs used to transduce each sample number. FIG. 45C is a table indicating the identity of the human protease used for each protease construct. FIG. 45D is a table indicating the amino acid sequence of the protease cleavage sites used.

### Two-Way STASH Select Using a Combination of CRISPR Knock-In and Retroviral Gene Delivery Methods

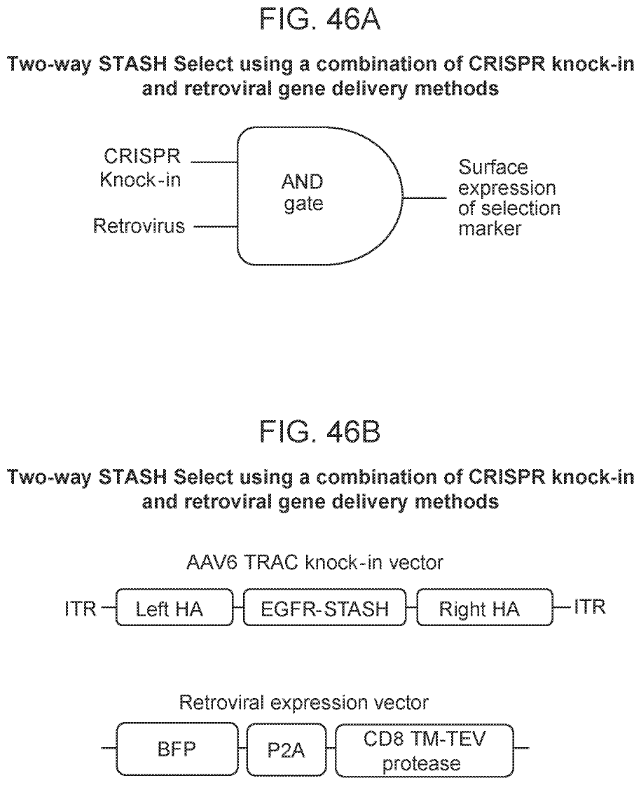

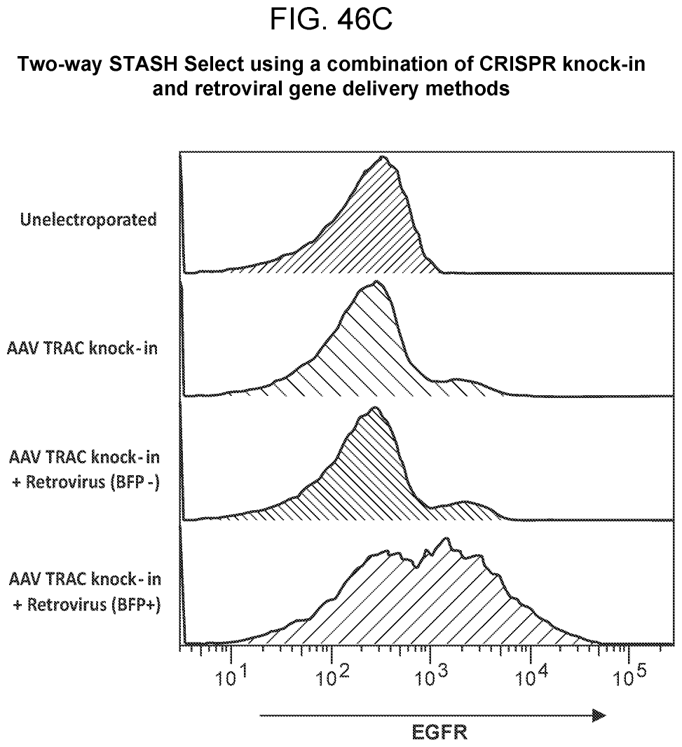

FIG. 46A is a schematic of AND gate logic that can be performed using the STASH Select system. Cells which satisfy the two input requirements (expression of vector A delivered through CRISPR knock-in and expression of vector B delivered through a retroviral vector) result in the output surface expression of the selection marker.

FIG. 46B is a schematic of the two-way STASH selection vectors used in this experiment. The first vector is construct 776, an AAV6 vector which contains a nucleotide sequence with a left homology arm for the TRAC locus, an EGFR-STASH 501 domain, and a right homology arm for the TRAC locus. The second vector is a retroviral expression vector 413 comprised of BFP-P2A-membrane tethered TEV protease fused to an ER retention tag.

FIG. 46C is a series of flow plots demonstrating surface EGFR expression in primary human T cells. Cells were electroporated with Cas9 ribonucleoprotein with a guide specific for the TRAC locus then exposed to the AAV6 vector alone or in combination with the retroviral vector shown in FIG. 46B. Non-electroporated cells serve as a negative control. As can be seen in the data, only cells which have been electroporated with Cas9 ribonucleoprotein with a guide specific for the TRAC locus and exposed to the AAV6 vector, and expressing BFP from the retroviral expression vector have high levels of surface EGFR. These data indicate that the STASH Select components can be delivered through a combination of a retroviral expression vector and site-specific CRISPR-based genome engineering.

Two-Way STASH Select with Various EGFR Truncations

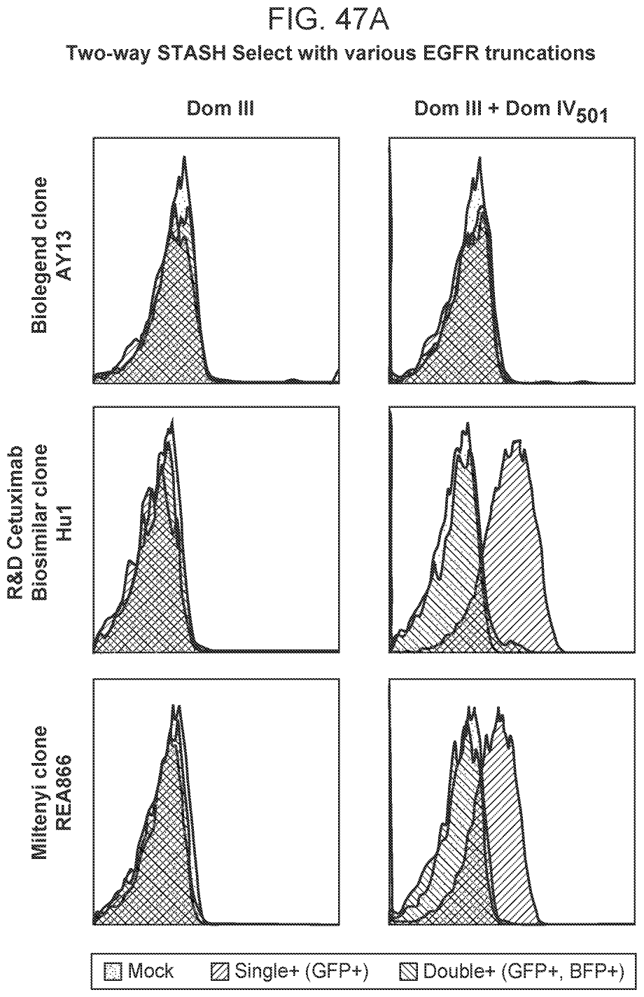

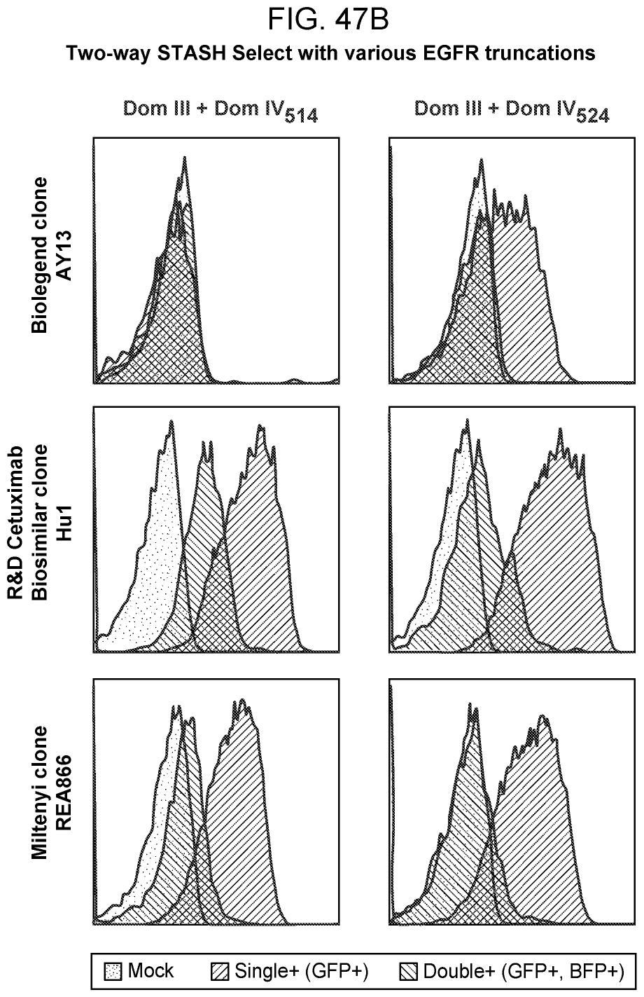

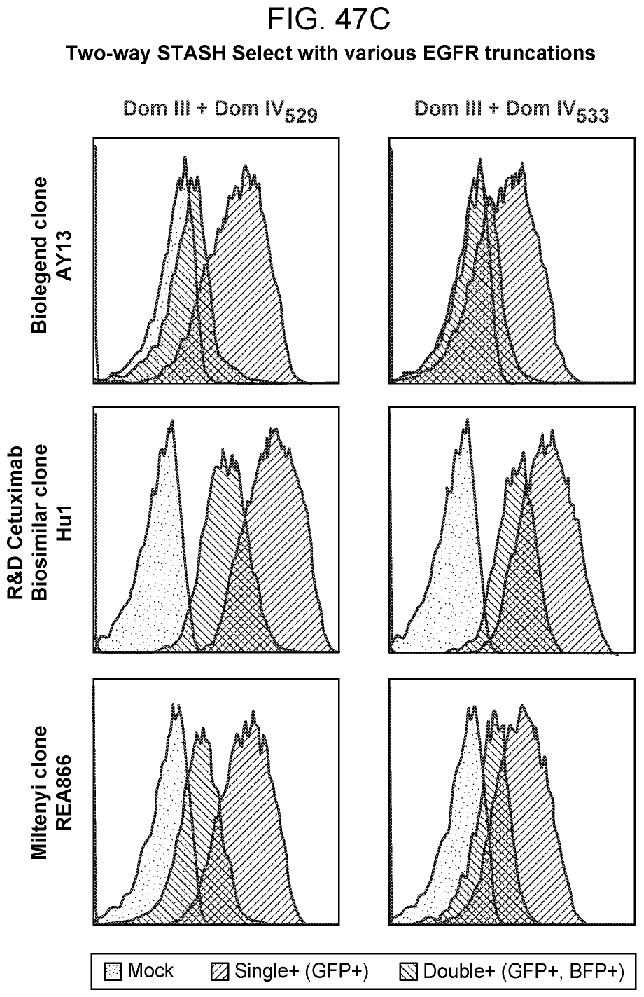

FIG. 47 is a series of flow plots demonstrating surface EGFR expression in primary human T cells transduced with truncated versions of EGFR STASH variant 501 and TEV protease construct #413. The specific truncations were made in domain IV of the EGFR extracellular domain and are indicated above each flow plot. The anti-EGFR antibody used for staining is indicated to the side of the flow plots. As can be seen in the data, a high degree of differential surface EGFR expression between single and double positive populations was achieved with various truncations of EGFR.

### Materials and Methods

### A. Construction of Protease Expression Constructs

### HCV NS3 Protease Expression Constructs for Two-Way STASH Selection

The protease module was expressed as a bicistronic fusion construct according to the following from N to C terminus: Payload protein (BFP, CAR, cJun, etc.), ribosome skip sequence (P2A), leader sequence (TCR-β Leader), protease detection domain (RQR8), transmembrane domain (CD8α hinge and Tm), linker, NS4A cofactor domain, linker, HCV NS3 protease, NS3 helicase fragment, linker, and ER retention tag (adenovirus E3-19K tag).

### TEV Protease Expression Constructs for Two-Way STASH Selection

The protease module was expressed as a bicistronic fusion construct according to the following from N to C terminus: Payload protein (BFP, tdTomato, CAR, cJun, etc.), ribosome skip sequence (P2A), leader sequence (TCR-Leader), protease detection domain (RQR8), transmembrane domain (CD8α hinge and Tm, CD28 hinge and Tm, or CISD2 Tm), linker, TEV protease, linker, and ER retention tag (adenovirus E3-19K tag or CISD2 intracellular domain).

### Human Protease Expression Constructs for Two-Way STASH Selection

The protease module was expressed as a bicistronic fusion construct according to the following from N to C terminus: Payload protein (BFP, tdTomato, CAR, cJun, etc.), ribosome skip sequence (P2A), leader sequence (TCR-β Leader), protease detection domain (RQR8), transmembrane domain (CD8α hinge and Tm, CD28 hinge and Tm, or CISD2 Tm), linker, human protease (such as Kallikrein-15 or enterokinase light chain), linker, and ER retention tag (adenovirus E3-19K tag or CISD2 intracellular domain).

nTEV Protease Expression Constructs for Three-Way STASH Selection

The protease module was expressed as a bicistronic fusion construct according to the following from N to C terminus: Payload protein (BFP, tdTomato, CAR, cJun, etc.), ribosome skip sequence (P2A), leader sequence (TCR-β Leader), protease detection domain (RQR8), transmembrane domain (CD8α hinge and Tm, CD28 hinge and Tm, or CISD2 Tm), linker, nTEV protease (N-terminal domain of split TEV protease comprising 118 N-terminal amino acids of the protease), linker, and ER retention tag (adenovirus E3-19K tag).

cTEV Protease Expression Constructs for Three-Way STASH Selection

The protease module was expressed as a bicistronic fusion construct according to the following from N to C terminus: Payload protein (BFP, tdTomato, CAR, cJun, etc.), ribosome skip sequence (P2A), leader sequence (TCR-β Leader), protease detection domain (RQR8), transmembrane domain (CD8α hinge and Tm, CD28 hinge and Tm, or CISD2 Tm), linker, cTEV protease (C-terminal domain of split TEV protease comprising 118 C-terminal amino acids of the protease), linker, and ER retention tag (adenovirus E3-19K tag).

nTEV Protease Expression Constructs for Five-Way STASH Selection

The protease module was expressed as a bicistronic fusion construct according to the following from N to C terminus: Payload protein (BFP, tdTomato, CAR, cJun, etc.), ribosome skip sequence (P2A), leucine zipper (SYNZIP1, SYNZIP2, SYNZIP1, or SYNZIP4), linker, and nTEV protease (N-terminal domain of split TEV protease comprising 118 N-terminal amino acids of the protease).

cTEV Protease Expression Constructs for Five-Way STASH Selection

The protease module was expressed as a bicistronic fusion construct according to the following from N to C terminus: Payload protein (BFP, tdTomato, CAR, cJun, etc.), ribosome skip sequence (P2A), leucine zipper (SYNZIP1, SYNZIP2, SYNZIP1, or SYNZIP4), linker, and cTEV protease (C-terminal domain of split TEV protease comprising 118 C-terminal amino acids of the protease).

### B. Construction of Protease-Recruiting Transmembrane Protein Expression Constructs

### Protease-Recruiting Transmembrane Protein 1 Expression Constructs for Five-Way STASH Selection

The protease-recruiting transmembrane protein module was expressed as a bicistronic fusion construct according to the following from N to C terminus: Payload protein (BFP, tdTomato, CAR, cJun, etc.), ribosome skip sequence (P2A), leader sequence (GM-CSFR Leader), detection tag (FLAG Tag or Myc Tag), transmembrane domain (CD8α hinge and Tm or CD28 hinge and Tm), linker, leucine zipper (SYNZIP1, SYNZIP2, SYNZIP1, or SYNZIP4), linker, and ER retention tag (adenovirus E3-19K tag).

### Protease-Recruiting Transmembrane Protein 2 Expression Constructs for Five-Way STASH Selection

The protease-recruiting transmembrane protein module was expressed as a bicistronic fusion construct according to the following from N to C terminus: Payload protein (BFP, tdTomato, CAR, cJun, etc.), ribosome skip sequence (P2A), leader sequence (GM-CSFR Leader), detection tag (HA Tag), transmembrane domain (CD8α hinge and Tm or CD28 hinge and Tm), linker, leucine zipper (SYNZIP1, SYNZIP2, SYNZIP1, or SYNZIP4), linker, and ER retention tag (adenovirus E3-19K tag).

### C. Construction of STASH Selection Marker Expression Constructs

### STASH Selection Marker Expression Constructs

The STASH selection module was expressed as a bicistronic fusion construct according to the following from N to C terminus: Payload protein (CAR, cJun, GFP, BFP, tdTomato, etc.), ribosome skip sequence (P2A), leader sequence (GM-CSFR leader), extracellular domain of epitope marker (EGFRt, CD34, Myc Tag, NGFRt), linker, transmembrane domain (CD8α hinge and Tm, CD28 hinge and Tm, CISD2 Tm, TMED4 Tm, Sel1L Tm, DDOST Tm, UGT2B17 Tm, UGT1A1 Tm, TAPBP Tm, TMED4 Tm, TRIQK Tm, mastadenovirus C E3 19K Tm, IBV S Tm, or Calnexin Tm), linker, protease cleavage site (TEV cleavage site, HCV NS3 cleavage site, or human enterokinase light chain cleavage site), linker, and ER retention Tag (adenovirus E3-19K tag, adenovirus E3-19K variant 1 tag, adenovirus E3-19K variant 2 tag, adenovirus E3-19K variant 3 tag, KDELR2 ICD, carboxypeptidase D ICD, Coronavirus infectious bronchitis virus (IBV) S protein ER retention motif, HCV NS3 helix, HCV helicase domain, CISD2 ICD, TMED4 ICD, Sel1L ICD, DDOST ICD, UGT2B17 ICD, UGT1A1 ICD, TAPBP ICD, TRIQK ICD, mastadenovirus C E3 19K ICD, IBV S ICD, or Calnexin ICD).

STASH Selection Marker with Degron Domain Expression Constructs

The STASH selection module was expressed as a bicistronic fusion construct according to the following from N to C terminus: Payload protein (CAR, cJun, GFP, BFP, tdTomato, etc.), ribosome skip sequence (P2A), leader sequence (GM-CSFR leader), extracellular domain of epitope marker (EGFRt, CD34, Myc Tag, NGFRt), linker, transmembrane domain (CD8α hinge and Tm, CD28 hinge and Tm), linker, protease cleavage site (TEV cleavage site), linker, degron domain (HCV NS4A degron domain), and ER retention Tag (adenovirus E3-19K tag).

STASH Selection Marker with Puromycin Resistance and Degron Domains Expression Constructs

The STASH selection module was expressed as a bicistronic fusion construct according to the following from N to C terminus: Payload protein (CAR, cJun, GFP, BFP, tdTomato, etc.), ribosome skip sequence (P2A), leader sequence (GM-CSFR leader), extracellular domain of epitope marker (EGFRt), linker, transmembrane domain (CD8α hinge and Tm), linker, puromycin-N-acetyltransferase (PuroR), linker, protease cleavage site (TEV cleavage site), linker, degron domain (HCV NS4A degron domain), and ER retention Tag (adenovirus E3-19K tag).

STASH Selection Marker with Puromycin Resistance, Degron Domains, and Dual Cleavage Sites Expression Constructs

The STASH selection module was expressed as a bicistronic fusion construct according to the following from N to C terminus: Payload protein (CAR, cJun, GFP, BFP, tdTomato, etc.), ribosome skip sequence (P2A), leader sequence (GM-CSFR leader), extracellular domain of epitope marker (EGFRt), linker, transmembrane domain (CD8α hinge and Tm), linker, protease cleavage site (TEV cleavage site), puromycin-N-acetyltransferase (PuroR), linker, protease cleavage site (TEV cleavage site), linker, degron domain (HCV NS4A degron domain), and ER retention Tag (adenovirus E3-19K tag).

D. Isolation of Primary Human T Cells from Blood Donors

Primary human T cells were extracted from buffy coats by negative selection using the RosetteSep Human T cell Enrichment kit (Stem Cell Technologies) and SepMate-50 tubes. T cells were cryopreserved at CryoStor CS10 cryopreservation media (Stem Cell Technologies) until use.

### E. Construction of Retroviral Plasmid Expression Constructs

DNA sequences were synthesized as gBlocks or oligonucleotides (Integrated DNA Technologies) and cloned into the MSGV1 retroviral expression construct by In-Fusion cloning. In-Fusion reaction products were transformed into chemically competent cells (Stellar Cell, Takara Bio) by heat shock method. Transformants were sequence verified by Sanger sequencing. Bacteria cultures from sequence verified clones were grown for 16 hours at 37C with shaking. Subsequently, the bacteria cells were harvested and DNA was extracted using a miniprep kit (QIAprep Spin Miniprep Kit, Qiagen).

### F. Virus Production

Retroviral supernatant was prepared using 293GP cells and the RD114 envelope plasmid. In brief, 22 ug of the corresponding MSGV1 transfer plasmid and 11 ug of RD114 and were delivered to 293GP cells, grown to about 80% confluency on poly-D-lysine dishes (Corning), by transient transfection using the Lipofectamine 2000 reagent (Thermo Fisher). 293GP cells were cultured in media (DMEM, 10% FBS, 10 mM HEPES, 2 mM L-glutamine, 100 U/mL penicillin, and 100 ug/mL streptomycin, Gibco) at 37° C. in a 5% CO2 environment. Media was replenished every 24 hours. Retroviral supernatant was harvested 48 and 72-hour post transfection, centrifuged to deplete dead cells and debris, and stored at −80 C until further use.

### G. Adeno-Associated Virus (AAV) Production

The EGFR-STASH TRAC knock-in template was cloned into an AAV plasmid backbone in the following configuration ITR, TRAC left homology arm, EF1a promoter, EGFR STASH variant 501, bGH poly (A) signal, TRAC right homology arm, and ITR. AAV was produced by transfecting five 150 mm plates of 293T cells with 30 ug template plasmid and 110 ug AAV6 helper plasmid (pDGM6). 293T cells were cultured in media (DMEM, 10% FBS, 10 mM HEPES, 2 mM L-glutamine, 100 U/mL penicillin, and 100 ug/mL streptomycin, Gibco) at 37° C. in a 5% CO2 environment. Media was replenished every 24 hours. After 72 hours, AAV6 particles were extracted using the AAVpro® Purification Kit Maxi kit (Takara, catalog #6666), according to the manufacturer's instructions. See Wiebking et al. Nat. Biotechnology 2020 for related methods.

### H. T Cell Retroviral Transductions

Primary human T cells were thawed at Day 0 and activated with anti-CD3/CD28 Human T-Expander Dynabeads (Thermo Fisher) at a bead to cell ratio of 3:1. On Day 2 virus coated culture plates were prepared on non TC-treated 12-well or 24 well plates that had been pre-coated with RetroNectin (Takara Bio) according to the manufacturer's instructions, by incubating with 0.1-1 mL of each retroviral component diluted in DMEM and centrifugation at 3200 RPM, 32° C. for about two hours. Subsequently, the supernatant was aspirated off of the wells and 0.25-0.5×10⁶ T cells were added in 1 mL of T cell media comprised of: AIM V (Thermo Fisher), 5% fetal bovine serum (FBS), 100 U/mL penicillin (Gibco), 2 mM L-glutamine (Gibco), 100 mg/ml streptomycin (Gibco), 10 mM HEPES (Gibco), and 100 U/mL rhIL-2 (Peprotech). After addition of the T cells, the plates were gently spun down at 1200 RPM for 2 min then incubated for 24 hrs at 37° C. 5% CO2. This transduction process was repeated at Day 3. For some experiments with greater than 3 expression constructs, this process was repeated on Day 4. Dynabeads were removed on Day 4 by magnetic separation. Cells were maintained between 0.4-2×10⁶ cells/mL and expanded until Day 7-21. T cells were transduced with 1-5 different viruses per transduction day.

### I. CRISPR and AAV6 Based Site Specific Knock-In

CRISPR guides were synthesized by Synthego and resuspended according to the manufacturer's instructions. Alt-R® S.p. Cas9 Nuclease V3 was purchased from IDT. To generate Cas9 ribonucleoproteins, 0.5 uL of sgRNA was added to 0.4 uL of Cas9, allowed to complex at room temperature, then placed on ice until electroporation. Primary human T cells were thawed at Day 0 and activated with anti-CD3/CD28 Human T-Expander Dynabeads (Thermo Fisher) at a bead to cell ratio of 3:1. On Day 2, beads were removed from T cells by magnetic separation. 1×106 T cells were resuspended in 20 uL P3 buffer (Lonza), added to cas9 ribonucleoprotein complex, transferred to electroporation strips, then electroporated using the Lonza nucleofector 4D system using program EH-115. Immediately after electroporation, cells were transferred to 96 well plates containing T cell culture media and AAV6 viral particles.

### J. Antibodies and Flow Cytometry

Recombinant CD19 idiotype antibody, HER2-Fc, and CD22-Fc, fluorescently labeled with the DyLight 650 Microscale Antibody Labeling Kit (Thermo Fisher), were used for CAR detection. The following antibodies were used for staining: anti-human EGFR Antibody (clone AY13, BioLegend), Myc-Tag (71D10) Rabbit mAb (Cell Signaling Technology), anti-human CD271 (NGFR) Antibody (clone ME20.4, BioLegend), CD34 Monoclonal Antibody (QBEND/10, Thermo Fisher Scientific), DYKDDDDK (SEQ ID NO:131) Tag (9A3) Mouse mAb (Cell Signaling Technology), anti-HA. 11 Epitope Tag Antibody (BioLegend), EGF Receptor Antibody anti-human Biotin (Miltenyi Biotec), and c-myc Antibody Biotin (Miltenyi Biotec). Flow cytometry was performed on a BD Fortessa instrument and analyzed by FlowJo software (Tree Star).

### K. Magnetic Activated Cell Sorting (MACS)

Cell were stained with the indicated biotinylated antibody according to the manufacturer's instructions. Subsequently, cells were labeled with magnetic microbeads (Streptavidin MicroBeads or Anti-Biotin MicroBeads UltraPure, Miltenyi Biotec) according to the manufacturer's instruction. Cells were loaded onto LS columns, washed with MACS buffer, and magnetically separated using the QuadroMACS separator (Miltenyi Biotec) according to the manufacturer's instructions.

### L. Puromycin Selection

Primary human T cells transduced with the indicated expression constructs were grown to Day 7-Day 10 post activation, as described above, then cultured in T cell media containing the indicated concentration or puromycin dihydrochloride (Thermo Fisher Scientific) for 48-96 hours.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method of selecting for cells that comprise two or more separate expression constructs, the method comprising:
contacting a population of cells with two or more separate expression constructs under conditions in which the two or more expression constructs are delivered to cells of the population of cells, wherein the two or more separate expression constructs comprise:
a first expression construct that encodes a fusion protein comprising a selection marker, a protein localization tag, and a protease cleavage site disposed between the selection marker and the protein localization tag; and
a second expression construct that encodes a protein required for cell surface expression of the selection marker; and
selecting for cells exhibiting cell surface expression of the selection marker.

2. The method according to claim 1, wherein the first expression construct further encodes a protein of interest.

3. The method according to claim 1 or claim 2, wherein the first expression construct site-specifically integrates into the genome of the cell.

4. The method according to claim 3, wherein site-specific integration of the first expression construct into the genome of the cell inactivates a target gene within the genome of the cell.

5. The method according to any one of claims 1 to 4, wherein the second expression construct further encodes a protein of interest.

6. The method according to any one of claims 1 to 5, wherein the second expression construct site-specifically integrates into the genome of the cell.

7. The method according to claim 6, wherein site-specific integration of the second expression construct into the genome of the cell inactivates a target gene within the genome of the cell.

8. The method according to any one of claims 1 to 7, wherein the protein localization tag is selected from the group consisting of: an endoplasmic reticulum (ER) localization tag, a Golgi apparatus (Golgi) localization tag, a lysosome localization tag, a plasma membrane localization tag, a mitochondria localization tag, a peroxisome localization tag, a cytosolic localization tag, and a nuclear localization tag.

9. The method according to any one of claims 1 to 7, wherein the protein localization tag is an ER localization tag.

10. The method according to claim 9, wherein the ER localization tag comprises the amino acid sequence KKMP.

11. The method according to claim 9, wherein the ER localization tag comprises 85% or greater, 90% or greater, or 100% amino acid sequence identity to an ER localization tag comprising, consisting of, or present within, an amino acid sequence selected from the group consisting of: LYKYKSRRSFIDEKKMP (SEQ ID NO:1); AEKDEL (SEQ ID NO:2); EQKLISEEDLKDEL (SEQ ID NO:3); GGGGSGGGGSKDEL (SEQ ID NO:4); GGGGSGGGGSGGGGSGGGGSKDEL (SEQ ID NO:5); GGGGSGGGGSGGGGSGGGGSAEKDEL (SEQ ID NO:6); KYKSRRSFIEEKKMP (SEQ ID NO: 7); LKYKSRRSFIEEKKMP (SEQ ID NO:8); LYKYKSRRSFIEEKKMP (SEQ ID NO:9); LYCKYKSRRSFIEEKKMP (SEQ ID NO:10); LYCNKYKSRRSFIEEKKMP (SEQ ID NO:11); LYCNKYKSRRSFIDEKKMP (SEQ ID NO:12); LYEQKLISEEDLKYKSRRSFIEEKKMP (SEQ ID NO: 13); LYCYPYDVPDYAKYKSRRSFIEEKKMP (SEQ ID NO:14); LYKKLETFKKTN (SEQ ID NO: 15); LYEQKLISEEDLKKLETFKKTN (SEQ ID NO:16); LYYQRL (SEQ ID NO:17); LYEQKLISEEDLYQRL (SEQ ID NO:18); LYKRKIIAFALEGKRSKVTRRPKASDYQRL (SEQ ID NO: 19); LYRNIKCD (SEQ ID NO:20); and LYEQKLISEEDLRNIKCD (SEQ ID NO:21).

12. The method according to claim 9, wherein the ER localization tag comprises 85% or greater, 90% or greater, 95% or greater, or 100% amino acid sequence identity to an ER localization tag comprising, consisting of, or present within, an amino acid sequence selected from the group consisting of:
(SEQ ID NO: 22)
PKKKQQKDSLINLKIQKENPKVVNEINIEDLCLTKAAYCRCWRSKTFPA
CDGSHNKHNELTGDNVGPLILKKKEV;
(SEQ ID NO: 23)
QMRHLKSFFEAKKLV;
(SEQ ID NO: 24)
AYRQRQHQDMPAPRPPGPRPAPPQQEGPPEQQPPQ;
(SEQ ID NO: 25)
HMKEKEKSD;
(SEQ ID NO: 26)
CFRKLAKTGKKKKRD;
(SEQ ID NO: 27)
KCCAYGYRKCLGKKGRVKKAHKSKTH;
(SEQ ID NO: 28)
YLSTCKDSKKKAE;
(SEQ ID NO: 29)
RLTTDVDPDLDQDED;
(SEQ ID NO: 30)
KYKSRRSFIDEKKMP;
(SEQ ID NO: 31)
MTGCCGCCCGCFGIIPLMSKCGKKSSYYTTFDNDVVIEQYRPKKSV;
(SEQ ID NO: 32)
NRSPRNRKPRRE;
(SEQ ID NO: 9)
LYKYKSRRSFIEEKKMP;
(SEQ ID NO: 33)
TKVLKGKKLSLPA;
(SEQ ID NO: 34)
KSNRHKDGFHRLRGHHDEYEDEIRMMSTGSKKSLLSHEFQDETDTEETL
YSSKH;
and
(SEQ ID NO: 35)
KCGKKSSYYTTFDNDVVIEQYRPKKSV.

13. The method according to claim 11 or claim 12, wherein the C-terminus of the ER localization tag comprises the four C-terminal residues of one of the sequences recited in claim 11 or claim 12.

14. The method according to claim 9, wherein the ER localization tag comprises a transmembrane (Tm) domain, an intracellular domain (ICD), or both, of an ER localization tag of a polypeptide set forth in Table 1, or a variant Tm and/or ICD thereof which retains the ability to localize a polypeptide to the ER.

15. The method according to claim 9, wherein the ER localization tag comprises a Tm domain, an ICD, or both, of an ER localization tag of a human ER-resident protein, or a variant Tm and/or ICD thereof which retains the ability to localize a polypeptide to the ER.

16. The method according to claim 15, wherein the human ER-resident protein is CDGSH iron sulfur domain 2 (CISD2).

17. The method according to claim 16, wherein the ER localization tag comprises the Tm domain, the ICD, or both, of the polypeptide set forth in SEQ ID NO:91, or a variant Tm and/or ICD thereof which retains the ability to localize a polypeptide to the ER.

18. The method according to claim 15, wherein the human ER-resident protein is UDP glucuronosyltransferase family 2 member B17 (UGT2B17).

19. The method according to claim 18, wherein the ER localization tag comprises the Tm domain, the ICD, or both, of the polypeptide set forth in SEQ ID NO:95, or a variant Tm and/or ICD thereof which retains the ability to localize a polypeptide to the ER.

20. The method according to any one of claims 1 to 7, wherein the protein localization tag is a Golgi localization tag.

21. The method according to claim 20, wherein the wherein the Golgi localization tag comprises the amino acid sequence YQRL (SEQ ID NO:36).

22. The method according to any one of claims 1 to 7, wherein the protein localization tag is a lysosome localization tag.

23. The method according to claim 22, wherein the lysosome localization tag comprises the amino acid sequence KFERQ (SEQ ID NO:37).

24. The method according to any one of claims 1 to 23, wherein the protease cleavage site is a viral protease cleavage site.

25. The method according to claim 24, wherein the viral protease cleavage site is a cleavage site for a potyviral family protease.

26. The method according to claim 25, wherein the potyviral family protease is Tobacco Etch Virus (TEV) protease, plum pox virus protease (PPVp), soybean mosaic virus protease (SbMVp), sunflower mild mosaic virus protease (SuMMVp), tobacco vein mottling virus protease (TVMVp), or West Nile virus protease (WNVp).

27. The method according to claim 25, wherein the viral protease cleavage site is a TEV protease cleavage site.

28. The method according to claim 24, wherein the viral protease cleavage site is for a viral protease derived from hepatitis C virus (HCV) nonstructural protein 3 (NS3).

29. The method according to claim 28, wherein the viral protease cleavage site is for a viral protease that further comprises a cofactor polypeptide derived from HCV nonstructural protein 4A (NS4A).

30. The method according to claim 28 or claim 29, wherein the viral protease cleavage site is selected from the group consisting of: an NS4A/4B junction cleavage site, an NS3/NS4A junction cleavage site, an NS4A/NS4B junction cleavage site, an NS4B/NS5A junction cleavage site, an NS5A/NS5B junction cleavage site, and variants thereof cleavable by the viral protease.

31. The method according to any one of claims 1 to 23, wherein the protease cleavage site is a human protease cleavage site.

32. The method according to claim 31, wherein the human protease cleavage site is a cleavage site for a human protease selected from the group consisting of: a human kallikrein (KLK) protease, human enterokinase protease, human thrombin, a human matrix metalloprotease (MMP), human urokinase-type plasminogen activator receptor (uPAR), human plasmin, and human cathepsin.

33. The method according to claim 32, wherein the human kallikrein protease is selected from the group consisting of: human KLK3, human KLK4, human KLK6, human KLK8, human KLK11, human KLK13, human KLK14, and human KLK15.

34. The method according to any one of claims 1 to 33, wherein the protein required for cell surface expression of the selection marker is a protease, wherein the protease cleavage site is a cleavage site for the protease.

35. The method according to claim 34, wherein the protease is fused to a protein localization tag that localizes the protease to the same cellular compartment as the fusion protein comprising the selection marker.

36. The method according to claim 35, wherein the protease is fused to a protein localization tag having the same amino acid sequence as that of the protein localization tag of the fusion protein comprising the selection marker.

37. The method according to claim 35 or claim 36, wherein the protease is fused to a membrane association domain.

38. The method according to claim 37, wherein the membrane association domain is a transmembrane domain.

39. The method according to claim 38, wherein the transmembrane domain is a CD8α transmembrane domain.

40. The method according to claim 38, wherein the transmembrane domain is a CD28 transmembrane domain.

41. The method according to claim 38, wherein the transmembrane domain comprises 80% or greater, 85% or greater, 90% or greater, 95% or greater, or 100% amino acid sequence identity to a transmembrane domain comprising, consisting of, or present within, an amino acid sequence selected from the group consisting of:
(SEQ ID NO: 42)
WLRLLPFLGVLALLGYLAVRPFL;
(SEQ ID NO: 43)
VLWWSIAQTVILILTGIW;
(SEQ ID NO: 44)
LGPEWDLYLMTIIALLLGTVI;
(SEQ ID NO: 45)
YYASAFSMMLGLFIFSIVFL;
(SEQ ID NO: 46)
IAFLLACVATMIFMITKCCLF;
(SEQ ID NO: 47)
VIGFLLAVVLTVAFITF;
(SEQ ID NO: 48)
GLFLSAFLLLGLFKALGWAAV;
(SEQ ID NO: 49)
VGLVLAAILALLLAFYAFFYL;
(SEQ ID NO: 50)
TFCSTALLITALALVCTLLYL;
(SEQ ID NO: 51)
WYVWLAIFFAIIIFILILGWVLL;
(SEQ ID NO: 52)
WLWVVYILT VALPVFLVILFC;
(SEQ ID NO: 53)
IYIWAPLAGTCGVLLLSLVITLYC;
and
(SEQ ID NO: 54)
FWVLVVVGGVLACYSLLVTVAFIIFWV.

42. The method according to claim 38 or claim 41, wherein the protease is fused to a hinge domain.

43. The method according to claim 42, wherein the hinge domain is a CD8α hinge domain.

44. The method according to claim 34, wherein the protease is fused to a dimerization domain.

45. The method according to claim 44, wherein the method comprises contacting the population of cells with a third expression construct that encodes a fusion protein comprising a membrane association domain, a dimerization domain that dimerizes with the dimerization domain fused to the protease, and a protein localization tag that localizes the dimerization domain to the same cellular compartment as the fusion protein comprising the selection marker.

46. The method according to claim 45, wherein the third expression construct further encodes a protein of interest.

47. The method according to claim 45 or claim 46, wherein the first expression construct site-specifically integrates into the genome of the cell.

48. The method according to claim 47, wherein site-specific integration of the first expression construct into the genome of the cell inactivates a target gene within the genome of the cell.

49. The method according to any one of claims 1 to 33, wherein the protein required for cell surface expression of the selection marker is a first complementary fragment of a protease, wherein the protease cleavage site is a cleavage site for the protease.

50. The method according to claim 49, wherein the two or more expression constructs comprise a third expression construct that encodes a second complementary fragment of the protease, wherein the first and second complementary fragments form an active protease complex.

51. The method according to claim 50, wherein the third expression construct further encodes a protein of interest.

52. The method according to claim 50 or claim 51, wherein the first expression construct site-specifically integrates into the genome of the cell.

53. The method according to claim 52, wherein site-specific integration of the first expression construct into the genome of the cell inactivates a target gene within the genome of the cell.

54. The method according to any one of claims 50 to 53, wherein the first and second complementary fragments are each fused to a protein localization tag that localizes the first and second complementary fragments to the same cellular compartment as the fusion protein comprising the selection marker.

55. The method according to claim 54, wherein the first and second complementary fragments are each fused to a protein localization tag having the same amino acid sequence as that of the protein localization tag of the fusion protein comprising the selection marker.

56. The method according to any one of claims 50 to 55, wherein the first and second complementary fragments are each fused to a membrane association domain.

57. The method according to claim 56, wherein the membrane association domain is transmembrane domain.

58. The method according to claim 57, wherein the transmembrane domain is as defined in any one of claims 39 to 41.

59. The method according to claim 50, wherein the first and second complementary fragments are each fused to a dimerization domain.

60. The method according to claim 59, wherein the two or more expression constructs comprise:
a fourth expression construct that encodes a fusion protein comprising a membrane association domain, a dimerization domain that dimerizes with the dimerization domain fused to the first complementary fragment, and a protein localization tag that localizes the dimerization domain to the same cellular compartment as the fusion protein comprising the selection marker; and
a fifth expression construct that encodes a fusion protein comprising a membrane association domain, a dimerization domain that dimerizes with the dimerization domain fused to the second complementary fragment, and a protein localization tag that localizes the dimerization domain to the same cellular compartment as the fusion protein comprising the selection marker.

61. The method according to claim 60, wherein the fourth expression construct further encodes a protein of interest.

62. The method according to claim 60 or claim 61, wherein the fourth expression construct site-specifically integrates into the genome of the cell.

63. The method according to claim 62, wherein site-specific integration of the fourth expression construct into the genome of the cell inactivates a target gene within the genome of the cell.

64. The method according to any one of claims 60 to 63, wherein the fifth expression construct further encodes a protein of interest.

65. The method according to any one of claims 60 to 64, wherein the fifth expression construct site-specifically integrates into the genome of the cell.

66. The method according to claim 65, wherein site-specific integration of the fourth expression construct into the genome of the cell inactivates a target gene within the genome of the cell.

67. The method according to any one of claims 60 to 66, wherein the membrane association domain of the fusion protein encoded by each of the fourth and fifth expression constructs is, independently, a transmembrane domain as defined in any one of claims 39 to 41.

68. The method according to any one of claims 44, 45, or 59 to 67, wherein the dimerization domain comprises a coiled coil structure.

69. The method according to claim 68, wherein the dimerization domain comprises a leucine zipper domain.

70. The method according to any one of claims 2 to 69, wherein a protein of interest further encoded by one or more expression constructs of the two or more separate expression constructs is independently selected from the group consisting of: a receptor, a ligand, a transcription factor, an antibody, a bispecific T-cell engager (BiTE), an enzyme, a cytokine, a chemokine, a toxin, a protein conferring resistance to cell exhaustion, and a suicide switch protein.

71. The method according to claim 70, wherein a protein of interest further encoded by one or more expression constructs of the two or more separate expression constructs is a receptor.

72. The method according to claim 71, wherein the receptor is a chimeric antigen receptor (CAR), a T cell receptor (TCR), a synthetic Notch (SynNotch) receptor, a Modular Extracellular Sensor Architecture (MESA) receptor, a Tango receptor, a ChaCha receptor, a generalized extracellular molecule sensor (GEMS) receptor, a cytokine receptor, a chemokine receptor, a switch receptor, an adhesion molecule, an integrin, an inhibitory receptor, a stimulatory receptor, an immunoreceptor tyrosine-based activation motif (ITAM)-containing receptor, or an immunoreceptor tyrosine-based inhibition motif (ITIM)-containing receptor.

73. The method according to claim 72, wherein the receptor is a CAR.

74. The method according to any one of claims 1 to 73, wherein the selection marker comprises a protein tag.

75. The method according to claim 74, wherein the protein tag is selected from the group consisting of: a Myc-tag, a His-tag, an HA-tag, a FLAG-tag, a Strep-tag, an NE-tag, an Xpress tag, an Avi-tag, a polyglutamate tag, and a polyarginine tag.

76. The method according to any one of claims 1 to 75, wherein the selection marker comprises a cluster of differentiation (CD) protein.

77. The method according to claim 76, wherein the CD protein is CD34.

78. The method according to any one of claims 1 to 75, wherein the selection marker comprises a truncated receptor comprising the extracellular domain of the receptor.

79. The method according to claim 78, wherein the truncated receptor is truncated epidermal growth factor receptor (EGFRt), a truncated nerve growth factor receptor (NGFRt), a truncated CD19 (CD19t), or a truncated CD20 (CD20t).

80. The method according to any one of claims 1 to 79, wherein the selection marker is fused to a membrane association domain.

81. The method according to claim 80, wherein the membrane association domain is a transmembrane domain as defined in any one of claims 39 to 41.

82. The method according to any one of claims 1 to 81, wherein the fusion protein encoded by the first expression construct further comprises a degron, wherein the protease cleavage site disposed between the selection marker and the degron.

83. The method according to any one of claims 1 to 82, wherein the fusion protein encoded by the first expression construct further comprises a domain that confers antibiotic resistance.

84. The method according to claim 83, wherein the domain that confers antibiotic resistance is disposed between the selection marker and the protease cleavage site.

85. The method according to claim 83 or claim 84, wherein the domain that confers antibiotic resistance confers puromycin resistance.

86. The method according to claim 85, wherein the domain that confers puromycin resistance comprises a puromycin-N-acetyltransferase (PuroR).

87. The method according to any one of claims 1 to 86, wherein the selecting comprises magnetic-activated cell sorting (MACS).

88. The method according to any one of claims 1 to 86, wherein the selecting comprises flow cytometry.

89. The method according to claim 88, wherein the flow cytometry comprises fluorescence-activated cell sorting (FACS).

90. The method according to any one of claims 1 to 89, wherein the population of cells is a population of mammalian cells.

91. The method according to claim 90, wherein the mammalian cells comprise immune cells.

92. The method according to claim 91, wherein the immune cells comprise T cells, B cells, natural killer (NK) cells, macrophages, monocytes, neutrophils, dendritic cells, mast cells, basophils, eosinophils, and any combination thereof.

93. The method according to claim 91, wherein the immune cells comprise T cells.

94. The method according to claim 93, wherein the T cells comprise naive T cells (T_{N}), cytotoxic T cells (T_{CTL}), memory T cells (T_{MEM}), T memory stem cells (T_{SCM}), central memory T cells (T_{CM}), effector memory T cells (T_{EM}), tissue resident memory T cells (T_{RM}), effector T cells (T_{EFF}), regulatory T cells (T_{REGs}), helper T cells, CD4+ T cells, CD8+ T cells, virus-specific T cells, alpha beta T cells (T_{αβ}), gamma delta T cells (T_{γδ}), and any combination thereof.

95. The method according to claim 90, wherein the mammalian cells comprise stem cells.

96. The method according to claim 95, wherein the stem cells comprise embryonic stem (ES) cells, adult stem cells, hematopoietic stem cells (HSCs), induced pluripotent stem cells (iPSCs), mesenchymal stem cells (MSCs), neural stem cells (NSCs), or any combination thereof.

97. A cell comprising two or more separate expression constructs, wherein the two or more separate expression constructs comprise:
a first expression construct that encodes a fusion protein comprising a selection marker, a protein localization tag, and a protease cleavage site disposed between the selection marker and the protein localization tag; and
a second expression construct that encodes a protein required for cell surface expression of the selection marker.

98. The cell of claim 97, wherein the first expression construct further encodes a protein of interest.

99. The cell of claim 97 or claim 98, wherein the first expression construct is site-specifically integrated into the genome of the cell.

100. The cell of claim 99, wherein a target gene within the genome of the cell is inactivated as a result of the site-specific integration of the first expression construct.

101. The cell of any one of claims 97 to 100, wherein the second expression construct further encodes a protein of interest.

102. The cell of any one of claims 97 to 101, wherein the second expression construct is site-specifically integrated into the genome of the cell.

103. The cell of claim 102, wherein a target gene within the genome of the cell is inactivated as a result of the site-specific integration of the second expression construct.

104. The cell of any one of claims 97 to 103, wherein the cell is a mammalian cell.

105. The cell of claim 104, wherein the mammalian cell is a human cell.

106. The cell of claim 104 or claim 105, wherein the cell is an immune cell.

107. The cell of claim 106, wherein the immune cell is a T cell, a B cell, a natural killer (NK) cell, a macrophage, a monocyte, a neutrophil, a dendritic cell, a mast cell, a basophil, or an eosinophil.

108. The cell of claim 106, wherein the immune cell is a T cell.

109. The cell of claim 108, wherein the T cell is a naive T cell (T_{N}), a cytotoxic T cell (T_{CTL}), a memory T cell (T_{MEM}), a T memory stem cell (TSCM), a central memory T cell (T_{CM}), an effector memory T cell (T_{EM}), a tissue resident memory T cell (T_{RM}), an effector T cell (T_{EFF}), a regulatory T cell (T_{REGs}), a helper T cell, a CD4+ T cell, a CD8+ T cell, a virus-specific T cell, an alpha beta T cell (T_{αβ}), or a gamma delta T cell (T_{γδ}).

110. The cell of claim 104 or claim 105, wherein the cell is a stem cell.

111. The cell of claim 110, wherein the stem cell is an embryonic stem (ES) cell, an adult stem cell, a hematopoietic stem cell (HSC), an induced pluripotent stem cell (iPSC), a mesenchymal stem cell (MSC), or a neural stem cell (NSC).

112. A kit comprising two or more separate expression constructs, wherein the two or more separate expression constructs comprise:
a first expression construct that encodes a fusion protein comprising a selection marker, a protein localization tag, and a protease cleavage site disposed between the selection marker and the protein localization tag; and
a second expression construct that encodes a protein required for cell surface expression of the selection marker.

113. The kit of claim 112, wherein the first expression construct further encodes a protein of interest.

114. The kit of claim 112, wherein the first expression construct comprises a cloning site for a nucleic acid encoding a protein of interest.

115. The kit of any one of claims 112 to 114, wherein the second expression construct further encodes a protein of interest.

116. The kit of any one of claims 112 to 114, wherein the second expression construct comprises a cloning site for a nucleic acid encoding a protein of interest.

117. The kit of any one of claims 112 to 116, further comprising instructions for contacting a population of cells with the two or more expression constructs under conditions in which the two or more expression constructs are delivered to cells of the population of cells.

118. The kit of any one of claims 112 to 117, further comprising instructions for selecting for cells exhibiting cell surface expression of the selection marker.

119. The cell of any one of claims 97 to 118, wherein the protein localization tag is as defined in any one of claims 8 to 23.

120. The cell or kit of any one of claims 97 to 119, wherein the protease cleavage site is as defined in any one of claims 24 to 33.

121. The cell or kit of any one of claims 97 to 120, wherein the protein required for cell surface expression of the selection marker is a protease, wherein the protease cleavage site is a cleavage site for the protease.

122. The cell or kit of claim 121, wherein the protease is fused to a protein localization tag that localizes the protease to the same cellular compartment as the fusion protein comprising the selection marker.

123. The cell or kit of claim 122, wherein the protease is fused to a protein localization tag having the same amino acid sequence as that of the protein localization tag of the fusion protein comprising the selection marker.

124. The cell or kit of claim 122 or claim 123, wherein the protease is fused to a membrane association domain.

125. The cell or kit of claim 124, wherein the membrane association domain is a transmembrane domain as defined in any one of claims 39 to 41.

126. The cell or kit of claim 121, wherein the protease is fused to a dimerization domain.

127. The cell or kit of claim 126, comprising a third expression construct that encodes a fusion protein comprising a transmembrane domain, a dimerization domain that dimerizes with the dimerization domain fused to the protease, and a protein localization tag that localizes the dimerization domain to the same cellular compartment as the fusion protein comprising the selection marker.

128. The cell or kit of any one of claims 97 to 120, wherein the protein required for cell surface expression of the selection marker is a first complementary fragment of a protease, wherein the protease cleavage site is a cleavage site for the protease.

129. The cell or kit of claim 128, comprising a third expression construct that encodes a second complementary fragment of the protease, wherein the first and second complementary fragments form an active protease complex.

130. The cell or kit of claim 129, wherein the third expression construct further encodes a protein of interest.

131. The cell or kit of claim 129 or claim 130, wherein the first and second complementary fragments are each fused to a protein localization tag that localizes the protease to the same cellular compartment as the fusion protein comprising the selection marker.

132. The cell or kit of claim 131, wherein the first and second complementary fragments are each fused to a protein localization tag having the same amino acid sequence as that of the protein localization tag of the fusion protein comprising the selection marker.

133. The cell or kit of any one of claims 129 to 132, wherein the first and second complementary fragments are each fused to a membrane association domain.

134. The cell or kit of claim 133, wherein the membrane association domain is a transmembrane domain.

135. The cell or kit of claim 134, wherein the transmembrane domain is as defined in any one of claims 39 to 41.

136. The cell or kit of any one of claims 129 to 132, wherein the first and second complementary fragments are each fused to a dimerization domain.

137. The cell or kit of claim 136, comprising:
a fourth expression construct that encodes a fusion protein comprising a membrane association domain, a dimerization domain that dimerizes with the dimerization domain fused to the first complementary fragment, and a protein localization tag that localizes the dimerization domain to the same cellular compartment as the fusion protein comprising the selection marker; and
a fifth expression construct that encodes a fusion protein comprising a membrane association domain, a dimerization domain that dimerizes with the dimerization domain fused to the second complementary fragment, and a protein localization tag that localizes the dimerization domain to the same cellular compartment as the fusion protein comprising the selection marker.

138. The cell or kit of claim 137, wherein the fourth expression construct further encodes a protein of interest.

139. The cell or kit of claim 137 or claim 138, wherein the fifth expression construct further encodes a protein of interest.

140. The cell or kit of any one of claims 137 to 139, wherein the membrane association domain of the fusion protein encoded by each of the fourth and fifth expression constructs is, independently, a transmembrane domain as defined in any one of claims 39 to 41.

141. The cell or kit of claims 126, 127, or 136 to 140, wherein the dimerization domain comprises a coiled coil structure.

142. The cell or kit of claim 141, wherein the dimerization domain comprises a leucine zipper domain.

143. The cell or kit of any one of claims 97 to 142, wherein a protein of interest further encoded by one or more expression constructs of the two or more separate expression constructs is independently selected from the group consisting of: a receptor, a ligand, a transcription factor, an antibody, a bispecific T-cell engager (BiTE), an enzyme, a cytokine, a chemokine, a toxin, a protein conferring resistance to cell exhaustion, and a suicide switch protein.

144. The cell or kit of claim 143, wherein a protein of interest further encoded by one or more expression constructs of the two or more separate expression constructs is a receptor.

145. The cell or kit of claim 144, wherein the receptor is a chimeric antigen receptor (CAR), a T cell receptor (TCR), a synthetic Notch (SynNotch) receptor, a Modular Extracellular Sensor Architecture (MESA) receptor, a Tango receptor, a ChaCha receptor, a generalized extracellular molecule sensor (GEMS) receptor, a cytokine receptor, a chemokine receptor, a switch receptor, an adhesion molecule, an integrin, an inhibitory receptor, a stimulatory receptor, an immunoreceptor tyrosine-based activation motif (ITAM)-containing receptor, or an immunoreceptor tyrosine-based inhibition motif (ITIM)-containing receptor.

146. The cell or kit of claim 144, wherein the receptor is a CAR.

147. The cell or kit of any one of claims 97 to 146, wherein the selection marker is as defined in any one of claims 74 to 81.

148. The cell or kit of any one of claims 97 to 147, wherein the fusion protein encoded by the first expression construct further comprises a degron, wherein the protease cleavage site disposed between the selection marker and the degron.

149. The cell or kit of any one of claims 97 to 148, wherein the fusion protein encoded by the first expression construct further comprises a domain that confers antibiotic resistance.

150. The cell or kit of claim 149, wherein the domain that confers antibiotic resistance is disposed between the selection marker and the protease cleavage site.

151. The cell or kit of claim 149 or claim 150, wherein the domain that confers antibiotic resistance confers puromycin resistance.

152. The cell or kit of claim 151, wherein the domain that confers puromycin resistance comprises a puromycin-N-acetyltransferase (PuroR).

153. A composition comprising cells or progeny thereof selected according to the method of any one of claims 1 to 96 present in a liquid medium.

154. A composition comprising the cell of any one of claims 97 to 111 or 119 to 152 present in a liquid medium.

155. The composition of claim 153 or claim 154, wherein the liquid medium is a cell culture medium.

156. The composition of claim 153 or claim 154, wherein the liquid medium is suitable for administration of the composition to an individual in need thereof.

157. The composition of claim 156 formulated for parenteral administration to the individual.

158. A method comprising administering a therapeutically effective amount of the composition of claim 156 or claim 157 to an individual in need thereof.

159. A fusion protein comprising a protein fused to an ER localization tag, wherein the ER localization tag comprises 80% or greater, 85% or greater, 90% or greater, 95% or greater, or 100% amino acid sequence identity to an ER localization tag comprising, consisting of, or present within, an amino acid sequence selected from the group consisting of:
(SEQ ID NO: 22)
PKKKQQKDSLINLKIQKENPKVVNEINIEDLCLTKAAYCRCWRSKTFPA
CDGSHNKHNELTGDNVGPLILKKKEV;
(SEQ ID NO: 23)
QMRHLKSFFEAKKLV;
(SEQ ID NO: 24)
AYRQRQHQDMPAPRPPGPRPAPPQQEGPPEQQPPQ;
(SEQ ID NO: 25)
HMKEKEKSD;
(SEQ ID NO: 26)
CFRKLAKTGKKKKRD;
(SEQ ID NO: 27)
KCCAYGYRKCLGKKGRVKKAHKSKTH;
(SEQ ID NO: 28)
YLSTCKDSKKKAE;
(SEQ ID NO: 29)
RLTTDVDPDLDQDED;
(SEQ ID NO: 30)
KYKSRRSFIDEKKMP;
(SEQ ID NO: 31)
MTGCCGCCCGCFGIIPLMSKCGKKSSYYTTFDNDVVIEQYRPKKSV;
(SEQ ID NO: 32)
NRSPRNRKPRRE;
(SEQ ID NO: 9)
LYKYKSRRSFIEEKKMP;
(SEQ ID NO: 33)
TKVLKGKKLSLPA;
(SEQ ID NO: 34)
KSNRHKDGFHRLRGHHDEYEDEIRMMSTGSKKSLLSHEFQDETDTEETL
YSSKH;
and
(SEQ ID NO: 35)
KCGKKSSYYTTFDNDVVIEQYRPKKSV.

160. The fusion protein of claim 159, wherein the C-terminus of the ER localization tag comprises the four C-terminal residues of one of the sequences recited in claim 159.

161. A fusion protein comprising a protein fused to an ER localization tag, wherein the ER localization tag comprises a Tm domain, an ICD, or both, of an ER localization tag of a polypeptide set forth in Table 1, or a variant Tm and/or ICD thereof which retains the ability to localize a polypeptide to the ER.

162. A fusion protein comprising a protein fused to an ER localization tag, wherein the ER localization tag comprises a Tm domain, an ICD, or both, of an ER localization tag of a human ER-resident protein, or a variant Tm and/or ICD thereof which retains the ability to localize a polypeptide to the ER.

163. The fusion protein of claim 162, wherein the human ER-resident protein is CISD2.

164. The fusion protein of claim 163, wherein the ER localization tag comprises the Tm domain, the ICD, or both, of the polypeptide set forth in SEQ ID NO:91, or a variant Tm and/or ICD thereof which retains the ability to localize a polypeptide to the ER.

165. The fusion protein of claim 162, wherein the human ER-resident protein is UGT2B17.

166. The fusion protein of claim 165, wherein the ER localization tag comprises the Tm domain, the ICD, or both, of the polypeptide set forth in SEQ ID NO:95, or a variant Tm and/or ICD thereof which retains the ability to localize a polypeptide to the ER.

167. The fusion protein of claim 159, wherein the protein is fused directly to the ER localization tag.

168. The fusion protein of claim 159, wherein the protein is fused indirectly to the ER localization tag.

169. The fusion protein of any one of claims 159 to 168, further comprising a protease cleavage site.

170. The fusion protein of claim 169, wherein the protease cleavage site is disposed between the protein and the ER localization tag.

171. The fusion protein of claim 169 or claim 170, wherein the protease cleavage site is as defined in any one of claims 24 to 33.

172. The fusion protein of any one of claims 159 to 171, further comprising a transmembrane domain.

173. The fusion protein of claim 172, wherein the transmembrane domain is as defined in any one of claims 39 to 41.

174. A fusion protein comprising a protein fused to a transmembrane domain, wherein the transmembrane domain comprises 80% or greater, 85% or greater, 90% or greater, 95% or greater, or 100% amino acid sequence identity to a transmembrane domain comprising, consisting of, or present within, an amino acid sequence selected from the group consisting of:
(SEQ ID NO: 42)
WLRLLPFLGVLALLGYLAVRPFL;
(SEQ ID NO: 43)
VLWWSIAQTVILILTGIW;
(SEQ ID NO: 44)
LGPEWDLYLMTIIALLLGTVI;
(SEQ ID NO: 45)
YYASAFSMMLGLFIFSIVFL;
(SEQ ID NO: 46)
IAFLLACVATMIFMITKCCLF;
(SEQ ID NO: 47)
VIGFLLAVVLTVAFITF;
(SEQ ID NO: 48)
GLFLSAFLLLGLFKALGWAAV;
(SEQ ID NO: 49)
VGLVLAAILALLLAFYAFFYL;
(SEQ ID NO: 50)
TFCSTALLITALALVCTLLYL;
(SEQ ID NO: 51)
WYVWLAIFFAIIIFILILGWVLL;
(SEQ ID NO: 52)
WLWVVYILT VALPVFLVILFC;
(SEQ ID NO: 53)
IYIWAPLAGTCGVLLLSLVITLYC;
and
(SEQ ID NO: 54)
FWVLVVVGGVLACYSLLVTVAFIIFWV.

175. The fusion protein of claim 174, wherein the protein is fused directly to the transmembrane domain.

176. The fusion protein of claim 174, wherein the protein is fused indirectly to the transmembrane domain.

177. The fusion protein of any one of claims 174 to 176, further comprising a protease cleavage site.

178. The fusion protein of claim 177, wherein the protease cleavage site is as defined in any one of claims 24 to 33.

179. The fusion protein of any one of claims 174 to 177, further comprising a protein localization tag.

180. The fusion protein of claim 179, wherein the protein localization tag is as defined in any one of claims 8 to 23.

181. The fusion protein of any one of claims 159 to 180, wherein the protein is a receptor, a ligand, a transcription factor, an antibody, a bispecific T-cell engager (BiTE), an enzyme, a cytokine, a chemokine, a toxin, a protein conferring resistance to cell exhaustion, and a suicide switch protein.

182. The fusion protein of any one of claims 159 to 180, wherein the protein is a receptor selected from the group consisting of: a chimeric antigen receptor (CAR), a T cell receptor (TCR), a synthetic Notch (SynNotch) receptor, a Modular Extracellular Sensor Architecture (MESA) receptor, a Tango receptor, a ChaCha receptor, a generalized extracellular molecule sensor (GEMS) receptor, a cytokine receptor, a chemokine receptor, a switch receptor, an adhesion molecule, an integrin, an inhibitory receptor, a stimulatory receptor, an immunoreceptor tyrosine-based activation motif (ITAM)-containing receptor, and an immunoreceptor tyrosine-based inhibition motif (ITIM)-containing receptor.

183. The fusion protein of claim 182, wherein the receptor is a CAR.

184. The fusion protein of any one of claims 159 to 176, wherein the protein is a selection marker.

185. A nucleic acid that encodes the fusion protein of any one of claims 159 to 184.

186. An expression construct comprising the nucleic acid of claim 185.

187. A cell comprising the nucleic acid of claim 185 or the expression construct of claim 186.

188. A method of producing the fusion protein of any one of claims 159 to 184, comprising culturing the cell of claim 186 or claim 187 under conditions suitable for the cell to express the fusion protein, wherein the fusion protein is produced.


SEQUENCE LISTING (137 sequences)

SEQ ID NO: 1 - protein, 17 aa, Artificial sequence
  synthetic sequence
LYKYKSRRSFIDEKKMP

SEQ ID NO: 2 - protein, 6 aa, Artificial sequence
  synthetic sequence
AEKDEL

SEQ ID NO: 3 - protein, 14 aa, Artificial sequence
  synthetic sequence
EQKLISEEDLKDEL

SEQ ID NO: 4 - protein, 14 aa, Artificial sequence
  synthetic sequence
GGGGSGGGGSKDEL

SEQ ID NO: 5 - protein, 24 aa, Artificial sequence
  synthetic sequence
GGGGSGGGGSGGGGSGGGGSKDEL

SEQ ID NO: 6 - protein, 26 aa, Artificial sequence
  synthetic sequence
GGGGSGGGGSGGGGSGGGGSAEKDEL

SEQ ID NO: 7 - protein, 15 aa, Artificial sequence
  synthetic sequence
KYKSRRSFIEEKKMP

SEQ ID NO: 8 - protein, 16 aa, Artificial sequence
  synthetic sequence
LKYKSRRSFIEEKKMP

SEQ ID NO: 9 - protein, 17 aa, Artificial sequence
  synthetic sequence
LYKYKSRRSFIEEKKMP

SEQ ID NO: 10 - protein, 18 aa, Artificial sequence
  synthetic sequence
LYCKYKSRRSFIEEKKMP

SEQ ID NO: 11 - protein, 19 aa, Artificial sequence
  synthetic sequence
LYCNKYKSRRSFIEEKKMP

SEQ ID NO: 12 - protein, 19 aa, Artificial sequence
  synthetic sequence
LYCNKYKSRRSFIDEKKMP

SEQ ID NO: 13 - protein, 27 aa, Artificial sequence
  synthetic sequence
LYEQKLISEEDLKYKSRRSFIEEKKMP

SEQ ID NO: 14 - protein, 27 aa, Artificial sequence
  synthetic sequence
LYCYPYDVPDYAKYKSRRSFIEEKKMP

SEQ ID NO: 15 - protein, 12 aa, Artificial sequence
  synthetic sequence
LYKKLETFKKTN

SEQ ID NO: 16 - protein, 22 aa, Artificial sequence
  synthetic sequence
LYEQKLISEEDLKKLETFKKTN

SEQ ID NO: 17 - protein, 6 aa, Artificial sequence
  synthetic sequence
LYYQRL

SEQ ID NO: 18 - protein, 16 aa, Artificial sequence
  synthetic sequence
LYEQKLISEEDLYQRL

SEQ ID NO: 19 - protein, 30 aa, Artificial sequence
  synthetic sequence
LYKRKIIAFALEGKRSKVTRRPKASDYQRL

SEQ ID NO: 20 - protein, 8 aa, Artificial sequence
  synthetic sequence
LYRNIKCD

SEQ ID NO: 21 - protein, 18 aa, Artificial sequence
  synthetic sequence
LYEQKLISEEDLRNIKCD

SEQ ID NO: 22 - protein, 75 aa, Artificial sequence
  synthetic sequence
PKKKQQKDSLINLKIQKENPKVVNEINIEDLCLTKAAYCRCWRSKTFPACDGSHNKHNELTGDNVGPLILKKKEV

SEQ ID NO: 23 - protein, 15 aa, Artificial sequence
  synthetic sequence
QMRHLKSFFEAKKLV

SEQ ID NO: 24 - protein, 35 aa, Artificial sequence
  synthetic sequence
AYRQRQHQDMPAPRPPGPRPAPPQQEGPPEQQPPQ

SEQ ID NO: 25 - protein, 9 aa, Artificial sequence
  synthetic sequence
HMKEKEKSD

SEQ ID NO: 26 - protein, 15 aa, Artificial sequence
  synthetic sequence
CFRKLAKTGKKKKRD

SEQ ID NO: 27 - protein, 26 aa, Artificial sequence
  synthetic sequence
KCCAYGYRKCLGKKGRVKKAHKSKTH

SEQ ID NO: 28 - protein, 13 aa, Artificial sequence
  synthetic sequence
YLSTCKDSKKKAE

SEQ ID NO: 29 - protein, 15 aa, Artificial sequence
  synthetic sequence
RLTTDVDPDLDQDED

SEQ ID NO: 30 - protein, 15 aa, Artificial sequence
  synthetic sequence
KYKSRRSFIDEKKMP

SEQ ID NO: 31 - protein, 46 aa, Artificial sequence
  synthetic sequence
MTGCCGCCCGCFGIIPLMSKCGKKSSYYTTFDNDVVIEQYRPKKSV

SEQ ID NO: 32 - protein, 12 aa, Artificial sequence
  synthetic sequence
NRSPRNRKPRRE

SEQ ID NO: 33 - protein, 13 aa, Artificial sequence
  synthetic sequence
TKVLKGKKLSLPA

SEQ ID NO: 34 - protein, 54 aa, Artificial sequence
  synthetic sequence
KSNRHKDGFHRLRGHHDEYEDEIRMMSTGSKKSLLSHEFQDETDTEETLYSSKH

SEQ ID NO: 35 - protein, 27 aa, Artificial sequence
  synthetic sequence
KCGKKSSYYTTFDNDVVIEQYRPKKSV

SEQ ID NO: 36 - protein, 4 aa, Artificial sequence
  synthetic sequence
YQRL

SEQ ID NO: 37 - protein, 5 aa, Artificial sequence
  synthetic sequence
KFERQ

SEQ ID NO: 38 - protein, 236 aa, Tobacco Etch Virus
GESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMN

SEQ ID NO: 39 - protein, 186 aa, Hepatitis C virus
APITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTD

SEQ ID NO: 40 - protein, 186 aa, Hepatitis C virus
APITAYAQQTRGLLGCIITSLTGRDKNQVEGEVQIMSTATQTFLATCINGVCWTVYHGAGTRTIASPKGPVIQMYTNVDQDLVGWPAPQGSRSLTPCTCGSSDLYLVTRHADVIPVRRRGDGRGSLLSPRPISYLKGSSGGPLLCPAGHAVGLFRAAVCTRGVAKAVDFIPVENLETTMRSPVFTD

SEQ ID NO: 41 - protein, 186 aa, Hepatitis C virus
APITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTD

SEQ ID NO: 42 - protein, 23 aa, Artificial sequence
  synthetic sequence
WLRLLPFLGVLALLGYLAVRPFL

SEQ ID NO: 43 - protein, 18 aa, Artificial sequence
  synthetic sequence
VLWWSIAQTVILILTGIW

SEQ ID NO: 44 - protein, 21 aa, Artificial sequence
  synthetic sequence
LGPEWDLYLMTIIALLLGTVI

SEQ ID NO: 45 - protein, 20 aa, Artificial sequence
  synthetic sequence
YYASAFSMMLGLFIFSIVFL

SEQ ID NO: 46 - protein, 21 aa, Artificial sequence
  synthetic sequence
IAFLLACVATMIFMITKCCLF

SEQ ID NO: 47 - protein, 17 aa, Artificial sequence
  synthetic sequence
VIGFLLAVVLTVAFITF

SEQ ID NO: 48 - protein, 21 aa, Artificial sequence
  synthetic sequence
GLFLSAFLLLGLFKALGWAAV

SEQ ID NO: 49 - protein, 21 aa, Artificial sequence
  synthetic sequence
VGLVLAAILALLLAFYAFFYL

SEQ ID NO: 50 - protein, 21 aa, Artificial sequence
  synthetic sequence
TFCSTALLITALALVCTLLYL

SEQ ID NO: 51 - protein, 23 aa, Artificial sequence
  synthetic sequence
WYVWLAIFFAIIIFILILGWVLL

SEQ ID NO: 52 - protein, 21 aa, Artificial sequence
  synthetic sequence
WLWVVYILTVALPVFLVILFC

SEQ ID NO: 53 - protein, 24 aa, Artificial sequence
  synthetic sequence
IYIWAPLAGTCGVLLLSLVITLYC

SEQ ID NO: 54 - protein, 27 aa, Artificial sequence
  synthetic sequence
FWVLVVVGGVLACYSLLVTVAFIIFWV

SEQ ID NO: 55 - protein, 42 aa, Hepatitis C virus
PITKIDTKYIMTCMSADLEVVTSTWVLVGGVLAALAAYCLST

SEQ ID NO: 56 - protein, 674 aa, Artificial sequence
  synthetic sequence
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNNASATNFSLLKQAGDVEENPGPMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSSGNSSGGSTGCVVIVGRIVLSGSGTSAPITAYAQQTRGLLGCIITSLTGRDKNQVEGEVQIMSTATQTFLATCINGVCWAVYHGAGTRTIASPKGPVIQMYTNVDQDLVGWPAPQGSRSLTPCTCGSSDLYLVTRHADVIPVRRRGDGRGSLLSPRPISYLKGSSGGPLLCPAGHAVGLFRAAVCTRGVAKAVDFIPVENLETTMRSPVFTDNSSPPAVTLTHAAASTGSSGGGGGSGGLYKYKSRRSFIDEKKMP

SEQ ID NO: 57 - protein, 713 aa, Artificial sequence
  synthetic sequence
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNNASATNFSLLKQAGDVEENPGPMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSSGNSSGGSGSTGSSGGSGGSGSSGGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNAAASTGSSGGGGGSGGLYKYKSRRSFIDEKKMP

SEQ ID NO: 58 - protein, 595 aa, Artificial sequence
  synthetic sequence
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNNASATNFSLLKQAGDVEENPGPMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSSGNSSGGSGSTGSSGGSGGSGSSGGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTAAASTGSSGGGGGSGGLYKYKSRRSFIDEKKMP

SEQ ID NO: 59 - protein, 839 aa, Artificial sequence
  synthetic sequence
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLGHGTGSTGSGSSGTASSEDNNMAVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLYGMDELYKNAGNSSIGATNFSLLKQAGDVEENPGPMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSSGNSSGGSGSTGSSGGSGGSGSSGGSKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNAAASTGSSGGGGGSGGLYKYKSRRSFIDEKKMP

SEQ ID NO: 60 - protein, 596 aa, Artificial sequence
  synthetic sequence
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNNASATNFSLLKQAGDVEENPGPMGTSLLCWMALCLLGADHADACPYSNPSLCSGGGGSELPTQGTFSNVSTNVSPAKPTTTACPYSNPSLCSGGGGSIEVMYPPPYLDNEKSNGTIIHVKGKHLCPSPLFPGPSKPFWVLVVVGGVLACYSLLVTVAFIIFWVGSSGGSSGSSGGGSSGSGSSGNSSGGSGSTGSSGGSGGSGSSGGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTAAASTGSSGGGGGSGGLYKYKSRRSFIDEKKMP

SEQ ID NO: 61 - protein, 840 aa, Artificial sequence
  synthetic sequence
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLGHGTGSTGSGSSGTASSEDNNMAVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLYGMDELYKNAGNSSIGATNFSLLKQAGDVEENPGPMGTSLLCWMALCLLGADHADACPYSNPSLCSGGGGSELPTQGTFSNVSTNVSPAKPTTTACPYSNPSLCSGGGGSIEVMYPPPYLDNEKSNGTIIHVKGKHLCPSPLFPGPSKPFWVLVVVGGVLACYSLLVTVAFIIFWVGSSGGSSGSSGGGSSGSGSSGNSSGGSGSTGSSGGSGGSGSSGGSKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNAAASTGSSGGGGGSGGLYKYKSRRSFIDEKKMP

SEQ ID NO: 62 - protein, 852 aa, Artificial sequence
  synthetic sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNASATNFSLLKQAGDVEENPGPMGTSLLCWMALCLLGADHADACPYSNPSLCSGGGGSELPTQGTFSNVSTNVSPAKPTTTACPYSNPSLCSGGGGSIEVMYPPPYLDNEKSNGTIIHVKGKHLCPSPLFPGPSKPFWVLVVVGGVLACYSLLVTVAFIIFWVGSSGGSSGSSGGGSSGSGSSGNSSGGSGSTGSSGGSGGSGSSGGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTAAASTGSSGGGGGSGGLYKYKSRRSFIDEKKMP

SEQ ID NO: 63 - protein, 849 aa, Artificial sequence
  synthetic sequence
MARSVTLVFLVLVSLTGLYAADIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIKGSTSGSGKPGSGEGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSIGATNFSLLKQAGDVEENPGPMGTSLLCWMALCLLGADHADACPYSNPSLCSGGGGSELPTQGTFSNVSTNVSPAKPTTTACPYSNPSLCSGGGGSIEVMYPPPYLDNEKSNGTIIHVKGKHLCPSPLFPGPSKPFWVLVVVGGVLACYSLLVTVAFIIFWVGSSGGSSGSSGGGSSGSGSSGNSSGGSGSTGSSGGSGGSGSSGGSKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNAAASTGSSGGGGGSGGLYKYKSRRSFIDEKKMP

SEQ ID NO: 64 - protein, 722 aa, Artificial sequence
  synthetic sequence
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNNASATNFSLLKQAGDVEENPGPSIGMGTSLLCWMALCLLGADHADGNSACPYSNPSLCSGGGGSELPTQGTFSNVSTNVSPAKPTTTACPYSNPSLCSGGGGSGGSWLRLLPFLGVLALLGYLAVRPFLAAAGSSGGSSGGSGSTGSSGGSGGSGSSGGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNGSSGGGGGSGGNASPKKKQQKDSLINLKIQKENPKVVNEINIEDLCLTKAAYCRCWRSKTFPACDGSHNKHNELTGDNVGPLILKKKEV

SEQ ID NO: 65 - protein, 712 aa, Artificial sequence
  synthetic sequence
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNNASATNFSLLKQAGDVEENPGPMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSSGNSSGGSGSTGSSGGSGGSGSSGGSIVGGSNAKEGAWPWVVGLYYGGRLLCGASLVSSDWLVSAAHCVYGRNLEPSKWTAILGLHMKSNLTSPQTVPRLIDEIVINPHYNRRRKDNDIAMMHLEFKVNYTDYIQPICLPEENQVFPPGRNCSIAGWGTVVYQGTTANILQEADVPLLSNERCQQQMPEYNITENMICAGYEEGGIDSCQGDSGGPLMCQENNRWFLAGVTSFGYKCALPNRPGVYARVSRFTEWIQSFLHAAASTGSSGGGGGSGGLYKYKSRRSFIDEKKMP

SEQ ID NO: 66 - protein, 388 aa, Artificial sequence
  synthetic sequence
MLLLVTSLLLCELPHPAFLLIPGGSEQKLISEEDLTTTPAPRPPTPAPTIASQPLSLRPEACRPAAGGAVHTRGLDFACDIYIWAPLAGTCGVLLLSLVITLYCAAAGSVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKSIGGGSGGSDEMEECSQHGSTGGSGGSLYKYKSRRSFIDEKKMP

SEQ ID NO: 67 - protein, 387 aa, Artificial sequence
  synthetic sequence
MLLLVTSLLLCELPHPAFLLIPGGSEQKLISEEDLTTTPAPRPPTPAPTIASQPLSLRPEACRPAAGGAVHTRGLDFACDIYIWAPLAGTCGVLLLSLVITLYCAAAGSVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKSIGGGSGGSTENLYFQSGSTGGSGGSLYKYKSRRSFIDEKKMP

SEQ ID NO: 68 - protein, 557 aa, Artificial sequence
  synthetic sequence
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRAAAGSVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKSIGGGSGGSTENLYFQSGSTGGSGGSLYKYKSRRSFIDEKKMP

SEQ ID NO: 69 - protein, 558 aa, Artificial sequence
  synthetic sequence
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRAAAGSVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKSIGGGSGGSDEMEECSQHGSTGGSGGSLYKYKSRRSFIDEKKMP

SEQ ID NO: 70 - protein, 429 aa, Artificial sequence
  synthetic sequence
MLLLVTSLLLCELPHPAFLLIPGGSACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCAAAGSVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKSIGGGSGGSTENLYFQSGSTGGSGGSLYKYKSRRSFIDEKKMP

SEQ ID NO: 71 - protein, 615 aa, Artificial sequence
  synthetic sequence
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNGGSEQKLISEEDLTTTPAPRPPTPAPTIASQPLSLRPEACRPAAGGAVHTRGLDFACDIYIWAPLAGTCGVLLLSLVITLYCAAAGSVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKSIGGGSGGSTENLYFQSGSTGGSGGSLYKYKSRRSFIDEKKMP

SEQ ID NO: 72 - protein, 384 aa, Artificial sequence
  synthetic sequence
MLLLVTSLLLCELPHPAFLLIPGGSEQKLISEEDLIEVMYPPPYLDNEKSNGTIIHVKGKHLCPSPLFPGPSKPFWVLVVVGGVLACYSLLVTVAFIIFWVAAAGSVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKSIGGGSGGSTENLYFQSGSTGGSGGSLYKYKSRRSFIDEKKMP

SEQ ID NO: 73 - protein, 429 aa, Artificial sequence
  synthetic sequence
MLLLVTSLLLCELPHPAFLLIPGGSACPYSNPSLCSGGGGSELPTQGTFSNVSTNVSPAKPTTTACPYSNPSLCSGGGGSIEVMYPPPYLDNEKSNGTIIHVKGKHLCPSPLFPGPSKPFWVLVVVGGVLACYSLLVTVAFIIFWVAAAGSVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKSIGGGSGGSTENLYFQSGSTGGSGGSLYKYKSRRSFIDEKKMP

SEQ ID NO: 74 - protein, 612 aa, Artificial sequence
  synthetic sequence
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNGGSEQKLISEEDLIEVMYPPPYLDNEKSNGTIIHVKGKHLCPSPLFPGPSKPFWVLVVVGGVLACYSLLVTVAFIIFWVAAAGSVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKSIGGGSGGSTENLYFQSGSTGGSGGSLYKYKSRRSFIDEKKMP

SEQ ID NO: 75 - protein, 196 aa, Artificial sequence
  synthetic sequence
MLLLVTSLLLCELPHPAFLLIPGGSDYKDDDDKGGTTTPAPRPPTPAPTIASQPLSLRPEACRPAAGGAVHTRGLDFACDIYIWAPLAGTCGVLLLSLVITLYCKGGGGSGGAAAGSNLVAQLENEVASLENENETLKKKNLHKKDLIAYLEKEIANLRKKIEEGGSGSSGSTGGSGGSLYKYKSRRSFIDEKKMP

SEQ ID NO: 76 - protein, 192 aa, Artificial sequence
  synthetic sequence
MLLLVTSLLLCELPHPAFLLIPGGSYPYDVPDYAGGTTTPAPRPPTPAPTIASQPLSLRPEACRPAAGGAVHTRGLDFACDIYIWAPLAGTCGVLLLSLVITLYCKGGGGSGGAAAGSNEVTTLENDAAFIENENAYLEKEIARLRKEKAALRNRLAHKKGGSGSSGSTGGSGGSLYKYKSRRSFIDEKKMP

SEQ ID NO: 77 - protein, 451 aa, Artificial sequence
  synthetic sequence
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNNASATNFSLLKQAGDVEENPGPGGSARNAYLRKKIARLKKDNLQLERDEQNLEKIIANLRDEIARLENEVASHEQGSTGSSGGSGGSGSSGGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTAAA

SEQ ID NO: 78 - protein, 699 aa, Artificial sequence
  synthetic sequence
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLGHGTGSTGSGSSGTASSEDNNMAVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLYGMDELYKNAGNSGGGATNFSLLKQAGDVEENPGPGGSQKVAELKNRVAVKLNRNEQLKNKVEELKNRNAYLKNELATLENEVARLENDVAEGSTGSSGGSGGSGSSGGSKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNAAA

SEQ ID NO: 79 - protein, 640 aa, Artificial sequence
  synthetic sequence
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMAAAGSVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKSIGGGSGGSTENLYFQSGSTGGSGGSLYKYKSRRSFIDEKKMP

SEQ ID NO: 80 - protein, 463 aa, Artificial sequence
  synthetic sequence
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSEQKLISEEDLTTTPAPRPPTPAPTIASQPLSLRPEACRPAAGGAVHTRGLDFACDIYIWAPLAGTCGVLLLSLVITLYCAAAGSIGGGSGGSTENLYFQSGSTGGSGGSLYKYKSRRSFIDEKKMP

SEQ ID NO: 81 - protein, 711 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSEQKLISEEDLTTTPAPRPPTPAPTIASQPLSLRPEACRPAAGGAVHTRGLDFACDIYIWAPLAGTCGVLLLSLVITLYCAAAGGTENLYFQSGSTLYKYKSRRSFIEEKKMP

SEQ ID NO: 82 - protein, 711 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSEQKLISEEDLTTTPAPRPPTPAPTIASQPLSLRPEACRPAAGGAVHTRGLDFACDIYIWAPLAGTCGVLLLSLVITLYCAAAGGTENLYFQSGSTLYKYKSRRSFIEEKKYN

SEQ ID NO: 83 - protein, 711 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSEQKLISEEDLTTTPAPRPPTPAPTIASQPLSLRPEACRPAAGGAVHTRGLDFACDIYIWAPLAGTCGVLLLSLVITLYCAAAGGTENLYFQSGSTLYKYKSRRSFIEEKKYL

SEQ ID NO: 84 - protein, 713 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSEQKLISEEDLTTTPAPRPPTPAPTIASQPLSLRPEACRPAAGGAVHTRGLDFACDIYIWAPLAGTCGVLLLSLVITLYCAAAGGTENLYFQSGSTLYKYKSRRSFIEEFLKKTN

SEQ ID NO: 85 - protein, 707 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSEQKLISEEDLTTTPAPRPPTPAPTIASQPLSLRPEACRPAAGGAVHTRGLDFACDIYIWAPLAGTCGVLLLSLVITLYCAAAGGTENLYFQSGSTTKVLKGKKLSLPA

SEQ ID NO: 86 - protein, 748 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSEQKLISEEDLTTTPAPRPPTPAPTIASQPLSLRPEACRPAAGGAVHTRGLDFACDIYIWAPLAGTCGVLLLSLVITLYCAAAGGTENLYFQSGSTKSNRHKDGFHRLRGHHDEYEDEIRMMSTGSKKSLLSHEFQDETDTEETLYSSKH

SEQ ID NO: 87 - protein, 753 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSEQKLISEEDLTTTPAPRPPTPAPTIASQPLSLRPEACRPAAGGAVHTRGLDFACDIYIWAPLAGTCGVLLLSLVITLYCAAAGGTENLYFQSGSTPITKIDTKYIMTCMSADLEVVTSTWVLVGGVLAALAAYCLSTLYKYKSRRSFIEEKKMP

SEQ ID NO: 88 - protein, 721 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSEQKLISEEDLTTTPAPRPPTPAPTIASQPLSLRPEACRPAAGGAVHTRGLDFACDIYIWAPLAGTCGVLLLSLVITLYCAAAGGTENLYFQSGSTKCGKKSSYYTTFDNDVVIEQYRPKKSV

SEQ ID NO: 89 - protein, 731 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSEQKLISEEDLTTTPAPRPPTPAPTIASQPLSLRPEACRPAAGGAVHTRGLDFACDIYIWAPLAGTCGVLLLSLVITLYCAAATRGLLGCIITSLTGRGGGSGGSTENLYFQSGSTLYKYKSRRSFIDEKKMP

SEQ ID NO: 90 - protein, 748 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSEQKLISEEDLTTTPAPRPPTPAPTIASQPLSLRPEACRPAAGGAVHTRGLDFACDIYIWAPLAGTCGVLLLSLVITLYCAAATRGLLGCIITSLTGRGGGSGGSTENLYFQSGSTGGSNSSPPAVTLTHGGSLYKYKSRRSFIDEKKMP

SEQ ID NO: 91 - protein, 713 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSWLRLLPFLGVLALLGYLAVRPFLAAAGGTENLYFQSGSTPKKKQQKDSLINLKIQKENPKVVNEINIEDLCLTKAAYCRCWRSKTFPACDGSHNKHNELTGDNVGPLILKKKEV

SEQ ID NO: 92 - protein, 648 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSVLWWSIAQTVILILTGIWAAAGGTENLYFQSGSTQMRHLKSFFEAKKLV

SEQ ID NO: 93 - protein, 671 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSLGPEWDLYLMTIIALLLGTVIAAAGGTENLYFQSGSTAYRQRQHQDMPAPRPPGPRPAPPQQEGPPEQQPPQ

SEQ ID NO: 94 - protein, 644 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSYYASAFSMMLGLFIFSIVFLAAAGGTENLYFQSGSTHMKEKEKSD

SEQ ID NO: 95 - protein, 651 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSIAFLLACVATMIFMITKCCLFAAAGGTENLYFQSGSTCFRKLAKTGKKKKRD

SEQ ID NO: 96 - protein, 658 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSVIGFLLAVVLTVAFITFAAAGGTENLYFQSGSTKCCAYGYRKCLGKKGRVKKAHKSKTH

SEQ ID NO: 97 - protein, 649 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSGLFLSAFLLLGLFKALGWAAVAAAGGTENLYFQSGSTYLSTCKDSKKKAE

SEQ ID NO: 98 - protein, 651 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSVGLVLAAILALLLAFYAFFYLAAAGGTENLYFQSGSTRLTTDVDPDLDQDED

SEQ ID NO: 99 - protein, 651 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSTFCSTALLITALALVCTLLYLAAAGGTENLYFQSGSTKYKSRRSFIDEKKMP

SEQ ID NO: 100 - protein, 684 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSWYVWLAIFFAIIIFILILGWVLLAAAGGTENLYFQSGSTMTGCCGCCCGCFGIIPLMSKCGKKSSYYTTFDNDVVIEQYRPKKSV

SEQ ID NO: 101 - protein, 648 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSWLWVVYILTVALPVFLVILFCAAAGGTENLYFQSGSTNRSPRNRKPRRE

SEQ ID NO: 102 - protein, 711 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSWLRLLPFLGVLALLGYLAVRPFLAAAGGTDDDDKGSTPKKKQQKDSLINLKIQKENPKVVNEINIEDLCLTKAAYCRCWRSKTFPACDGSHNKHNELTGDNVGPLILKKKEV

SEQ ID NO: 103 - protein, 711 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSWLRLLPFLGVLALLGYLAVRPFLAAAGGTDDDDRGSTPKKKQQKDSLINLKIQKENPKVVNEINIEDLCLTKAAYCRCWRSKTFPACDGSHNKHNELTGDNVGPLILKKKEV

SEQ ID NO: 104 - protein, 740 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSIEVMYPPPYLDNEKSNGTIIHVKGKHLCPSPLFPGPSKPFWVLVVVGGVLACYSLLVTVAFIIFWVAAAGGTENLYFQSGSTPITKIDTKYIMTCMSADLEVVTSTWVLVGGVLAALAAYCLSTLYKYKSRRSFIEEKKMP

SEQ ID NO: 105 - protein, 756 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSIEVMYPPPYLDNEKSNGTIIHVKGKHLCPSPLFPGPSKPFWVLVVVGGVLACYSLLVTVAFIIFWVAAAGGTENLYFQSGSTPKKKQQKDSLINLKIQKENPKVVNEINIEDLCLTKAAYCRCWRSKTFPACDGSHNKHNELTGDNVGPLILKKKEV

SEQ ID NO: 106 - protein, 696 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSIEVMYPPPYLDNEKSNGTIIHVKGKHLCPSPLFPGPSKPFWVLVVVGGVLACYSLLVTVAFIIFWVAAAGGTENLYFQSGSTCFRKLAKTGKKKKRD

SEQ ID NO: 107 - protein, 727 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSIEVMYPPPYLDNEKSNGTIIHVKGKHLCPSPLFPGPSKPFWVLVVVGGVLACYSLLVTVAFIIFWVAAAGGTENLYFQSGSTMTGCCGCCCGCFGIIPLMSKCGKKSSYYTTFDNDVVIEQYRPKKSV

SEQ ID NO: 108 - protein, 960 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSEQKLISEEDLTTTPAPRPPTPAPTIASQPLSLRPEACRPAAGGAVHTRGLDFACDIYIWAPLAGTCGVLLLSLVITLYCAAGAGGSGGTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAAAAGGTENLYFQSGSTPITKIDTKYIMTCMSADLEVVTSTWVLVGGVLAALAAYCLSTLYKYKSRRSFIEEKKMP

SEQ ID NO: 109 - protein, 967 aa, Artificial sequence
  synthetic sequence
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYKNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSEQKLISEEDLTTTPAPRPPTPAPTIASQPLSLRPEACRPAAGGAVHTRGLDFACDIYIWAPLAGTCGVLLLSLVITLYCAAGAGGSGGENLYFQFTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGAAAAGGTENLYFQSGSTPITKIDTKYIMTCMSADLEVVTSTWVLVGGVLAALAAYCLSTLYKYKSRRSFIEEKKMP

SEQ ID NO: 110 - protein, 961 aa, Artificial sequence
  synthetic sequence
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSWLRLLPFLGVLALLGYLAVRPFLAAAGGTENLYFQSGSTPKKKQQKDSLINLKIQKENPKVVNEINIEDLCLTKAAYCRCWRSKTFPACDGSHNKHNELTGDNVGPLILKKKEV

SEQ ID NO: 111 - protein, 932 aa, Artificial sequence
  synthetic sequence
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSWYVWLAIFFAIIIFILILGWVLLAAAGGTENLYFQSGSTMTGCCGCCCGCFGIIPLMSKCGKKSSYYTTFDNDVVIEQYRPKKSV

SEQ ID NO: 112 - protein, 808 aa, Artificial sequence
  synthetic sequence
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTFNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSWLRLLPFLGVLALLGYLAVRPFLAAAGGTENLYFQSGSTPKKKQQKDSLINLKIQKENPKVVNEINIEDLCLTKAAYCRCWRSKTFPACDGSHNKHNELTGDNVGPLILKKKEV

SEQ ID NO: 113 - protein, 779 aa, Artificial sequence
  synthetic sequence
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTFNASGATNFSLLKQAGDVEENPGPIASMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSGGSWYVWLAIFFAIIIFILILGWVLLAAAGGTENLYFQSGSTMTGCCGCCCGCFGIIPLMSKCGKKSSYYTTFDNDVVIEQYRPKKSV

SEQ ID NO: 114 - protein, 575 aa, Artificial sequence
  synthetic sequence
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNNASATNFSLLKQAGDVEENPGPMGTSLLCWMALCLLGADHADGGSIAFLLACVATMIFMITKCCLFGNSGSSGGSGGSGSSGGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNGSTCFRKLAKTGKKKKRD

SEQ ID NO: 115 - protein, 714 aa, Artificial sequence
  synthetic sequence
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNNASATNFSLLKQAGDVEENPGPMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSSGNSSGGSGSTGSSGGSGGSGSSGGSIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANPAAASTGSSGGGGGSGGLYKYKSRRSFIDEKKMP

SEQ ID NO: 116 - protein, 701 aa, Artificial sequence
  synthetic sequence
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNNASATNFSLLKQAGDVEENPGPMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSSGNSSGGSGSTGSSGGSGGSGSSGGSIINGEDCSPHSQPWQAALVMENELFCSGVLVHPQWVLSAAHCFQNSYTIGLGLHSLEADQEPGSQMVEASLSVRHPEYNRPLLANDLMLIKLDESVSESDTIRSISIASQCPTAGNSCLVSGWGLLANGRMPTVLQCVNVSVVSEEVCSKLYDPLYHPSMFCAGGGHDQKDSCNGDSGGPLICNGYLQGLVSFGKAPCGQVGVPGVYTNLCKFTEWIEKTVQASAAASTGSSGGGGGSGGLYKYKSRRSFIDEKKMP

SEQ ID NO: 117 - protein, 700 aa, Artificial sequence
  synthetic sequence
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNNASATNFSLLKQAGDVEENPGPMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSSGNSSGGSGSTGSSGGSGGSGSSGGSLVHGGPCDKTSHPYQAALYTSGHLLCGGVLIHPLWVLTAAHCKKPNLQVFLGKHNLRQRESSQEQSSVVRAVIHPDYDAASHDQDIMLLRLARPAKLSELIQPLPLERDCSANTTSCHILGWGKTADGDFPDTIQCAYIHLVSREECEHAYPGQITQNMLCAGDEKYGKDSCQGDSGGPLVCGDHLRGLVSWGNIPCGSKEKPGVYTNVCRYTNWIQKTIQAKAAASTGSSGGGGGSGGLYKYKSRRSFIDEKKMP

SEQ ID NO: 118 - protein, 705 aa, Artificial sequence
  synthetic sequence
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNNASATNFSLLKQAGDVEENPGPMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSSGNSSGGSGSTGSSGGSGGSGSSGGSVLGGHECQPHSQPWQAALFQGQQLLCGGVLVGGNWVLTAAHCKKPKYTVRLGDHSLQNKDGPEQEIPVVQSIPHPCYNSSDVEDHNHDLMLLQLRDQASLGSKVKPISLADHCTQPGQKCTVSGWGTVTSPRENFPDTLNCAEVKIFPQKKCEDAYPGQITDGMVCAGSSKGADTCQGDSGGPLVCDGALQGITSWGSDPCGRSDKPGVYTNICRYLDWIKKIIGSKGAAASTGSSGGGGGSGGLYKYKSRRSFIDEKKMP

SEQ ID NO: 119 - protein, 706 aa, Artificial sequence
  synthetic sequence
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNNASATNFSLLKQAGDVEENPGPMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSSGNSSGGSGSTGSSGGSGGSGSSGGSIIKGFECKPHSQPWQAALFEKTRLLCGATLIAPRWLLTAAHCLKPRYIVHLGQHNLQKEEGCEQTRTATESFPHPGFNNSLPNKDHRNDIMLVKMASPVSITWAVRPLTLSSRCVTAGTSCLISGWGSTSSPQLRLPHTLRCANITIIEHQKCENAYPGNITDTMVCASVQEGGKDSCQGDSGGPLVCNQSLQGIISWGQDPCAITRKPGVYTKVCKYVDWIQETMKNNAAASTGSSGGGGGSGGLYKYKSRRSFIDEKKMP

SEQ ID NO: 120 - protein, 738 aa, Artificial sequence
  synthetic sequence
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNNASATNFSLLKQAGDVEENPGPMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSSGNSSGGSGSTGSSGGSGGSGSSGGSGGVSQESSKVLNTNGTSGFLPGGYTCFPHSQPWQAALLVQGRLLCGGVLVHPKWVLTAAHCLKEGLKVYLGKHALGRVEAGEQVREVVHSIPHPEYRRSPTHLNHDHDIMLLELQSPVQLTGYIQTLPLSHNNRLTPGTTCRVSGWGTTTSPQVNYPKTLQCANIQLRSDEECRQVYPGKITDNMLCAGTKEGGKDSCEGDSGGPLVCNRTLYGIVSWGDFPCGQPDRPGVYTRVSRYVLWIRETIRKYETQQQKWLKGPQAAASTGSSGGGGGSGGLYKYKSRRSFIDEKKMP

SEQ ID NO: 121 - protein, 704 aa, Artificial sequence
  synthetic sequence
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNNASATNFSLLKQAGDVEENPGPMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSSGNSSGGSGSTGSSGGSGGSGSSGGSIIGGHTCTRSSQPWQAALLAGPRRRFLCGGALLSGQWVITAAHCGRPILQVALGKHNLRRWEATQQVLRVVRQVTHPNYNSRTHDNDLMLLQLQQPARIGRAVRPIEVTQACASPGTSCRVSGWGTISSPIARYPASLQCVNINISPDEVCQKAYPRTITPGMVCAGVPQGGKDSCQGDSGGPLVCRGQLQGLVSWGMERCALPGYPGVYTNLCKYRSWIEETMRDKAAASTGSSGGGGGSGGLYKYKSRRSFIDEKKMP

SEQ ID NO: 122 - protein, 712 aa, Artificial sequence
  synthetic sequence
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNNASATNFSLLKQAGDVEENPGPMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVVGSSGNSSGGSGSTGSSGGSGGSGSSGGSLLEGDECAPHSQPWQVALYERGRFNCGASLISPHWVLSAAHCQSRFMRVRLGEHNLRKRDGPEQLRTTSRVIPHPRYEARSHRNDIMLLRLVQPARLNPQVRPAVLPTRCPHPGEACVVSGWGLVSHNEPGTAGSPRSQVSLPDTLHCANISIISDTSCDKSYPGRLTNTMVCAGAEGRGAESCEGDSGGPLVCGGILQGIVSWGDVPCDNTTKPGVYTKVCHYLEWIRETMKRNAAASTGSSGGGGGSGGLYKYKSRRSFIDEKKMP

SEQ ID NO: 123 - DNA, 2850 bp, Artificial sequence
  synthetic sequence
cctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccaacgcgtcttagaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctaatcgcgagcgccgccaccatgcttctccttgtaacgtctctccttttgtgcgaacttccccatccggccttccttttgataccccgcaaggtgtgtaacggaattgggattggtgaattcaaggattcacttagtataaatgccacaaacattaagcattttaagaactgtacgtccatttcaggagacctgcatattcttcccgtagctttccggggcgattcattcacccacacaccgccacttgatccacaagaacttgacatcctgaaaacggttaaggagataacaggattcctcctgatacaggcctggcccgagaatagaaccgacttgcacgcctttgaaaatttggagataattcggggtcggactaagcaacatggacaattttcactggcggtagtttctttgaatattacgagcctcggccttagatctctcaaggagatctcagacggcgacgttataatatctgggaacaagaacctgtgctacgctaacacaatcaattggaaaaagctgttcggcacgtctggacaaaagacaaagataatttcaaatcgaggcgaaaatagctgcaaggctacgggacaggtttgtcacgccctctgtagcccagagggctgttggggacccgagccaagagattgcgtctcatgtcggaatgtgtcccgaggccgagaatgtgtcgataaatgcaatcttctggagggagaaccacgggaattcgttgaaaacagtgagtgcattcaatgtcacccggagtgccttccgcaagcgatgaatattacatgtacaggccggggtcccgataattgcatccagtgtgctcattatattgacggaccacactgtgtaaagacatgccctgccggcgttatgggtgaaaacaatacgctggtctggaagtatgcagacgcaggacatgtttgtcacctgtgccatcctaactgcacgtatggctgtacaggaccgggtctggaaggctgccctacgaatggtcccaaaataccatcaggaggatccatagccttcctcctcgcatgcgtcgctaccatgatcttcatgataactaaatgctgtctcttcgcggccgcaggtggtacagagaacctctattttcagtcagggtcgacatgtttcaggaaactggcgaagacaggtaagaaaaaaaaaagagactaatgatgagtataccctgcaggttaattagctaacaattgcacagtaagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcgttaagttaacattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatg

SEQ ID NO: 124 - protein, 171 aa, Artificial sequence
  synthetic sequence
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQ

SEQ ID NO: 125 - protein, 192 aa, Artificial sequence
  synthetic sequence
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVS

SEQ ID NO: 126 - protein, 205 aa, Artificial sequence
  synthetic sequence
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDK

SEQ ID NO: 127 - protein, 215 aa, Artificial sequence
  synthetic sequence
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPRE

SEQ ID NO: 128 - protein, 220 aa, Artificial sequence
  synthetic sequence
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENS

SEQ ID NO: 129 - protein, 224 aa, Artificial sequence
  synthetic sequence
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQ

SEQ ID NO: 130 - DNA, 20 bp, Artificial sequence
  synthetic sequence
gggaatcaaaatcggtgaat

SEQ ID NO: 131 - protein, 8 aa, Artificial sequence
  synthetic sequence
DYKDDDDK

SEQ ID NO: 132 - protein, 7 aa, Artificial sequence
  synthetic sequence
KLFSSKQ

SEQ ID NO: 133 - protein, 14 aa, Artificial sequence
  synthetic sequence
GISSFYSSTEERLW

SEQ ID NO: 134 - protein, 4 aa, Artificial sequence
  synthetic sequence
AAPR

SEQ ID NO: 135 - protein, 10 aa, Artificial sequence
  synthetic sequence
WEAVRYSAMW

SEQ ID NO: 136 - protein, 4 aa, Artificial sequence
  synthetic sequence
IEGR

SEQ ID NO: 137 - protein, 4 aa, Artificial sequence
  synthetic sequence
YASR